(19) United States
(12) Patent Application Publication
(10) Pub. No.: US 2024/0239767 A1
(43) Pub. Date: Jul. 18, 2024

(54) PCSK9 INHIBITORS AND METHODS OF USE THEREOF

(71) Applicant: AstraZeneca AB, Södertälje (SE)

(72) Inventors: Gavin Donal O’MAHONY, Gothenburg (SE); Olaf PANKNIN, Gothenburg (SE); Andrey FROLOV, Gothenburg (SE); Anna TOMBERG, Gothenburg (SE); Mateusz Piotr PLESNIAK, Gothenburg (SE); Jens Peter BRANDT, Södertälje (SE); Erik WEIS, Gothenburg (SE); Jens Patrik JOHANSSON, Södertälje (SE); Emma BRATT, Gothenburg (SE)

(21) Appl. No.: 18/539,471

(22) Filed: Dec. 14, 2023

Related U.S. Application Data
(60) Provisional application No. 63/387,731, filed on Dec. 16, 2022.
(60) Provisional application No. 63/603,163, filed on Nov. 28, 2023.

(51) Int. Cl. C07D 401/14; A61P 9/00; C07D 413/14; C07D 417/14; C07D 471/04; C07D 498/04; C07D 513/04; C07D 519/00

(52) U.S. Cl. CPC C07D 401/14; A61P 9/00; C07D 413/14; C07D 417/14; C07D 471/04; C07D 498/04; C07D 513/04; C07D 519/00

(57) ABSTRACT

A compound with the Formula (I):

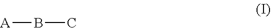

wherein A is of the following formula:

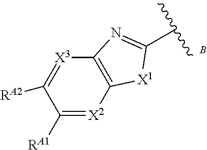

and
X¹ is selected from O, S or NH;
X² is either N or C—H
X³ is either N or C—R^{A3};
wherein if X¹ is NH and X² is C—H then X³ is C—R^{A3};
B is of formula (B-1) or (B-2)

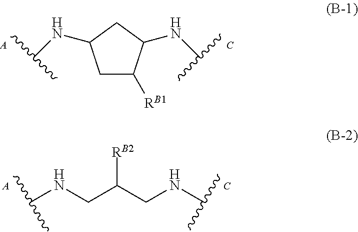

C is selected from the group consisting of optionally substituted C₆₋₁₀ carboaryl, C₅₋₆ heteroaryl or C₅₋₁₀ heterocyclyl, and their use as PCSK9 inhibitors.

The present disclosure relates to compounds which inhibit PCSK9 and their use in methods of treatment. The application claims the benefit of priority to U.S. application No. 63/387,731 filed on 16 Dec. 2022 and U.S. application No. 63/603,163 filed on 28 Nov. 2023. Each of the above listed applications is incorporated by reference herein in its entirety for all purposes.

### BACKGROUND

PCSK9, also referred to as “proprotein convertase subtilisin/kexin 9”, is a member of the secretory proprotein convertase family and plays an important role in cholesterol metabolism. PCSK9 increases the levels of circulating LDL cholesterol LDL-C via the enhanced degradation of the LDLRs independently of its catalytic activity. Secreted PCSK9 binds to the Epidermal Growth Factor domain A (EGFA) of the LDL receptor (LDLR) at the cell surface and the PCSK9/LDLR complex is internalized into endosomal/lysosomal compartments. The enhanced binding affinity of PCSK9 to the LDLR at the acidic pH of late endosomes/lysosomes reduces LDLR receptor recycling and instead targets LDLR for lysosomal degradation. Genetic association studies have demonstrated that loss-of-function mutations in PCSK9 are associated with low plasma LDL-C levels and a reduction in the incidence of adverse cardiovascular events.

For cardiovascular disease, few options exist for inhibiting PCSK9. Statins actually upregulate PCSK9 in HepG2 cells and in human primary hepatocytes through the increased expression of SREBP-2, a transcription factor that upregulates both the LDLR and PCSK9 genes. Since an elevated level of PCSK9 decreases the abundance of LDLR on the cell surface, increasing doses of statins have failed to achieve proportional LDL-C lowering effects.

Two monoclonal antibodies (mAbs) that bind selectively to extracellular PCSK9 and prevent its interaction with the LDLR, alirocumab and evolocumab, have recently received FDA approval for lowering LDL-C levels. In clinical trials, alirocumab showed an about 50% decrease in LDL levels compared to placebo (Elbitar 2016). Patients taking evolocumab showed an about 60-75% decrease in LDL levels. The potency of these drugs demonstrates the potential for inhibitors of PCSK9 to be effective treatments for those with hypercholesterolemia and other cardiovascular diseases. However, both antibody drugs require intravenous administration and can cause allergic reactions or other deleterious immune responses in the body.

Cardiovascular diseases often require management over a person's lifetime, unlike an infection that could be episodic. Thus, ease of dosing and administration become important factors for patient compliance with maintenance drug treatments. There is a need for PCSK9 inhibitors with increased efficacy and greater ease of administration, which can be achieved with small molecule PCSK9 inhibitors.

WO 2020/150473 A2 relates to heteroaryl compounds and pharmaceutical preparations thereof. It also relates to methods of treating or preventing cardiovascular diseases, and methods of treating sepsis or septic shock, using the described novel heterocyclic compounds.

WO 2020/15474 A1 relates to an inhibitor pharmacophore of PCSK9 and heteroaryl compounds that bind the PCSK9 protein.

### SUMMARY

A first aspect provides a compound with the Formula (I):

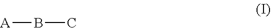

or a pharmaceutically acceptable salt, tautomeric forms or stereoisomers thereof,
wherein A is of the following formula:

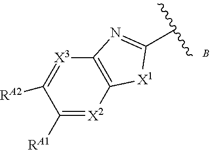

wherein the wavy line indicates the point of attachment to B;
X¹ is selected from O, S or NH;
X² is either N or C—H
X³ is either N or C—R^{A3};
wherein if X¹ is NH and X² is C—H then X³ is C—R^{A3};
when X¹ is NH, R^{A1} is X⁴ and R^{A2} is X⁵;
when X² is N and X¹ is O or S, R^{A1} is X⁴ and R^{A2} is X⁵;
when X² is CH and X¹ is S, R^{A1} is X⁴ and R^{A2} is X⁵;
when X² is CH and X¹ is O, R^{A1} is X⁵ and R^{A2} is X⁴;
X⁴ is selected from the group consisting of:
(i) H;
(ii) halo;
(iii) CN;
(iv) C₁ alkyl optionally substituted by one or more OH, CN, or one or more halo groups;
(v) C₁ alkoxy, optionally substituted by one or more halo groups;
X⁵ is selected from the group consisting of:
(i) H;
(ii) halo;
(iii) CN;
(iv) C₁₋₆ hydrocarbon, optionally substituted by OH, CN, C₁₋₆ alkyl acyl, C₁₋₆ alkoxy or one or more halo groups;
(v) C₁₋₆ alkoxy, optionally substituted by C₁₋₆ alkyl amido, C₁₋₆ alkyl phosphonyl, or one or more halo groups;
(vi) C₁₋₆ alkylamino
(vii) C₁₋₆ thioalkyl,
(viii) C₁₋₆ alkyl phosphinyl; or
(ix) C₁₋₆ alkyl phosphonyl;
R^{A3} is selected from the group consisting of:
(i) H;
(ii) halo;
(iii) CN;
(iv) C₁₋₆ hydrocarbon, optionally substituted by OH, CN, C₁₋₆ thioalkyl, C₁₋₆ alkoxy, C₁₋₆ alkyl acyl, C₁₋₆ acyloxy, C(═O)OH, C₁₋₆ alkyl ester, C₁₋₆ alkylamino; —C(═O)NH₂, C₁₋₆ alkyl amido, C₁₋₆ acylamido, C₁₋₆ alkyl sulfinyl, C₁₋₆ alkyl sulfonyl or one or more halo groups;
(v) C₁₋₆ alkoxy, optionally substituted by NH₂, C₄ heterocyclyl and one or more halo groups;
(vi) C₄ heterocycyl;
(vii) C₁₋₆ alkylamino, optionally substituted by CN, OH, C₄ heterocyclyl;
(viii) C₁₋₆ dialkylamino, optionally substituted by —NH₂; or
(ix) C₁₋₆ thioalkyl, optionally substituted by OH or —NH₂;
wherein B is of formula (B-1) or (B-2)

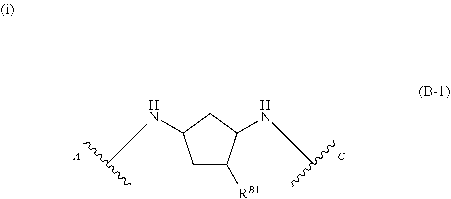

wherein the wavy lines indicate the point of attachment to A and C;
R^{B1} is H, OH, ═CHCH₂—OH, —O—C₁₋₄ alkyl or C₁₋₄ alkyl, wherein the C₁₋₄ alkyl is optionally substituted by OH or OMe;

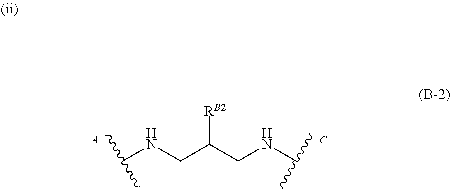

wherein the wavy lines indicate the point of attachment to A and C;
R^{B2} is C₁₋₂ alkyl-OH, CH₂C(═O)NHMe or C₁₋₃ alkyl;
wherein when R^{A1} is H or halo, R^{B2} is C₁₋₂ alkyl-OH or CH₂C(═O)NHMe;
wherein C is selected from the group consisting of C₆₋₁₀ carboaryl, C₅₋₆ heteroaryl or C₅₋₁₀ heterocyclyl, which groups are optionally substituted by:
(i) C₆₋₁₀ carboaryl, C₄₋₁₀ carbocyclyl, C₅₋₁₀ heteroaryl, C₄₋₁₀ heterocyclyl, or C₅₋₁₀ bridged heterocyclyl, spiro C₆₋₁₂heterocyclyl or a spiro C₆₋₁₂carbocyclyl,
   which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl optionally substituted with C₁₋₆ alkyl;
i) C₄₋₁₀ carbocyclyl with an optional methyl or ═O substituent;
j) C₆₋₁₀ carboaryl with optional substituents of one or more halo groups;
l) P(═O)Me₂;
m) C(═O)OH or CH₂C(═O)OH; and/or
n) tetrazolyl, CH₂-tetrazolyl, 5-oxo-4H-1,2,4-oxadiazol-3-yl;
(ii) one or more groups selected from C(═O)OH, CN, halo, nitro, C₁₋₆ alkyl, C₁₋₆ thioalkyl, C₁₋₆ alkoxy, C₁₋₆ alkyl acyl, C₁₋₆ alkyl amido, di-C₁₋₆ alkyl amido, C₁₋₆ alkyl sulfonamido, and di-C₁₋₆ alkyl sulfonamido.

In some embodiments X⁵ is selected from the group consisting of:
(i) H;
(ii) halo;
(iii) CN;
(iv) C₁₋₆ hydrocarbon, optionally substituted by OH, CN, C₁₋₆ alkyl acyl, C₁₋₆ alkoxy or one or more halo groups;
(v) C₁₋₆ alkoxy, optionally substituted by C₁₋₆ alkyl amido, or one or more halo groups;
(vi) C₁₋₆ alkylamino
(vii) C₁₋₆ thioalkyl, or
(viii) C₁₋₆ alkyl phosphinyl.

A second aspect provides a pharmaceutical composition comprising the compound of the first aspect and a pharmaceutically acceptable diluent, carrier or excipient.

The third aspect provides the compound of the first aspect for use in a method of therapy. The third aspect also provides the use of a compound of the first aspect in the manufacture of a medicament for treating a cardiovascular disease. The third aspect also provides a compound of the first aspect for use in the treatment of a cardiovascular disease. The third aspect also provides a method of treating a cardiovascular disease comprising administering a therapeutically effective amount of a compound of the first aspect or a composition according to the second aspect to a patient in need thereof.

This disclosure includes the combination of the aspects and preferred features described except where such a combination is clearly impermissible or expressly avoided.

### DETAILED DESCRIPTION

Aspects and embodiments will now be discussed. Further aspects and embodiments will be apparent to those skilled in the art. All documents mentioned in this text are incorporated herein by reference.

Compounds of Formula (I) and its use in the treatment of cardiovascular diseases are described herein. The compounds disclosed herein are PCSK9 inhibitors. The compounds may have higher inhibition of PCSK9, lower hERG activity, improved secondary pharmacology profile including GSK3β and/or other kinases, good stability, and/or improved activity in the treatment of cardiovascular diseases. The compounds may have an improved secondary pharmacology profile or an improved off-target profile.

### Definitions

### Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

Unless otherwise stated, halo is selected from chloro (Cl), fluoro (F), bromo (Br) and iodo (I).

Cyano (nitrile, carbonitrile): —CN.

Hydroxy: —OH.

Oxo: ═O (oxygen double bonded to the rest of the molecule).

C₁₋₆ hydrocarbon: The term “C₁₋₆ hydrocarbon” as used herein pertains to a monovalent moiety obtained by removing a hydrogen atom from a carbon atom of a hydrocarbon compound having from 1 to 6 carbon atoms, which may be aliphatic or alicyclic, which may be saturated or unsaturated (e.g. partially unsaturated, fully unsaturated) and may also be branched. Thus, the term “hydrocarbon” includes the terms alkyl, alkenyl, alkynyl, cycloalkyl, etc., discussed below.

C₁₋₆ alkyl: The term “C₁₋₆ alkyl” as used herein, pertains to a monovalent moiety obtained by removing a hydrogen atom from a carbon atom of a hydrocarbon compound having from 1 to 6 carbon atoms, which are saturated and may also be branched. The term “C₁₋₄ alkyl” as used herein, pertains to a monovalent moiety obtained by removing a hydrogen atom from a carbon atom of a hydrocarbon compound having from 1 to 4 carbon atoms, which are saturated.

Examples of saturated alkyl groups include, but are not limited to, methyl (C₁), ethyl (C₂), propyl (C₃), butyl (C₄), pentyl (C₅) and hexyl (C₆).

Examples of saturated linear alkyl groups include, but are not limited to, methyl (C₁), ethyl (C₂), n-propyl (C₃), n-butyl (C₄), n-pentyl (amyl) (C₅) and n-hexyl (C₆).

Examples of saturated branched alkyl groups include isopropyl (C₃), iso-butyl (C₄), sec-butyl (C₄), tert-butyl (C₄), iso-pentyl (C₅), and neopentyl (C₅).

C₂₋₆ Alkenyl: The term “C₂₋₆ alkenyl” as used herein, pertains to a hydrocarbon group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, CHCH═CH₂), iso-propenyl (1-methylvinyl, C(CH₃)═CH₂), butenyl (C₄), pentenyl (C₅), and hexenyl (C₆).

C₂₋₆ alkynyl: The term “C₂₋₆ alkynyl” as used herein, pertains to a hydrocarbon group having one or more carbon-carbon triple bonds.

Examples of unsaturated alkynyl groups include, but are not limited to, ethynyl (—C═CH) and 2-propynyl (propargyl, —CH₂C═CH).

C₁₋₆ alkoxy: The term C₁₋₆ alkoxy as used herein, pertains to an OR group, wherein R is an C₁₋₆ hydrocarbon group. Examples of C₁₋₆ alkoxy groups include, but are not limited to, OMe, OEt (ethoxy), —O(nPr) (n-propoxy), —O(iPr) (iso-propoxy), O(nBu) (n-butoxy), O(sBu) (sec-butoxy), O(iBu) (iso-butoxy), and O(tBu) (tert-butoxy).

C₁₋₆ acyloxy: The term C₁₋₆ acyloxy (reverse ester) as used herein, pertains to an OC(═O)R, wherein R is a C₁₋₆ hydrocarbon group. Examples of acyloxy groups include, but are not limited to, OC(═O)CH₃ (acetoxy), OC(═O)CH₂CH₃, or OC(═O)C(CH₃)₃.

Amino: NR¹R², wherein R¹ and R² are independently amino substituents, for example, hydrogen, a C₁₋₆ hydrocarbon group (also referred to as C₁₋₆ alkylamino or C₁₋₆ dialkylamino), or, in the case of a “cyclic” amino group, R¹ and R², taken together with the nitrogen atom to which they are attached, form a heterocyclic ring having from 4 to 6 ring atoms. Amino groups may be primary (—NH₂), secondary (—NHR¹), or tertiary (—NHR¹R²), and in cationic form, may be quaternary (—+NR¹R²R³). Examples of amino groups include, but are not limited to NH₂, NHCH₃, NHC(CH₃)₂, N(CH₃)₂, N(CH₂CH₃)₂, and NHPh. Examples of cyclic amino groups include, but are not limited to, aziridino, azetidino, pyrrolidino, piperidino, piperazino, morpholino, and thiomorpholino.

C₁₋₆ acylamido: Acylamido (acylamino): NR¹C(═O)R², wherein R¹ is an amide substituent, for example, hydrogen or a C₁₋₆ hydrocarbon group, and R² is an acyl substituent, for example, a C₁₋₆ hydrocarbon group. Examples of acylamide groups include, but are not limited to, NHC(═O)CH₃ and NHC(═O)CH₂CH₃. In some embodiments R¹ and R² may together form a cyclic or bicyclic structure and form a cyclic acylamido group. Examples of such groups include succinimidyl, maleimidyl, phthalimidyl, 2-oxo-3H-benzimidazol-1-yl, 3-methyl-2-oxo-benzimidazol-1-yl, 1-methyl-2-oxoimidazo[4,5-b]pyridin-3-yl, 2,5-dioxoimidazolidin-1-yl and 2,4-dioxoimidazolidin-1-yl:

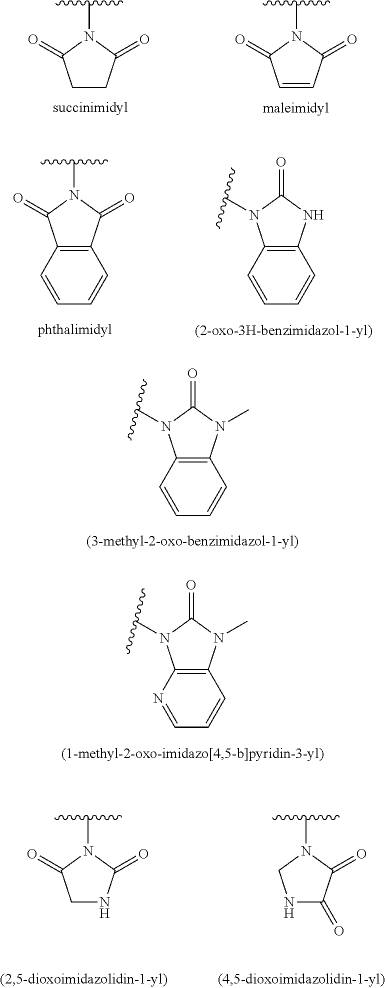

Carbaimidoyl: —C(═NH)NH₂.

Methyl-carbaimidoyl: —C(═N—CH₃)NH₂.

Carboxyamino: —N(H)(C(═O)OH).

C₁₋₆ thioalkyl: The term C₁₋₆ thioalkyl as used herein, pertains to an SR, wherein R is a C₁₋₆ hydrocarbon group. Examples of C₁₋₆ alkylthio groups include, but are not limited to, SCH₃ and SCH₂CH₃.

C₁₋₆ alkyl sulfinyl: The term C₁₋₆ alkyl sulfinyl pertains to a sulfine (sulfinyl, sulfoxide) which has the structure S(═O)R—, wherein R is a C₁₋₆ hydrocarbon group. Examples of C₁₋₆ alkyl sulfinyl groups include, but are not limited to, S(═—O)CH₃ and S(═—O)CH₂CH₃.

C₁₋₆ alkyl sulfonyl: The term C₁₋₆ alkyl sulfonyl as used herein pertains to an —S(═O)₂R, group wherein R is a C₁₋₆ hydrocarbon group, including, for example, a fluorinated or perfluorinated C₁₋₆ alkyl group. Examples of C₁₋₆ alkyl sulfonyl groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl) and —S(═O)₂CH₂CF₃ (tresyl).

C₁₋₆ sulfonimodyl: The term C₁₋₆ sulfonimodyl is also referred to as Sulfonamido (sulfinamoyl; sulfonic acid amide; sulfonamide or di-C₁₋₆ alkyl sulfonamido) and has the structure S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃) and —S(═O)₂N(CH₂CH₃)₂.

Sulfonamino: NR¹S(═O)₂R, wherein R¹ is an amino substituent, as defined for amino groups, and R is a sulfonamino substituent, for example, a C₁₋₆ alkyl group, a C₃₋₂₀ heterocyclyl group, or a C₅₋₂₀ aryl group, preferably a C₁₋₆ alkyl group. Examples of sulfonamino groups include, but are not limited to, NHS(═O)₂CH₃ and N(CH₃)S(═O)₂C₆H₅.

C₁₋₆ alkyl phosphinyl: The term C₁₋₆ alkyl phosphinyl (phosphine oxide) has the structure —P(═O)R₂, wherein each R is independently a C₁₋₆ hydrocarbon group. Examples of C₁₋₆ alkyl phosphinyl groups include, but are not limited to, P(═O)Me₂, P(═O)(CH₂CH₃)₂ and P(═O)(tBu)₂. Wherein each R group can be the same or different groups.

C₁₋₆ alkyl phosphonyl: The term C₁₋₆ alkyl phosphonyl has the structure —P(═O)₂R, wherein R is a C₁₋₆ hydrocarbon group. Examples of C₁₋₆ alkyl phosphonyl groups include, but are not limited to, P(═O)₂(CH₃), P(═O)₂(CH₂CH₃) and P(═O)₂(tBu).

Carboxy (carboxylic acid): —C(═O)OH.

C₁₋₆ alkyl ester: The term C₁₋₆ alkyl ester (carboxylate, carboxylic acid ester, oxycarbonyl) has the structure C(═O)OR, wherein R is a C₁₋₆ hydrocarbon group. Examples of ester groups include, but are not limited to, C(═O)OCH₃, C(═O)OCH₂CH₃ and C(═O)OC(CH₃)₃.

C₁₋₆ alkyl acyl: The term C₁₋₆ alkyl acyl also known as Acyl (keto) has the structure C(═O)R, wherein R is a C₁₋₆ hydrocarbon group. Examples of C₁₋₆ alkyl acyl groups include, but are not limited to, C(═O)CH₃ (acetyl), C(═O)CH₂CH₃ (propionyl) or C(═O)C(CH₃)₃ (tert-butyryl).

C₁₋₆ alkyl amido: The term C₁₋₆ alkyl amido (also referred to as carbamoyl, carbamyl, aminocarbonyl, carboxamide) has the structure C(═O)NR¹R², wherein R¹ and R² are independently amino substituents, as defined for amino groups for example, hydrogen, a C₁₋₆ hydrocarbon group (also referred to as C₁₋₆ alkyl amido or C₁₋₆ dialkyl amido), or, in the case of a “cyclic” amido group, R¹ and R², taken together with the nitrogen atom to which they are attached, form a heterocyclic ring having from 4 to 6 ring atoms. Examples of amido groups include, but are not limited to, C(═O)NH₂, C(═O)NHCH₃, C(═O)N(CH₃)₂, C(═O)NHCH₂CH₃, and C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms. The carbocyclic ring may be saturated or unsaturated and may be bridged or unbridged. The ring may be a fused ring or a single ring.

Examples of cycloalkyl groups include, but are not limited to, those derived from:
saturated monocyclic hydrocarbon compounds:
cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:
cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:
norcarane (C₇), norpinane (C₇), norbornane (C₇).

C₃₋₁₀ heterocyclyl: The term “C₃₋₁₀ heterocyclyl” as used herein, pertains to a monovalent moiety obtained by removing a hydrogen atom from a ring atom of a heterocyclic compound, which moiety has from 3 to 10 ring atoms, of which from 1 to 5 are ring heteroatoms. In certain embodiments, each ring has from 3 to 7 ring atoms, of which from 1 to 4 are ring heteroatoms. The ring may be saturated or unsaturated, and may be bridged or unbridged. The ring may be a fused ring or a single ring. For the avoidance of doubt, substituents on the heterocycloalkyl ring may be linked via either a carbon atom or a heteroatom.

In this context the term ‘heteroatom’ means O, S, N, Si or B (Boron).

In this context, the prefixes (e.g. C₃₋₁₀ C₃₋₇, C₅₋₆, etc.) denote the number of ring atoms, or range of number of ring atoms, whether carbon atoms or heteroatoms. For example, the term “C₅₋₆ heterocyclyl”, as used herein, pertains to a heterocyclyl group having 5 or 6 ring atoms.

Examples of monocyclic heterocyclyl groups include, but are not limited to, those derived from:
N₁: aziridine (C₃), azetidine (C₄), pyrrolidine (tetrahydropyrrole) (C₅), pyrroline (e.g. 2,5-dihydro-1H-pyrrole) (C₅), 2H-pyrrole or 3H-pyrrole, isoazole (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆).

Examples of bicyclic heterocyclyl groups include, but are not limited to those derived from:

| Compound | Structure |
|---|---|
| 7- azabicyclo[4.2.0]octane (N₁) C₈ | 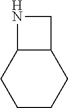 |
| 3- azabicyclo[3.1.0]hexane (N₁) C₆ | 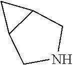 |
| 6- azabicyclo[3.2.0]heptane (N₁) C₇ | 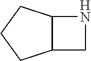 |
| 2,3,3a,4,5,6,7,7a- octahydrofuro[2,3- c]pyridine (N₁O₁) C₉ | 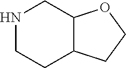 |

C₆₋₁₀ carboaryl: The term “C₆₋₁₀ carboaryl”, as used herein, pertains to a monovalent moiety obtained by removing a hydrogen atom from an aromatic ring atom of an aromatic compound, which moiety has from 6 to 10 ring atoms and the ring atoms are all carbon atoms, as in “carboaryl groups”. The ring may be a fused ring or a single ring. Examples of carboaryl groups include, but are not limited to, those derived from benzene (i.e. phenyl) (C₆), naphthalene (C₁₀) and azulene (C₁₀).

In this context, the prefixes (e.g. C₅₋₇, C₅₋₆, C₅₋₁₀, etc.) denote the number of ring atoms, or range of number of ring atoms. For example, the term “C₅₋₆ aryl” as used herein, pertains to an aryl group having 5 or 6 ring atoms.

Examples of carboaryl groups which comprise fused rings, at least one of which is an aromatic ring, include, but are not limited to, groups derived from indane (e.g. 2,3-dihydro-1H-indene) (C₉), indene (C₉), isoindene (C₉) and tetraline (1,2,3,4-tetrahydronaphthalene) (C₁₀).

C₅₋₁₀ heteroaryl: The term “C₅₋₁₀ heteroaryl”, as used herein, pertains to a monovalent moiety obtained by removing a hydrogen atom from an aromatic ring atom of an aromatic compound, which moiety has from 5 to 10 ring atoms of which from 1 to 5 are ring heteroatoms. In certain embodiments, each ring has from 5 to 7 ring atoms, of which from 1 to 4 are ring heteroatoms. For the avoidance of doubt, substituents on the heteroaryl ring may be linked via either a carbon atom or a heteroatom. The ring may be a fused ring or a single ring.

In this context the term ‘heteroatom’ means O, S, N, Si or B (Boron).

Examples of monocyclic heteroaryl groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: 1H-imidazole (1,3-diazole) (C₅), 1H-pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to C₉ heteroaryl (with 2 fused rings) derived from:

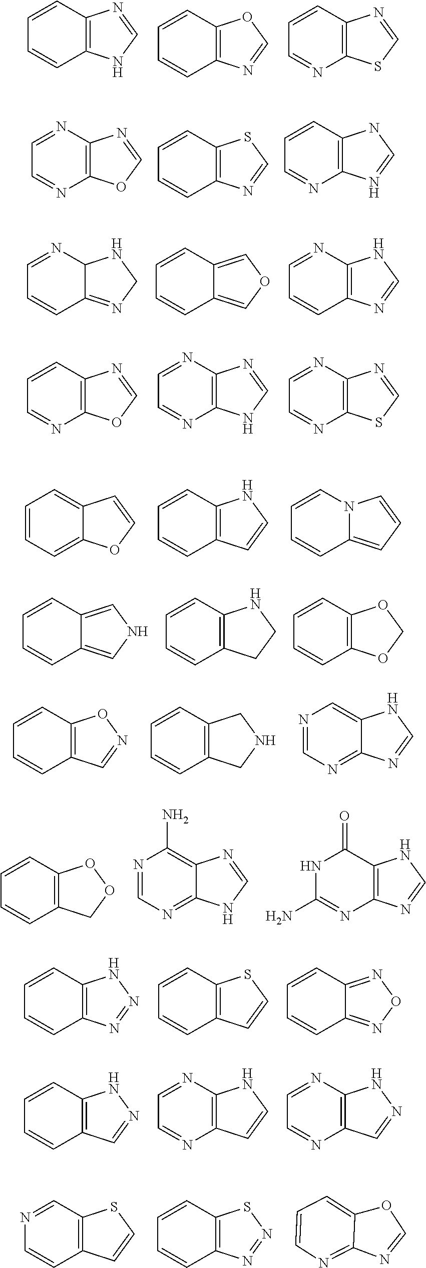

Examples of heteroaryl which comprise fused rings, include, but are not limited to C₁₀ heteroaryl (with 2 fused rings) derived from:

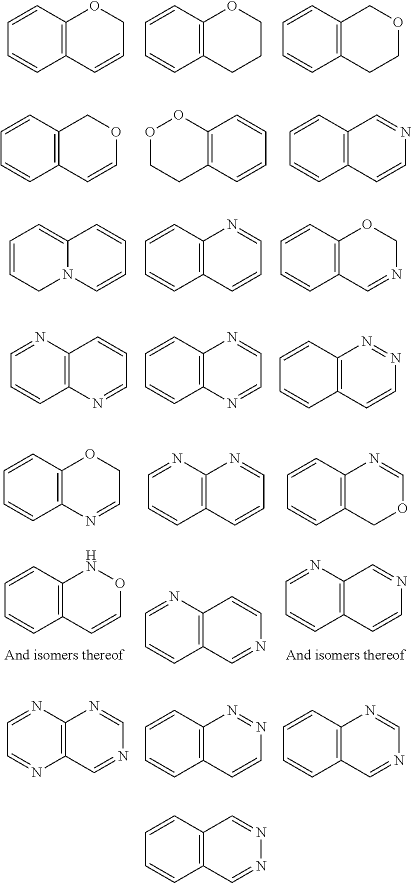

Examples of heteroaryl or heterocyclic compounds include but are not limited to those derived from:

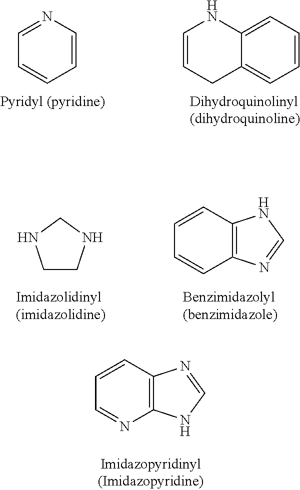 [SMILES: c1ccncc1 ; C1=CNc2ccccc2C1 ; C1CNCN1 ; c1ccc2[nH]cnc2c1 ; c1cnc2[nH]cnc2c1]

Spiro C₆₋₁₂ carbocyclyl: The term Spiro C₆₋₁₂ carbocyclyl as used herein pertains to a moiety that has at least two molecular rings with only one common atom. The simplest spiro compounds are bicyclic (having just two rings), or have a bicyclic portion as part of the larger ring system, in either case with the two rings connected through the defining single common atom. Spiro C₆₋₁₂ carbocyclyl pertains to a cyclyl group; that is, a monovalent moiety obtained by removing a hydrogen atom from an alicyclic ring atom of a cyclic hydrocarbon (carbocyclic) compound, which moiety has from 6 to 12 carbon atoms, including from 3 to 7 ring atoms wherein the rings share a common atom.

Spiro C₆₋₁₂ heterocyclyl: The term Spiro C₆₋₁₂ heterocyclyl as used herein pertains to a moiety that has at least two molecular rings with only one common atom. The simplest spiro compounds are bicyclic (having just two rings), or have a bicyclic portion as part of the larger ring system, in either case with the two rings connected through the defining single common atom. The spiro C₆₋₁₂ heterocyclyl moiety pertains to a monovalent moiety obtained by removing a hydrogen atom from a ring atom of a heterocyclic compound, which moiety has from 8 to 12 ring atoms of which from 1 to 3 are ring heteroatoms wherein the rings share a common atom. In certain embodiments, each ring has from 9 to 11 ring atoms, of which from 1 to 2 are ring heteroatoms. For the avoidance of doubt, substituents on the heteroaryl ring may be linked via either a carbon atom or a heteroatom.

For the avoidance of doubt, where multiple substituents are independently selected from a given group, the selected substituents may comprise the same substituents or different substituents from within the given group.

### Pharmaceutically Acceptable Salt

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich: Wiley-VCH/VHCA, 2002. A suitable pharmaceutically acceptable salt of a compound of Formula (I) is, for example, an acid addition salt. An acid addition salt of a compound of Formula (I) may be formed by bringing the compound into contact with a suitable inorganic or organic acid under conditions known to the skilled person. An acid addition salt may for example be formed using an inorganic acid selected from the group consisting of hydrochloric acid, hydrobromic acid, sulphuric acid and phosphoric acid. An acid addition salt may also be formed using an organic acid selected from the group consisting of trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

Therefore, in one embodiment there is provided a compound of Formula (I) or a pharmaceutically acceptable salt thereof, where the pharmaceutically acceptable salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt.

### Other Forms

Compounds and salts described in this specification may exist in solvated forms and unsolvated forms. For example, a solvated form may be a hydrated form, such as a hemihydrate, a monohydrate, a dihydrate, a trihydrate or an alternative quantity thereof. The compounds of Formula (I) encompass all such solvated and unsolvated forms of compounds of Formula (I), particularly to the extent that such forms possess PCSK9 inhibitory activity, as for example measured using the tests described herein.

Compounds and salts described in this specification include one or more chiral (i.e. asymmetric) centres. To the extent a structure or chemical name in this specification does not indicate the chirality, the structure or name is intended to encompass any single stereoisomer (i.e. any single chiral isomer) corresponding to that structure or name, as well as any mixture of stereoisomers (e.g. a racemate). In some embodiments, a single stereoisomer is obtained by isolating it from a mixture of isomers (e.g. a racemate) using, for example, chiral chromatographic separation. In other embodiments, a single stereoisomer is obtained through direct synthesis from, for example, a chiral starting material.

A particular enantiomer of a compound described herein may be more active than other enantiomers of the same compound.

According to one embodiment there is provided a compound of Formula (I), or a pharmaceutically acceptable salt thereof, which is a single enantiomer being in an enantiomeric excess (% ee) of ≥95, ≥98% or ≥99%. Conveniently, the single enantiomer is present in an enantiomeric excess (% ee) of ≥99%.

According to another embodiment there is provided a pharmaceutical composition, which comprises a compound of Formula (I), which is a single enantiomer being in an enantiomeric excess (% ee) of ≥95, ≥98% or ≥99% or a pharmaceutically acceptable salt thereof, in association with one or more pharmaceutically acceptable excipients. Conveniently, the single enantiomer is present in an enantiomeric excess (% ee) of ≥99%.

### Isotopes

Atoms of the compounds and salts described in this specification may exist as their isotopes. The compound of Formula (I) encompasses all compounds of Formula (I) where an atom is replaced by one or more of its isotopes (for example a compound of Formula (I) where one or more carbon atom is an ¹¹C or ¹³C carbon isotope, or where one or more hydrogen atoms is a ²H or ³H isotope).

### Tautomers

Compounds and salts described in this specification may exist as a mixture of tautomers. “Tautomers” are structural isomers that exist in equilibrium resulting from the migration of a hydrogen atom. The compound of Formula (I) includes all tautomers of compounds of Formula (I) particularly to the extent that such tautomers possess PCSK9 inhibitory activity.

### Crystalline Forms

Compounds and salts described in this specification may be crystalline and may exhibit one or more crystalline forms. The compound of Formula (I) encompasses any crystalline or amorphous form of a compound of Formula (I), or mixture of such forms, which possesses PCSK9 inhibitory activity.

It is generally known that crystalline materials may be characterised using conventional techniques such as X-Ray Powder Diffraction (XRPD), Differential Scanning Calorimetry (DSC), Thermal Gravimetric Analysis (TGA), Diffuse Reflectance Infrared Fourier Transform (DRIFT) spectroscopy, Near Infrared (NIR) spectroscopy, solution and/or solid state nuclear magnetic resonance spectroscopy. The water content of crystalline materials may be determined by Karl Fischer analysis.

### Therapy, Prophylaxis and Related Terms

The term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

The term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

The term “subject” to which administration is contemplated includes, but is not limited to, humans (i.e., a male or female of any age group, e.g., a paediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of the disease to be treated.

### Pharmaceutical Compositions

The compounds of Formula (I), and pharmaceutically acceptable salts thereof, may be administered as pharmaceutical compositions, comprising one or more pharmaceutically acceptable excipients.

Therefore, in one embodiment there is provided a pharmaceutical composition comprising a compound of Formula (I), or a pharmaceutically acceptable salt thereof, and at least one pharmaceutically acceptable excipient.

The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth edition, Pharmaceutical Press, edited by Rowe, Ray C; Sheskey, Paul J; Quinn, Marian. Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The pharmaceutical compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous or intramuscular dosing), or as a suppository for rectal dosing. The compositions may be obtained by conventional procedures well known in the art. Compositions intended for oral use may contain additional components, for example, one or more colouring, sweetening, flavouring and/or preservative agents.

Suitable daily doses of the compounds disclosed herein, or a pharmaceutically acceptable salt thereof, in therapeutic treatment of humans are about 0.0001-100 mg/kg body weight. Pharmaceutical formulations as described herein may be formulated by methods known to those skilled in the art to provide doses of the active compound in the range of 0.1 mg to 1000 mg. The daily dose will necessarily be varied depending upon the host treated, the particular route of administration, any therapies being co-administered, and the severity of the illness being treated. Accordingly, the practitioner who is treating any particular patient may determine the optimum dosage.

The pharmaceutical compositions described herein comprise compounds of Formula (I), or a pharmaceutically acceptable salt thereof, and are therefore expected to be useful in therapy.

As such, in one embodiment there is provided a pharmaceutical composition for use in therapy, comprising a compound of Formula (I), or a pharmaceutically acceptable salt thereof, and at least one pharmaceutically acceptable excipient.

In one embodiment there is provided a pharmaceutical composition for use in the treatment of a disease in which inhibition of PCSK9 is beneficial, comprising a compound of Formula (I), or a pharmaceutically acceptable salt thereof, and at least one pharmaceutically acceptable excipient. In one embodiment there is provided a pharmaceutical composition for use in the treatment of a cardiovascular disease, comprising a compound of Formula (I), or a pharmaceutically acceptable salt thereof, and at least one pharmaceutically acceptable excipient. In one embodiment there is provided a pharmaceutical composition for use in the treatment of a cardiovascular disease in which inhibition of PCSK9 is beneficial, comprising a compound of Formula (I), or a pharmaceutically acceptable salt thereof, and at least one pharmaceutically acceptable excipient.

### Methods of Use

The compounds described herein may be used in a method of therapy. Also provided is a method of treatment, comprising administering to a subject in need of treatment a therapeutically effective amount of a compound of Formula (I). The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A compound may be administered alone or in combination with other treatments, either simultaneously or sequentially dependent upon the condition to be treated.

In one embodiment there is provided a compound of Formula (I), or a pharmaceutically acceptable salt thereof, or a pharmaceutical composition comprising the compound of Formula (I) for use in therapy. In one embodiment there is provided the use of the compound of Formula (I), or a pharmaceutically acceptable salt thereof, or a pharmaceutical composition comprising the compound of Formula (I) for the manufacture of a medicament. In another embodiment there is provided a method of treatment comprising administering to a subject the compound of Formula (I), or a pharmaceutically acceptable salt thereof, or a pharmaceutical composition comprising the compound of Formula (I).

The compounds described herein are PCSK9 inhibitors. The PCSK9 gene was identified using genetic mapping techniques on DNA from subjects with autosomal dominant hypercholesterolemia (Abifadel 2003). The encoded protein is a serine protease that is mostly expressed in the liver, gut, kidney, and nervous system and circulates in plasma. While not wishing to be bound by any particular theory, studies on mutations in the gene indicated that its putative role was in reducing LDLR at the cell surface independently of its catalytic activity (Abifadel 2010). Binding of PCSK9 to the LDLR results in their lysosomal degradation. This enhanced LDLR degradation results in increases in the amount of circulating low-density lipoprotein (LDL). PCSK9 is upregulated by statins, SREBP-1a and SREBP-2, LXR agonist, and insulin, but downregulated by dietary cholesterol, glucagon, ethinylestradiol, chenodeoxycholic acid and the bile acid-activated farnesoid X receptor (FXR) (Maxwell 2003; Persson 2009; Langhi 2008). Since an elevated level of PCSK9 decreases the abundance of LDLR on the cell surface, increasing doses of statins fail to achieve proportional LDL-C lowering results. Thus, disclosed herein are methods for treating a wide range of cardiovascular diseases and conditions that benefit from inhibiting PCSK9 thereby lowering LDL-C.

In certain embodiments, the method of inhibiting PCSK9 occurs in a subject in need thereof, thereby treating a disease or disorder mediated by PCSK9. Also, disclosed herein are methods of treating or preventing a disease or a disorder mediated by PCSK9 comprising administering a compound of Formula (I) or a pharmaceutically acceptable salt thereof. In certain embodiments, disclosed herein are methods of treating a disease or a disorder mediated by PCSK9 comprising administering a compound of Formula (I) or a pharmaceutically acceptable salt thereof. In certain embodiments, disclosed herein are methods of preventing a disease or a disorder mediated by PCSK9 comprising administering a compound of Formula (I) or a pharmaceutically acceptable salt thereof. The prevention of cardiovascular events through the inhibition of PCSK9 has been described, e.g., in Robinson 2015.

In some embodiments there is provided a method of treating a cardiovascular disease comprising administering to a subject a compound of Formula (I), or a pharmaceutical composition comprising a compound of Formula (I). In some embodiments there is provided a compound of Formula (I), or a pharmaceutically acceptable salt thereof, or a pharmaceutical composition comprising a compound of Formula (I) for use in the treatment of a cardiovascular disease. In some embodiments there is provided a compound of Formula (I), or a pharmaceutically acceptable salt thereof, or a pharmaceutical composition comprising a compound of Formula (I) for the manufacture of a medicament for the treatment of a cardiovascular disease.

Exemplary cardiovascular diseases and conditions include, but are not limited to, dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease, heart failure or congestive heart failure. In certain embodiments, exemplary cardiovascular diseases and conditions include, but are not limited to, hypercholesterolemia, hyperlipidemia, hyperlipoproteinemia, hypertriglyceridemia, dyslipidemia, dyslipoproteinemia, atherosclerosis, hepatic steatosis, metabolic syndrome and coronary artery disease. In certain embodiments, the disease is hypercholesterolemia, such as familial hypercholesterolemia or autosomal dominant hypercholesterolemia. In certain embodiments, the disease is hyperlipidemia. In certain embodiments, the disease is coronary artery disease.

In certain embodiments, the disclosed methods of treatment can decrease high levels of circulating serum cholesterol, such as LDL-C and VLDL-Cholesterol. In addition, the disclosed methods are useful for decreasing circulating serum triglycerides, circulating serum lipoprotein A, circulating serum LDL-C and atherogenic lipoproteins. In certain embodiments, the diseases or conditions treated with the disclosed compounds and compositions include atherosclerosis and atherosclerotic plaque formation. Subjects having a gain-of-function mutation in the PCSK9 gene also benefit with treatment with the disclosed compounds and compositions counteracting the mutation through their inhibition of PCSK9.

### Combination Treatments

Disclosed compounds and compositions may be conjointly administered with other therapeutic agents, such as other agents suitable for the treatment of high levels of LDL-C and triglycerides. In certain embodiments, conjointly administering one or more additional therapeutic agents with a compound described herein provides a synergistic effect. In certain embodiments, conjointly administering one or more additional therapeutic agents provides an additive effect.

In some embodiments in which a combination therapy is used, the amount of the compound or salt described in this specification and the amount of the other pharmaceutically active agent(s) are, when combined, therapeutically effective to treat a targeted disorder in the animal patient. In this context, the combined amounts are “therapeutically effective amounts” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound or salt and an approved or otherwise published dosage range(s) of the other pharmaceutically active compound(s).

A pharmaceutical composition of the specification may comprise one or more further active ingredients, as appropriate, examples of combinations of a compound of the specification (or a pharmaceutically acceptable salt thereof) and one or more additional active ingredients are described herein.

The specification further relates to a combination therapy wherein a compound of the specification, or a pharmaceutically acceptable salt thereof, and a second active ingredient are administered concurrently, sequentially or in admixture, for the treatment of one or more of the conditions listed above. Such a combination may be used in combination with one or more further active ingredients.

In one aspect there is provided a combination (for example, for use as a medicament for the treatment of one of the diseases or conditions listed herein, such as a cardiovascular disease) comprising a compound of the specification, or a pharmaceutically acceptable salt thereof, and at least one active ingredient selected from:
i) a statin;
ii) a cholesterol absorption inhibitor;
iii) a SGLT2 inhibitor:
iv) a P2Y12 inhibitor;
v) a citrate lyase inhibitor; and
vi) anti-hypertensive drugs.

In a further aspect of the present specification there is provided a pharmaceutical composition (for example, for use as a medicament for the treatment of one of the diseases or conditions listed herein, such as a cardiovascular disease) comprising a compound of the specification, or a pharmaceutically acceptable salt thereof, and at least one active ingredient selected from:
i) a statin;
ii) a cholesterol absorption inhibitor;
iii) a SGLT2 inhibitor;
iv) a P2Y12 inhibitor;
v) citrate lyase inhibitor;
vi) anti-hypertensive drugs.

In another embodiment there is provided a compound of Formula (I), or a pharmaceutically acceptable salt thereof, and at least one additional active ingredient selected from a statin, wherein the statin is selected from Atorvastatin, Fluvastatin, Lovastatin, Mevastatin, Pitavastatin, Pravastatin, Rosuvastatin or Simvastatin. In another aspect the statin is Rosuvastatin (Crestor).

In another embodiment there is provided a compound of Formula (I), or a pharmaceutically acceptable salt thereof, and at least one additional active ingredient selected from a cholesterol absorption inhibitor, wherein the cholesterol absorption inhibitor is selected from Ezetimibe (Ezetrol).

In another embodiment there is provided a compound of Formula (I), or a pharmaceutically acceptable salt thereof, and at least one additional active ingredient selected from a SGLT2 inhibitor, wherein the SGLT2 inhibitor is selected from Canagliflozin, Dapagliflozin, Empagliflozin, Ertugliflozin, Ipragliflozin, Luseogliflozin, Remogliflozin etabonate, Sergliflozin etabonate, Sotagliflozin or Tofogliflozin. In some aspects the SGLT2 inhibitor is selected from Dapagliflozin (Farxiga or Forxiga).

In another embodiment there is provided a compound of Formula (I), or a pharmaceutically acceptable salt thereof, and at least one additional active ingredient selected from a P2Y12 inhibitor, wherein the P2Y12 inhibitor is selected from Brilinta (Ticagrelor) and Clopidogrel (Plavix).

In another embodiment there is provided a compound of Formula (I), or a pharmaceutically acceptable salt thereof, and at least one additional active ingredient selected from a citrate lyase inhibitor, wherein the citrate lyase inhibitor is Bempedoic acid (Nexletol).

In another embodiment there is provided a compound of Formula (I), or a pharmaceutically acceptable salt thereof, and at least one additional active ingredient selected from Ezetimibe, Rosuvastatin, Dapagliflozin and Ticagrelor. In one embodiment there is one additional active ingredient. In another embodiment there are two additional active ingredients. In one embodiment the additional active ingredient is Ezetimibe, Rosuvastatin, Dapagliflozin or Ticagrelor. In another embodiment the additional two active ingredients are Ezetimibe and Rosuvastatin or Dapagliflozin and Rosuvastatin.

In another embodiment there is provided a compound of Formula (I), or a pharmaceutically acceptable salt thereof, and at least one additional active ingredient selected from anti-hypertensive drugs. In some aspects, the antihypertensive drug is selected from Valsartan (Diovan), Metoprolol (Lopressor), HCTZ (Hydrochlorothiazide), Olmesartan (Benicar), Lisinopril (Prinivil, Zestril), Amlodipine besylate (Norvasc), Candesartan, or a calcium channel blocker or a combination thereof. In another aspect there is provided the compound of Formula (I) or a pharmaceutically acceptable salt thereof in combination with:
i) Valsartan;
ii) Metoprolol;
iii) Valsartan and HCTZ;
iv) Olmesartan;
v) Olmesartan and HCTZ;
vi) Lisinopril;
vii) Amlodipine;
viii) Candesartan;
ix) a calcium channel blocker; or
x) HCTZ.

In one embodiment there is provided a compound of Formula (I), or a pharmaceutically acceptable salt thereof, and at least one additional active ingredient for use in the simultaneous, separate or sequential treatment of a cardiovascular disease. In one embodiment there is provided a compound of Formula (I), or a pharmaceutically acceptable salt thereof, for use in the treatment of a cardiovascular disease, where the compound of Formula (I), or a pharmaceutically acceptable salt thereof, is administered simultaneously, separately or sequentially with at least one an additional active substance selected from Ezetimibe, Rosuvastatin, Dapagliflozin and Ticagrelor.

In another embodiment there is provided a method of treating a cardiovascular disease in a subject, which comprises administering to said subject a compound of Formula (I) or a pharmaceutically acceptable salt thereof, and simultaneously, separately or sequentially administering at least one additional active substance, wherein the at least one additional active substance is selected from Ezetimibe, Rosuvastatin, Dapagliflozin and Ticagrelor.

### Further Embodiments

The following embodiments may apply to all aspects as described above or may relate to a single aspect. The embodiments may be combined together in any combination.

### A

A is of the following formula:

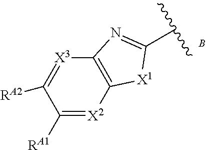

wherein the wavy line indicates the point of attachment to B.
X¹ is selected from O, S or NH.
X² is either N or C—H.
X³ is either N or C—R^{A3}.
When X¹ is NH and X² is C—H then X³ is C—R^{A3}.

X¹

In some embodiments X¹ is O.

In some embodiments X¹ is S.

In some embodiments X¹ is NH and when X¹ is NH and X² is C—H then X³ is C—R^{A3}.

X²

In some embodiments X² is N.

In some embodiments X² is CH.

X³

In some embodiments X³ is N.

In some embodiments X³ is C—R^{A3}.

In some embodiments A is selected from one of the following formulae A1-A3:

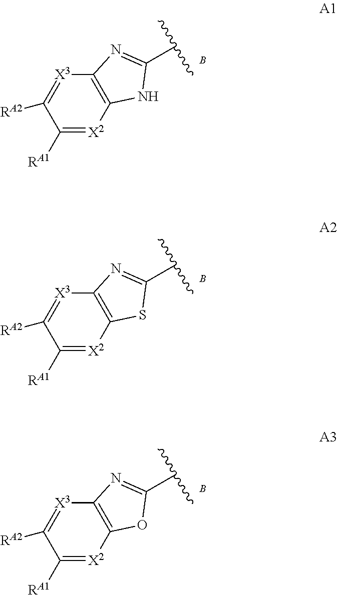

wherein the wavy line indicates the point of attachment to B.

In further embodiments A is selected from one of the following formulae A4-A14:

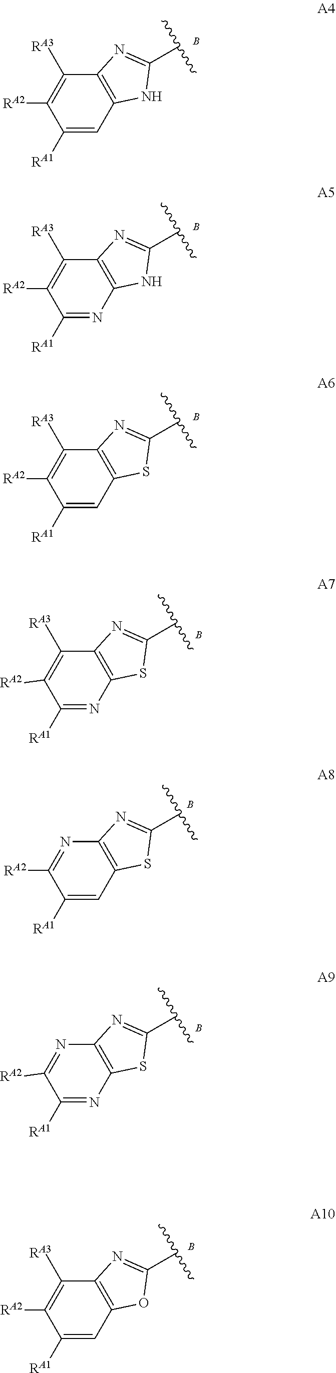

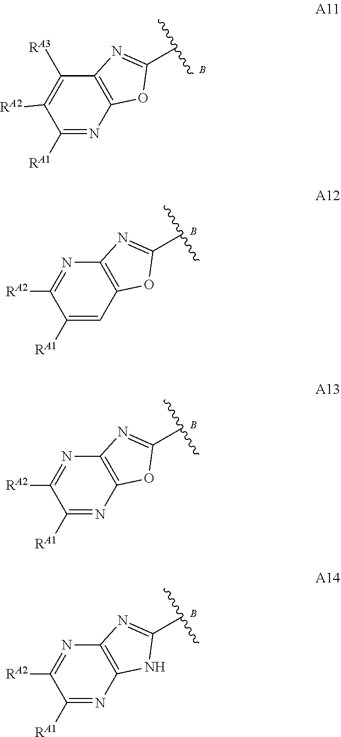

When X¹ is NH, R^{A1} is X⁴ and R^{A2} is X⁵.

When X² is N and X¹ is O or S, R^{A1} is X⁴ and R^{A2} is X⁵.

When X² is CH and X¹ is S, R^{A1} is X⁴ and R^{A2} is X⁵.

When X² is CH and X¹ is O, R^{A1} is X⁵ and R^{A2} is X⁴.

X⁴

X⁴ is selected from the group consisting of:
(i) H;
(ii) halo
(iii) CN;
(iv) C₁ alkyl optionally substituted by one or more OH, CN, or one or more halo groups; or
(v) C₁ alkoxy, optionally substituted by one or more halo groups.

In some embodiments when X⁴ is halo it is Cl, Br or F. In some embodiments when X⁴ is halo it is Br. In some embodiments when X⁴ is an optionally substituted C₁ alkyl it is methyl, CH₂OH, CH₂F, CHF₂, CH₂Br, CHBr₂, CH₂C₁ or CHCl₂. In some embodiments when X⁴ is optionally substituted C₁ alkoxy it is OMe, OCHF₂ or OCF₃.

In some embodiments X⁴ is selected from H, halo, CN, or methyl optionally substituted by one or more OH groups or one or more halo groups.

In some embodiments X⁴ is selected from H, CN, CH₂OH, Br or methyl.

In some embodiments X⁴ is H.

X⁵

In some embodiments X⁵ is selected from the group consisting of:
(i) H;
(ii) halo;
(iii) CN;
(iv) C₁₋₆ hydrocarbon, optionally substituted by OH, CN, C₁₋₆ alkyl acyl, C₁₋₆ alkoxy or one or more halo groups;
(v) C₁₋₆ alkoxy, optionally substituted by C₁₋₆ alkyl amido, C₁₋₆ alkyl phosphonyl, or one or more halo groups;
(vi) C₁₋₆ alkylamino
(vii) C₁₋₆ thioalkyl,
(viii) C₁₋₆ alkyl phosphinyl; or
(ix) C₁₋₆ alkyl phosphonyl.

In some embodiments X⁵ is selected from the group consisting of:
(i) H;
(ii) halo;
(iii) CN;
(iv) C₁₋₆ hydrocarbon, optionally substituted by OH, CN, C₁₋₆ alkyl acyl, C₁₋₆ alkoxy or one or more halo groups;
(v) C₁₋₆ alkoxy, optionally substituted by C₁₋₆ alkyl amido, or one or more halo groups;
(vi) C₁₋₆ alkylamino
(vii) C₁₋₆ thioalkyl, or
(viii) C₁₋₆ alkyl phosphinyl.

In some embodiments X⁵ is selected from:
(i) H;
(ii) halo;
(iii) C₁₋₆ alkyl, optionally substituted by one or more OH or one or more halo groups;
(iv) C₁₋₆ alkoxy, optionally substituted by one or more halo groups;
(v) C₃₋₅ cycloalkyl;
(vi) C₁₋₆ thioalkyl; or
(vii) C₁₋₆ alkyl phosphinyl.

In some embodiments X⁵ is H.

In some embodiments when X⁵ is halo it is F, Cl or Br.

In some embodiments when X⁵ is an optionally substituted C₁₋₆ alkyl it is a methyl, ethyl, propyl, CH₂OH, CH₂F, CHF₂, CF₃. In further embodiments it is methyl, ethyl, CH₂OH or CF₃.

In some embodiments when X⁵ is an optionally substituted C₁₋₆ alkoxy it is OMe, O-ethyl, O-propyl, OCF₂H, OCF₃, OCFH₂. In further embodiments it is OMe, OCF₃, OCF₂H.

In some embodiments when X⁵ is a cycloalkyl it is cyclopropyl or cyclobutyl. In some embodiments it is cyclopropyl.

In some embodiments when X⁵ is a C₁₋₆ thioalkyl it is S—CH₃, S—CH₂CH₃ or S—CH₂CH₂CH₃. In some embodiments it is S—CH₃.

In some embodiments when X⁵ is a C₁₋₆ alkyl phosphinyl it is P(═O)Me₂, P(═O)(CH₂CH₂)₂ or P(═O)(CH₃)(CH₂CH₂). In some embodiments it is P(═O)Me₂.

In further embodiments X⁵ is selected from H, CH₂OH, OCF₂H, OCF₃, CF₃, F, Cl, Br, ethyl, cyclopropyl, methyl, P(═O)Me₂, S—CH₃ or OMe.

R^{A3}

In some embodiments R^{A3} is selected from:
(i) H;
(ii) halo
(iii) CN;
(iv) C₁₋₆ alkyl optionally substituted by OH, or one or more halo groups;
(v) C₂₋₆ alkenyl optionally substituted by OH, or one or more halo groups;
(vi) C₂₋₆ alkynyl optionally substituted by OH, or one or more halo groups; or (vii) C₁₋₆ alkoxy, optionally substituted by one or more halo groups.

In some embodiments R^{A3} is H.

In some embodiments R^{A3} is CN.

In some embodiments when R^{A3} is halo it is selected from Cl, Br or F.

In some embodiments when R^{A3} is an optionally substituted C₁₋₆ alkyl it is methyl, ethyl, propyl, CF₃, CF₂H, CH₂—CF₂H or CH₂-cyclopropyl. In further embodiments it is CF₃, CH₂—CF₂H, CF₂H or CH₂-cyclopropyl.

In some embodiments when R^{A3} is C₂₋₆ alkenyl it is CH═CH₂, CH═CHCH₃ or CH₂CH═CH₂. In some embodiments it is CH═CH₂.

In some embodiments when R^{A3} is C₂₋₆alkynyl it is propargyl, acetylene or 1-butyne. In some embodiments it is propargyl.

In some embodiments when R^{A3} is optionally substituted C₁₋₆ alkoxy it is OCF₃, OCF₂H or OMe.

In some embodiments R^{A3} is selected from H, Cl, Br, OMe, CH═CH₂, OCF₃, OCF₂H, F, CH₂—CF₂H, CF₃, CF₂H, CN, propargyl, CH₂-cyclopropyl. In further embodiments R^{A3} is selected from H, Cl, Br or OMe.

In some embodiments A is of the following formula:

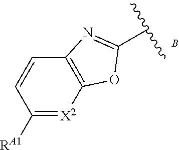

wherein X² is selected from N and C—H and R^{A1} is selected from H, CN, CH₂OH, OCHF₂, methyl or Br.

In further embodiments A is of the following formula:

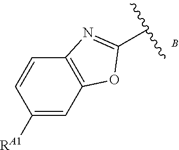

wherein R^{A1} is selected from H, CN, CH₂OH, methyl, OCHF₂, or Br.

In some embodiments A is of the following formula:

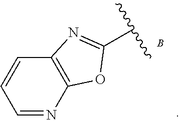

In some embodiments A is of the following formula:

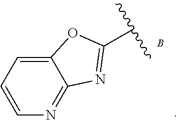

In some embodiments A is formula (A2):

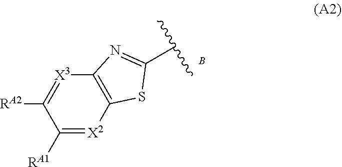

wherein X² is selected from N or C—H, and when X² is N, X³ is either N or C—R^{A3}, and when X² is C—H, X³ is C—R^{A3} or N, R^{A1} is selected from H, methyl or Br, R^{A2} is H, CN or CH₂OH and R^{A3} is H.

In some embodiments A is of the following formula:

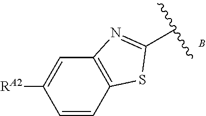

wherein R^{A2} is selected from:
(i) H;
(ii) halo;
(iii) C₁₋₆ alkyl, optionally substituted by one or more OH or one or more halo groups,
(iv) C₁₋₆ alkoxy, optionally substituted by one or more halo groups;
(v) C₃₋₅ cycloalkyl;
(vi) C₁₋₆ thioalkyl;
(vii) C₁₋₆ alkyl phosphinyl; or
(viii) CN.

In further embodiments when A is of the formula above R^{A2} is selected from H, Br, CH₂OH, C₁₋₆ alkyl and CN.

In further embodiments when A is of the formula above R^{A2} is selected from H, CH₂OH, C₁₋₆ alkyl and CN.

In some embodiments A is of the following formula:

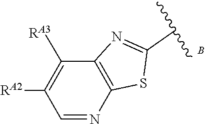

wherein R^{A3} is selected from H, halo or OMe and R^{A2} is selected from H, Br and CH₂OH.

In some embodiments A is of the following formula:

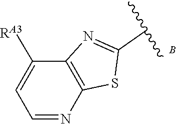

wherein R^{A3} is selected from H, halo or OMe.

In further embodiments when A is of the formula above, R^{A3} is H.

In some embodiments A is of the following formula:

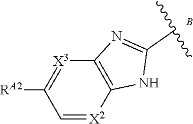

wherein X² is N or C—H, when X² is N, X³ is either N or C—R^{A3}, when X² is C—H, X³ is C—R^{A3}, wherein R^{A3} is selected from H, Cl, Br or OMe, and R^{A2} is selected from H, CH₂OH, OCH₃, OCHF₂, OCF₃, CF₃, F, Cl, Br, ethyl, cyclopropyl, methyl, —P(═O)Me₂ or —S—CH₃.

In some embodiments A is of the following formula:

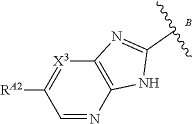

wherein X³ is N or C—R^{A3}, wherein R^{A3} is selected from H, Cl, Br or OMe, R^{A2} is selected from H, CH₂OH, OCH₃, OCHF₂, OCF₃, CF₃, F, Cl, Br, ethyl, cyclopropyl, methyl, —P(═O)Me₂ or —S—CH₃.

In some embodiments A is selected from any one of the following groups listed in the table:

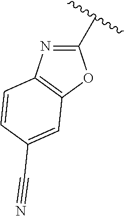 [SMILES: *c1nc2ccc(C#N)cc2o1]

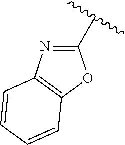 [SMILES: *c1nc2ccccc2o1]

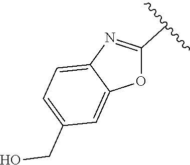 [SMILES: *c1nc2ccc(CO)cc2o1]

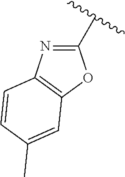 [SMILES: *c1nc2ccc(C)cc2o1]

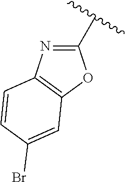 [SMILES: *c1nc2ccc(Br)cc2o1]

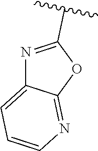 [SMILES: *c1nc2cccnc2o1]

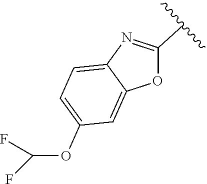 [SMILES: *c1nc2ccc(OC(F)F)cc2o1]

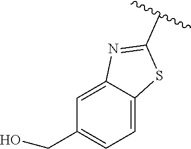 [SMILES: *c1nc2cc(CO)ccc2s1]

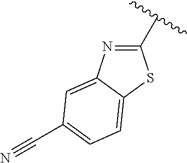 [SMILES: *c1nc2cc(C#N)ccc2s1]

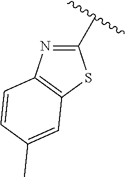 [SMILES: *c1nc2ccc(C)cc2s1]

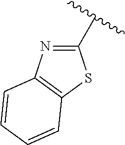 [SMILES: *c1nc2ccccc2s1]

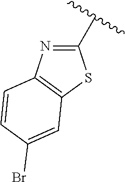 [SMILES: *c1nc2ccc(Br)cc2s1]

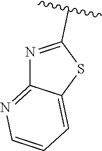 [SMILES: *c1nc2ncccc2s1]

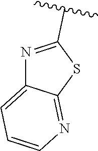 [SMILES: *c1nc2cccnc2s1]

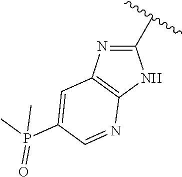 [SMILES: *c1nc2cc(P(C)(C)=O)cnc2[nH]1]

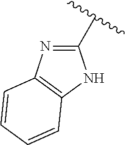 [SMILES: *c1nc2ccccc2[nH]1]

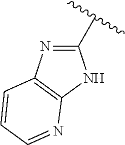 [SMILES: *c1nc2cccnc2[nH]1]

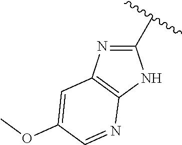 [SMILES: *c1nc2cc(OC)cnc2[nH]1]

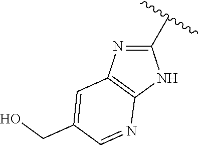 [SMILES: *c1nc2cc(CO)cnc2[nH]1]

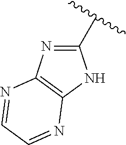 [SMILES: *c1nc2nccnc2[nH]1]

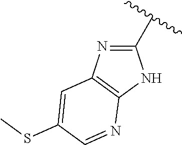 [SMILES: *c1nc2cc(SC)cnc2[nH]1]

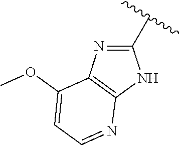 [SMILES: *c1nc2c(OC)ccnc2[nH]1]

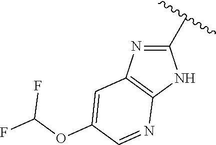 [SMILES: *c1nc2cc(OC(F)F)cnc2[nH]1]

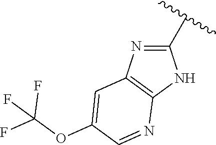 [SMILES: *c1nc2cc(OC(F)(F)F)cnc2[nH]1]

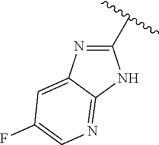 [SMILES: *c1nc2cc(F)cnc2[nH]1]

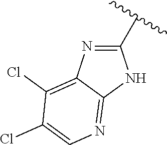 [SMILES: *c1nc2c(Cl)c(Cl)cnc2[nH]1]

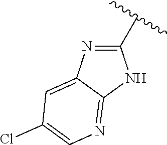 [SMILES: *c1nc2cc(Cl)cnc2[nH]1]

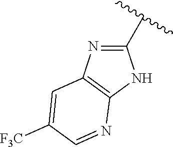 [SMILES: *c1nc2cc(C(F)(F)F)cnc2[nH]1]

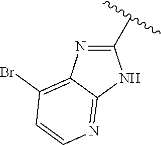 [SMILES: *c1nc2c(Br)ccnc2[nH]1]

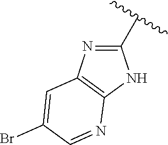 [SMILES: *c1nc2cc(Br)cnc2[nH]1]

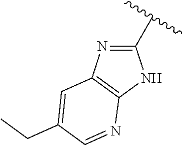 [SMILES: *c1nc2cc(CC)cnc2[nH]1]

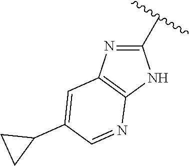 [SMILES: *c1nc2cc(C3CC3)cnc2[nH]1]

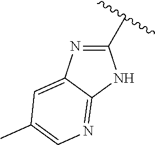 [SMILES: *c1nc2cc(C)cnc2[nH]1]

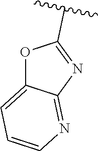 [SMILES: *c1nc2ncccc2o1]

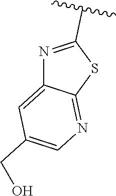 [SMILES: *c1nc2cc(CO)cnc2s1]

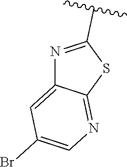 [SMILES: *c1nc2cc(Br)cnc2s1]

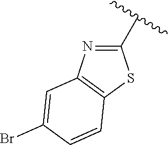 [SMILES: *c1nc2cc(Br)ccc2s1]

wherein the wavy line indicates the point of attachment to B.

### B

In some embodiments B is of formula (B-1):

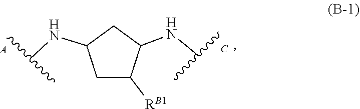

wherein the wavy line indicates the point of attachment to A and C; wherein R^{B1} is H, OH, —OMe, —O-ethyl, —CH₂OH, —CH₂CH₂OH or ═CHCH₂—OH.

In other embodiments R^{B1} is H, —CH₂OH, —CH₂CH₂OH or ═CHCH₂—OH.

In other embodiments R^{B1} is —CH₂OH, —CH₂CH₂OH or ═CHCH₂—OH.

In other embodiments R^{B1} is H.

In another embodiment B is of the formula (B-1a):

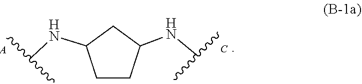

In further embodiments B is of the formula (B-1b):

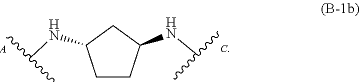

Therefore, in some embodiments the compounds of Formula (I) is the S,S-enantiomer.

In some embodiments B is of formula (B-2):

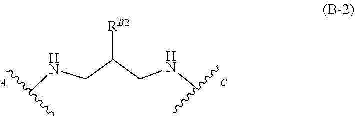

wherein the wavy lines indicate the point of attachment to A and C;
R^{B2} is C₁₋₂ alkyl-OH, CH₂CONHMe or C₁₋₃ alkyl,
wherein when R^{A1} is H or halo, R^{B2} is C₁₋₂ alkyl-OH or CH₂C(═O)NHMe,

In some of these embodiments, R^{B2} is C₁₋₂ alkyl-OH, or C₁₋₃ alkyl.

In some of these embodiments R^{B2} is C₁₋₂ alkyl-OH or CH₂C(═O)NHMe.

In some embodiments when X¹ is O or S and R^{A1} is H or halo, R^{B2} is C₁₋₂ alkyl-OH or CH₂C(═O)NHMe.

In some embodiments when B is of the formula (B-2) it is of the following formula (B-2a):

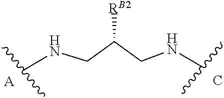

wherein the wavy lines indicate the point of attachment to A and C; and R^{B2} is C₁₋₂alkyl-OH, CH₂CONHMe or C₁₋₂alkyl, wherein when R^{A1} is H or halo, R^{B2} is C₁₋₂ alkyl-OH or CH₂C(═O)NHMe.

### C

When C is an optionally substituted C₅₋₆ heteroaryl in some embodiments it is an optionally substituted C₆ heteroaryl. In other embodiments it is an optionally substituted pyridinyl, pyrazinyl or pyrimidinyl. In other embodiments it is an optionally substituted pyridinyl.

When C is an optionally substituted C₅₋₆ heteroaryl it can be optionally substituted by:
(i) C₆₋₁₀ carboaryl, C₄₋₁₀ carbocyclyl, C₅₋₁₀ heteroaryl, C₅₋₁₀ heterocyclyl, or C₅₋₁₀ bridged heterocyclyl, spiro C₆₋₁₂heterocyclyl or a spiro C₆₋₁₂carbocyclyl, which are themselves optionally substituted by one or more of the groups selected from:
a) one or two ═O groups;
b) one or more halo groups;
c) CN, NH₂, OH;
d) C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl optionally substituted by C₁₋₆ alkyl;
i) C₄₋₁₀ carbocyclyl with an optional methyl or ═O substituent;
j) C₆₋₁₀ carboaryl with optional substituents of one or more halo groups;
l) P(═O)Me₂;
m) C(═O)OH or CH₂C(═O)OH and/or
n) tetrazolyl, CH₂-tetrazolyl, 5-oxo-4H-1,2,4-oxadiazol-3-yl;
(ii) one or more groups selected from C(═O)OH, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido and di-C₁₋₆ alkyl sulfonamido.

Where C is substituted by C₆₋₁₀ carboaryl, C₅₋₁₀ heteroaryl or C₅₋₁₀ heterocyclyl, it may bear a number of substituent groups. The substituents are selected from:
a) one or two ═O groups;
b) one or more halo groups, CN, NH₂;
c) one or more C₁₋₆ alkyl, C₁₋₆ alkoxy, or C₁₋₆ alkylester, wherein each is optionally substituted by one or more halo groups;
d) C₅₋₆ heterocyclyl or C₅₋₆ heteroaryl with an optional methyl substituent;
e) C₆₋₁₀ carboaryl optionally substituted by one or more halo atoms;
f) P(═O)Me₂; or
g) C(═O)OH or CH₂C(═O)OH.

In some embodiments one substituent of C is at the para position.

In some embodiments when C is an optionally substituted C₅₋₆ heteroaryl, the optional substituents are selected from C₁₋₆ alkyl and halo. In further embodiments the optional substituent is methyl. In further embodiments C is substituted by methyl at the meta position.

In other embodiments where C is substituted by an optionally substituted C₆₋₁₀ carboaryl, C₅₋₁₀ heteroaryl or C₅₋₁₀ heterocyclyl, it may be substituted by an optionally substituted phenyl, an optionally substituted pyridyl, an optionally substituted imidazolidinyl, an optionally substituted dihydroquinolinyl, an optionally substituted benzimidazolyl or an optionally substituted imidazopyridinyl. These group themselves may be optionally substituted by one or two ═O groups, halo, CN, one or more C₁₋₆ alkyl, C₁₋₆ alkoxy, C₁₋₆ alkylester, C₅₋₆ heterocyclyl (with an optional methyl substituent), phenyl substituted by F at the para position, C(═O)OH, CH₂C(═O)OH, tetrazolyl, pyrazolyl, triazolyl or P(═O)Me₂.

In other embodiments when C is an optionally substituted pyridinyl, pyrazinyl or pyrimidinyl it may bear a number of substituent groups. The substituents are selected from an optionally substituted C₅₋₁₀ heteroaryl or C₅₋₁₀ heterocyclyl, which are themselves optionally substituted by one or two ═O groups, one or more halo groups, CN, one or more C₁₋₆ alkyl, C₁₋₆ alkoxy, C₁₋₆ alkylester, C₅₋₆ heterocyclyl (with an optional methyl substituent), phenyl optionally substituted with one or two halo groups, C(═O)OH, CH₂C(═O)OH, tetrazolyl, pyrazolyl, triazolyl, a pyridine ring, NH₂ or OH. In some embodiments the substituents are an optionally substituted phenyl, an optionally substituted pyridyl, an optionally substituted imidazolidine, an optionally substituted dihydroquinoline, an optionally substituted benzimidazolyl or an optionally substituted imidazopyridinyl.

In some embodiments C is an optionally substituted pyridinyl, pyrazinyl or pyrimidinyl, wherein the optional substituents are selected from:
i) C₆₋₁₀ carboaryl, C₅₋₁₀ heteroaryl, C₅₋₁₀ heterocyclyl, which are themselves optionally substituted by one or more of the following groups:
   a) one or two ═O groups;
   b) one or more halo groups;
   c) CN, NH₂, OH;
   d) one or more C₁₋₆ alkyl groups including branched and cyclic and with an optional substituent selected from OH or one or more halo groups;
   e) C₁₋₆ alkoxy with optional substituents of one or more halo groups;
   f) C₁₋₆ alkylester;
   g) C₅₋₆ heterocyclyl with an optional methyl, OH or ═O substituent;
   h) C₅₋₆ heteroaryl optionally substituted by C₁₋₆ alkyl;
   i) C₄₋₁₀ carbocyclyl with an optional methyl or ═O substituent;
   j) C₆₋₁₀ carboaryl with optional substituents of one or more halo groups;
   l) P(═O)Me₂;
   m) C(═O)OH, CH₂C(═O)OH; and/or
   n) tetrazolyl, CH₂-tetrazolyl, 5-oxo-4H-1,2,4-oxadiazol-3-yl;
ii) one or more groups selected from C(═O)OH, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido and di-C₁₋₆ alkyl sulfonamido.

In some embodiments C is an optionally substituted pyridinyl, pyrazinyl or pyrimidinyl, wherein the optional substituents are selected from C₆₋₁₀ carboaryl, C₅₋₁₀ heteroaryl, C₅₋₁₀ heterocyclyl, which are themselves optionally substituted by one or more groups selected from the following:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl optionally substituted by C₁₋₆ alkyl;
i) C₄₋₁₀ carbocyclyl with an optional methyl or ═O substituent;
j) C₆₋₁₀ carboaryl with optional substituents of one or more halo groups;
l) P(═O)Me₂;
m) C(═O)OH, CH₂C(═O)OH; and/or
n) tetrazolyl, CH₂-tetrazolyl, 5-oxo-4H-1,2,4-oxadiazol-3-yl.

In some embodiments C is an optionally substituted pyridinyl, pyrazinyl or pyrimidinyl, wherein the optional substituents are selected from C₆₋₁₀ carboaryl, C₅₋₁₀ heteroaryl, C₅₋₁₀ heterocyclyl, which are themselves optionally substituted by one or more groups selected from the following:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₅₋₆ heteroaryl optionally substituted by C₁₋₆ alkyl; and/or
g) C(═O)OH, CH₂C(═O)OH.

In another embodiment C is of the formula (C-1):

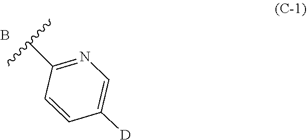

wherein D is C₆₋₁₀ carboaryl, C₅₋₁₀ heteroaryl or C₅₋₁₀ heterocyclyl, which are themselves optionally substituted by one or two ═O groups, one or more halo groups, CN, NH₂, OH, one or more C₁₋₄ alkyl groups, which alkyl is optionally substituted by halo, C₁₋₆ alkoxy which is optionally substituted by halo, C₁₋₆ alkylester, C₅₋₆ heterocyclyl (with an optional methyl substituent), C(═O)OH, CH₂C(═O)OH, P(═O)Me₂, tetrazolyl, pyrazolyl or triazolyl. In some embodiments the optional substituents are selected from ═O, CN, F, Cl, Br, methyl, ethyl, OMe, C(═O)OH, ethoxy, O—CF₃, OCHF₂, CHF₂, CH₂CF₃, CF₃, P(═O)Me₂ and C₁₋₂ alkyl ester. In further embodiments the optional substituents are selected from ═O, methyl, CN, Cl, F, OMe, CF₃, C(═O)OH, OCF₃, OCHF₂, or pyrimidinyl with a methyl substituent.

When C is (C-1) in some embodiments D is an optionally substituted pyridin-2(1H)-one. In some embodiments the optional substituents are selected from OMe, Cl, one or more F groups, methyl, CF₃, I, OCF₃, OCHF₂, C(═O)OH, CN, pyrazolyl, triazolyl, tetrazolyl, phenyl with an optional F substituent at the para position, piperazinyl with a methyl substituent or pyrimidinyl with a methyl substituent. In some embodiments the pyridin-2(1H)-one is unsubstituted.

When C is (C-1), in some embodiments D is an optionally substituted 6 membered heteroaryl which contains one or two N atoms, one of which is bonded to the pyridyl in (C-1), which is substituted by ═O at the ortho position and the other optional substituents are selected from methyl, OMe, piperazine substituted by methyl, pyrimidinyl with a methyl substituent, C(═O)OH, Cl, phenyl substituted by fluoro, CN, CF₃, one or more F groups, pyrazolyl, triazolyl, tetrazolyl, OCHF₂ or O—CF₃.

In some embodiments when C is (C-1), D is an optionally substituted 6 membered heteroaryl which contains 1 or 2 N atoms, one of which is bonded to the pyridyl in (C-1), which is substituted by ═O at the ortho position wherein the optional substituents are selected from:
i) one ═O groups;
ii) one or two C₁₋₄ alkyl groups which can be branched;
iii) OMe;
iv) piperazinyl, or pyrimidinyl optionally substituted by methyl;
v) C(═O)OH;
vi) C₁;
vii) one or more F;
viii) phenyl, optionally substituted by one or more fluoro;
ix) CN;
x) CF₃;
xi) OCF₃;
xii) OCHF₂;
xiii) tetrazolyl, pyrazolyl, triazolyl;
xiv) NH₂;
xv) pyridinyl;
xvi)CH₂OH;
xvii) OH; or
xviii) P(═O)Me₂.

In some embodiments when C is (C-1), D is either an optionally substituted phenyl or an optionally substituted 6 membered heteroaryl which contains 1 or 2 N atoms, one of which is bonded to the pyridyl in (C-1), which is substituted by ═O at the ortho position wherein the optional substituents are selected from:
i) methyl;
ii) OMe;
iii) pyrimidinyl substituted by methyl;
iv) C(═O)OH;
v) Cl;
vi) CN;
vii) one or more F groups;
viii) CF₃;
ix) —O—CF₃; or
x) —OCHF₂.

In some embodiments C is of the formula (C-1) and D is an optionally substituted phenyl or pyridyl wherein there are one or two optional substituents selected from ═O, methyl, OMe, Cl, CN, one or more F groups, C(═O)OH, CF₃, OCF₃, OCHF₂, or pyrimidinyl substituted with methyl.

In another embodiment D is of the formula (D-1):

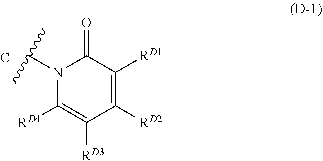

wherein one or two of R^{D1}, R^{D2}, R^{D3} and R^{D4} are selected from a C₁₋₆ alkyl optionally substituted by one or more halo groups; C₁₋₆ alkoxy optionally substituted by one or more halo groups; C₅₋₆ heterocyclyl or C₅₋₆ heteroaryl with an optional methyl substituent; C(═O)OH; ═O; halo; NH₂; CN; or phenyl optionally substituted by one or more halo atoms;
or wherein R^{D3} and R^{D4} form an optionally substituted 6 membered carboaromatic, heterocyclic or heteroaromatic ring wherein the optional substituents are selected from OH, methyl, OMe, halo, CN, P(═O)Me₂ and C(═O)OH;
or wherein R^{D1} and R^{D2} form an optionally substituted 5 or 6 membered carboaromatic, heterocyclic or heteroaromatic ring, wherein the optional substituents are selected from OH, methyl, OMe, CN, P(═O)Me₂, halo and C(═O)OH;
or wherein R^{D1}, R^{D2}, R^{D3} and R^{D4} are all H.

In some embodiments R^{D2} and R^{D3} form an optionally substituted 5 or 6 membered carboaromatic, heterocyclic or heteroaromatic ring, wherein the optional substituents are selected from halo; CN; OH; P(═O)Me₂; C(═O)OH; C₁₋₆ alkoxy optionally substituted by one or more halo groups; or C₁₋₆ alkyl optionally substituted by one or more halo groups. In further embodiments the optional substituents are selected from OH, methyl, OMe, halo, CN, P(═O)Me₂ and C(═O)OH.

In another embodiment one or two of R^{D1}, R^{D2}, R^{D3} and R^{D4} are selected from C₁₋₆ alkyl optionally substituted by one or more halo groups, C₁₋₆ alkoxy optionally substituted by one or more halo groups, C₅₋₆ heterocyclyl or C₅₋₆ heteroaryl with an optional methyl substituent, C(═O)OH, halo or CN, and the rest are H.

In some embodiments D is of the formula (D-1) and one or two of R^{D1}, R^{D2}, R^{D3} and R^{D4} are selected from:
i) methyl;
ii) OMe;
iii) pyrimidinyl substituted by methyl;
iv) tetrazolyl;
v) C(═O)OH;
vi) halo;
vii) CN;
viii) CF₃;
ix) —O—CF₃; or
x) —OCHF₂,
and the rest of R^{D1}, R^{D2}, R^{D3} and R^{D4} are H.

In another embodiment one of R^{D1}, R^{D2}, R^{D3} and R^{D4} are selected from methyl, OMe, halo, C(═O)OH, CN, CF₃, tetrazolyl, pyrimidinyl substituted by methyl, OCF₃ and OCHF₂ and the rest of R^{D1}, R^{D2}, R^{D3} and R^{D4} are H.

In one embodiment all of R^{D1}, R^{D2}, R^{D3} and R^{D4} are H.

In another embodiment R^{D3} is selected from H, methyl, OMe, —C(═O)OH, C, CN, OCHF₂, OCH₂F, tetrazolyl, and pyrimidinyl optionally substituted by methyl wherein R^{D1}, R^{D2} and R^{D4} are all H.

In other embodiments R^{D1} is selected from H, methyl, OMe, C₁, CF₃, OCF₃, OCHF₂ and CN and R^{D2}, R^{D3} and R^{D4} are all H.

In another embodiment R^{D3} and R^{D4} form an optionally substituted 6 membered carboaromatic, heterocyclic or heteroaromatic ring wherein the optional substituents are selected from OH, CN, P(═O)Me₂, methyl, OMe, halo and C(═O)OH.

In some embodiments R^{D3} and R^{D4} form an optionally substituted phenyl ring or an optionally substituted pyridine ring.

In some embodiments R^{D3} and R^{D4} form an unsubstituted phenyl ring or an unsubstituted pyridine ring.

In another embodiment R^{D1} and R^{D2} form an optionally substituted 5 or 6 membered carboaromatic, heterocyclic or heteroaromatic ring, wherein the optional substituents are selected from OH, methyl, OMe, halo, CN and C(═O)OH.

In some embodiments R^{D1} and R^{D2} form an optionally substituted 5 membered heterocyclic or heteroaromatic ring. In some R^{D1} and R^{D2} form a 5 membered heterocyclic or heteroaromatic ring containing one sulphur atom. In further embodiments R^{D1} and R^{D2} form an unsubstituted thiophene.

In some embodiments C is of the formula (C-1) and D is an optionally substituted phenyl or piperidyl, wherein there are one or two optional substituents selected from F, OCHF₂ OCF₃, or OMe. In some embodiments the optionally substituted phenyl is substituted by one or two substituents at the ortho position.

In some embodiments D is an optionally substituted 6 membered heterocyclyl or heteroaryl containing two N groups, wherein the optional substituents are selected from ═O, methyl, —OMe, halo, —C(═O)OH, CN, CF₃, —OCHF₂. In some embodiments the optional substituent is ═O. In some embodiments D is a 6 membered heteroaryl containing two nitrogen atoms with an ═O substituent at the ortho position.

In some embodiments C is of the following formulae:

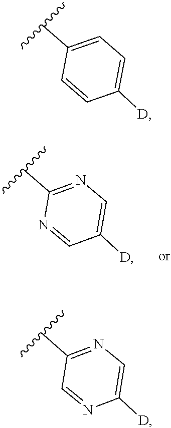

and D is as described in the embodiments above.

In another embodiment D is of the formula (D-2):

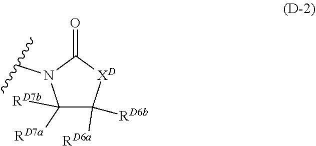

wherein X^{D} is NR^{D5a} or CR^{D5a}R^{D5b};
R^{D5a} is selected from H or methyl;
either R^{D5b} and R^{D6b} are both H or together they are CH₂—;
R^{D6a} is selected from H, ═O, methyl, CH₂OH or C(═O)OH, wherein when R^{D6a} is ═O, R^{D6b} is absent;
R^{D7a} is selected from H, ═O, methyl, CH₂OH or C(═O)OH;
R^{D7b} is H, wherein when R^{D7a} is ═O, R^{D7b} is absent;
or wherein R^{D6a} and R^{D7a} together form a phenyl ring or a C₆ heteroaromatic ring which is optionally substituted by CN, P(═O)Me₂ or C(═O)OH and R^{D6b} and R^{D7b} are absent.

In some embodiments C is of the formula (C-1) and D is of the formula (D-2) and X^{D} is NR^{D5a} and R^{D5a} is methyl.

In some embodiments C is of the formula (C-1) and D is of the formula (D-2) and R^{D6a} and R^{D6b} are both H and R^{D7a} is selected from ═O, —CH₂OH or —C(═O)OH and R^{D7b} is H or when R^{D7a} is ═O, R^{D7b} is absent. In further embodiments R^{D7a} is ═O.

In some embodiments C is of the formula (C-1) and D is of the formula (D-2) and X^{D} is NR^{D5a} and R^{D5a} is methyl wherein R^{D7a} is selected from H and ═O and R^{D6a} is selected from H and ═O, wherein when R^{D7a} is ═O, R^{D6a} and R^{D6b} are H and R^{D7b} is absent and wherein when R^{D6a} is ═O, R^{D7a} and R^{D7b} are H and R^{D6b} is absent.

In another embodiment R^{D6a} and R^{D7a} together form a phenyl ring or a C₆ heteroaromatic ring which is optionally substituted by CN, P(═O)Me₂ or —C(═O)OH, and R^{D6b} and R^{D7b} are absent.

In some embodiments R^{D6a} and R^{D7a} form an unsubstituted phenyl ring and R^{D6b} and R^{D7b} are absent. In some embodiments R^{D6a} and R^{D7a} form an unsubstituted pyridine ring and R^{D6b} and R^{D7b} are absent.

In some embodiments X^{D} is NR^{D5a} and R^{D5a} is selected from H or methyl and R^{D6a} and R^{D7a} form an unsubstituted phenyl ring and R^{D6b} and R^{D7b} are absent.

In some embodiments X^{D} is NR^{D5a} and R^{D5a} is H or methyl.

In some embodiments X^{D} is CR^{D5a}R^{D5b} and R^{D5a} is H or methyl and R^{D5b} is H. In further embodiments both R^{D5a} and R^{D5b} are H.

In some embodiments R^{D7a} is H, C(═O)OH, —CH₂OH or ═O. In some embodiments R^{D7a} is ═O or H. R^{D7b} is H, or when R^{D7a} is ═O, R^{D7b} is absent.

In some embodiments R^{D6a} is H or ═O. In some embodiments R^{D6a} is ═O and R^{D6b} is absent.

In some embodiments where R^{D7a} is ═O, R^{D6a} and R^{D6b} are H and R^{D7b}-is absent, and where R^{D6a} is ═O, R^{D7a} and R^{D7b} are H and R^{D6b} is absent.

In some embodiments X^{D} is NR^{D5a} and R^{D5a} is H or methyl, R^{D6a} and R^{D7a} together form a pyridine ring or a phenyl ring which is optionally substituted by —C(═O)OH or CN or P(═O)Me₂ and R^{D6b} and R^{D7b} are absent.

In some embodiments C is of the formula (C-1) and D is of the formula (D-3):

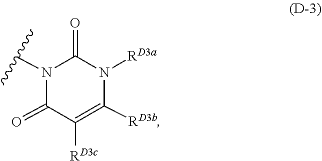

wherein R^{D3a} is selected from H and methyl;
wherein either R^{D3b} and R^{D3c} are independently selected from H; halo; CN; OH; P(═O)Me₂; C(═O)OH; C₁₋₆ alkoxy optionally substituted by one or more halo groups; or C₁₋₆ alkyl optionally substituted by one or more halo groups; or
R^{D3b} and R^{D3c} together form a C₅₋₆ heteroaryl ring.

In further embodiments R^{D3a} is methyl and R^{D3b} and R^{D3c} are H.

In another embodiment R^{D3a} is H and R^{D3b} and R^{D3c} together form a thiophene ring.

In some embodiments D is selected from any one of the following groups listed in the table:

| Name | Structure |
|---|---|
| 2-oxo-1-pyridyl | 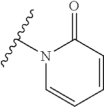 [SMILES: *n1ccccc1=O] |
| 3-methyl-2-oxo-1- pyridyl | 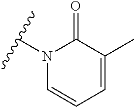 [SMILES: *n1cccc(C)c1=O] |
| 3-methoxy-2-oxo-1- pyridyl | 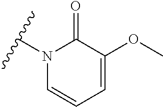 [SMILES: *n1cccc(OC)c1=O] |
| 5-methoxy-2-oxo-1- pyridyl | 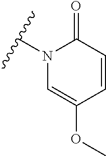 [SMILES: *n1cc(OC)ccc1=O] |
| 5-chloro-2-oxo-1-pyridyl | 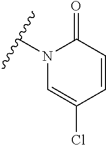 [SMILES: *n1cc(Cl)ccc1=O] |
| 2-oxo-1,8-naphthyridin- 1-yl | 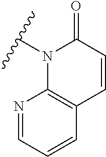 [SMILES: *n1c(=O)ccc2cccnc21] |
| 2-oxo-1-quinolyl | 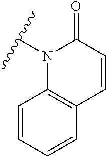 [SMILES: *n1c(=O)ccc2ccccc21] |
| 3-methyl-2-oxo- benzimidazol-1-yl | 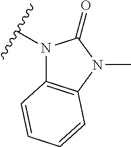 [SMILES: *n1c(=O)n(C)c2ccccc21] |
| 3-methyl-2,4-dioxo- imidazolidin-1-yl | 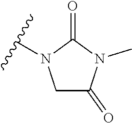 [SMILES: *N1CC(=O)N(C)C1=O] |
| 3-methyl-2,5-dioxo- imidazolidin-1-yl | 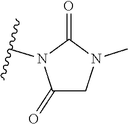 [SMILES: *N1C(=O)CN(C)C1=O] |
| 2,4-dioxoimidazolidin-1- yl | 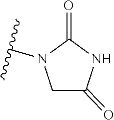 [SMILES: *N1CC(=O)NC1=O] |
| 2,5-dioxoimidazolidin-1- yl | 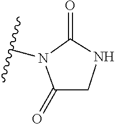 [SMILES: *N1C(=O)CNC1=O] |
| 3-chloro-2-oxo-1-pyridyl | 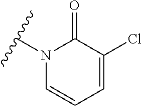 [SMILES: *n1cccc(Cl)c1=O] |
| 3-cyano-2-oxo-1-pyridyl | 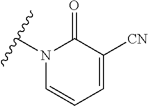 [SMILES: *n1cccc(C#N)c1=O] |
| 5-cyano-2-oxo-1-pyridyl | 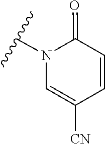 [SMILES: *n1cc(C#N)ccc1=O] |
| 3-fluoro-2-oxopyridin- 1(2H)-yl | 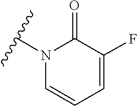 [SMILES: *n1cccc(F)c1=O] |
| 5-methyl-2-oxo-1- pyridyl | 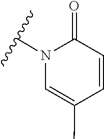 [SMILES: *n1cc(C)ccc1=O] |
| 5-carboxy-2-oxo-1- pyridyl | 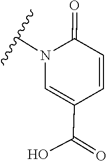 [SMILES: *n1cc(C(=O)O)ccc1=O] |
| 2-oxo-5-(1H-pyrazol-4- yl)-1-pyridyl | 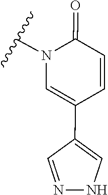 [SMILES: *n1cc(-c2cn[nH]c2)ccc1=O] |
| 2-oxo-5-(1H-triazol-4- yl)-1-pyridyl | 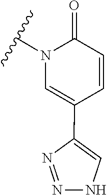 [SMILES: *n1cc(-c2c[nH]nn2)ccc1=O] |
| 5-cyano-3-methyl-2-oxo- benzimidazol-1-yl | 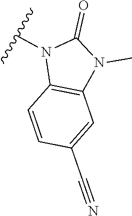 [SMILES: *n1c(=O)n(C)c2cc(C#N)ccc21] |
| 5-carboxy-3-methyl-2- oxo-benzimidazol-1-yl | 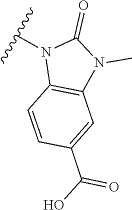 [SMILES: *n1c(=O)n(C)c2cc(C(=O)O)ccc21] |
| 6-carboxy-3-methyl-2- oxo-benzimidazol-1-yl | 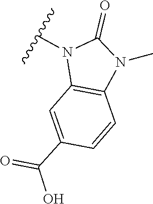 [SMILES: *n1c(=O)n(C)c2ccc(C(=O)O)cc21] |
| (6-dimethylphosphoryl- 3-methyl-2-oxo- benzimidazo-1-yl) | 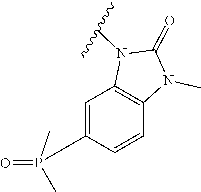 [SMILES: *n1c(=O)n(C)c2ccc(P(C)(C)=O)cc21] |
| 2-oxo-3H-benzimidazol- 1-yl | 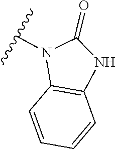 [SMILES: *n1c(=O)[nH]c2ccccc21] |
| 2-oxo-1H-imidazo[4,5- b]pyridin-3-yl | 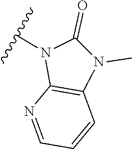 [SMILES: *n1c(=O)n(C)c2cccnc21] |
| 6-oxopyridazin-1-yl | 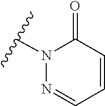 [SMILES: *n1ncccc1=O] |
| 6-oxopyrimidin-1-yl | 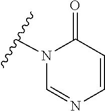 [SMILES: *n1cnccc1=O] |
| 1-methyl-2-oxo-3- pyridyl | 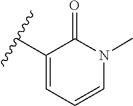 [SMILES: *c1cccn(C)c1=O] |
| 2-oxo-3- (trifluoromethyl)-1- pyridyl | 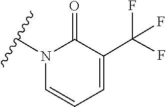 [SMILES: *n1cccc(C(F)(F)F)c1=O] |
| 2-oxo-3- (trifluoromethoxy)-1- pyridyl | 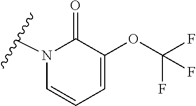 [SMILES: *n1cccc(OC(F)(F)F)c1=O] |
| 2-oxo-5-(1H-tetrazol-5- yl)-1-pyridyl | 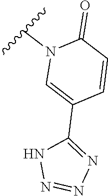 [SMILES: *n1cc(-c2nnn[nH]2)ccc1=O] |
| 2-fluorophenyl | 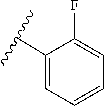 [SMILES: *c1ccccc1F] |
| 5-(2-methylpyrimidin-5- yl)-2-oxo-1-pyridyl | 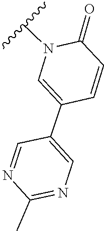 [SMILES: *n1cc(-c2cnc(C)nc2)ccc1=O] |
| 2-(difluoromethoxy)-6- fluoro-phenyl | 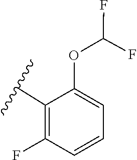 [SMILES: *c1c(F)cccc1OC(F)F] |
| 2-methoxyphenyl | 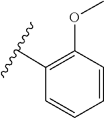 [SMILES: *c1ccccc1OC] |
| 2,6-difluorophenyl | 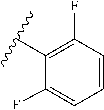 [SMILES: *c1c(F)cccc1F] |
| 2-fluoro-6-methoxy- phenyl | 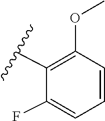 [SMILES: *c1c(F)cccc1OC] |
| 3-fluoro-2-pyridyl | 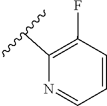 [SMILES: *c1ncccc1F] |
| 7-oxothieno[2,3- c]pyridin-6-yl | 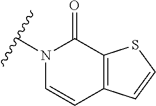 [SMILES: *n1ccc2ccsc2c1=O] |
| 2-fluoro-6- (trifluoromethoxy)phenyl | 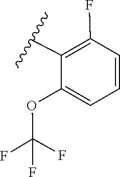 [SMILES: *c1c(F)cccc1OC(F)(F)F] |
| [5-(difluoromethoxy)-2- oxo-1-pyridyl] | 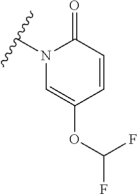 [SMILES: *n1cc(OC(F)F)ccc1=O] |
| 3-(difluoromethoxy)-2- oxo-1-pyridyl | 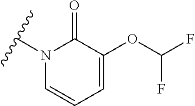 [SMILES: *n1cccc(OC(F)F)c1=O] |
| 3-methyl-2,6-dioxo- pyrimidin-1-yl | 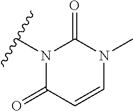 [SMILES: *n1c(=O)ccn(C)c1=O] |
| 2,4-dioxo-1H-thieno[3,2- d]pyrimidin-3-yl | 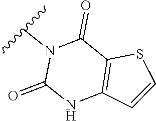 [SMILES: *n1c(=O)[nH]c2ccsc2c1=O] |
| 2-oxo-5-(2H-tetrazol-5- yl)-1-pyridyl | 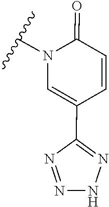 [SMILES: *n1cc(-c2nn[nH]n2)ccc1=O] |

In further embodiments D is selected from any one of the following groups listed in the table:

| Name | Structure |
|---|---|
| 2-oxo-1-pyridyl | 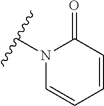 [SMILES: *n1ccccc1=O] |
| 3-methyl-2-oxo-1- pyridyl | 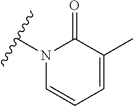 [SMILES: *n1cccc(C)c1=O] |
| 3-methoxy-2-oxo-1- pyridyl | 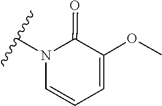 [SMILES: *n1cccc(OC)c1=O] |
| 5-methoxy-2-oxo-1- pyridyl | 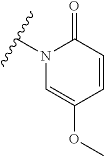 [SMILES: *n1cc(OC)ccc1=O] |
| 5-chloro-2-oxo-1-pyridyl | 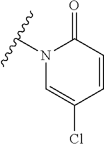 [SMILES: *n1cc(Cl)ccc1=O] |
| 2-oxo-1,8-naphthyridin- 1-yl | 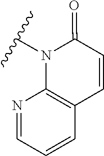 [SMILES: *n1c(=O)ccc2cccnc21] |
| 2-oxo-1-quinolyl | 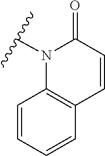 [SMILES: *n1c(=O)ccc2ccccc21] |
| 3-methyl-2-oxo- benzimidazol-1-yl | 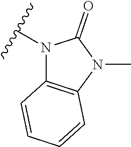 [SMILES: *n1c(=O)n(C)c2ccccc21] |
| 3-methyl-2,4-dioxo- imidazolidin-1-yl | 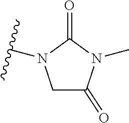 [SMILES: *N1CC(=O)N(C)C1=O] |
| 3-methyl-2,5-dioxo- imidazolidin-1-yl | 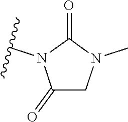 [SMILES: *N1C(=O)CN(C)C1=O] |
| 3-chloro-2-oxo-1-pyridyl | 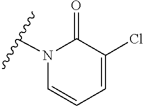 [SMILES: *n1cccc(Cl)c1=O] |
| 3-cyano-2-oxo-1-pyridyl | 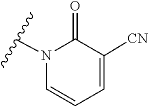 [SMILES: *n1cccc(C#N)c1=O] |
| 5-cyano-2-oxo-1-pyridyl | 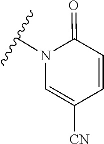 [SMILES: *n1cc(C#N)ccc1=O] |
| 3-fluoro-2-oxopyridin- 1(2H)-yl | 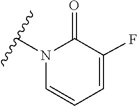 [SMILES: *n1cccc(F)c1=O] |
| 2-fluoro-6-methoxy- phenyl | 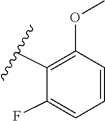 [SMILES: *c1c(F)cccc1OC] |
| 5-carboxy-2-oxo-1- pyridyl | 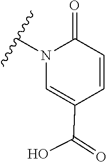 [SMILES: *n1cc(C(=O)O)ccc1=O] |
| 2-oxo-3H-benzimidazol- 1-yl | 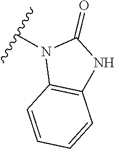 [SMILES: *n1c(=O)[nH]c2ccccc21] |
| 2-oxo-1H-imidazo[4,5- b]pyridin-3-yl | 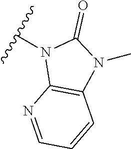 [SMILES: *n1c(=O)n(C)c2cccnc21] |
| 6-oxopyridazin-1-yl | 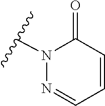 [SMILES: *n1ncccc1=O] |
| 6-oxopyrimidin-1-yl | 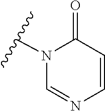 [SMILES: *n1cnccc1=O] |
| 1-methyl-2-oxo-3- pyridyl | 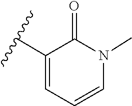 [SMILES: *c1cccn(C)c1=O] |
| 2-oxo-3- (trifluoromethoxy)-1- pyridyl | 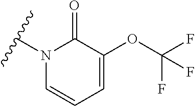 [SMILES: *n1cccc(OC(F)(F)F)c1=O] |
| 2-fluorophenyl | 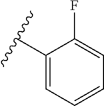 [SMILES: *c1ccccc1F] |
| 2-methoxyphenyl | 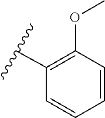 [SMILES: *c1ccccc1OC] |
| 2-(difluoromethoxy)-6- fluoro-phenyl | 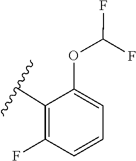 [SMILES: *c1c(F)cccc1OC(F)F] |
| 5-(2-methylpyrimidin-5- yl)-2-oxo-1-pyridyl | 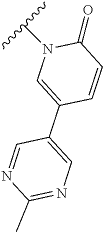 |
| 2,6-difluorophenyl | 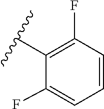 [SMILES: *c1c(F)cccc1F] |
| 3-fluoro-2-pyridyl | 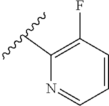 [SMILES: *c1ncccc1F] |
| 7-oxothieno[2,3- c]pyridin-6-yl | 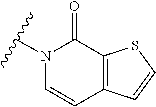 [SMILES: *n1ccc2ccsc2c1=O] |
| 2-oxo-3- (trifluoromethyl)-1- pyridyl | 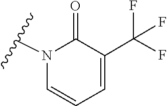 [SMILES: *n1cccc(C(F)(F)F)c1=O] |
| 2-fluoro-6- (trifluoromethoxy)phenyl | 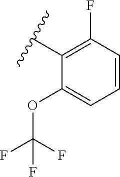 [SMILES: *c1c(F)cccc1OC(F)(F)F] |
| [5-(difluoromethoxy)-2- oxo-1-pyridyl] | 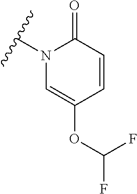 [SMILES: *n1cc(OC(F)F)ccc1=O] |
| 3-(difluoromethoxy)-2- oxo-1-pyridyl | 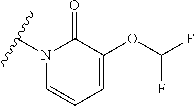 [SMILES: *n1cccc(OC(F)F)c1=O] |
| 3-methyl-2,6-dioxo- pyrimidin-1-yl | 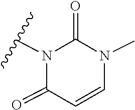 [SMILES: *n1c(=O)ccn(C)c1=O] |
| 2,4-dioxo-1H-thieno[3,2- d]pyrimidin-3-yl | 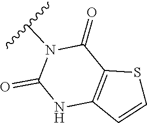 [SMILES: *n1c(=O)[nH]c2ccsc2c1=O] |
| 2-oxo-5-(2H-tetrazol-5- yl)-1-pyridyl | 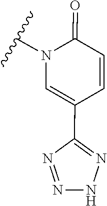 [SMILES: *n1cc(-c2nn[nH]n2)ccc1=O] |
| 2,4-dioxoimidazolidin-1- yl | 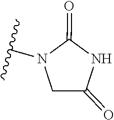 [SMILES: *N1CC(=O)NC1=O] |

In another embodiment C has the formula (C-2):

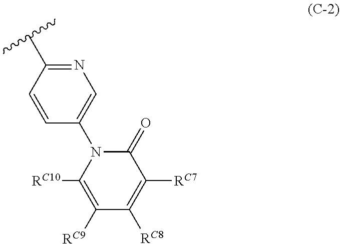

wherein one or two of R^{C7}, R^{C8}, R^{C9} and R^{C10} are selected from methyl, OMe, halo, OCHF₂, C(═O)OH, piperazine or pyrimidinyl (optionally substituted by methyl), optionally substituted phenyl (wherein the optional substituent is methyl or halo), CN, CF₃, —OCF₃, tetrazolyl, pyrazolyl, or triazolyl and the rest of R^{C7}, R^{C8}, R^{C9} and R^{C10} are H; or R^{C9} and R^{C10} form an optionally substituted 6 membered carboaromatic, heterocyclic or heteroaromatic ring wherein the optional substituents are selected from OH, methyl, OMe, halo, CN, P(═O)Me₂ or —C(═O)OH; or wherein R^{C7}, R^{C8}, R^{C9} and R^{C10} are all H.

In one embodiment C has the formula (C-2) and all of R^{C7}, R^{C8}, R^{C9} and R^{C10} are H.

In another embodiment one or two of R^{C7}, R^{C8}, R^{C9} and R^{C10} are independently selected from methyl, Cl, OMe, phenyl substituted with F at para position, —C(═O)OH, CN, OCF₃, OCHF₂, CF₃, F, pyrazolyl, triazolyl, tetrazolyl, pyrimidinyl substituted by methyl, piperazinyl substituted by methyl and the rest of R^{C7}, R^{C8}, R^{C9} and R^{C10} are H. In further embodiments R^{C7}, R^{C8}, R^{C9} and R^{C10} are independently selected from methyl, Cl, OMe, CN, OCF₃, CF₃, OCHF₂, C(═O)OH or pyrimidinyl substituted by methyl, and the rest of R^{C7}, R^{C8}, R^{C9} and R^{C10} are H.

In another embodiment one of R^{C7} and R^{C9} is independently selected from H, OCHF₂, methyl, —OMe, C, —C(═O)OH, piperazinyl optionally substituted by methyl, pyrimidinyl optionally substituted by methyl, optionally substituted phenyl (wherein the optional substituent is F), CN, CF₃, —OCF₃, F, pyrazolyl, triazolyl or tetrazolyl and the other is H and R^{C10} and R^{C8} are H. In another embodiment one of R^{C7} and R^{C9} is independently selected from H, methyl, Cl, CF₃, OCHF₂, OMe, ON, OCF₃, ON, C(═O)OH or pyrimidinyl substituted by methyl, and the other is H and R^{C10} and R^{C8} are H.

In another embodiment, R^{C7} is selected from H, methyl, Cl, OMe, ON, OCF₃, OCHF₂, CF₃, and R^{C8}, R^{C9} and R^{C10} are all H.

In another embodiment R^{C9} is selected from H, ON, OMe, OCHF₂, Cl, C(═O)OH, tetrazolyl, or pyrimidinyl substituted by methyl, and R^{C7}, R^{C8} and R^{C10} are all H.

In another embodiment R^{C9} and R^{C10} together form a phenyl ring or a pyridyl ring. In another embodiment R^{C7} and R^{C8} together form a thiophene ring.

In another embodiment C is selected from any one of the following groups listed in the table:

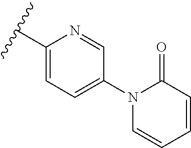 [SMILES: *c1ccc(-n2ccccc2=O)cn1]

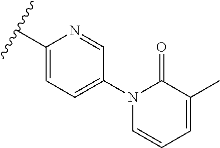 [SMILES: *c1ccc(-n2cccc(C)c2=O)cn1]

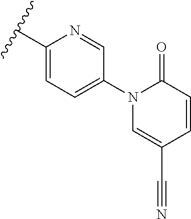 [SMILES: *c1ccc(-n2cc(C#N)ccc2=O)cn1]

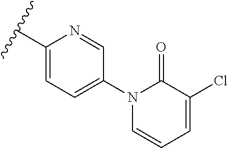 [SMILES: *c1ccc(-n2cccc(Cl)c2=O)cn1]

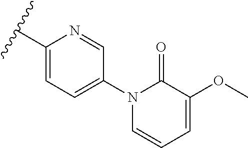 [SMILES: *c1ccc(-n2cccc(OC)c2=O)cn1]

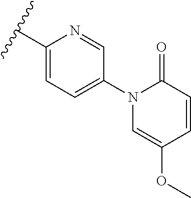 [SMILES: *c1ccc(-n2cc(OC)ccc2=O)cn1]

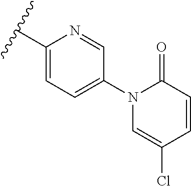 [SMILES: *c1ccc(-n2cc(Cl)ccc2=O)cn1]

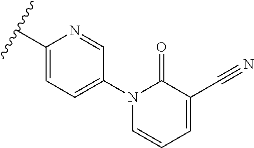 [SMILES: *c1ccc(-n2cccc(C#N)c2=O)cn1]

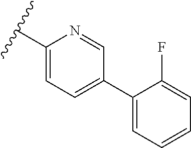 [SMILES: *c1ccc(-c2ccccc2F)cn1]

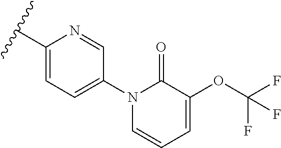 [SMILES: *c1ccc(-n2cccc(OC(F)(F)F)c2=O)cn1]

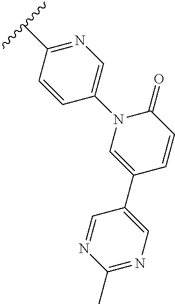 [SMILES: *c1ccc(-n2cc(-c3cnc(C)nc3)ccc2=O)cn1]

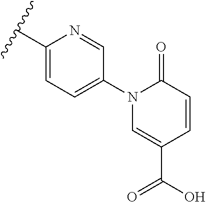 [SMILES: *c1ccc(-n2cc(C(=O)O)ccc2=O)cn1]

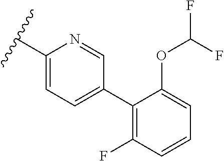 [SMILES: *c1ccc(-c2c(F)cccc2OC(F)F)cn1]

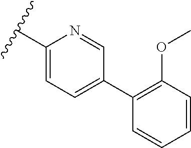 [SMILES: *c1ccc(-c2ccccc2OC)cn1]

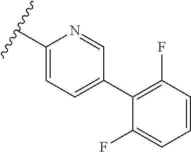 [SMILES: *c1ccc(-c2c(F)cccc2F)cn1]

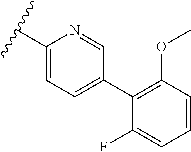 [SMILES: *c1ccc(-c2c(F)cccc2OC)cn1]

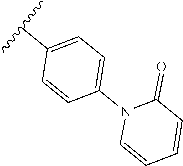 [SMILES: *c1ccc(-n2ccccc2=O)cc1]

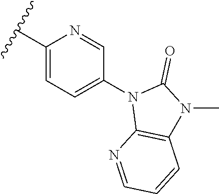 [SMILES: *c1ccc(-n2c(=O)n(C)c3cccnc32)cn1]

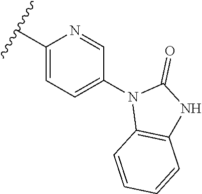 [SMILES: *c1ccc(-n2c(=O)[nH]c3ccccc32)cn1]

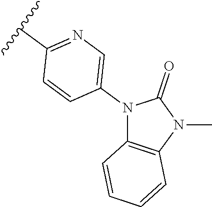 [SMILES: *c1ccc(-n2c(=O)n(C)c3ccccc32)cn1]

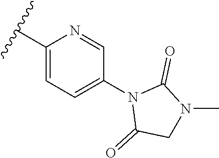 [SMILES: *c1ccc(N2C(=O)CN(C)C2=O)cn1]

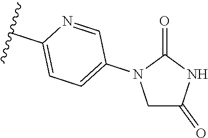 [SMILES: *c1ccc(N2CC(=O)NC2=O)cn1]

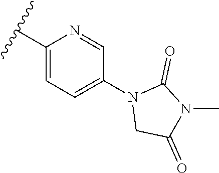 [SMILES: *c1ccc(N2CC(=O)N(C)C2=O)cn1]

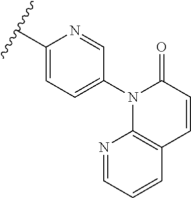 [SMILES: *c1ccc(-n2c(=O)ccc3cccnc32)cn1]

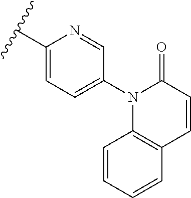 [SMILES: *c1ccc(-n2c(=O)ccc3ccccc32)cn1]

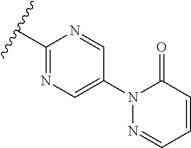 [SMILES: *c1ncc(-n2ncccc2=O)cn1]

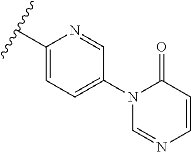 [SMILES: *c1ccc(-n2cnccc2=O)cn1]

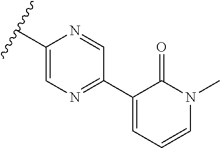 [SMILES: *c1cnc(-c2cccn(C)c2=O)cn1]

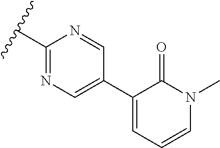 [SMILES: *c1ncc(-c2cccn(C)c2=O)cn1]

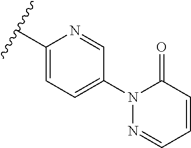 [SMILES: *c1ccc(-n2ncccc2=O)cn1]

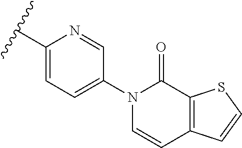 [SMILES: *c1ccc(-n2ccc3ccsc3c2=O)cn1]

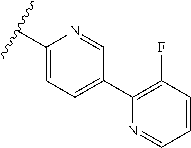 [SMILES: *c1ccc(-c2ncccc2F)cn1]

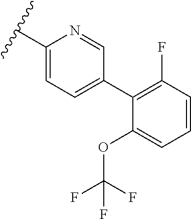 [SMILES: *c1ccc(-c2c(F)cccc2OC(F)(F)F)cn1]

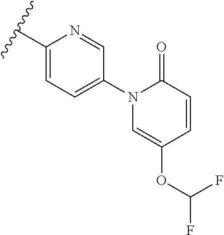 [SMILES: *c1ccc(-n2cc(OC(F)F)ccc2=O)cn1]

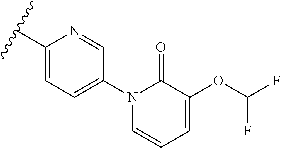 [SMILES: *c1ccc(-n2cccc(OC(F)F)c2=O)cn1]

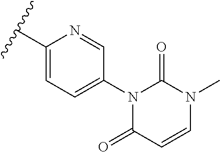 [SMILES: *c1ccc(-n2c(=O)ccn(C)c2=O)cn1]

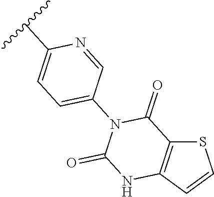 [SMILES: *c1ccc(-n2c(=O)[nH]c3ccsc3c2=O)cn1]

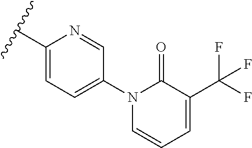 [SMILES: *c1ccc(-n2cccc(C(F)(F)F)c2=O)cn1]

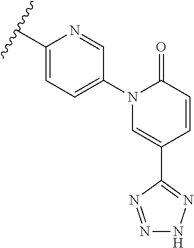 [SMILES: *c1ccc(-n2cc(-c3nn[nH]n3)ccc2=O)cn1]

### A-B-C

In other embodiments the compound of formula A-B-C is of the formula (I-A):

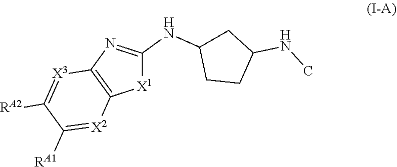

wherein X¹, X², X³, R^{A1}, R^{A2} and C are as defined above.

In further embodiments when the compounds of formula A-B-C is of the formula (I-A) it is of the formulae (I-A1), (I-A2) or (I-A3).

In some embodiments formula (I-A) is formula (I-A1):

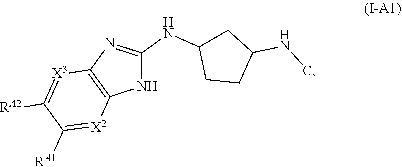

wherein X² is either N or C—H, X³ is either N or C—R^{A3} and when X² is C—H then X³ is C—R^{A3}. R^{A1} is X⁴ and R^{A2} is X⁵.

In some embodiments formula (I-A) is formula (I-A2):

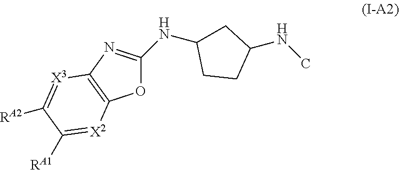

wherein X² is either N or C—H, and when X² is CH, R^{A1} is X⁵ and R^{A2} is X⁴ and when X² is N, R^{A1} is X⁴ and R^{A2} is X⁵. X³ is either N or C—R^{A3}.

In some embodiments formula (I-A) is formula (I-A3):

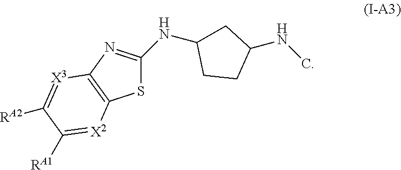

wherein X² is either N or C—H, and when X² is N, R^{A1} is X⁴ and R^{A2} is X⁵, when X² is CH, R^{A1} is X⁴ and R^{A2} is X⁵. X³ is either N or C—R^{A3}.

In some embodiments when A-B-C is of formula (I-A), (I-A1), (I-A2) or (I-A3) X⁴ is selected from the group consisting of H, halo, CN, C₁ alkyl optionally substituted by one or more OH, CN, or one or more halo groups or C₁ alkoxy, optionally substituted by one or more halo groups. In further embodiments X⁴ is selected from H, CN, CH₂OH, Br or methyl.

In some embodiments when A-B-C is of formula (I-A), (I-A1), (I-A2) or (I-A3) X⁵ is selected from the group consisting of H, one or more halo groups, CN, C₁₋₆ hydrocarbon, optionally substituted by OH, CN, C₁₋₆ alkyl acyl, C₁₋₆ alkoxy or one or more halo groups, C₁₋₆ alkoxy, optionally substituted by C₁₋₆ alkyl amido, C₁₋₆ alkyl phosphonyl, or one or more halo groups, C₁₋₆ alkylamino, C₁₋₆ thioalkyl, C₁₋₆ alkyl phosphinyl, or C₁₋₆ alkyl phosphonyl. In further embodiments X⁵ is selected from H, CH₂OH, OCF₂H, OCF₃, CF₃, F, Cl, Br, ethyl, cyclopropyl, methyl, P(═O)Me₂, S—CH₃ CN, or OMe.

In some embodiments when A-B-C is of formula (I-A), (I-A1), (I-A2) or (I-A3) X⁵ is selected from the group consisting of H, one or more halo groups, CN, C₁₋₆ hydrocarbon, optionally substituted by OH, CN, C₁₋₆ alkyl acyl, C₁₋₆ alkoxy or one or more halo groups, C₁₋₆ alkoxy, optionally substituted by C₁₋₆ alkyl amido, or one or more halo groups, C₁₋₆ alkylamino, C₁₋₆ thioalkyl, or C₁₋₆ alkyl phosphinyl. In further embodiments X⁵ is selected from H, CH₂OH, OCF₂H, OCF₃, CF₃, F, Cl, Br, ethyl, cyclopropyl, methyl, P(═O)Me₂, S—CH₃ CN, or OMe.

In some embodiments when A-B-C is of formula (I-A), (I-A1), (I-A2) or (I-A3), R^{A3} is selected from H, halo, CN, C₁₋₆ alkyl optionally substituted by OH or one or more halo groups, C₂₋₆ alkenyl optionally substituted by OH or one or more halo groups, C₂₋₆ alkynyl optionally substituted by OH or one or more halo groups, or C₁₋₆ alkoxy, optionally substituted by one or more halo groups. In further embodiments R^{A3} is selected from H, Cl, Br, OMe, CH═CH₂, OCF₃, OCF₂H, F, CH₂—CF₂H, CF₃, CF₂H, CN, propargyl, CH₂-cyclopropyl. In further embodiments R^{A3} is selected from H, Cl, Br or OMe.

In some embodiments when A-B-C is of formula (I-A), (I-A1), (I-A2) or (I-A3) C is selected from the group consisting of C₆₋₁₀ carboaryl, C₅₋₆ heteroaryl or C₅₋₁₀ heterocyclyl, which groups are optionally substituted by:
(i) C₆₋₁₀ carboaryl, C₄₋₁₀ carbocyclyl, C₅₋₁₀ heteroaryl, C₄₋₁₀ heterocyclyl, or C₅₋₁₀ bridged heterocyclic, spiro C₆₋₁₂ heterocyclyl or a spiro C₆₋₁₂ carbocyclyl,
   which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkyl ester;
g) C₅₋₆ heterocyclyl with an optional methyl, OH or ═O substituent;
h) C₅₋₆ heteroaryl optionally substituted by C₁₋₆ alkyl;
i) C₄₋₁₀ carbocyclyl with an optional methyl or ═O substituent;
j) C₆₋₁₀ carboaryl with optional substituents of one or more halo groups;
l) P(═O)Me₂;
m) C(═O)OH or CH₂C(═O)OH; and/or
n) tetrazolyl, CH₂-tetrazolyl, 5-oxo-4H-1,2,4-oxadiazol-3-yl;
(ii) one or more groups selected from C(═O)OH, CN, halo, nitro, C₁₋₆ alkyl, C₁₋₆ thioalkyl, C₁₋₆ alkoxy, C₁₋₆ alkyl acyl, C₁₋₆ alkyl amido, di-C₁₋₆ alkyl amido, C₁₋₆ alkyl sulfonamido, and di-C₁₋₆ alkyl sulfonamido.

In other embodiments A-B-C is of the formula (I-B):

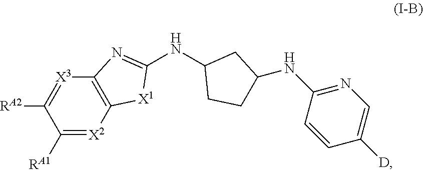

wherein X¹, X², X³, R^{A1}, R^{A2} and D are as defined above.

In further embodiments when A-B-C is of formula (I-B) it is of formula (I-B1):

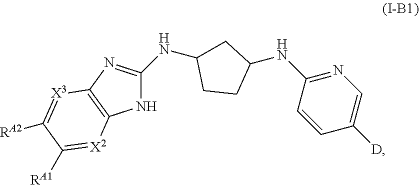

wherein X² is either N or C—H, X³ is either N or C—R^{A3} and when X² is C—H then X³ is C—R^{A3}. R^{A1} is X⁴ and R^{A2} is X⁵. Wherein R^{A3}, X⁴ and X⁵ are as defined above.

In further embodiments when A-B-C is of formula (I-B) it is of formula (I-B2):

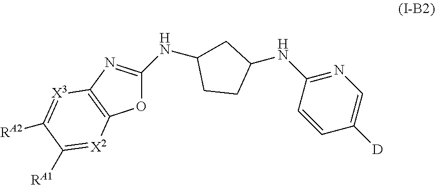

wherein X² is either N or C—H, and when X² is CH, R^{A1} is X⁵ and R^{A2} is X⁴ and when X² is N, R^{A1} is X⁴ and R^{A2} is X⁵. X³ is either N or C—R^{A3}. Wherein R^{A3}, X⁴ and X⁵ are as defined above.

In further embodiments when A-B-C is of formula (I-B) it is of formula (I-B3):

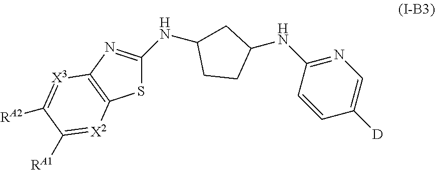

wherein X² is either N or C—H, and when X² is N, R^{A1} is X⁴ and R^{A2} is X⁵, and when X² is OH, R^{A1} is X⁴ and R^{A2} is X⁵. X³ is either N or C—R^{A3}. Wherein R^{A3}, X⁴ and X⁵ are as defined above.

In some embodiments, when A-B-C is of formula (I-B), (I-B1), (I-B2) or (I-B3) then D is C₆₋₁₀ carboaryl, C₅₋₁₀ heteroaryl or C₅₋₁₀ heterocyclyl, each which are themselves optionally substituted by:
one or two ═O groups; one or two C₁₋₄ alkyl groups which can be branched; CHF₂, CH₂CF₃, OCHF₂, OMe; piperazinyl, or pyrimidinyl optionally substituted by methyl; C(═O)OH; Cl; one or more F; phenyl, optionally substituted by one or more fluoro; CN; CF₃; OCF₃; tetrazolyl, pyrazolyl, triazolyl; NH₂; pyridinyl; CH₂OH; OH; or P(═O)Me₂. In further embodiments D is an optionally substituted phenyl or an optionally substituted 6 membered heteroaryl containing one or two N atoms. In further embodiments the optional substituents are selected from ═O, methyl; OMe; pyrimidinyl substituted by methyl; C(═O)OH; Cl; CN; ethyl; one or more F groups; CF₃; CHF₂; OCF₃; or —OCHF₂.

In other embodiments A-B-C is of the formula (I-C):

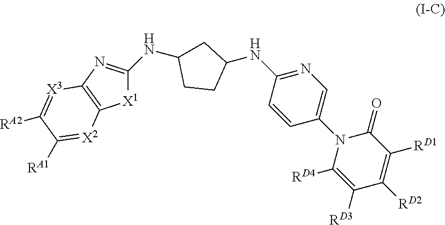

wherein X¹, X², X³, R^{A1}, R^{A2}, R^{D1}, R^{D2}, R^{D3} and R^{D4} are as defined above.

In further embodiments when A-B-C is of formula (I-C) it is of formula (I-C1):

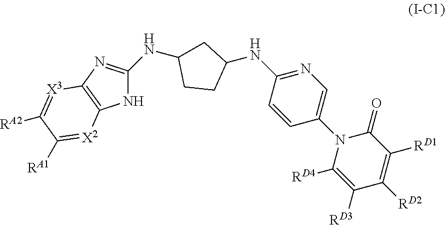

wherein X² is either N or C—H, X³ is either N or C—R^{A3} and when X² is C—H then X³ is C—R^{A3}. R^{A1} is X⁴ and R^{A2} is X⁵. Wherein R^{A3}, X⁴ and X⁵ are as defined above.

In further embodiments when A-B-C is of formula (I-C) it is of formula (I-C2):

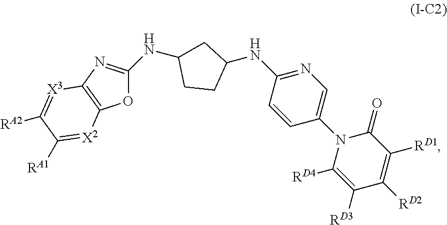

wherein X² is either N or C—H, and when X² is CH, R^{A1} is X⁵ and R^{A2} is X⁴ and when X² is N, R^{A1} is X⁴ and R^{A2} is X⁵. X³ is either N or C—R^{A3}. Wherein R^{A3}, X⁴ and X⁵ are as defined above.

In further embodiments when A-B-C is of formula (I-C) it is of formula (I-C3):

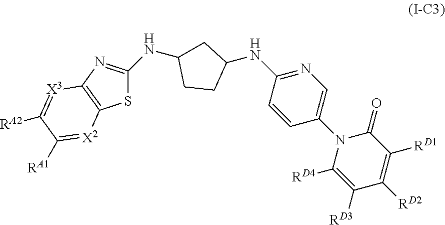

wherein X² is either N or CH, and when X² is N, R^{A1} is X⁴ and R^{A2} is X⁵, when X² is CH, R^{A1} is X⁴ and R^{A2} is X⁵. X³ is either N or C—R^{A3}. Wherein R^{A3}, X⁴ and X⁵ are as defined above.

Wherein when A-B-C is of formula (I-C), (I-C1), (I-C2) or (I-C3) in one embodiment one or two of R^{D1}, R^{D2}, R^{D3} and R^{D4} are selected from C₁₋₆ alkyl, optionally substituted by one or more halo groups; C₁₋₆ alkoxy, optionally substituted by one or more halo groups; C₅₋₆ heterocyclyl or C₅₋₆ heteroaryl with an optional methyl substituent; C(═O)OH or CH₂C(═O)OH; ═O; halo; NH₂ or CN; phenyl, optionally substituted by one or more halo atoms; and the rest are H; or
R^{D3} and R^{D4} form an optionally substituted 6 membered carboaromatic, heterocyclic or heteroaromatic ring, wherein the optional substituents are selected from OH, methyl, OMe, halo and C(═O)OH; or
R^{D1} and R^{D2} form an optionally substituted 5 or 6 membered carboaromatic, heterocyclic or heteroaromatic ring, wherein the optional substituents are selected from OH, methyl, OMe, halo CN, and C(═O)OH.

In further embodiments one or two of R^{D1}, R^{D2}, R^{D3} and R^{D4} are selected from methyl, OMe, pyrimidinyl substituted by methyl, tetrazolyl, C(═O)OH, halo, CF₃, CN, OCF₃, —OCHF₂.

In some embodiments R^{D1}, R^{D2}, R^{D3} and R^{D4} are all H.

In some embodiments R^{D3} and R^{D4} form an unsubstituted phenyl ring or an unsubstituted pyridine ring.

In some embodiments R^{D1} and R^{D2} form an unsubstituted 5 membered heterocyclic or heteroaromatic ring.

In some embodiments A-B-C is of formula (I-D):

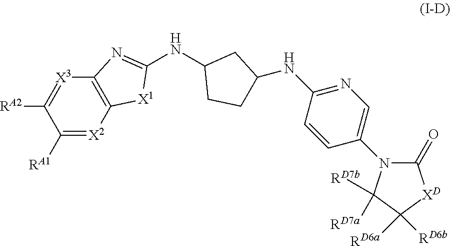

wherein X¹, X², X³, R^{A1}, R^{A2}, X^{D}, R^{D6a}, R^{D6b}, R^{D7a} and R^{D7b} are as defined above.

In some embodiments when A-B-C is of formula (I-D) it is of formula (I-D1).

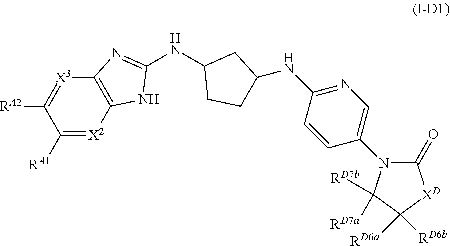

wherein X² is either N or C—H, X³ is either N or C—R^{A3} and when X² is C—H then X³ is C—R^{A3}. R^{A1} is X⁴ and R^{A2} is X⁵. Wherein R^{A3}, X⁴ and X⁵ are as defined above.

In some embodiments when A-B-C is of formula (I-D) it is of formula (I-D2).

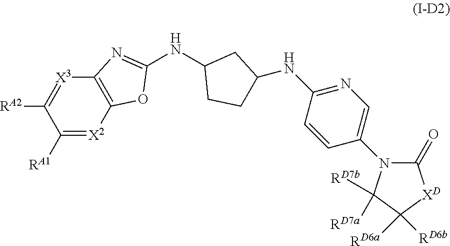

wherein X² is either N or C—H, when X² is C—H, R^{A1} is X⁵ and R^{A2} is X⁴ and when X² is N, R^{A1} is X⁴ and R^{A2} is X⁵. X³ is either N or C—R^{A3}. Wherein R^{A3}, X⁴ and X⁵ are as defined above.

In some embodiments when A-B-C is of formula (I-D) it is of formula (I-D3).

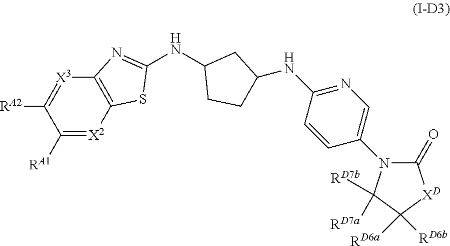

wherein X² is either N or C—H, when X² is N, R^{A1} is X⁴ and R^{A2} is X⁵, when X² is C—H, R^{A1} is X⁴ and R^{A2} is X⁵. X³ is either N or C—R^{A3}. Wherein R^{A3}, X⁴ and X⁵ are as defined above.

In some embodiments when A-B-C is (I-D), (I-D1), (I-D2) or (I-D3), X^{D} is NR^{D5a} or CR^{D5a}R^{D5b}; R^{D5a} is selected from H or methyl; either R^{D5b} and R^{D6b} are both H or together they are —CH₂—; R^{D6a} is selected from H, ═O, methyl, —CH₂OH or —C(═O)OH, wherein when R^{D6a} is ═O, R^{D6b} is absent; R^{D7a} is selected from H, ═O, methyl, —CH₂OH or —C(═O)OH, R^{D7b} is H or when R^{D7a} is ═O, R^{D7b} is absent; or wherein R^{D6a} and R^{D7a} together form a phenyl ring or a C₆ heteroaromatic ring which is optionally substituted by CN, P(═O)Me₂ or C(═O)OH and R^{D6b} and R^{D7b} are absent.

In further embodiments when R^{D6a} and R^{D6b} are H, R^{D7a} is selected from ═O, C(═O)OH or CH₂OH, when R^{D7a} is ═O, R^{D7b} is absent and when R^{D7a} is C(═O)OH or CH₂OH, R^{D7b} is H. In another embodiment when R^{D7a} is H, R^{D7b} is H, R^{D6a} is ═O and R^{D6b} is absent.

In some embodiments R^{D6a} and R^{D7a} together form a phenyl ring or a C₆ heteroaromatic ring which is optionally substituted by CN, C(═O)OH or P(═O)Me₂ and R^{D6b} and R^{D7b} are absent.

In some embodiments R^{D6a} and R^{D7a} form an unsubstituted phenyl ring or an unsubstituted pyridine ring and R^{D6b} and R^{D7b} are absent.

In some embodiments A-B-C is of formula (I-E):

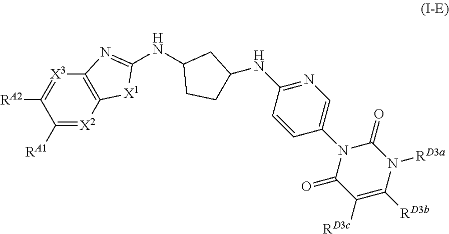

wherein X¹, X², X³, R^{A1}, R^{A2}, R^{D3a}, R^{D3b} and R^{D3c} are as defined above.

In some embodiments when A-B-C is of formula (I-E) it is of formula (I-E1):

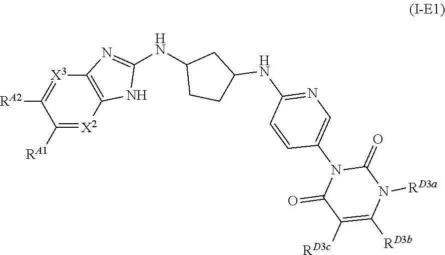

wherein X² is either N or C—H, X³ is either N or C—R^{A3} and when X² is C—H then X³ is C—R^{A3}. R^{A1} is X⁴ and R^{A2} is X⁵. Wherein R^{A3}, X⁴ and X⁵ are as defined above.

In some embodiments when A-B-C is of formula (I-E) it is of formula (I-E2):

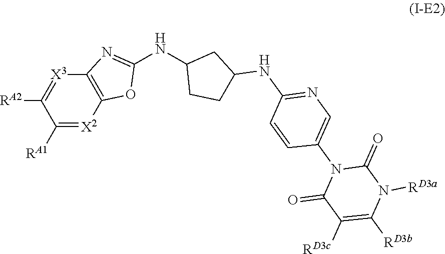

wherein X² is either N or C—H, when X² is C—H, R^{A1} is X⁵ and R^{A2} is X⁴ and when X² is N, R^{A1} is X⁴ and R^{A2} is X⁵. X³ is either N or C—R^{A3}. Wherein R^{A3}, X⁴ and X⁵ are as defined above.

In some embodiments when A-B-C is of formula (I-E) it is of formula (I-E3):

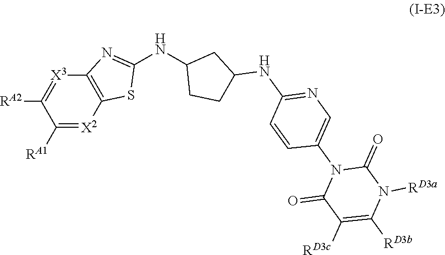

wherein X² is either N or C—H, when X² is N, R^{A1} is X⁴ and R^{A2} is X⁵, when X² is C—H, R^{A1} is X⁴ and R^{A2} is X⁵. X³ is either N or C—R^{A3}. Wherein R^{A3}, X⁴ and X⁵ are as defined above.

In some embodiments when A-B-C is (I-E), (I-E1), (I-E2) or (I-E3), R^{D3a} is selected from H and methyl; and either
R^{D3b} and R^{D3c} are independently selected from H; halo; CN; OH; P(═O)Me₂; C(═O)OH; C₁₋₆ alkoxy optionally substituted by one or more halo groups; or C₁₋₆ alkyl optionally substituted by one or more halo groups; or

R^{D3b} and R^{D3c} together form a C₅₋₆ heteroaryl ring.

In some embodiments R^{D3a} is methyl and R^{D3b} and R^{D3c} are H.

In some embodiments R^{D3a} is H and when R^{D3b} and R^{D3c} together form a C₅₋₆ heteroaryl ring they form a thiophene ring.

In some embodiments the compound of Formula (I) is selected from the following in Table 1.

TABLE 1

| Example | | |
|---|---|---|
| No | Structure | Example Name (PCSKFU) |
| 46 | 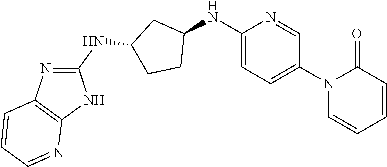 [SMILES: O=c1ccccn1-c1ccc(N[C@H]2CC[C@H](Nc3nc4cccnc4[nH]3)C2)nc1] | 6′-(((1S,3S)-3-((1H-Imidazo[4,5- b]pyridin-2-yl)amino)cyclopentyl)amino)- 2H-[1,3′-bipyridin]-2-one |
| 10 | 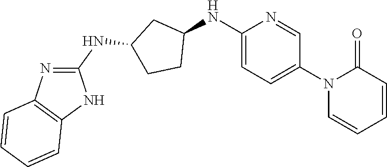 [SMILES: O=c1ccccn1-c1ccc(N[C@H]2CC[C@H](Nc3nc4ccccc4[nH]3)C2)nc1] | 6′-(((1S,3S)-3-((1H-Benzo[d]imidazol-2- yl)amino)cyclopentyl)amino)-2H-[1,3′- bipyridin]-2-one |
| 33 | 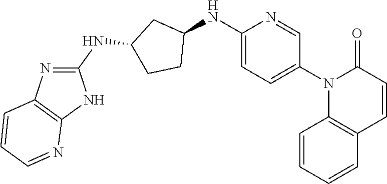 [SMILES: O=c1ccc2ccccc2n1-c1ccc(N[C@H]2CC[C@H](Nc3nc4cccnc4[nH]3)C2)nc1] | 1-(6-(((1S,3S)-3-((3H-Imidazo[4,5- b]pyridin-2- yl)amino)cyclopentyl)amino)pyridin-3- yl)quinolin-2(1H)-one |
| 1 | 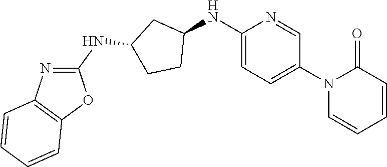 [SMILES: O=c1ccccn1-c1ccc(N[C@H]2CC[C@H](Nc3nc4ccccc4o3)C2)nc1] | 6′-(((1S,3S)-3-(Benzo[d]oxazol-2- ylamino)cyclopentyl)amino)-2H-[1,3′- bipyridin]-2-one |
| 6 | 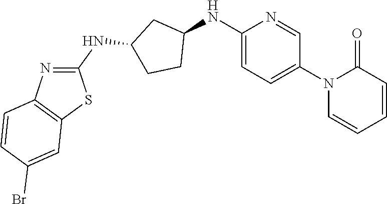 [SMILES: O=c1ccccn1-c1ccc(N[C@H]2CC[C@H](Nc3nc4ccc(Br)cc4s3)C2)nc1] | 6′-(((1S,3S)-3-((6-Bromobenzo[d]thiazol- 2-yl)amino)cyclopentyl)amino)-2H-[1,3′- bipyridin]-2-one |
| 7 | 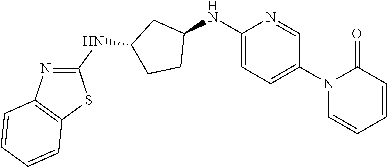 [SMILES: O=c1ccccn1-c1ccc(N[C@H]2CC[C@H](Nc3nc4ccccc4s3)C2)nc1] | 6′-(((1S,3S)-3-(Benzo[d]thiazol-2- ylamino)cyclopentyl)amino)-2H-[1,3′- bipyridin]-2-one |
| 3 | 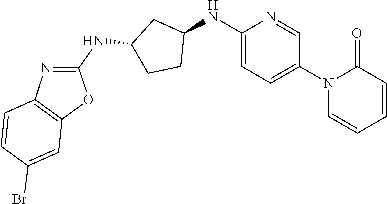 [SMILES: O=c1ccccn1-c1ccc(N[C@H]2CC[C@H](Nc3nc4ccc(Br)cc4o3)C2)nc1] | 6′-(((1S,3S)-3-((6-Bromobenzo[d]oxazol- 2-yl)amino)cyclopentyl)amino)-2H-[1,3′- bipyridin]-2-one |
| 8 | 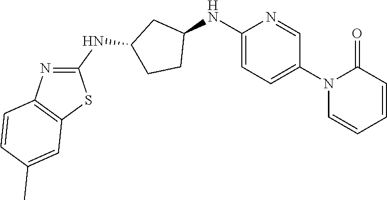 [SMILES: Cc1ccc2nc(N[C@H]3CC[C@H](Nc4ccc(-n5ccccc5=O)cn4)C3)sc2c1] | 6′-(((1S,3S)-3-((6-Methylbenzo[d]thiazol- 2-yl)amino)cyclopentyl)amino)-2H-[1,3′- bipyridin]-2-one |
| 2 | 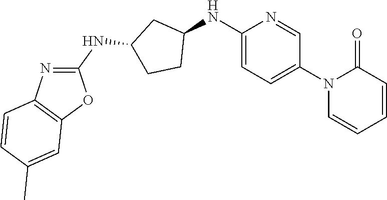 [SMILES: Cc1ccc2nc(N[C@H]3CC[C@H](Nc4ccc(-n5ccccc5=O)cn4)C3)oc2c1] | 6′-(((1S,3S)-3-((6-Methylbenzo[d]oxazol- 2-yl)amino)cyclopentyl)amino)-2H-[1,3′- bipyridin]-2-one |
| 34 | 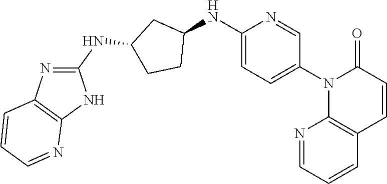 [SMILES: O=c1ccc2cccnc2n1-c1ccc(N[C@H]2CC[C@H](Nc3nc4cccnc4[nH]3)C2)nc1] | 1-(6-(((1S,3S)-3-((3H-Imidazo[4,5- b]pyridin-2- yl)amino)cyclopentyl)amino)pyridin-3-yl)- 1,8-naphthyridin-2(1H)-one |
| 47 | 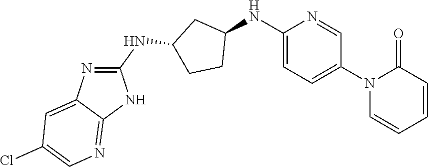 [SMILES: O=c1ccccn1-c1ccc(N[C@H]2CC[C@H](Nc3nc4cc(Cl)cnc4[nH]3)C2)nc1] | 6′-(((1S,3S)-3-((6-Chloro-3H- imidazo[4,5-b]pyridin-2- yl)amino)cyclopentyl)amino)-2H-[1,3′- bipyridin]-2-one |
| 50 | 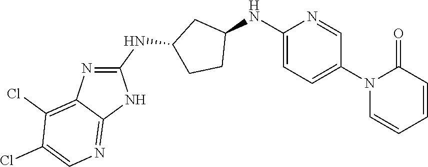 [SMILES: O=c1ccccn1-c1ccc(N[C@H]2CC[C@H](Nc3nc4c(Cl)c(Cl)cnc4[nH]3)C2)nc1] | 6′-(((1S,3S)-3-((6,7-Dichloro-3H- imidazo[4,5-b]pyridin-2- yl)amino)cyclopentyl)amino)-2H-[1,3′- bipyridin]-2-one |
| 9 | 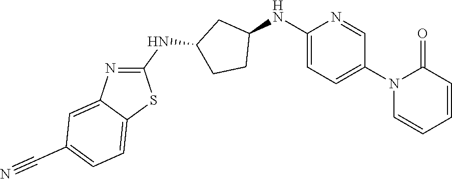 [SMILES: N#Cc1ccc2sc(N[C@H]3CC[C@H](Nc4ccc(-n5ccccc5=O)cn4)C3)nc2c1] | 2-(((1S,3S)-3-((2-Oxo-2H-[1,3′- bipyridin]-6′- yl)amino)cyclopentyl)amino)benzo[d] thiazole-5-carbonitrile |
| 36 | 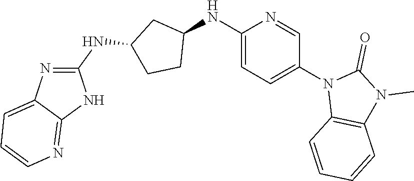 [SMILES: Cn1c(=O)n(-c2ccc(N[C@H]3CC[C@H](Nc4nc5cccnc5[nH]4)C3)nc2)c2ccccc21] | 1-(6-(((1S,3S)-3-((3H-Imidazo[4,5- b]pyridin-2- yl)amino)cyclopentyl)amino)pyridin-3-yl)- 3-methyl-1,3-dihydro-2H- benzo[d]imidazol-2-one |
| 48 | 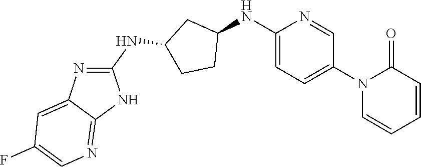 [SMILES: O=c1ccccn1-c1ccc(N[C@H]2CC[C@H](Nc3nc4cc(F)cnc4[nH]3)C2)nc1] | 6′-(((1S,3S)-3-((6-Fluoro-3H-imidazo[4,5- b]pyridin-2-yl)amino)cyclopentyl)amino)- 2H-[1,3′-bipyridin]-2-one |
| 4 | 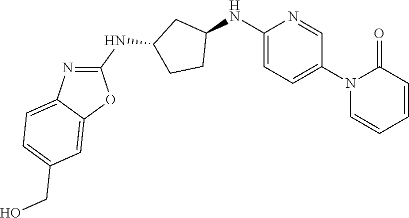 [SMILES: O=c1ccccn1-c1ccc(N[C@H]2CC[C@H](Nc3nc4ccc(CO)cc4o3)C2)nc1] | 6′-(((1S,3S)-3-((6- (Hydroxymethyl)benzo[d]oxazol-2- yl)amino)cyclopentyl)amino)-2H-[1,3′- bipyridin]-2-one |
| 49 | 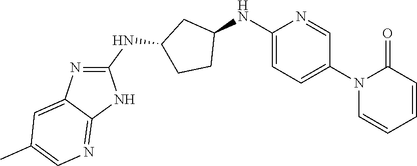 [SMILES: Cc1cnc2[nH]c(N[C@H]3CC[C@H](Nc4ccc(-n5ccccc5=O)cn4)C3)nc2c1] | 6′-(((1S,3S)-3-((6-Methyl-3H- imidazo[4,5-b]pyridin-2- yl)amino)cyclopentyl)amino)-2H-[1,3′- bipyridin]-2-one |
| 62 | 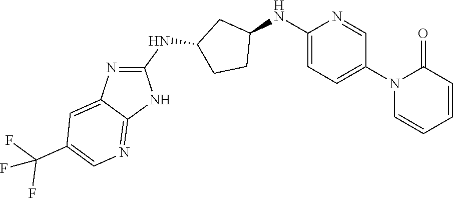 [SMILES: O=c1ccccn1-c1ccc(N[C@H]2CC[C@H](Nc3nc4cc(C(F)(F)F)cnc4[nH]3)C2)nc1] | 6′-(((1S,3S)-3-((6-(Trifluoromethyl)-3H- imidazo[4,5-b]pyridin-2- yl)amino)cyclopentyl)amino)-2H-[1,3′- bipyridin]-2-one |
| 40 | 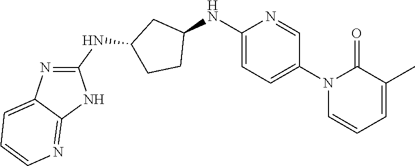 [SMILES: Cc1cccn(-c2ccc(N[C@H]3CC[C@H](Nc4nc5cccnc5[nH]4)C3)nc2)c1=O] | 6′-(((1S,3S)-3-((3H-Imidazo[4,5- b]pyridin-2-yl)amino)cyclopentyl)amino)- 3-methyl-2H-[1,3′-bipyridin]-2-one |
| 54 | 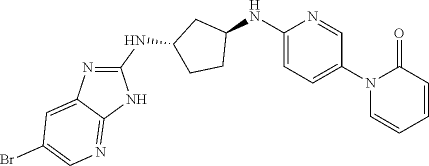 [SMILES: O=c1ccccn1-c1ccc(N[C@H]2CC[C@H](Nc3nc4cc(Br)cnc4[nH]3)C2)nc1] | 6′-(((1S,3S)-3-((6-Bromo-3H- imidazo[4,5-b]pyridin-2- yl)amino)cyclopentyl)amino)-2H-[1,3′- bipyridin]-2-one |
| 55 | 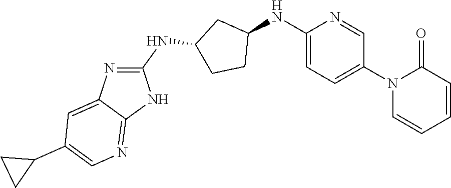 [SMILES: O=c1ccccn1-c1ccc(N[C@H]2CC[C@H](Nc3nc4cc(C5CC5)cnc4[nH]3)C2)nc1] | 6′-(((1S,3S)-3-((6-Cyclopropyl-3H- imidazo[4,5-b]pyridin-2- yl)amino)cyclopentyl)amino)-2H-[1,3′- bipyridin]-2-one |
| 71 | 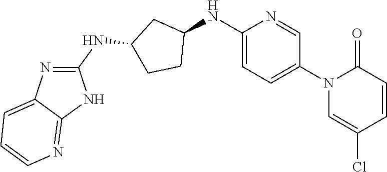 [SMILES: O=c1ccc(Cl)cn1-c1ccc(N[C@H]2CC[C@H](Nc3nc4cccnc4[nH]3)C2)nc1] | 6′-(((1S,3S)-3-((3H-Imidazo[4,5- b]pyridin-2-yl)amino)cyclopentyl)amino)- 5-chloro-2H-[1,3′-bipyridin]-2-one |
| 44 | 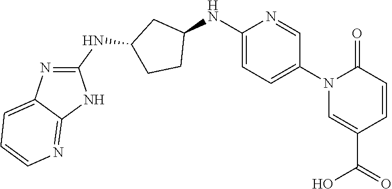 [SMILES: O=C(O)c1ccc(=O)n(-c2ccc(N[C@H]3CC[C@H](Nc4nc5cccnc5[nH]4)C3)nc2)c1] | 6′-(((1S,3S)-3-((3H-Imidazo[4,5- b]pyridin-2-yl)amino)cyclopentyl)amino)- 2-oxo-2H-[1,3′-bipyridine]-5-carboxylic acid |
| 56 | 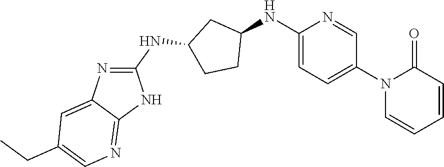 [SMILES: CCc1cnc2[nH]c(N[C@H]3CC[C@H](Nc4ccc(-n5ccccc5=O)cn4)C3)nc2c1] | 6′-(((1S,3S)-3-((6-Ethyl-3H-imidazo[4,5- b]pyridin-2-yl)amino)cyclopentyl)amino)- 2H-[1,3′-bipyridin]-2-one |
| 57 | 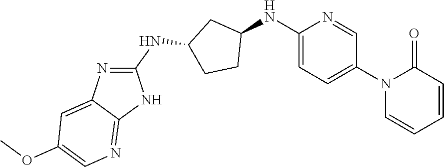 [SMILES: COc1cnc2[nH]c(N[C@H]3CC[C@H](Nc4ccc(-n5ccccc5=O)cn4)C3)nc2c1] | 6′-(((1S,3S)-3-((6-Methoxy-3H- imidazo[4,5-b]pyridin-2- yl)amino)cyclopentyl)amino)-2H-[1,3′- bipyridin]-2-one |
| 37 | 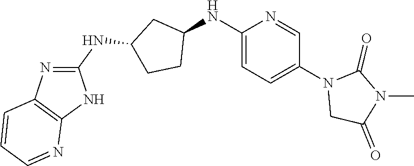 [SMILES: CN1C(=O)CN(c2ccc(N[C@H]3CC[C@H](Nc4nc5cccnc5[nH]4)C3)nc2)C1=O] | 1-(6-(((1S,3S)-3-((3H-Imidazo[4,5- b]pyridin-2- yl)amino)cyclopentyl)amino)pyridin-3-yl)- 3-methylimidazolidine-2,4-dione |
| 72 | 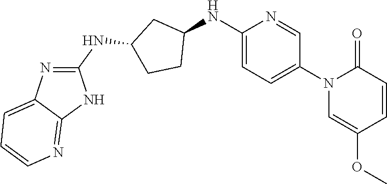 [SMILES: COc1ccc(=O)n(-c2ccc(N[C@H]3CC[C@H](Nc4nc5cccnc5[nH]4)C3)nc2)c1] | 6′-(((1S,3S)-3-((3H-Imidazo[4,5- b]pyridin-2-yl)amino)cyclopentyl)amino)- 5-methoxy-2H-[1,3′-bipyridin]-2-one |
| 35 | 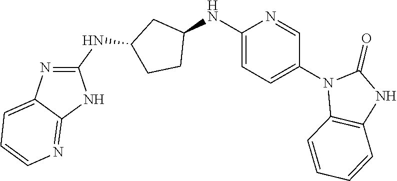 [SMILES: O=c1[nH]c2ccccc2n1-c1ccc(N[C@H]2CC[C@H](Nc3nc4cccnc4[nH]3)C2)nc1] | 1-(6-(((1S,3S)-3-((3H-Imidazo[4,5- b]pyridin-2- yl)amino)cyclopentyl)amino)pyridin-3-yl)- 1,3-dihydro-2H-benzo[d]imidazol-2-one |
| 60 | 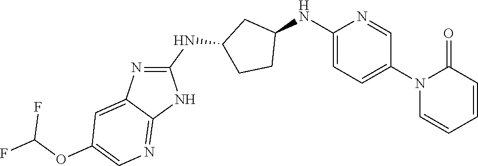 [SMILES: O=c1ccccn1-c1ccc(N[C@H]2CC[C@H](Nc3nc4cc(OC(F)F)cnc4[nH]3)C2)nc1] | 6′-(((1S,3S)-3-((6-(Difluoromethoxy)-3H- imidazo[4,5-b]pyridin-2- yl)amino)cyclopentyl)amino)-2H-[1,3′- bipyridin]-2-one |
| 58 | 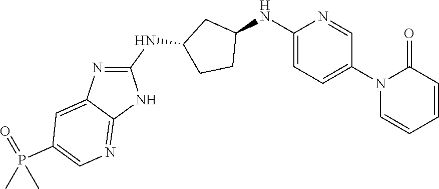 [SMILES: CP(C)(=O)c1cnc2[nH]c(N[C@H]3CC[C@H](Nc4ccc(-n5ccccc5=O)cn4)C3)nc2c1] | 6′-(((1S,3S)-3-((6-(Dimethylphosphoryl)- 3H-imidazo[4,5-b]pyridin-2- yl)amino)cyclopentyl)amino)-2H-[1,3′- bipyridin]-2-one |
| 41 | 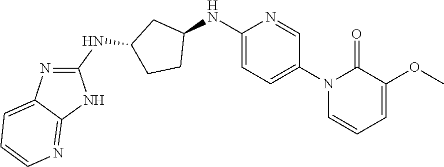 [SMILES: COc1cccn(-c2ccc(N[C@H]3CC[C@H](Nc4nc5cccnc5[nH]4)C3)nc2)c1=O] | 6′-(((1S,3S)-3-((3H-Imidazo[4,5- b]pyridin-2-yl)amino)cyclopentyl)amino)- 3-methoxy-2H-[1,3′-bipyridin]-2-one |
| 59 | 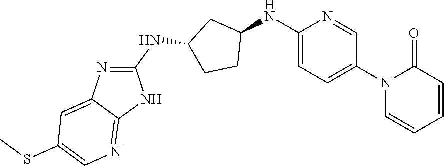 [SMILES: CSc1cnc2[nH]c(N[C@H]3CC[C@H](Nc4ccc(-n5ccccc5=O)cn4)C3)nc2c1] | 6′-(((1S,3S)-3-((6-(Methylthio)-3H- imidazo[4,5-b]pyridin-2- yl)amino)cyclopentyl)amino)-2H-[1,3′- bipyridin]-2-one |
| 65 | 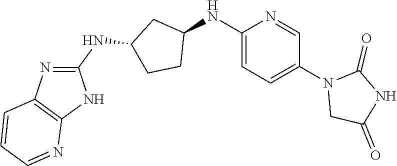 [SMILES: O=C1CN(c2ccc(N[C@H]3CC[C@H](Nc4nc5cccnc5[nH]4)C3)nc2)C(=O)N1] | 1-(6-(((1S,3S)-3-((3H-Imidazo[4,5- b]pyridin-2- yl)amino)cyclopentyl)amino)pyridin-3- yl)imidazolidine-2,4-dione |
| 42 | 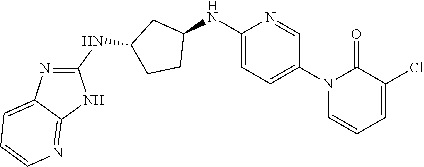 [SMILES: O=c1c(Cl)cccn1-c1ccc(N[C@H]2CC[C@H](Nc3nc4cccnc4[nH]3)C2)nc1] | 6′-(((1S,3S)-3-((3H-Imidazo[4,5- b]pyridin-2-yl)amino)cyclopentyl)amino)- 3-chloro-2H-[1,3′-bipyridin]-2-one |
| 45 | 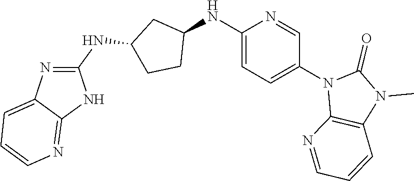 [SMILES: Cn1c(=O)n(-c2ccc(N[C@H]3CC[C@H](Nc4nc5cccnc5[nH]4)C3)nc2)c2ncccc21] | 3-(6-(((1S,3S)-3-((3H-Imidazo[4,5- b]pyridin-2- yl)amino)cyclopentyl)amino)pyridin-3-yl)- 1-methyl-1,3-dihydro-2H-imidazo[4,5- b]pyridin-2-one |
| 38 | 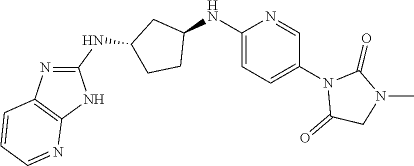 [SMILES: CN1CC(=O)N(c2ccc(N[C@H]3CC[C@H](Nc4nc5cccnc5[nH]4)C3)nc2)C1=O] | 3-(6-(((1S,3S)-3-((3H-Imidazo[4,5- b]pyridin-2- yl)amino)cyclopentyl)amino)pyridin-3-yl)- 1-methylimidazolidine-2,4-dione |
| 43 | 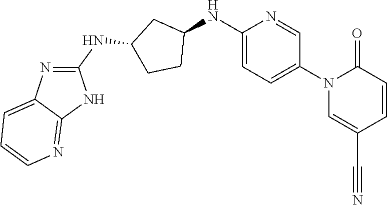 [SMILES: N#Cc1ccc(=O)n(-c2ccc(N[C@H]3CC[C@H](Nc4nc5cccnc5[nH]4)C3)nc2)c1] | 6′-(((1S,3S)-3-((3H-Imidazo[4,5- b]pyridin-2-yl)amino)cyclopentyl)amino)- 2-oxo-2H-[1,3′-bipyridine]-5-carbonitrile |
| 52 | 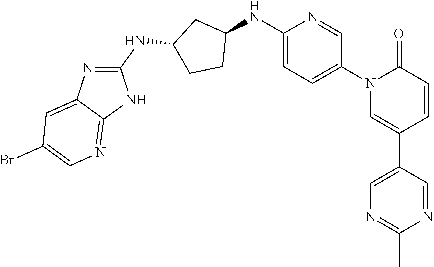 [SMILES: Cc1ncc(-c2ccc(=O)n(-c3ccc(N[C@H]4CC[C@H](Nc5nc6cc(Br)cnc6[nH]5)C4)nc3)c2)cn1] | 6′-(((1S,3S)-3-((6-Bromo-3H- imidazo[4,5-b]pyridin-2- yl)amino)cyclopentyl)amino)-5-(2- methylpyrimidin-5-yl)-2H-[1,3′-bipyridin]- 2-one |
| 53 | 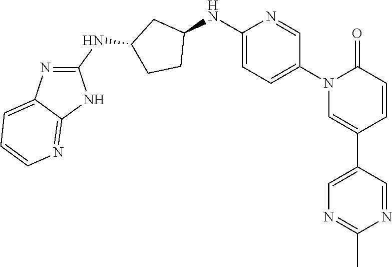 [SMILES: Cc1ncc(-c2ccc(=O)n(-c3ccc(N[C@H]4CC[C@H](Nc5nc6cccnc6[nH]5)C4)nc3)c2)cn1] | 6′-(((1S,3S)-3-((3H-Imidazo[4,5- b]pyridin-2-yl)amino)cyclopentyl)amino)- 5-(2-methylpyrimidin-5-yl)-2H-[1,3′- bipyridin]-2-one |
| 28 | 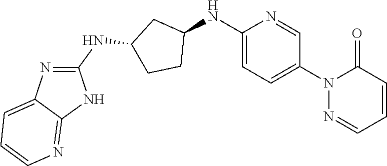 [SMILES: O=c1cccnn1-c1ccc(N[C@H]2CC[C@H](Nc3nc4cccnc4[nH]3)C2)nc1] | 2-(6-(((1S,3S)-3-((1H-Imidazo[4,5- b]pyridin-2- yl)amino)cyclopentyl)amino)pyridin-3- yl)pyridazin-3(2H)-one |
| 39 | 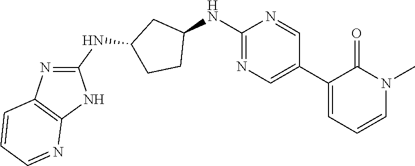 [SMILES: Cn1cccc(-c2cnc(N[C@H]3CC[C@H](Nc4nc5cccnc5[nH]4)C3)nc2)c1=O] | 3-(2-(((1S,3S)-3-((3H-Imidazo[4,5- b]pyridin-2- yl)amino)cyclopentyl)amino)pyrimidin-5- yl)-1-methylpyridin-2(1H)-one |
| 51 | 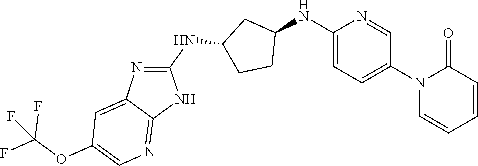 [SMILES: O=c1ccccn1-c1ccc(N[C@H]2CC[C@H](Nc3nc4cc(OC(F)(F)F)cnc4[nH]3)C2)nc1] | 6′-(((1S,3S)-3-((6-(Trifluoromethoxy)-3H- imidazo[4,5-b]pyridin-2- yl)amino)cyclopentyl)amino)-2H-[1,3′- bipyridin]-2-one |
| 75 | 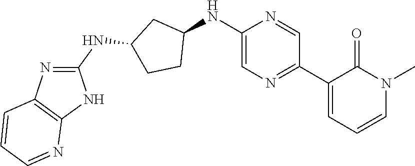 [SMILES: Cn1cccc(-c2cnc(N[C@H]3CC[C@H](Nc4nc5cccnc5[nH]4)C3)cn2)c1=O] | 3-(5-(((1S,3S)-3-((3H-Imidazo[4,5- b]pyridin-2- yl)amino)cyclopentyl)amino)pyrazin-2- yl)-1-methylpyridin-2(1H)-one |
| 67 | 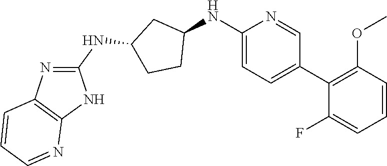 [SMILES: COc1cccc(F)c1-c1ccc(N[C@H]2CC[C@H](Nc3nc4cccnc4[nH]3)C2)nc1] | (1S,3S)-N1-(5-(2-Fluoro-6- methoxyphenyl)pyridin-2-yl)-N3-(3H- imidazo[4,5-b]pyridin-2-yl)cyclopentane- 1,3-diamine |
| 73 | 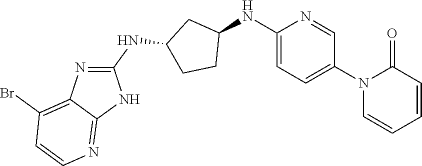 [SMILES: O=c1ccccn1-c1ccc(N[C@H]2CC[C@H](Nc3nc4c(Br)ccnc4[nH]3)C2)nc1] | 6′-(((1S,3S)-3-((7-Bromo-3H- imidazo[4,5-b]pyridin-2- yl)amino)cyclopentyl)amino)-2H-[1,3′- bipyridin]-2-one |
| 27 | 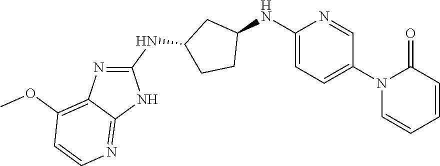 [SMILES: COc1ccnc2[nH]c(N[C@H]3CC[C@H](Nc4ccc(-n5ccccc5=O)cn4)C3)nc12] | 6′-(((1S,3S)-3-((7-Methoxy-3H- imidazo[4,5-b]pyridin-2- yl)amino)cyclopentyl)amino)-2H-[1,3′- bipyridin]-2-one |
| 64 | 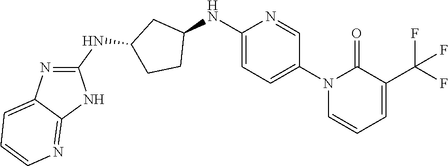 [SMILES: O=c1c(C(F)(F)F)cccn1-c1ccc(N[C@H]2CC[C@H](Nc3nc4cccnc4[nH]3)C2)nc1] | 6′-(((1S,3S)-3-((3H-Imidazo[4,5- b]pyridin-2-yl)amino)cyclopentyl)amino)- 3-(trifluoromethyl)-2H-[1,3′-bipyridin]-2- one |
| 63 | 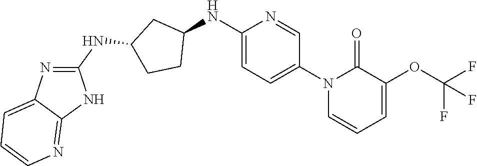 [SMILES: O=c1c(OC(F)(F)F)cccn1-c1ccc(N[C@H]2CC[C@H](Nc3nc4cccnc4[nH]3)C2)nc1] | 6′-(((1S,3S)-3-((3H-Imidazo[4,5- b]pyridin-2-yl)amino)cyclopentyl)amino)- 3-(trifluoromethoxy)-2H-[1,3′-bipyridin]-2- one |
| 68 | 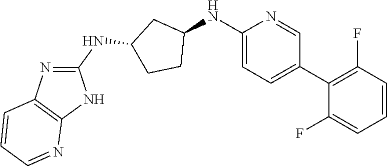 [SMILES: Fc1cccc(F)c1-c1ccc(N[C@H]2CC[C@H](Nc3nc4cccnc4[nH]3)C2)nc1] | (1S,3S)-N1-(5-(2,6- Difluorophenyl)pyridin-2-yl)-N3-(3H- imidazo[4,5-b]pyridin-2-yl)cyclopentane- 1,3-diamine |
| 66 | 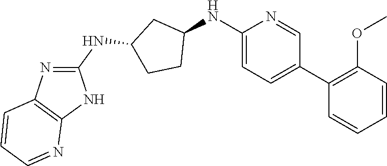 [SMILES: COc1ccccc1-c1ccc(N[C@H]2CC[C@H](Nc3nc4cccnc4[nH]3)C2)nc1] | (1S,3S)-N1-(3H-Imidazo[4,5-b]pyridin-2- yl)-N3-(5-(2-methoxyphenyl)pyridin-2- yl)cyclopentane-1,3-diamine |
| 29 | 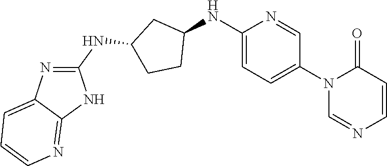 [SMILES: O=c1ccncn1-c1ccc(N[C@H]2CC[C@H](Nc3nc4cccnc4[nH]3)C2)nc1] | 3-(6-(((1S,3S)-3-((3H-Imidazo[4,5- b]pyridin-2- yl)amino)cyclopentyl)amino)pyridin-3- yl)pyrimidin-4(3H)-one |
| 80 | 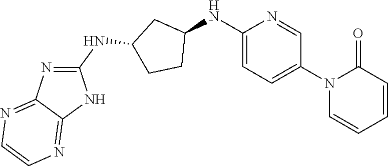 [SMILES: O=c1ccccn1-c1ccc(N[C@H]2CC[C@H](Nc3nc4nccnc4[nH]3)C2)nc1] | 6′-(((1S,3S)-3-((1H-Imidazo[4,5- b]pyrazin-2-yl)amino)cyclopentyl)amino)- 2H-[1,3′-bipyridin]-2-one |
| 61 | 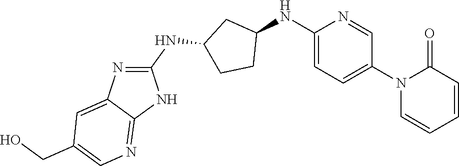 [SMILES: O=c1ccccn1-c1ccc(N[C@H]2CC[C@H](Nc3nc4cc(CO)cnc4[nH]3)C2)nc1] | 6′-(((1S,3S)-3-((6-(Hydroxymethyl)-3H- imidazo[4,5-b]pyridin-2- yl)amino)cyclopentyl)amino)-2H-[1,3′- bipyridin]-2-one |
| 31 | 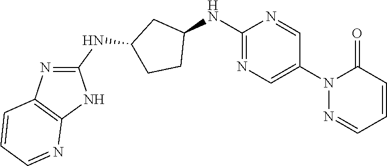 [SMILES: O=c1cccnn1-c1cnc(N[C@H]2CC[C@H](Nc3nc4cccnc4[nH]3)C2)nc1] | 2-(2-(((1S,3S)-3-((3H-Imidazo[4,5- b]pyridin-2- yl)amino)cyclopentyl)amino)pyrimidin-5- yl)pyridazin-3(2H)-one |
| 74 | 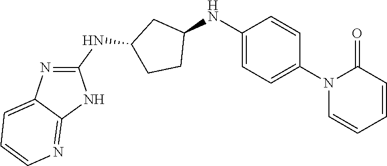 [SMILES: O=c1ccccn1-c1ccc(N[C@H]2CC[C@H](Nc3nc4cccnc4[nH]3)C2)cc1] | 1-(4-(((1S,3S)-3-((3H-Imidazo[4,5- b]pyridin-2- yl)amino)cyclopentyl)amino)phenyl)pyridin- 2(1H)-one |
| 11 | 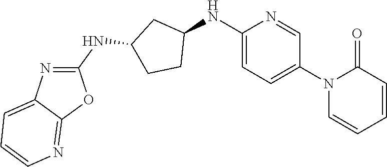 [SMILES: O=c1ccccn1-c1ccc(N[C@H]2CC[C@H](Nc3nc4cccnc4o3)C2)nc1] | 6′-(((1S,3S)-3-(Oxazolo[5,4-b]pyridin-2- ylamino)cyclopentyl)amino)-2H-[1,3′- bipyridin]-2-one |
| 19 | 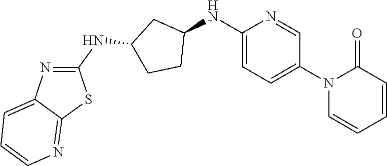 [SMILES: O=c1ccccn1-c1ccc(N[C@H]2CC[C@H](Nc3nc4cccnc4s3)C2)nc1] | 6′-(((1S,3S)-3-(Thiazolo[5,4-b]pyridin-2- ylamino)cyclopentyl)amino)-2H-[1,3′- bipyridin]-2-one |
| 69 | 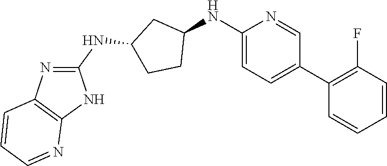 [SMILES: Fc1ccccc1-c1ccc(N[C@H]2CC[C@H](Nc3nc4cccnc4[nH]3)C2)nc1] | (1S,3S)-N1-(5-(2-Fluorophenyl)pyridin-2- yl)-N3-(3H-imidazo[4,5-b]pyridin-2- yl)cyclopentane-1,3-diamine |
| 23 | 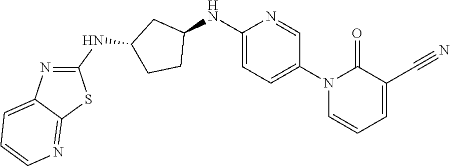 [SMILES: N#Cc1cccn(-c2ccc(N[C@H]3CC[C@H](Nc4nc5cccnc5s4)C3)nc2)c1=O] | 2-Oxo-6′-(((1S,3S)-3-(thiazolo[5,4- b]pyridin-2-ylamino)cyclopentyl)amino)- 2H-[1,3′-bipyridine]-3-carbonitrile |
| 24 | 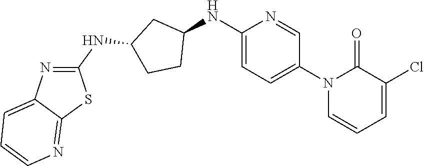 [SMILES: O=c1c(Cl)cccn1-c1ccc(N[C@H]2CC[C@H](Nc3nc4cccnc4s3)C2)nc1] | 3-Chloro-6′-(((1S,3S)-3-(thiazolo[5,4- b]pyridin-2-ylamino)cyclopentyl)amino)- 2H-[1,3′-bipyridin]-2-one |
| 15 | 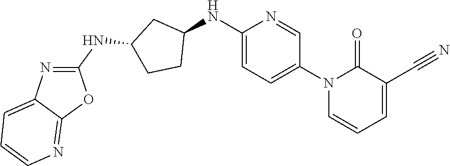 [SMILES: N#Cc1cccn(-c2ccc(N[C@H]3CC[C@H](Nc4nc5cccnc5o4)C3)nc2)c1=O] | 6′-(((1S,3S)-3-(Oxazolo[5,4-b]pyridin-2- ylamino)cyclopentyl)amino)-2-oxo-2H- [1,3′-bipyridine]-3-carbonitrile |
| 21 | 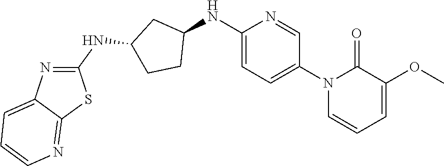 [SMILES: COc1cccn(-c2ccc(N[C@H]3CC[C@H](Nc4nc5cccnc5s4)C3)nc2)c1=O] | 3-Methoxy-6′-(((1S,3S)-3-(thiazolo[5,4- b]pyridin-2-ylamino)cyclopentyl)amino)- 2H-[1,3′-bipyridin]-2-one |
| 13 | 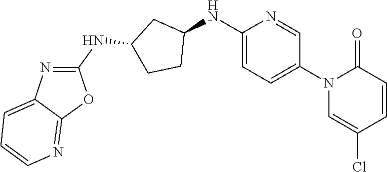 [SMILES: O=c1ccc(Cl)cn1-c1ccc(N[C@H]2CC[C@H](Nc3nc4cccnc4o3)C2)nc1] | 5-Chloro-6′-(((1S,3S)-3-(oxazolo[5,4- b]pyridin-2-ylamino)cyclopentyl)amino)- 2H-[1,3′-bipyridin]-2-one |
| 14 | 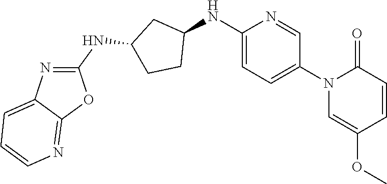 [SMILES: COc1ccc(=O)n(-c2ccc(N[C@H]3CC[C@H](Nc4nc5cccnc5o4)C3)nc2)c1] | 5-Methoxy-6′-(((1S,3S)-3-(oxazolo[5,4- b]pyridin-2-ylamino)cyclopentyl)amino)- 2H-[1,3′-bipyridin]-2-one |
| 18 | 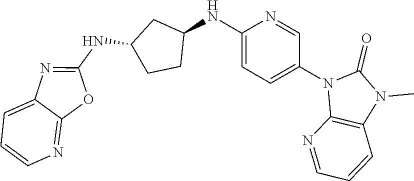 [SMILES: Cn1c(=O)n(-c2ccc(N[C@H]3CC[C@H](Nc4nc5cccnc5o4)C3)nc2)c2ncccc21] | 1-Methyl-3-(6-(((1S,3S)-3-(oxazolo[5,4- b]pyridin-2- ylamino)cyclopentyl)amino)pyridin-3-yl)- 1,3-dihydro-2H-imidazo[4,5-b]pyridin-2- one |
| 25 | 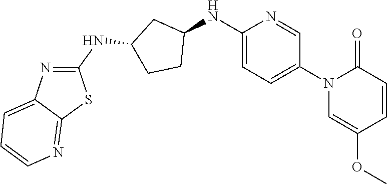 [SMILES: COc1ccc(=O)n(-c2ccc(N[C@H]3CC[C@H](Nc4nc5cccnc5s4)C3)nc2)c1] | 5-Methoxy-6′-(((1S,3S)-3-(thiazolo[5,4- b]pyridin-2-ylamino)cyclopentyl)amino)- 2H-[1,3′-bipyridin]-2-one |
| 12 | 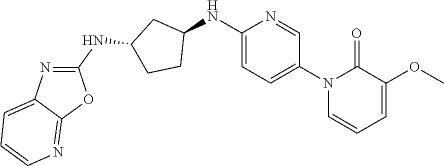 [SMILES: COc1cccn(-c2ccc(N[C@H]3CC[C@H](Nc4nc5cccnc5o4)C3)nc2)c1=O] | 3-Methoxy-6′-(((1S,3S)-3-(oxazolo[5,4- b]pyridin-2-ylamino)cyclopentyl)amino)- 2H-[1,3′-bipyridin]-2-one |
| 16 | 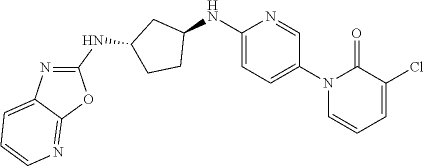 [SMILES: O=c1c(Cl)cccn1-c1ccc(N[C@H]2CC[C@H](Nc3nc4cccnc4o3)C2)nc1] | 3-Chloro-6′-(((1S,3S)-3-(oxazolo[5,4- b]pyridin-2-ylamino)cyclopentyl)amino)- 2H-[1,3′-bipyridin]-2-one |
| 32 | 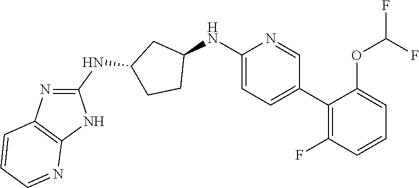 [SMILES: Fc1cccc(OC(F)F)c1-c1ccc(N[C@H]2CC[C@H](Nc3nc4cccnc4[nH]3)C2)nc1] | (1S,3S)-N1-(5-(2-(Difluoromethoxy)-6- fluorophenyl)pyridin-2-yl)-N3-(3H- imidazo[4,5-b]pyridin-2-yl)cyclopentane- 1,3-diamine |
| 26 | 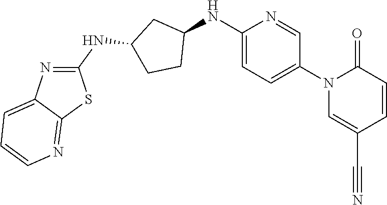 [SMILES: N#Cc1ccc(=O)n(-c2ccc(N[C@H]3CC[C@H](Nc4nc5cccnc5s4)C3)nc2)c1] | 2-Oxo-6′-(((1S,3S)-3-(thiazolo[5,4- b]pyridin-2-ylamino)cyclopentyl)amino)- 2H-[1,3′-bipyridine]-5-carbonitrile |
| 17 | 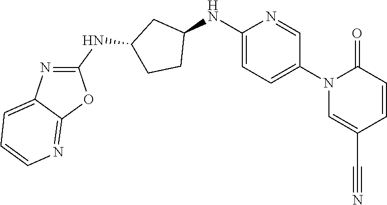 [SMILES: N#Cc1ccc(=O)n(-c2ccc(N[C@H]3CC[C@H](Nc4nc5cccnc5o4)C3)nc2)c1] | 6′-(((1S,3S)-3-(Oxazolo[5,4-b]pyridin-2- ylamino)cyclopentyl)amino)-2-oxo-2H- [1,3′-bipyridine]-5-carbonitrile |
| 22 | 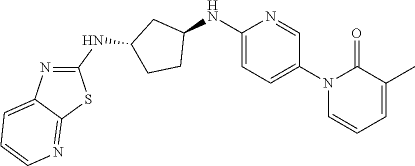 [SMILES: Cc1cccn(-c2ccc(N[C@H]3CC[C@H](Nc4nc5cccnc5s4)C3)nc2)c1=O] | 3-Methyl-6′-(((1S,3S)-3-(thiazolo[5,4- b]pyridin-2-ylamino)cyclopentyl)amino)- 2H-[1,3′-bipyridin]-2-one |
| 20 | 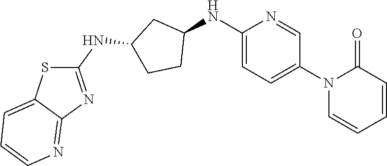 [SMILES: O=c1ccccn1-c1ccc(N[C@H]2CC[C@H](Nc3nc4ncccc4s3)C2)nc1] | 6′-(((1S,3S)-3-(Thiazolo[4,5-b]pyridin-2- ylamino)cyclopentyl)amino)-2H-[1,3′- bipyridin]-2-one |
| 5 | 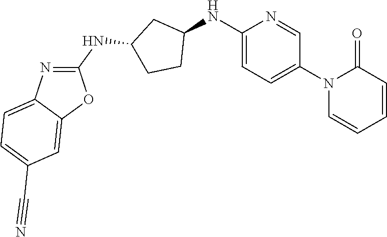 [SMILES: N#Cc1ccc2nc(N[C@H]3CC[C@H](Nc4ccc(-n5ccccc5=O)cn4)C3)oc2c1] | 2-(((1S,3S)-3-((2-Oxo-2H-[1,3′-bipyridin]- 6′-yl)amino)cyclopentyl) amino)benzo[d]oxazole- 6-carbonitrile |
| 30 | 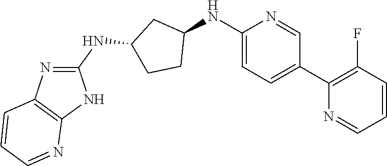 [SMILES: Fc1cccnc1-c1ccc(N[C@H]2CC[C@H](Nc3nc4cccnc4[nH]3)C2)nc1] | (1S,3S)-N1-(3-Fluoro-[2,3′-bipyridin]-6′- yl)-N3-(3H-imidazo[4,5-b]pyridin-2- yl)cyclopentane-1,3-diamine |
| 70 | 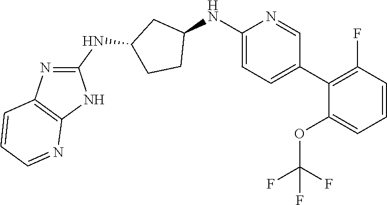 [SMILES: Fc1cccc(OC(F)(F)F)c1-c1ccc(N[C@H]2CC[C@H](Nc3nc4cccnc4[nH]3)C2)nc1] | (1S,3S)-N1-(5-(2-Fluoro-6- (trifluoromethoxy)phenyl)pyridin-2-yl)- N3-(3H-imidazo[4,5-b]pyridin-2- yl)cyclopentane-1,3-diamine |
| 76 | 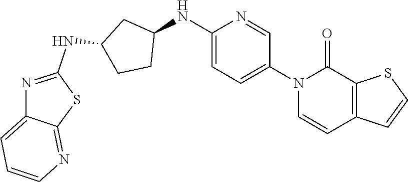 [SMILES: O=c1c2sccc2ccn1-c1ccc(N[C@H]2CC[C@H](Nc3nc4cccnc4s3)C2)nc1] | 6-(6-(((1S,3S)-3-(Thiazolo[5,4-b]pyridin- 2-ylamino)cyclopentyl)amino)pyridin-3- yl)thieno[2,3-c]pyridin-7(6H)-one |
| 77 | 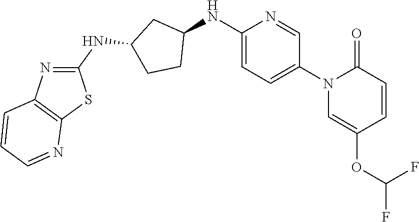 [SMILES: O=c1ccc(OC(F)F)cn1-c1ccc(N[C@H]2CC[C@H](Nc3nc4cccnc4s3)C2)nc1] | 5-(Difluoromethoxy)-6′-(((1S,3S)-3- (thiazolo[5,4-b]pyridin-2- ylamino)cyclopentyl)amino)-2H-[1,3′- bipyridin]-2-one |
| 78 | 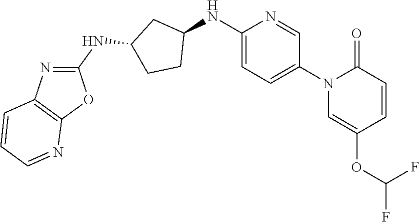 [SMILES: O=c1ccc(OC(F)F)cn1-c1ccc(N[C@H]2CC[C@H](Nc3nc4cccnc4o3)C2)nc1] | 5-(Difluoromethoxy)-6′-(((1S,3S)-3- (oxazolo[5,4-b]pyridin-2- ylamino)cyclopentyl)amino)-2H-[1,3′- bipyridin]-2-one |
| 79 | 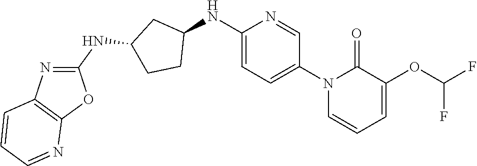 [SMILES: O=c1c(OC(F)F)cccn1-c1ccc(N[C@H]2CC[C@H](Nc3nc4cccnc4o3)C2)nc1] | 3-(Difluoromethoxy)-6′-(((1S,3S)-3- (oxazolo[5,4-b]pyridin-2- ylamino)cyclopentyl)amino)-2H-[1,3′- bipyridin]-2-one |
| 81 | 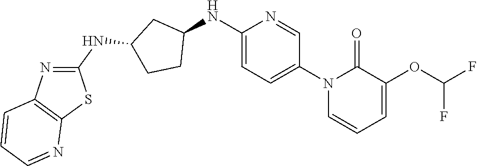 [SMILES: O=c1c(OC(F)F)cccn1-c1ccc(N[C@H]2CC[C@H](Nc3nc4cccnc4s3)C2)nc1] | 3-(Difluoromethoxy)-6′-(((1S,3S)-3- (thiazolo[5,4-b]pyridin-2- ylamino)cyclopentyl)amino)-2H-[1,3′- bipyridin]-2-one |
| 82 | 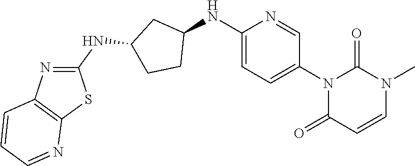 [SMILES: Cn1ccc(=O)n(-c2ccc(N[C@H]3CC[C@H](Nc4nc5cccnc5s4)C3)nc2)c1=O] | 1-Methyl-3-(6-(((1S,3S)-3-(thiazolo[5,4- b]pyridin-2- ylamino)cyclopentyl)amino)pyridin-3- yl)pyrimidine-2,4(1H,3H)-dione |
| 83 | 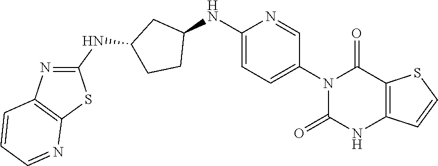 [SMILES: O=c1[nH]c2ccsc2c(=O)n1-c1ccc(N[C@H]2CC[C@H](Nc3nc4cccnc4s3)C2)nc1] | 3-(6-(((1S,3S)-3-(Thiazolo[5,4-b]pyridin- 2-ylamino)cyclopentyl)amino)pyridin-3- yl)thieno[3,2-d]pyrimidine-2,4(1H,3H)- dione |
| 84 | 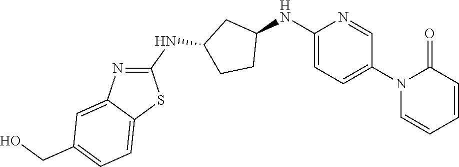 [SMILES: O=c1ccccn1-c1ccc(N[C@H]2CC[C@H](Nc3nc4cc(CO)ccc4s3)C2)nc1] | 6′-(((1S,3S)-3-((5- (Hydroxymethyl)benzo[d]thiazol-2- yl)amino)cyclopentyl)amino)-2H-[1,3′- bipyridin]-2-one |
| 85 | 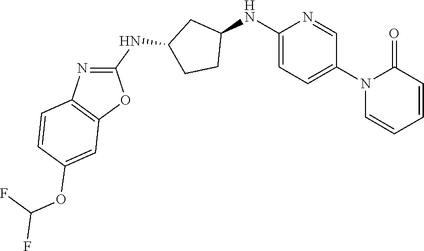 [SMILES: O=c1ccccn1-c1ccc(N[C@H]2CC[C@H](Nc3nc4ccc(OC(F)F)cc4o3)C2)nc1] | 6′-(((1S,3S)-3-((6- (Difluoromethoxy)benzo[d]oxazol-2- yl)amino)cyclopentyl)amino)-2H-[1,3′- bipyridin]-2-one |
| 86 | 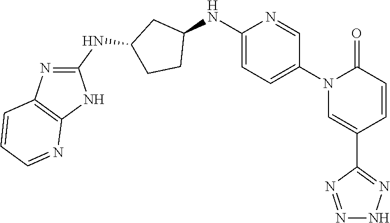 [SMILES: O=c1ccc(-c2nn[nH]n2)cn1-c1ccc(N[C@H]2CC[C@H](Nc3nc4cccnc4[nH]3)C2)nc1] | 6′-(((1S,3S)-3-((3H-Imidazo[4,5- b]pyridin-2-yl)amino)cyclopentyl)amino)- 5-(2H-tetrazol-5-yl)-2H-[1,3′-bipyridin]-2- one |
| 87 | 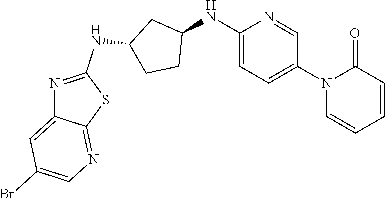 [SMILES: O=c1ccccn1-c1ccc(N[C@H]2CC[C@H](Nc3nc4cc(Br)cnc4s3)C2)nc1] | 6′-(((1S,3S)-3-((6-Bromothiazolo[5,4- b]pyridin-2-yl)amino)cyclopentyl)amino)- 2H-[1,3′-bipyridin]-2-one |
| 88 | 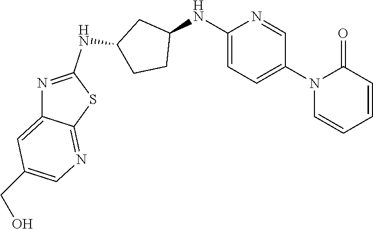 [SMILES: O=c1ccccn1-c1ccc(N[C@H]2CC[C@H](Nc3nc4cc(CO)cnc4s3)C2)nc1] | 6′-(((1S,3S)-3-((6- (Hydroxymethyl)thiazolo[5,4-b]pyridin-2- yl)amino)cyclopentyl)amino)-2H-[1,3′- bipyridin]-2-one |
| 89 | 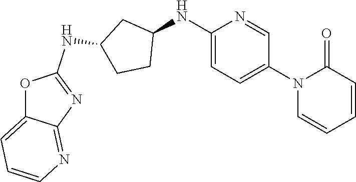 [SMILES: O=c1ccccn1-c1ccc(N[C@H]2CC[C@H](Nc3nc4ncccc4o3)C2)nc1] | 6′-(((1S,3S)-3-(Oxazolo[4,5-b]pyridin-2- ylamino)cyclopentyl)amino)-2H-[1,3′- bipyridin]-2-one |

In some embodiments the compound is selected from 73, 15, 21, 12, 82 and 83.

### General Synthesis

The compounds of general formula (I-B) can be prepared according to the following schemes 1, 2, 3 and 4. The schemes and procedures described below illustrate synthetic routes to the compounds of general formula (I-B) and are not intended to be limiting. It is understood that the order of transformations as exemplified in schemes 1, 2, 3 and 4 can be modified in various ways. The order of transformations exemplified in these schemes is therefore not intended to be limiting.

Routes for the preparation of compounds of general formula (I-B) and corresponding intermediates are described in schemes 1, 2, 3 and 4.

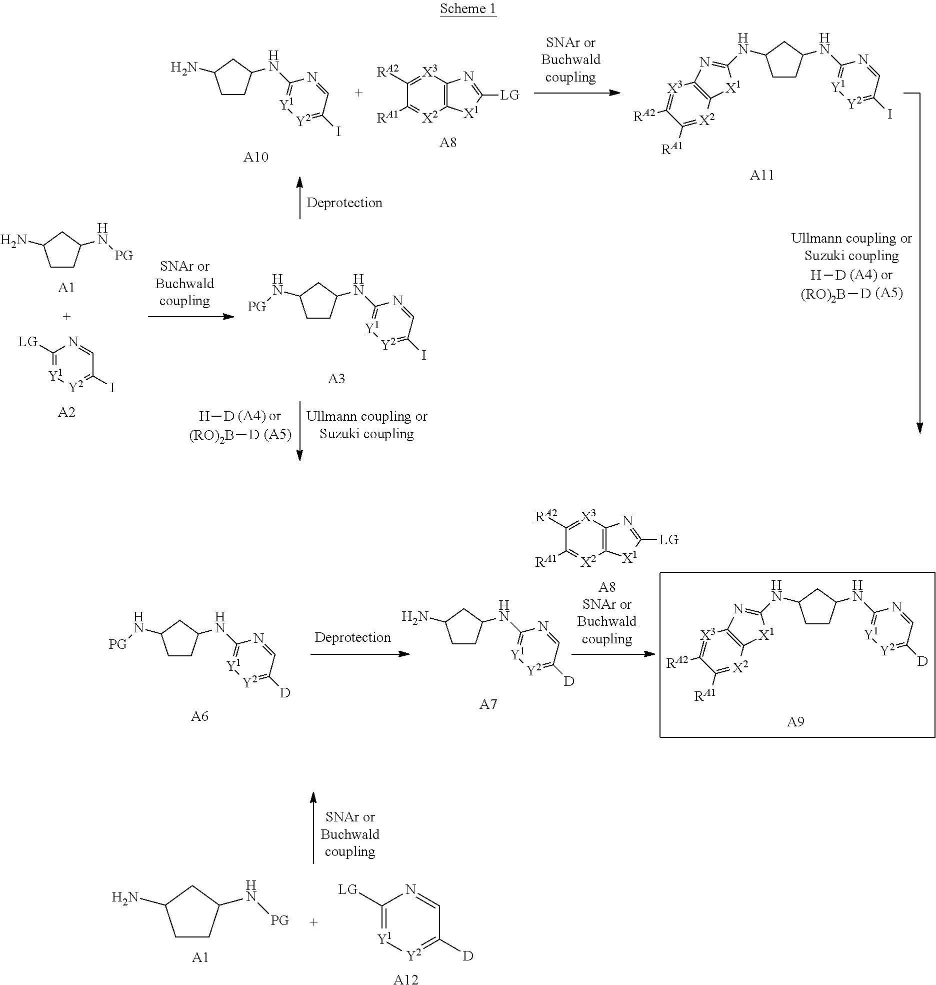

Scheme 1: Routes for the preparation of compounds of general formula (A9) are described in the scheme in which LG is a leaving group, PG is a protective group, Y¹ and Y² are CH or N (provided that when Y¹ is N, Y² is CH and vice versa) and D, X¹, X², X³, R^{A1} and R^{A2} have the meaning as given for general formula (I-B), supra.

Monoarylated diamines of general formula (A3) can be obtained via nucleophilic aromatic substitution (S_{N}Ar) or palladium catalyzed Buchwald-Hartwig amination between monoprotected diamines (A1), or their corresponding salts, and heteroaryls (A2) with LG being a leaving group like halogen, e.g. fluorine, chlorine or bromine, or —S(O)₁₋₂Me as depicted in Scheme 1. For S_{N}Ar approaches with LG being groups like for example fluorine, chlorine or —S(O)Me, diamines (A1) may be reacted with (A2) in the presence of inorganic bases like K₂CO₃, Na₂CO₃ or CS₂CO₃ or in the presence of organic bases like TEA or DIPEA, or without any additional base in polar solvents such as for example DMSO, NMP, nBuOH or 1,4-dioxane at temperatures between 100-130° C. The reaction times may vary between 1 h and 24 h. In certain instances, it can be beneficial to apply microwave heating.

For palladium catalyzed Buchwald-Hartwig aminations all methods that are known in the art may be applied. For example, diamines (A1) may be reacted with (A2) in the presence of a palladium catalyst like Pd PEPPSI-IpentCl [CAS 1612891-29-8], Pd₂(dba)₃, tBuXPhos Pd G3 [1447963-75-8] or tBuBrettPhos G3 and a base like Cs₂CO₃, NaOtBu or MTBD in aprotic solvents like 1,4-dioxane, DMF, toluene, NMP or DMA at temperatures between room temperature and 130° C., preferably at 65-100° C., for 15-24 h.

Diamines of general formula (A1) and heteroaryls of general formula (A2) are either commercially available or can be prepared according to procedures available from the public domain. For the synthesis of diamines (A1) see for example WO2004004726 and references therein.

Arylated diamines of general formula (A6) can be obtained from (A3) via copper catalyzed Ullmann couplings with heterocycles (A4) or via palladium catalyzed Suzuki couplings with boronic acid derivatives (A5). For Ullmann couplings all methods that are known in the art may be applied. For example, (A3) may be reacted with (A4) in the presence of a copper catalyst like Cu(I)I, Cu(OTf)₂ or Cu(OAc)₂ and a base like Cs₂CO₃ or K₂CO₃ in polar, aprotic solvents like 1,4-dioxane, DMF or pyridine at temperatures between room temperature and 120° C., preferably at 100° C. for 15-20 h. In some instances a ligand like DMCDA, TMEDA, N¹,N²-dimethylethane-1,2-diamine or N,N-dimethylglycine might be added to the reaction mixture.

For Suzuki couplings towards (A6) all methods that are known in the art may be applied. For example, (A3) may be reacted with boronic acid derivatives (A5) in the presence of a palladium catalyst like Pd(dtbpf)Cl₂ [CAS 95408-45-0] or Pd(dppf)Cl₂ [CAS 72287-26-4] and a base like Cs₂CO₃, K₂CO₃ or K₃PO₄ in polar solvents such as 1,4-dioxane, THF and water or mixtures thereof at temperatures between room temperature and 120° C. for 2-15 h.

Heterocycles of general formula (A4) and boronic acid derivatives of general formula (A5) are either commercially available or can be prepared according to procedures available from the public domain.

Primary amines of general formula (A7) can be obtained from monoprotected diamines of general formula (A6) via deprotection methods. Depending on the protective group applied these can be for example acidic, basic, oxidative or hydrogenation methods. Appropriate protective moieties for amino groups and their introduction and cleavage are well-known in the art. For an overview of protective group chemistry see for example P. G. M. Wuts, T. W. Greene, Greene's Protective Groups in Organic Synthesis 4^{th} ed., J. Wiley & Sons, 2006.

Final compounds of general formula (A9) can be synthesized from primary amines of general formula (A7) via S_{N}Ar or palladium catalyzed Buchwald-Hartwig amination. Primary amines of general formula (A7) can be reacted with heteroaryls of general formula (A8) with LG being a leaving group like halogen, such as chlorine or bromine, or —S(O)₂Me applying procedures in analogy to those described for the synthesis of (A3) from (A1) and (A2) in Scheme 1.

Heteroaryls of general formula (A8) are either commercially available or can be prepared according to procedures available from the public domain.

An alternative route to compounds of general formula (A9) starts with deprotection of diamines of general formula (A3) to give primary amines of general formula (A10) as depicted in Scheme 1. For deprotection the same procedures apply as described for the synthesis of (A7) from (A6).

Primary amines of general formula (A10) in turn can be reacted with heteroaryls of general formula (A8) via S_{N}Ar or palladium catalyzed Buchwald-Hartwig amination to give aryl iodides of general formula (A11) applying procedures in analogy to those described for the synthesis of (A3) from (A1) and (A2) in Scheme 1.

Final compounds of general formula (A9) can be synthesized from aryl iodides of general formula (A11) via copper catalyzed Ullmann couplings with heterocycles H-D (A4) or via palladium catalyzed Suzuki couplings with boronic acid derivatives (A5) applying procedures in analogy to those described for the synthesis of compounds (A6) from (A3) in Scheme 1.

Yet another approach to compounds of general formula (A9) starts from monoprotected diamines (A1) or their corresponding salts and preassembled heteroaryls (A12) with LG being a leaving group like halogen, e.g. fluorine, chlorine or bromine, or —S(O)₁₋₂Me via S_{N}Ar or palladium catalyzed Buchwald-Hartwig amination to give arylated diamines of general formula (A6). The procedures that can be applied are in analogy to those described for the synthesis of (A3) from (A1) and (A2) in Scheme 1. Heteroaryls of general formula (A12) are either commercially available or can be prepared according to procedures available from the public domain (for example via Chan-Lam coupling, see for example Adv. Synth. Catal. 2020, 362, 3311-3331).

An alternative route for the preparation of compounds of general formula (A9) and intermediates of general formula (A11) is depicted in Scheme 2.

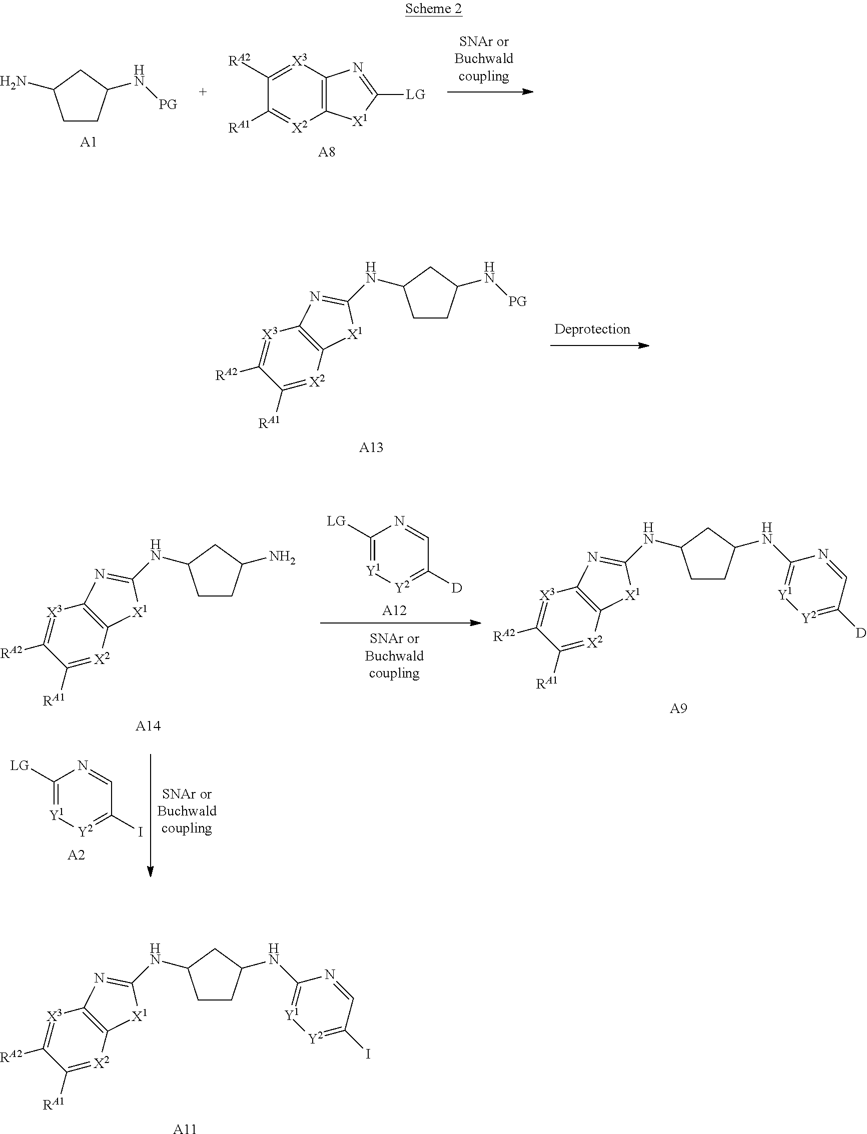

Scheme 2: Routes for the preparation of compounds of general formula (A9) and intermediates (A11) are described in the scheme in which LG is a leaving group, PG is a protective group, Y¹ and Y² are CH or N (provided that when Y¹ is N, Y² is CH and vice versa) and D, X¹, X², X³, R^{A1} and R^{A2} have the meaning as given for general formula (I-B), supra.

Monoarylated diamines of general formula (A13) can be obtained via S_{N}Ar or palladium catalyzed Buchwald-Hartwig amination between monoprotected diamines (A1) or their corresponding salts and heteroaryls (A8) with LG being a leaving group like halogen, such as chlorine or bromine, or —S(O)₂Me. The procedures that can be applied are in analogy to those described for the synthesis of (A3) from (A1) and (A2) in Scheme 1.

Deprotection of diamines of general formula (A13) can give primary amines of general formula (A14). For deprotection the same procedures apply as described for the synthesis of (A7) from (A6) in Scheme 1.

Final compounds of general formula (A9) in turn can be synthesized from primary amines (A14) or their corresponding salts and preassembled heteroaryls (A12) with LG being a leaving group like halogen, e.g. fluorine, chlorine or bromine, or —S(O)₁₋₂Me via S_{N}Ar or palladium catalyzed Buchwald-Hartwig amination. The procedures that can be applied are in analogy to those described for the synthesis of (A6) from (A1) and (A12) in Scheme 1.

For the synthesis of intermediates of general formula (A11) primary amines (A14) or their corresponding salts may be reacted with heteroaryls (A2) with LG being a leaving group like halogen, e.g. fluorine, chlorine or bromine, or —S(O)₁₋₂Me in an S_{N}Ar or palladium catalyzed Buchwald-Hartwig amination. The procedures that can be applied are in analogy to those described for the synthesis of (A3) from (A1) and (A2) in Scheme 1.

Final compounds of general formula (A17) may be synthesized according to the routes depicted in Scheme 3. Primary amines of general formula (A7) or their corresponding salts (prepared according to Scheme 1) can be reacted with one carbon equivalents like CDI or TCDI in the presence of inorganic bases like sodium hydroxide or in the presence of organic bases like TEA or DIPEA, or without any additional base in polar, aprotic solvents like DMF at temperatures between rt and the boiling point of the solvent, preferably at 100° C. for 1-2 h to give an acylimidazole intermediate. This intermediate may be reacted in situ with 1,2-dianilines (A15) in the presence of a carbodiimide reagent like EDC at temperatures between rt and the boiling point of the solvent, preferably at 100° C. for 15-24 h to give (A17).

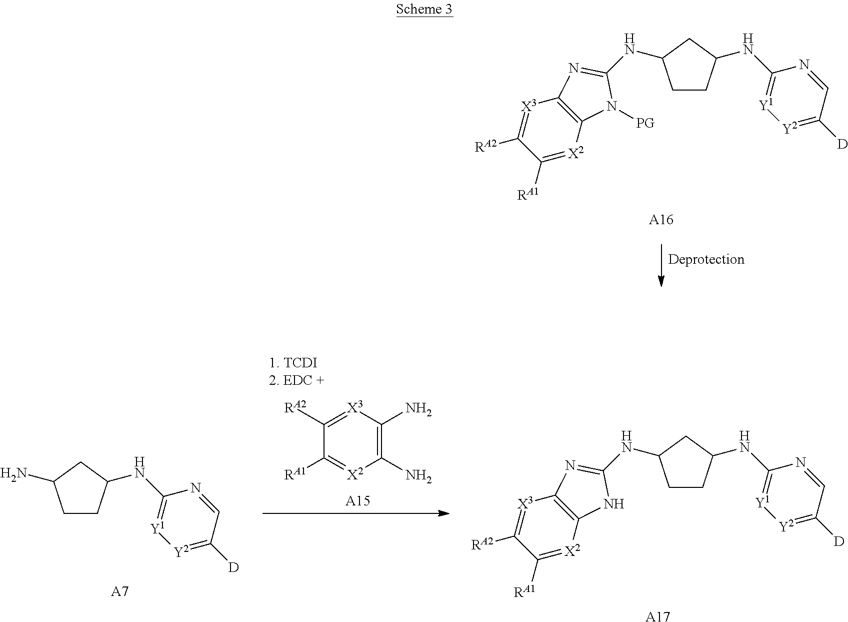

Scheme 3: Routes for the preparation of compounds of general formula (A17) in which Y¹ and Y² are CH or N (provided that when Y¹ is N, Y² is CH and vice versa) and D, X², X³, R^{A1} and R^{A2} have the meaning as given for general formula (I-B1), supra.

Alternatively, protected intermediates of general formula (A16), prepared according to the routes depicted in Schemes 1 or 2, may be deprotected to give compounds of general formula (A17). Depending on the protective group applied these can be for example acidic, basic, oxidative or hydrogenation methods. Suitable protective groups may be groups such as paramethoxybenzyl (PMB), 4-methylbenzenesulfonyl (Ts) or benzyl (Bn). Deprotection of a PMB group for example could be achieved by reaction with acids such as TFA in solvents like DCM, or without any additional solvent, at temperatures between rt and the boiling point of the solvent, preferably at 60-100° C. for 15 min to 18 h. Deprotection of a Ts group may be performed by reaction with a base such as K₂CO₃ or Na₂CO₃ in polar, protic solvent such as MeOH or EtOH at temperatures between rt and the boiling point of the solvent, preferably at 60° C. for 1-4 h.

Yet another approach to compounds of general formula (A9) is depicted in Scheme 4. Aryl bromides of general formula (A18), prepared according to the procedures depicted in Schemes 1-3, can be functionalized under metal or metallaphotoredox catalysis (see for example Chem. Rev. 2022, 122, 1485-1542), e.g. via late-stage functionalisation, with nucleophiles of general formula (A19) (see for example Angew. Chem. Int. Ed. 2003, 42, 5400-5449) or boronic acid derivatives (A20) (see for example Chem. Rev. 1995, 95, 2457-2483) or stannanes (A21) (see for example ACS Catal. 2015, 5, 3040-3053) to give final compounds of general formula (A9). For metal or metallaphotoredox catalysis all methods that are known in the art may be applied.

Nucleophiles of general formula (A19) and boronic acid derivatives of general formula (A20) and stannanes (A21) are either commercially available or can be prepared according to procedures available from the public domain.

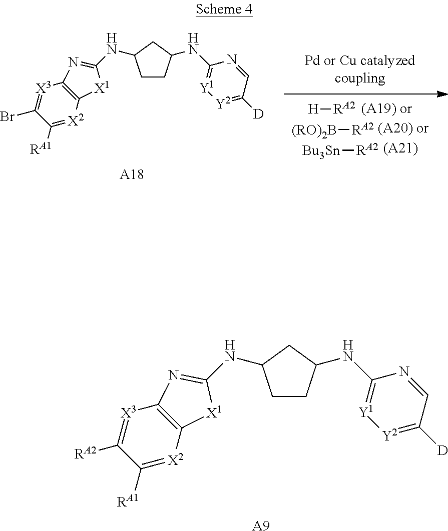

Scheme 4: Route for the preparation of compounds of general formula (A9) in which Y¹ and Y² are CH or N (provided that when Y¹ is N, Y² is CH and vice versa) and D, X¹, X², X³, R^{A1} and R^{A2} have the meaning as given for general formula (I-B), supra (provided that R^{A2} is not halogen). The compounds of general formula (B-2) can be prepared according to the following schemes 5 and 6. The schemes and procedures described below illustrate synthetic routes to the compounds of general formula (B-2) and are not intended to be limiting. It is understood that the order of transformations as exemplified in schemes 5 and 6 can be modified in various ways. The order of transformations exemplified in these schemes is therefore not intended to be limiting.

Routes for the preparation of compounds of general formula (B-2) and corresponding intermediates are described in schemes 5 and 6.

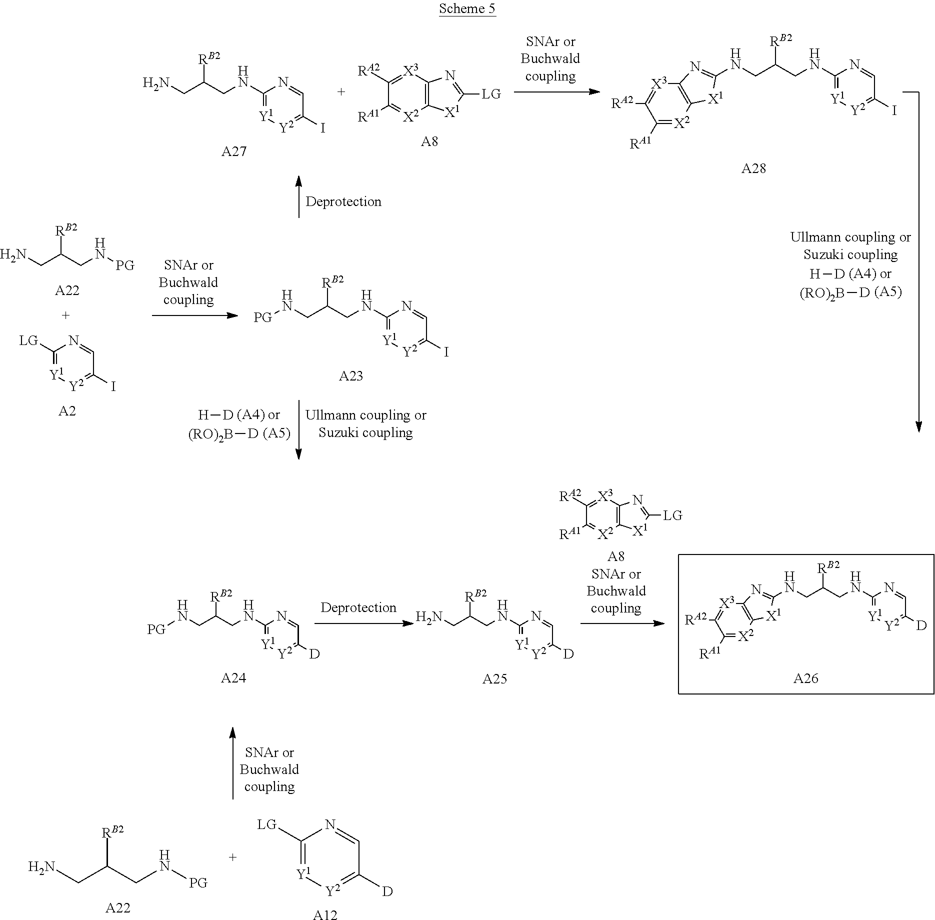

Scheme 5: Routes for the preparation of compounds of general formula (A26) are described in the scheme in which LG is a leaving group, PG is a protective group, Y¹ and Y² are CH or N (provided that when Y¹ is N, Y² is CH and vice versa) and D, X¹, X², X³, R^{A1}, R^{A2} and R^{B2} have the meaning as given for general formula (B-2), supra.

For the synthesis of final compounds of general formula (A26) routes and methods comparable to the ones described in Schemes 1-4 may be applied. Without being intended to be limiting the routes to compounds of general formula (A26) in Schemes 5-6 are given for further illustration. Diamines of general formula (A22) are either commercially available or can be prepared according to procedures available from the public domain.

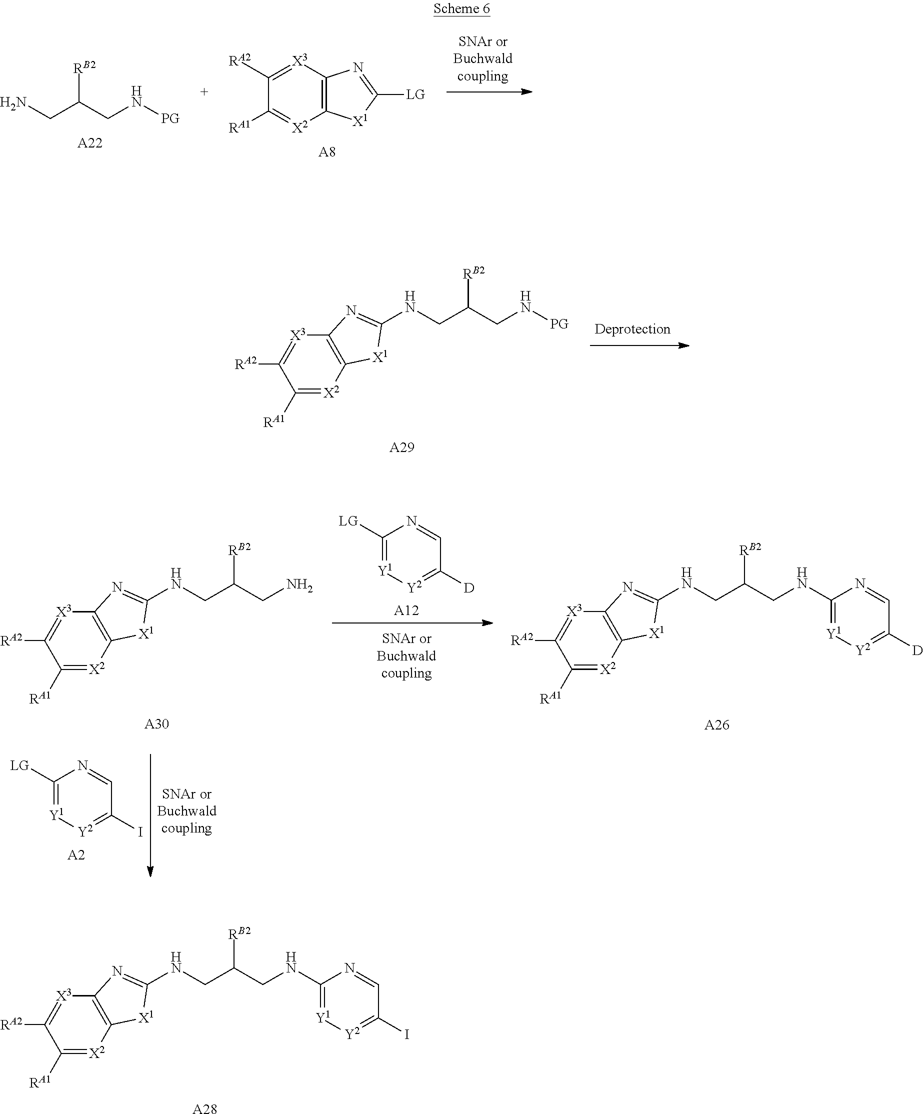

Scheme 6: Routes for the preparation of compounds of general formula (A26) and intermediates (A28) are described in the scheme in which LG is a leaving group, PG is a protective group, Y¹ and Y² are CH or N (provided that when Y¹ is N, Y² is CH and vice versa) and D, X¹, X², X³, R^{A1}, R^{A2} and R^{B2} have the meaning as given for general formula (B-2), supra.

Further compounds with different formulae as described above can be prepared by similar methods.

Persons skilled in the art will appreciate that in order to obtain compounds of the disclosure in an alternative, and on some occasions, more convenient, manner, the individual process steps mentioned hereinbefore may be performed in different order, and/or the individual reactions may be performed at a different stage in the overall route (i.e. substituents may be added to and/or chemical transformations performed upon different intermediates to those mentioned hereinbefore in conjunction with a particular reaction). This may negate, or render necessary, the need for protecting groups.

Persons skilled in the art will appreciate that chiral isomers of compounds herein can be resolved at any stage in the synthetic process using chiral resolving agents described in the literature and known to person skilled in the art, or using chiral chromatography methods described in the literature and known to person skilled in the art. Stereo centers may also be introduced by asymmetric synthesis. All stereoisomers are included within the scope of the disclosure.

Persons skilled in the art will appreciate that starting materials for any of the above processes can in some cases be commercially available.

Persons skilled in the art will appreciate that processes for some starting materials above could be found in the general common knowledge.

It will also be understood that some of the compounds described in the processes above may exhibit the phenomenon of tautomerism and the processes described above include any tautomeric form.

All novel intermediates form a further aspect of the disclosure.

### EXPERIMENTAL SECTION

NMR peak forms are stated as they appear in the spectra, possible higher order effects have not been considered.

The following table lists the abbreviations used in this paragraph and in the examples section as far as they are not explained within the text body. Other abbreviations have their meanings customary per se to the skilled person.

### Abbreviations

AcOH Acetic acid
aq Aqueous
Boc tert-Butoxycarbonyl
Brine Saturated aqueous sodium chloride solution
Bu Butyl
tBu tert-Butyl
tBuBrettPhos Di-tert-butyl(2′,4′,6′-triisopropyl-3,6-dimethoxy-[1,1′-biphenyl]-2-yl)phosphine
n-BuOH 1-Butanol
tBuXPhos Pd G3 [(2-Di-tert-butylphosphino-2′,4′,6′-triisopropyl-1,1′-biphenyl)-2-(2′-amino-1,1′-biphenyl)] palladium(II) methanesulfonate (CAS Registry Number 1447963-75-8)
calcd Calculated
CataCXium A Di(adamantan-1-yl)(butyl)phosphane (CAS Registry number 321921-71-5)
CataCXium A Pd G3 Methanesulfonato(diadamantyl-n-butylphosphino)-2′-amino-1,1′-biphenyl-2-yl)palladium(II) precatalyst generation 3
CDI 1,1-Carbonyldiimidazole
CHAPS 3-[(3-Cholamidopropyl)dimethylammonio]-1-propanesulfonate
m-CPBA 3-Chlorobenzoperoxoic acid
DCM Dichloromethane
DCE Dichloroethane
DCM Dichloromethane
DIPEA N-Ethyl-N-isopropyl-propan-2-amine
DMA N,N-Dimethylacetamide
DMAP N,N-Dimethylpyridin-4-amine
DMCDA rac-(1R,2R)—N¹,N²-Dimethylcyclohexane-1,2-diamine
DME 1,2-Dimethoxyethane
DMF N,N-Dimethylformamide
DMSO Dimethyl sulfoxide
Dppf 1,1′-Bis(diphenylphosphino)ferrocene
EDC 3-(((Ethylimino)methylene)amino)-N,N-dimethylpropan-1-amine hydrochloride
eq Equivalent(s)
ESI Electrospray ionization
Et Ethyl
Et₂O Diethyl ether
EtOAc Ethyl acetate
EtOH Ethanol
FA Formic acid
(g) Gas
G2 Generation 2
G3 Generation 3
HATU (1-(Bis(dimethylamino)methylene]-1H-1,2,3-triazolo[4,5-b]pyridinium-3-oxo hexafluorophosphate
HEPES (4-(2-Hydroxyethyl)-1-piperazineethanesulfonic acid)
HPLC High performance liquid chromatography
HRMS High resolution mass spectrometry
IC₅₀ Half maximal inhibitory concentration
K_{D} Dissociation constant
LC Liquid chromatography
LG Leaving group
Me CH₃
MeCN Acetonitrile
MeOH Methanol
MS Mass spectrometry
MTBD 7-Methyl-1,5,7-triazabicyclo[4.4.0]dec-5-ene
MTBE Methyl tert-butyl ether
m/z Mass to charge ratio
NaOtBu Sodium tert-butoxide
NMP 1-Methylpyrrolidin-2-one
NMR Nuclear magnetic resonance
OAc O(CO)CH₃
OEt OCH₂CH₃
OTf Trifluoromethanesulfonate
OtBu OC(CH₃)₃
Pd/C Palladium on charcoal
Pd₂(dba)₃ Tris(dibenzylideneacetone)dipalladium(0)
Pd(dppf)Cl₂·DCM [1,1′-bis(diphenylphosphino)ferrocene]dichloropalladium(II) CH₂Cl₂ (1:1) Pd(dtbpf)Cl₂ [1,1′-Bis(di-tert-butylphosphino)ferrocene]dichloropalladium(II)
Pd(OAc)₂ Palladium (II) acetate
Pd-PEPPSI-IpentCl
2-methylpyridine (SP-4-1)-[1,3-Bis[2,6-bis(1-ethylpropyl)phenyl]-4,5-dichloro-1,3-dihydro-2H-imidazol-2-ylidene]dichloro(2-methylpyridine)palla-dium (CAS Registry Number 1612891-29-8)
Pd(PPh₃)₄ Pd[(C₆H₅)₃P]₄
PE Petroleum ether
PG Protective group
Ph Phenyl
PMB Paramethoxybenzyl
PPh₃ Triphenylphosphane
qToF Quadrupole time-of-flight
Rt Room temperature
(s) Solid
sat Saturated
SFC Supercritical fluid chromatography
S_{N}Ar Nucleophilic aromatic substitution
tBuBrettPhos G3 2-(Di-tert-butylphosphino)-2′,4′,6′-triisopropyl-3,6-dimethoxy-1,1′-biphenyl)-2-(2′-amino-1,1′-biphenyl)]palladium(II) methanesulfonate (CAS Registry Number: 1536473-72-9)
TCDI di(1H-imidazol-1-yl)methanethione
TEA triethylamine
TFA trifluoroacetic acid
TFAA 2,2,2-trifluoroacetic anhydride
THF tetrahydrofuran
TMEDA N¹,N¹,N²,N²-tetramethylethane-1,2-diamine
TLC thin layer chromatography
Ts Tosyl or 4-methylbenzenesulfonyl
UPLC ultra performance liquid chromatography
UV ultraviolet
XPhos dicyclohexyl(2′,4′,6′-triisopropyl-[1,1′-biphenyl]-2-yl)phosphane
XPhos Pd G2 Chloro(2-dicyclohexylphosphino-2′,4′,6′-triisopropyl-1,1′-biphenyl)[2-(2′-amino-1,1′-biphenyl)]palladium(II) (CAS Reg. No 1310584-14-5)
XantPhos 4,5-bis(diphenylphosphino)-9,9-dimethylxanthene, (9,9-dimethyl-9H-xanthene-4,5-diyl)bis(diphenylphosphine)

### Units

Å Ångström
atm atmosphere
C Celcius
g gram
h hour(s)
L litre
M mole per liter
mg milligram
MHz megahertz
min minute(s)
mL milliliter
mm millimeter
mol mole
mmol millimole(s)
ρCi microcurie
μm micrometer
μmol micromole(s)
μL microlitre
nm nanometer
ppm parts per million
v/v volume by volume
W watt
w/v weight by volume

The various embodiments described in this application are illustrated by the following examples which are not meant to limit the compound of Formula (I) in any way.

The example testing experiments described herein serve to illustrate the present embodiments and is not limited to the examples given.

### Experimental Section—General Part

### General Conditions

(i) operations were carried out at room temperature (rt), i.e. in the range 17 to 28° C. and where needed under an atmosphere of an inert gas such as N₂ or Ar; optionally reactions were carried out using a MBRAUN UNILab Plus ECO or a MBRAUN UNILab SP Eco glovebox workstation, in which case it is indicated;
(ii) where reactions refer to being degassed or purged, this can be performed for example by purging the reaction solvent with a constant flow of nitrogen for a suitable period of time (for example 5 to 10 min) or by repeatedly evacuating the vessel and backfill with appropriate inert atmosphere (for example N₂(g) or Ar(g));
(iii) where reactions refer to the use of a microwave reactor, one of the following microwave reactors were used: Biotage Initiator, Personal Chemistry Emrys Optimizer, Personal Chemistry Smith Creator or CEM Explorer;
(iv) in general, the course of reactions was followed by thin layer chromatography (TLC) and/or analytical high performance liquid chromatography (HPLC or UPLC) which was usually coupled to a mass spectrometer (LCMS).
(v) when necessary, organic solutions were dried over anhydrous MgSO₄ or Na₂SO₄, or by using ISOLUTE® Phase Separator, and work-up procedures were carried out using traditional phase separating techniques. When a drying agent such as e.g. MgSO₄ or Na₂SO₄ is used for drying an organic layer, it is understood that said organic layer is filtered before concentration of said layer.
(vi) it is understood that washing solutions used in the work-up procedures or reagent used for acidifying such as e.g. brine (sat aq NaCl solution), NaHCO₃, NH₄Cl, HCl, NaH₂PO₄ are presumed to be aqueous solutions unless otherwise stated;
(vii) evaporations were carried out either by rotary evaporation in vacuo or in a Genevac HT-4/EZ-2 or Biotage V10;
(viii) unless otherwise stated, flash column chromatography was performed on normal phase silica, using either Merck Silica Gel (Art. 9385) or prep-packed cartridges such as Biotage® SNAP cartridges (40-63 μm silica, 4-330 g), Biotage® Sfar Silica HC D cartridges (20 μm, 10-100 g), Interchim puriFlash™ cartridges (25 μm, 4-120 g), Interchim puriFlash™ cartridges (50 μm, 25-330 g), Grace™ GraceResolv™ Silica Flash Cartridges (4-120 g) or Agela Flash Colum Silica-CS cartridges (80-330 g), or on reversed phase silica using Agela Technologies C-18, spherical cartridges (20-35 μm, 100 A, 80-330 g), manually or automated using a Grace Reveleris® X2 Flash system or similar system;
(ix) purification using ion exchange columns were performed on standard ion exchange columns e.g. ISOLUTE SCX-2 columns from Biotage;
(x) preparative reversed phase HPLC and preparative reversed phase SFC were performed using standard HPLC and SFC instruments, respectively, equipped with either a MS and/or UV triggered fraction collecting instrument, using either isocratic or a gradient of the mobile phase as described in the experimental section and using one of the following methods:
PrepMethod A: The compound was purified by preparative HPLC on a YMC-Actus Triart C18 column (5 μm, 150×30 mm ID) using a gradient of MeCN in H₂O/NH₄HCO₃ (10 mM)/NH₃ (0.1%, aq) buffer system as mobile phase;
PrepMethod B: The compound was purified by preparative HPLC on a XBridge™ C18 column (10 μm, 250×19 mm ID) using a gradient of MeCN in H₂O/MeCN/NH₃ (95/5/0.2) as mobile phase;
PrepMethod C: The compound was purified by preparative HPLC on a XBridge™ C18 column (10 μm, 250×50 mm ID) using a gradient of MeCN in H₂O/MeCN/NH₃ (95/5/0.2) buffer system as mobile phase;
PrepMethod D: The compound was purified by preparative HPLC on a Kromasil C₈ column (10 μm, 250×50 mm ID) using a gradient of MeCN in H₂O/MeCN/FA (95/5/0.2) buffer system as mobile phase;
PrepMethod E: The compound was purified by preparative HPLC on a XBridge™ OBD C18 column (5 μm, 150×30 mm ID) using a gradient of MeCN in NH₃/H₂O (0.2%) as mobile phase;
PrepMethod F: The compound was purified by preparative HPLC on a XBridge™ C18 OBD column (5 μm, 150×30 mm ID) using a gradient of MeCN in a H₂O/NH₄HCO₃ (10 mM)/NH₃ (0.1%, aq) buffer system as mobile phase;
PrepMethod G: The compound was purified by preparative HPLC on a XBridge C18 OBD column (5 μm, 150×19 mm ID) using a gradient of MeCN in H₂O/FA (0.1%) buffer system as mobile phase;
PrepMethod H: The compound was purified by preparative HPLC on a XBridge™ Shield 018 column (5 μm, 150×30 mm ID) using a gradient of MeCN in a H₂O/NH₄HCO₃ (10 mM)/NH₃ (0.1%, aq) buffer system as mobile phase;
PrepMethod I: The compound was purified by preparative HPLC on a Waters™ Sunfire™ 018 OBD column (5 μm, 150×30 mm ID) using a gradient of MeCN in H₂O/FA (0.1%) as mobile phase;
PrepMethod J: The compound was purified by preparative HPLC on an Atlantis Prep T3 OBD column (10 μm, 250×19 mm ID) using a gradient of MeCN in H₂O/FA (0.1%) as mobile phase;
PrepMethod K: The compound was purified by preparative HPLC on a Waters Xselect CSH C18 OBD column (5 μm, 150×30 mm ID) using a gradient of MeCN in H₂O/FA (0.1%) buffer system as mobile phase;
PrepMethod L: The compound was purified by preparative HPLC on a XBridge C18 OBD column (5 μm, 150×19 mm ID) using a gradient of MeCN in H₂O/TFA (0.05%) buffer system as mobile phase;
PrepMethod M: The compound was purified by preparative HPLC on a XSelect CSH OBD column (5 μm, 150×30 mm ID) using a gradient of MeCN in H₂O/FA (0.1%) as mobile phase;
PrepMethod N: The compound was purified by preparative HPLC on a XSelect CSH OBD column (5 μm, 150×30 mm ID) using a gradient of MeCN in a H₂O/NH₄HCO₃ (10 mM)/NH₃ (0.05%, aq) buffer system as mobile phase;
PrepMethod O: The compound was purified by preparative HPLC on a XBridge™ OBD C18 column (5 μm, 150×30 mm ID) using a gradient of MeCN in NH₃/H₂O (0.05%) as mobile phase;
PrepMethod P: The compound was purified by preparative HPLC on a YMC-Actus Triart C18 column (3 μm, 75×30 mm ID) using a gradient of MeCN in H₂O/NH₄HCO₃ (10 mM)/NH₃ (0.05%, aq) buffer system as mobile phase;
PrepMethod Q: The compound was purified by preparative HPLC on a XBridge™ OBD Phenyl column (5 μm, 150×19 mm ID) using a gradient of MeOH in a H₂O/NH₄HCO₃ (10 mM)/NH₃ (0.1%, aq) buffer system as mobile phase;
PrepMethod R: The compound was purified by preparative HPLC on a XBridge™ OBD C18 column (5 μm, 150×30 mm ID) using a gradient of MeCN in H₂O as mobile phase;
PrepMethod S: The compound was purified by preparative HPLC on a YMC-Actus Triart C18 ExRS column (5 μm, 150×30 mm ID) using a gradient of MeCN in H₂O/NH₄HCO₃ (10 mM)/NH3 (0.1%, aq) as mobile phase;
PrepMethod T: The compound was purified by preparative HPLC on a XSelect CSH OBD column (5 μm, 100×30 mm ID) using a gradient of MeCN in H₂O/FA (0.1%) as mobile phase;
PrepMethod U: The compound was purified by preparative HPLC on an Atlantis Prep T3 OBD column (10 μm, 150×19 mm ID) using a gradient of MeCN in H2O (0.1% FA) buffer system as mobile phase;
PrepMethod V: The compound was purified by preparative HPLC on a Waters™ Sunfire™ 018 OBD column (5 μm, 150×30 mm ID) using a gradient of MeCN in H₂O/FA (10 mM) as mobile phase;
PrepMethod W: The compound was purified by preparative HPLC on a XBridge™ C18 OBD column (5 μm, 150×30 mm ID) using a gradient of MeCN in a H₂O/NH₄HCO₃ (10 mM)/NH₃ (0.05%, aq) buffer system as mobile phase;
PrepMethod X: The compound was purified by preparative HPLC on a XBridge™ C18 OBD column (5 μm, 150×30 mm ID) using a gradient of 20 mM NaOH+10% MeCN in a H₂O/NH₄HCO₃ (10 mM)/NH₃ (0.05%, aq) buffer system as mobile phase;

Relevant fractions were collected, combined, and freeze-dried or evaporated to give the purified compound or relevant fractions were collected, combined, and concentrated at reduced pressure, the aqueous layer was extracted with DCM or EtOAc, and the organic layer was dried, either over Na₂SO₄ or by using a phase-separator, and then concentrated at reduced pressure and when needed dried in vacuo, to give the purified compound.
(xi) yields, where present, are not necessarily the maximum attainable, and when necessary, reactions were repeated if a larger amount of the reaction product was required;
(xii) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data; where stated the salts were treated according to literature-known processes to generate the corresponding free base prior to being used;
(xiii) in general, the structures of the end-products of the Formula (I) were confirmed by NMR and/or mass spectral techniques; proton NMR chemical shift values were measured on the delta scale using Bruker Avance Ill 300, 400, 500 and 600 spectrometers, operating at ¹H frequencies of 300, 400, 500 and 600 MHz, respectively. The experiments were typically recorded at 25° C. Chemical shifts are given in ppm with the solvent as internal reference. Protons on heteroatoms such as NH and OH protons are only reported when detected in NMR and can therefore be missing. In certain instances, protons can be masked or partially masked by solvent peaks and will therefore either be missing and not reported or reported as multiplets overlapping with solvent. The following abbreviations have been used (and derivatives thereof), e.g. s, singlet; d, doublet; t, triplet; q, quartet; m, multiplet; br, broad; qn, quintet; p, pentet; h heptet; dd, doublet of doublets, etc. It is understood, where the NMR spectra contains residual impurities and/or residual solvent(s), this is not reported unless it partially coincides with peaks of intermediates and/or structures of Formula (I), in which case said peaks of intermediates and/or structures of Formula (I) are reported as multiplets partially overlapping with said solvent or impurity, and the integral is omitted. In some cases, compounds of Formula (I) appear as tautomers in the NMR-spectrum, in which instances only peaks of the major tautomer are reported.

In some cases, compounds of Formula (I) appear as tautomers in a more equal relationship, in such instances the peaks of such tautomers are either reported as multiplets, if the signals of said tautomer are partially overlapping with other peaks, or as individual peaks, if the signals of said tautomers are well separated. The integral of such peaks are reported as fractions of protons, indicating the ratio of the tautomer in the mixture.
(xiv) Electrospray mass spectral data were obtained using a Waters Acquity UPLC coupled to a Waters single quadrupole mass spectrometer or similar equipment, acquiring both positive and negative ion data, and generally, only ions relating to the parent structure are reported; high resolution electrospray mass spectral data were obtained using a Waters XEVO qToF mass spectrometer or similar equipment, coupled to a Waters Acquity UPLC, acquiring either positive and negative ion data, and generally, only ions relating to the parent structure are reported;
(xv) intermediates were not necessarily fully purified but their structures and purity were assessed by TLC, analytical HPLC/UPLC, and/or NMR analysis and/or mass spectrometry;
(xvi) in general Examples and intermediate compounds are named using ChemDraw Professional version 20.1.1.125 or version 21.0.0 from PerkinElmer. ChemDraw Professional version 20.1.1.125 or version 21.0.0 generates the names of chemical structures using the Cahn-Ingold-Prelog (CIP) rules for stereochemistry and follows IUPAC rules as closely as possible when generating chemical names. Stereoisomers are differentiated from each other by stereodescriptors cited in names and assigned in accordance with the CIP rules.

### INTERMEDIATES

### Intermediate 1

### tert-Butyl ((1S,3S)-3-((5-iodopyridin-2-yl)amino)cyclopentyl)carbamate

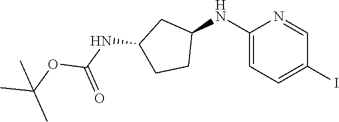 [SMILES: CC(C)(C)OC(=O)N[C@H]1CC[C@H](Nc2ccc(I)cn2)C1]

2-Fluoro-5-iodopyridine (2.23 g, 9.99 mmol) was added to tert-butyl ((1S,3S)-3-aminocyclopentyl)carbamate (2.00 g, 9.99 mmol) and K₂CO₃ (2.76 g, 20 mmol) in DMSO (30 mL). The resulting solution was stirred at 125° C. for 1 h under a nitrogen atmosphere. The reaction mixture was diluted with EtOAc (50 mL) and washed with water (3×75 mL). The organic layer was dried over Na₂SO₄, filtered and evaporated. The crude material was purified by flash chromatography on silica (gradient: 0-50% EtOAc in PE) to give the title compound (2.70 g, 67%) as a pale yellow solid; MS (ESI) m/z [M+H]⁺ 403.9.

### Intermediate 2

### tert-Butyl ((1S,3S)-3-((2-oxo-2H-[1,3′-bipyridin]-6′-yl)amino)cyclopentyl)carbamate

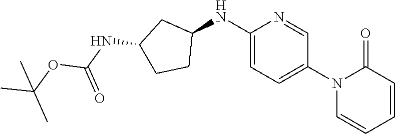 [SMILES: CC(C)(C)OC(=O)N[C@H]1CC[C@H](Nc2ccc(-n3ccccc3=O)cn2)C1]

rel-(1R,2R)—N¹,N²-Dimethylcyclohexane-1,2-diamine (0.212 g, 1.49 mmol) and Cu(I)I (0.283 g, 1.49 mmol) were added to tert-butyl ((1 S,3S)-3-((5-iodopyridin-2-yl)amino)cyclopentyl)carbamate Intermediate 1 (3.0 g, 7.44 mmol), K₂CO₃ (3.08 g, 22.3 mmol) and pyridin-2(1H)-one (1.42 g, 14.9 mmol) in 1,4-dioxane (20 mL). The resulting solution was stirred at 110° C. for 18 h under a nitrogen atmosphere. The reaction mixture was diluted with EtOAc (25 mL) and washed sequentially with water (3×25 mL). The organic layer was dried over Na₂SO₄, filtered and evaporated. The crude material was triturated with EtOAc:PE (5:1) to give a solid. The solid was collected by filtration and dried in vacuo to give the title compound (2.70 g, 98%) as a yellow solid; MS (ESI) m/z [M+H]⁺ 371.2.

### Intermediate 3

### 6′-(((1S,3S)-3-Aminocyclopentyl)amino)-2H-[1,3′-bipyridin]-2-one

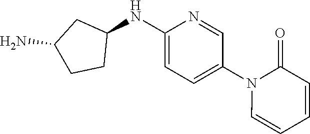 [SMILES: N[C@H]1CC[C@H](Nc2ccc(-n3ccccc3=O)cn2)C1]

2 M HCl in Et₂O (27 mL, 54 mmol) was added slowly to tert-butyl ((1 S,3S)-3-((2-oxo-2H-[1,3′-bipyridin]-6′-yl)amino)cyclopentyl)carbamate Intermediate 2 (1 g, 2.70 mmol) in DCM (10 mL) at 25° C. The resulting mixture was stirred at 25° C. for 3 h. This synthesis procedure was repeated for a second batch of tert-butyl ((1 S,3S)-3-((2-oxo-2H-[1,3′-bipyridin]-6′-yl)amino)cyclopentyl)carbamate Intermediate 2 (1.7 g, 4.6 mmol). The two batches were combined and concentrated. The crude product was recrystallised from EtOAc:PE (5:1) to give a solid which was collected by filtration and dried in vacuo to give an unspecified HCl salt of the title compound (2.5 g, 100%) as a yellow solid; MS (ESI) m/z [M+H]⁺ 270.9.

### Intermediate 4

### tert-Butyl ((1 S,3S)-3-((3-methoxy-2-oxo-2H-[1,3′-bipyridin]-6′-yl)amino)cyclopentyl)carbamate

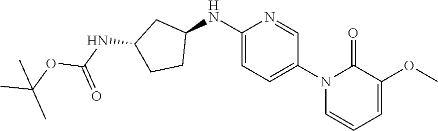 [SMILES: COc1cccn(-c2ccc(N[C@H]3CC[C@H](NC(=O)OC(C)(C)C)C3)nc2)c1=O]

tert-Butyl ((1 S,3S)-3-((5-iodopyridin-2-yl)amino)cyclopentyl)carbamate Intermediate 1 (500 mg, 1.24 mmol) was added to 3-methoxypyridin-2(1H)-one (776 mg, 6.20 mmol), DMCDA (176 mg, 1.24 mmol), Cs₂CO₃ (2.02 g, 6.20 mmol) and Cu(I)I (236 mg, 1.24 mmol) in 1,4-dioxane (5 mL). The resulting mixture was stirred at 100° C. for 15 h under a nitrogen atmosphere. The reaction mixture was diluted with water (150 mL). The aqueous layer was extracted with EtOAc (7×150 mL). The organic layers were dried over Na₂SO₄, filtered and evaporated. The residue was purified by preparative TLC (MeOH:DCM, 1:20) to give the title compound (387 mg, 78%) as a brown solid; MS (ESI) m/z [M+H]⁺ 401.3.

### Intermediate 5

### 6′-(((1S,3S)-3-Aminocyclopentyl)amino)-3-methoxy-2H-[1,3′-bipyridin]-2-one

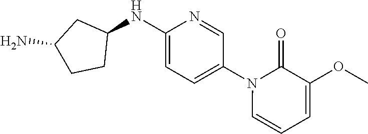 [SMILES: COc1cccn(-c2ccc(N[C@H]3CC[C@H](N)C3)nc2)c1=O]

TFA (2 mL, 25.96 mmol) was added to tert-butyl ((1S,3S)-3-((3-methoxy-2-oxo-2H-[1,3′-bipyridin]-6′-yl)amino)cyclopentyl)carbamate Intermediate 4 (310 mg, 0.77 mmol) in DCM (8 mL) at 30° C. The resulting mixture was stirred at 30° C. for 2 h. The solvent was removed under reduced pressure to give the TFA salt of the title compound (572 mg, 98%) as a brown gum; MS (ESI) m/z [M+H]⁺ 301.0.

### Intermediate 6

### tert-Butyl ((1S,3S)-3-((5-chloro-2-oxo-2H-[1,3′-bipyridin]-6′-yl)amino)cyclopentyl)carbamate

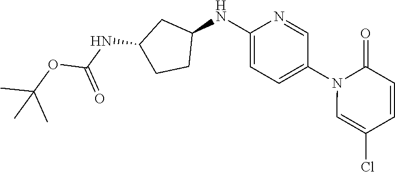 [SMILES: CC(C)(C)OC(=O)N[C@H]1CC[C@H](Nc2ccc(-n3cc(Cl)ccc3=O)cn2)C1]

DMCDA (106 mg, 0.74 mmol) was added to tert-butyl ((1S,3S)-3-((5-iodopyridin-2-yl)amino)cyclopentyl)carbamate Intermediate 1 (300 mg, 0.74 mmol), 5-chloropyridin-2(1H)-one (193 mg, 1.49 mmol), Cu(I)I (142 mg, 0.74 mmol) and Cs₂CO₃ (727 mg, 2.23 mmol) in 1,4-dioxane (25 mL) at 26° C. The resulting solution was stirred at 100° C. for 18 h. The reaction mixture was diluted with EtOAc (75 mL) and washed sequentially with water (3×25 mL) and sat brine (3×20 mL). The organic layer was dried over Na₂SO₄, filtered and evaporated. The residue was purified by preparative TLC (EtOAc:PE, 2:1) to give the title compound (298 mg, 99%) as a green solid; MS (ESI) m/z [M+H]⁺ 405.0.

### Intermediate 7

### 6′-(((1S,3S)-3-Aminocyclopentyl)amino)-5-chloro-2H-[1,3′-bipyridin]-2-one

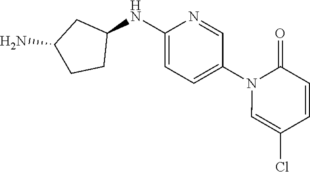 [SMILES: N[C@H]1CC[C@H](Nc2ccc(-n3cc(Cl)ccc3=O)cn2)C1]

TFA (5 mL, 64.90 mmol) was added to tert-butyl ((1 S,3S)-3-((5-chloro-2-oxo-2H-[1,3′-bipyridin]-6′-yl)amino)cyclopentyl)carbamate Intermediate 6 (220 mg, 0.54 mmol) in DCM (20 mL) at 25° C. The resulting solution was stirred at 100° C. for 18 h. The solvent was removed under reduced pressure to give the TFA salt of the crude title compound (432 mg, 91%) as a brown gum; MS (ESI) m/z [M+H]⁺ 304.9.

### Intermediate 8

### tert-Butyl ((1S,3S)-3-((5-methoxy-2-oxo-2H-[1,3′-bipyridin]-6′-yl)amino)cyclopentyl)carbamate

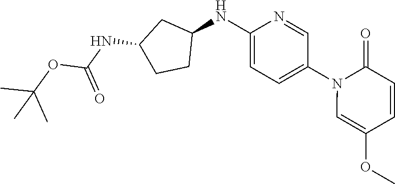 [SMILES: COc1ccc(=O)n(-c2ccc(N[C@H]3CC[C@H](NC(=O)OC(C)(C)C)C3)nc2)c1]

DMCDA (52.9 mg, 0.37 mmol) was added to tert-butyl ((1 S,3S)-3-((5-iodopyridin-2-yl)amino)cyclopentyl)carbamate Intermediate 1 (300 mg, 0.74 mmol), 5-methoxypyridin-2-ol (233 mg, 1.86 mmol), Cs₂CO₃ (727 mg, 2.23 mmol) and CuI (70.8 mg, 0.37 mmol) in 1,4-dioxane (15 mL) at 25° C. The resulting suspension was stirred at 100° C. for 18 h under a nitrogen atmosphere. The reaction mixture was diluted with EtOAc (50 mL) and washed sequentially with water (3×75 mL). The organic layer was dried over Na₂SO₄, filtered and evaporated. The residue was purified by preparative TLC (EtOAc) to give the title compound (263 mg, 88%) as a brown solid; MS (ESI) m/z [M+H]⁺ 401.0.

### Intermediate 9

### 6′-(((1S,3S)-3-Aminocyclopentyl)amino)-5-methoxy-2H-[1,3′-bipyridin]-2-one

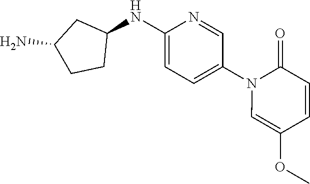 [SMILES: COc1ccc(=O)n(-c2ccc(N[C@H]3CC[C@H](N)C3)nc2)c1]

TFA (5 ml, 64.90 mmol) was added to tert-butyl ((1S,3S)-3-((5-methoxy-2-oxo-2H-[1,3′-bipyridin]-6′-yl)amino)cyclopentyl)carbamate Intermediate 8 (400 mg, 1.00 mmol) in DCM (15 mL) at 25° C. The resulting suspension was stirred at 25° C. for 18 h. The solvent was removed under reduced pressure to give the TFA salt of the crude title compound (1.1 g, 100%) as a black gum; MS (ESI) m/z [M+H]⁺ 301.0.

### Intermediate 10

### 6′-Chloro-2-oxo-2H-[1,3′-bipyridine]-3-carbonitrile

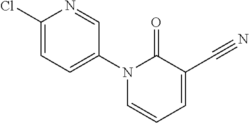 [SMILES: N#Cc1cccn(-c2ccc(Cl)nc2)c1=O]

Pyridine (0.808 mL, 9.99 mmol) was added to a mixture of 2-oxo-1,2-dihydropyridine-3-carbonitrile (600 mg, 5.00 mmol), (6-chloropyridin-3-yl)boronic acid (1572 mg, 9.99 mmol), Cu(OAc)₂ (1815 mg, 9.99 mmol) and 5 Å molecular sieves (500 mg, dried at 200° C. for 24 h) in DCM (100 mL) and DMF (15 mL) at 25° C. Air was allowed to diffuse into the reaction mixture via a drying tube containing CaCl₂(s). The resulting mixture was stirred at 25° C. for 15 h. The mixture was filtered through Celite. The filter cake was washed with DCM (3×10 mL) and the filtrate was concentrated under reduced pressure. The residue was partitioned between EtOAc (400 mL) and water (150 mL). The aqueous layer was extracted with EtOAc (5×200 mL). The organic layers were combined and washed with sat brine (5×100 mL), dried over Na₂SO₄, filtered and evaporated. The dry solid was triturated with DMF (3×5 mL) and the filter cake was washed with MTBE (3×5 mL). The solid was dried in vacuo to give the title compound (105 mg, 9%) as a grey solid; MS (ESI) m/z [M+H]⁺ 232.1.

### Intermediate 11

### tert-Butyl ((1S,3S)-3-((3-cyano-2-oxo-2H-[1,3′-bipyridin]-6′-yl)amino)cyclopentyl)carbamate

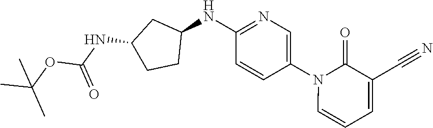 [SMILES: CC(C)(C)OC(=O)N[C@H]1CC[C@H](Nc2ccc(-n3cccc(C#N)c3=O)cn2)C1]

Cs₂CO₃ (1843 mg, 5.66 mmol) was added to 6′-chloro-2-oxo-2H-[1,3′-bipyridine]-3-carbonitrile Intermediate 10 (560 mg, 1.89 mmol), tert-butyl ((1S,3S)-3-aminocyclopentyl)carbamate (1133 mg, 5.66 mmol) and Pd-PEPPSI-IpentCl 2-methylpyridine (79 mg, 0.09 mmol) in DMF (30 mL) at 20° C. The resulting mixture was stirred at 100° C. for 15 h under a nitrogen atmosphere. The mixture was filtered through a pad of Celite. The filter cake was washed with DCM (3×10 mL). The filtrate was concentrated and the residue purified by reversed phase flash chromatography on a C18 column (gradient: 10-100% MeCN in water (+0.1% NH₃(aq)) and then by preparative TLC (EtOAc:PE, 3:1) to give the title compound (174 mg, 23%) as a grey solid; MS (ESI) m/z [M+H]⁺ 396.2.

### Intermediate 12

### 6′-(((1S,3S)-3-Aminocyclopentyl)amino)-2-oxo-2H-[1,3′-bipyridine]-3-carbonitrile

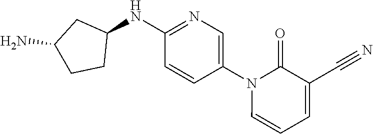 [SMILES: N#Cc1cccn(-c2ccc(N[C@H]3CC[C@H](N)C3)nc2)c1=O]

4 M HCl in MeOH (8 mL, 32.0 mmol) was added to a stirred solution of tert-butyl ((1S,3S)-3-((3-cyano-2-oxo-2H-[1,3′-bipyridin]-6′-yl)amino)cyclopentyl)carbamate Intermediate 11 (865 mg, 2.19 mmol) in MeOH (20 mL) at 25° C. The resulting solution was stirred at 80° C. for 2 h. The solvent was removed under reduced pressure to give the HCl salt of the crude title compound (966 mg, 100%) as a brown gum; MS (ESI) m/z [M+H]⁺ 296.0.

### Intermediate 13

### tert-Butyl ((1 S,3S)-3-(oxazolo[5,4-b]pyridin-2-ylamino)cyclopentyl)carbamate

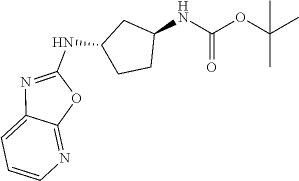 [SMILES: CC(C)(C)OC(=O)N[C@H]1CC[C@H](Nc2nc3cccnc3o2)C1]

2-Chlorooxazolo[5,4-b]pyridine (120 mg, 0.78 mmol) was added to tert-butyl ((1 S,3S)-3-aminocyclopentyl)carbamate (156 mg, 0.78 mmol), Pd-PEPPSI-IpentCl 2-methylpyridine (32.7 mg, 0.04 mmol) and Cs₂CO₃ (759 mg, 2.33 mmol) in 1,4-dioxane (10 mL). The resulting mixture was stirred at 100° C. for 15 h under a nitrogen atmosphere. The reaction mixture was filtered through Celite. The filter cake was washed with EtOAc (3×20 mL). The filtrate was concentrated under reduced pressure and the residue was purified by preparative TLC (EtOAc:PE, 3:1) to give the title compound (200 mg, 81%) as a yellow oil; MS (ESI) m/z [M+H]⁺ 319.2.

### Intermediate 14

### (1S,3S)—N′-(Oxazolo[5,4-b]pyridin-2-yl)cyclopentane-1,3-diamine

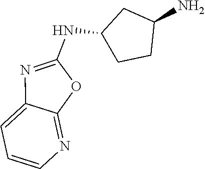 [SMILES: N[C@H]1CC[C@H](Nc2nc3cccnc3o2)C1]

tert-Butyl ((1 S,3S)-3-(oxazolo[5,4-b]pyridin-2-ylamino)cyclopentyl)carbamate Intermediate 13 (180 mg, 0.57 mmol) was added to TFA (2 mL) and DCM (6 mL). The resulting mixture was stirred at 25° C. for 3 h. The reaction mixture was concentrated in vacuo to give the TFA salt of the crude title compound (310 mg, 98%) as a brown oil; MS (ESI) m/z [M+H]⁺ 219.1.

### Intermediate 15

### Methyl 6′-chloro-2-oxo-2H-[1,3′-bipyridine]-5-carboxylate

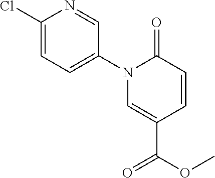 [SMILES: COC(=O)c1ccc(=O)n(-c2ccc(Cl)nc2)c1]

6-Chloropyridin-3-amine (1.0 g, 7.8 mmol) was added to a solution of methyl 2-oxo-2H-pyran-5-carboxylate (1.2 g, 7.8 mmol) in EtOH (20 mL) at 15° C. and the reaction mixture was stirred at 80° C. for 16 h. The reaction mixture was diluted with EtOH (20 mL). The resulting suspension solution was filtered and the filter cake was collected to give the crude title compound (1.53 g, 74%) as a purple solid; MS (ESI) m/z [M+H]⁺ 265.2.

### Intermediate 16

### 6′-Chloro-2-oxo-2H-[1,3′-bipyridine]-5-carboxylic acid

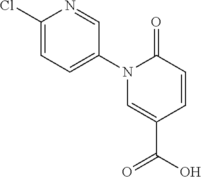 [SMILES: O=C(O)c1ccc(=O)n(-c2ccc(Cl)nc2)c1]

Methyl 6′-chloro-2-oxo-2H-[1,3′-bipyridine]-5-carboxylate Intermediate 15 (300 mg, 1.13 mmol) was added to NaOH (91 mg, 2.27 mmol) in THF (8 mL) and water (2 mL) at 20° C. The resulting solution was stirred at 20° C. for 15 h. The reaction mixture was concentrated at reduced pressure. The residue was diluted with water (5 mL) and the pH was adjusted to pH<7 with 1 M HCl. The mixture was diluted with water (100 mL) and extracted with EtOAc (3×75 mL). The organic layer was dried over Na₂SO₄, filtered and concentrated at reduced pressure to give crude title compound (230 mg, 81%) as a white solid; MS (ESI) m/z [M+H]⁺ 251.

### Intermediate 17

### 6′-Chloro-2-oxo-2H-[1,3′-bipyridine]-5-carboxamide

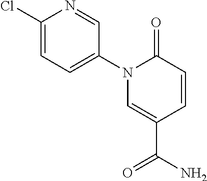 [SMILES: NC(=O)c1ccc(=O)n(-c2ccc(Cl)nc2)c1]

6′-Chloro-2-oxo-2H-[1,3′-bipyridine]-5-carboxylic acid Intermediate 16 (160 mg, 0.64 mmol) was added to HATU (485 mg, 1.28 mmol), TEA (0.712 mL, 5.11 mmol) and NH₄Cl (137 mg, 2.55 mmol) in DMF (10 mL) under a nitrogen atmosphere. The resulting mixture was stirred at 60° C. for 3 h. The reaction was quenched with sat brine (100 mL) and the mixture was extracted with EtOAc (3×75 mL). The organic layer was dried over Na₂SO₄, filtered and evaporated. The residue was purified by preparative TLC (DCM:MeOH, 10:1) to give the title compound (130 mg, 82%) as a white solid; MS (ESI) m/z [M+H]⁺ 250.

### Intermediate 18

### 6′-Chloro-2-oxo-2H-[1,3′-bipyridine]-5-carbonitrile

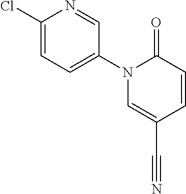 [SMILES: N#Cc1ccc(=O)n(-c2ccc(Cl)nc2)c1]

6′-Chloro-2-oxo-2H-[1,3′-bipyridine]-5-carboxamide Intermediate 17 (700 mg, 2.80 mmol) was added to pyridine (0.680 mL, 8.41 mmol) in DCM (10 mL). TFAA (1.188 mL, 8.41 mmol) was slowly added to the mixture at 0° C. The resulting mixture was stirred at 20° C. for 1 h. The reaction mixture was concentrated at reduced pressure. The synthesis procedure was repeated for a second batch of 6′-chloro-2-oxo-2H-[1,3′-bipyridine]-5-carboxamide Intermediate 17 (100 mg, 0.40 mmol). The two batches were combined and purified by preparative TLC (EtOAc:PE, 3:1) to give the title compound (540 mg, 72%) as a white solid; MS (ESI) m/z [M+H]⁺ 232.1.

### Intermediate 19

### 3-(6-Chloropyridin-3-yl)-1-methyl-1,3-dihydro-2H-imidazo[4,5-b]pyridin-2-one

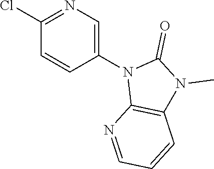 [SMILES: Cn1c(=O)n(-c2ccc(Cl)nc2)c2ncccc21]

TMEDA (2.59 g, 22.3 mmol) was added to a mixture of (6-chloropyridin-3-yl)boronic acid (2.93 g, 18.6 mmol), 1-methyl-1,3-dihydro-2H-imidazo[4,5-b]pyridin-2-one (1.11 g, 7.44 mmol) and Cu(OTf)₂ (4.04 g, 11.2 mmol) in dioxane (100 mL) and the resulting solution was stirred at 100° C. for 16 h. The mixture was filtered through a pad of Celite and the filtrate was concentrated at reduced pressure. The residue was partitioned between EtOAc (300 mL) and water (50 mL). The phases were separated, and the aqueous layer was extracted with EtOAc (5×150 mL). The combined organic layers were washed with water (3×50 mL), dried over Na₂SO₄, filtered and evaporated to afford a crude product which was purified by reversed phase flash chromatography on a C18 column (gradient: of 5-40% MeCN in water (containing 0.1% NH₃, aq)) to give the title compound (0.235 g, 12%) as a white solid; MS (ESI) m/z [M+H]⁺ 261.

### Intermediate 20

### tert-Butyl ((1S,3S)-3-((5-(1-methyl-2-oxo-1,2-dihydro-3H-imidazo[4,5-b]pyridin-3-yl)pyridin-2-yl)amino)cyclopentyl)carbamate

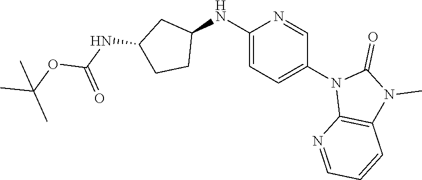 [SMILES: Cn1c(=O)n(-c2ccc(N[C@H]3CC[C@H](NC(=O)OC(C)(C)C)C3)nc2)c2ncccc21]

3-(6-Chloropyridin-3-yl)-1-methyl-1,3-dihydro-2H-imidazo[4,5-b]pyridin-2-one Intermediate 19 (200 mg, 0.77 mmol) was added to tert-butyl ((1S,3S)-3-aminocyclopentyl)carbamate (307 mg, 1.53 mmol), Cs₂CO₃ (750 mg, 2.30 mmol) and Pd-PEPPSI-IpentCl 2-methylpyridine (32 mg, 0.04 mmol) in 1,4-dioxane (10 mL) at 30° C. The resulting mixture was stirred at 100° C. for 15 h under a nitrogen atmosphere. The reaction mixture was diluted with water (50 mL) and the aqueous layer was extracted with EtOAc (8×30 mL). The organic layer was dried over Na₂SO₄, filtered and evaporated. The residue was purified by preparative TLC (EtOAc) to give the title compound (233 mg, 71%) as a yellow solid; MS (ESI) m/z [M+H]⁺ 425.1.

### Intermediate 21

### 3-(6-(((1S,3S)-3-Aminocyclopentyl)amino)pyridin-3-yl)-1-methyl-1,3-dihydro-2H-imidazo[4,5-b]pyridin-2-one

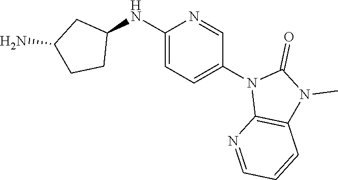 [SMILES: Cn1c(=O)n(-c2ccc(N[C@H]3CC[C@H](N)C3)nc2)c2ncccc21]

TFA (2 mL, 25.96 mmol) was added to tert-butyl ((1S,3S)-3-((5-(1-methyl-2-oxo-1,2-dihydro-3H-imidazo[4,5-b]pyridin-3-yl)pyridin-2-yl)amino)cyclopentyl)carbamate Intermediate 20 (220 mg, 0.52 mmol) in DCM (8 mL) at 30° C. The resulting mixture was stirred at 30° C. for 2 h. The solvent was removed under reduced pressure to give the TFA salt of the crude title compound (347 mg, 100%) as a brown gum; MS (ESI) m/z [M+H]⁺ 325.0.

### Intermediate 22

### tert-Butyl ((1S,3S)-3-((3-methyl-2-oxo-2H-[1,3′-bipyridin]-6′-yl)amino)cyclopentyl)carbamate

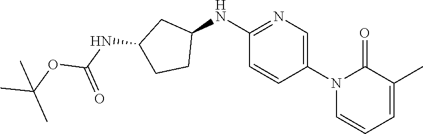 [SMILES: Cc1cccn(-c2ccc(N[C@H]3CC[C@H](NC(=O)OC(C)(C)C)C3)nc2)c1=O]

tert-Butyl ((1 S,3S)-3-((5-iodopyridin-2-yl)amino)cyclopentyl)carbamate Intermediate 1 (400 mg, 0.99 mmol) was added to 3-methylpyridin-2(1H)-one (1082 mg, 9.92 mmol), DMCDA (282 mg, 1.98 mmol), Cs₂CO₃ (3232 mg, 9.92 mmol) and Cu(I)I (378 mg, 1.98 mmol) in 1,4-dioxane (15 mL) at 20° C. under a nitrogen atmosphere. The resulting mixture was stirred at 100° C. for 15 h under a nitrogen atmosphere. The reaction mixture was diluted with sat brine (150 mL) and the aqueous layer extracted with EtOAc (3×100 mL). The organic layer was dried over Na₂SO₄, filtered and evaporated. The residue was purified by preparative TLC (MeOH:DCM, 1:15) to give the title compound (375 mg, 98%) as a yellow solid; MS (ESI) m/z [M+H]⁺ 385.3.

### Intermediate 23

### 6′-(((1S,3S)-3-Aminocyclopentyl)amino)-3-methyl-2H-[1,3′-bipyridin]-2-one

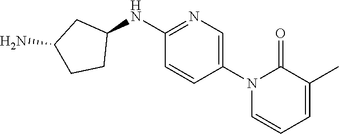 [SMILES: Cc1cccn(-c2ccc(N[C@H]3CC[C@H](N)C3)nc2)c1=O]

tert-Butyl ((1S,3S)-3-((3-methyl-2-oxo-2H-[1,3′-bipyridin]-6′-yl)amino)cyclopentyl)carbamate Intermediate 22 (360 mg, 0.94 mmol) was added to 4 M HCl in MeOH (10 mL, 40.0 mmol) in MeOH (15 mL). The resulting mixture was stirred at 60° C. for 2 h. The solvent was removed at reduced pressure to afford the HCl salt of the crude title compound (356 mg, 97%) as a yellow solid; MS (ESI) m/z [M+H]⁺ 285.2.

### Intermediate 24

### 3-Chloro-6′-fluoro-2H-[1,3′-bipyridin]-2-one

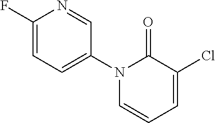 [SMILES: O=c1c(Cl)cccn1-c1ccc(F)nc1]

DMCDA (357 mg, 2.51 mmol) was added to 2-fluoro-5-iodopyridine (560 mg, 2.51 mmol), 3-chloropyridin-2(1H)-one (651 mg, 5.02 mmol), Cu(I)I (478 mg, 2.51 mmol) and K₃PO₄ (1.60 g, 7.53 mmol) in 1,4-dioxane (60 mL) at 25° C. The solution was stirred at 100° C. for 18 h under a nitrogen atmosphere. The reaction mixture was poured into sat brine (350 mL) and filtered through Celite. The filtrate was extracted with EtOAc (3×250 mL). The organic layer was dried over Na₂SO₄, filtered and evaporated. The crude product was purified by flash chromatography on silica (gradient: 0-30% EtOAc in PE) to give the title compound (258 mg, 45%) as a pale yellow solid; MS (ESI) m/z [M+H]⁺ 224.9.

### Intermediate 25

### tert-Butyl ((1S,3S)-3-((3-chloro-2-oxo-2H-[1,3′-bipyridin]-6′-yl)amino)cyclopentyl)carbamate

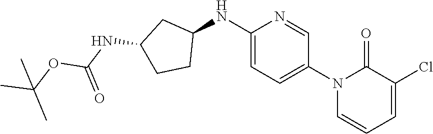 [SMILES: CC(C)(C)OC(=O)N[C@H]1CC[C@H](Nc2ccc(-n3cccc(Cl)c3=O)cn2)C1]

tert-Butyl ((1 S,3S)-3-aminocyclopentyl)carbamate (467 mg, 2.33 mmol) was added to 3-chloro-6′-fluoro-2H-[1,3′-bipyridin]-2-one Intermediate 24 (262 mg, 1.17 mmol) and Na₂CO₃ (371 mg, 3.50 mmol) in DMSO (20 mL) at 25° C. The resulting mixture was stirred at 120° C. for 15 h. The reaction mixture was concentrated and diluted with EtOAc (250 mL) and washed with sat brine (4×50 mL). The organic layer was dried over Na₂SO₄, filtered and evaporated. The residue was purified by preparative TLC (EtOAc:PE, 2:1) to give the title compound (456 mg, 97%) as a white solid; MS (ESI) m/z [M+H]⁺ 404.9.

### Intermediate 26

### 6′-(((1S,3S)-3-Aminocyclopentyl)amino)-3-chloro-2H-[1,3′-bipyridin]-2-one

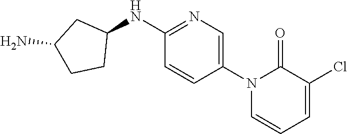 [SMILES: N[C@H]1CC[C@H](Nc2ccc(-n3cccc(Cl)c3=O)cn2)C1]

4 M HCl in dioxane (2 mL, 8.00 mmol) was added to tert-butyl ((1S,3S)-3-((3-chloro-2-oxo-2H-[1,3′-bipyridin]-6′-yl)amino)cyclopentyl)carbamate Intermediate 25 (425 mg, 1.05 mmol) in MeOH (20 mL) at 30° C. The resulting solution was stirred at 30° C. for 2 h. The solvent was removed under reduced pressure to give the HCl salt of the crude title compound (329 mg, 83%) as a yellow oil; MS (ESI) m/z [M+H]⁺ 305.0.

### Intermediate 27

### tert-Butyl ((1S,3S)-3-(thiazolo[5,4-b]pyridin-2-ylamino)cyclopentyl)carbamate

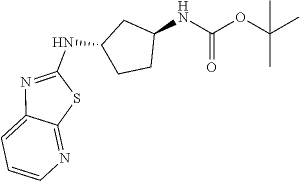 [SMILES: CC(C)(C)OC(=O)N[C@H]1CC[C@H](Nc2nc3cccnc3s2)C1]

2-Bromothiazolo[5,4-b]pyridine (200 mg, 0.93 mmol) was added to tert-butyl ((1 S,3S)-3-aminocyclopentyl)carbamate (186 mg, 0.93 mmol), Pd-PEPPSI-IpentCl 2-methylpyridine (39.1 mg, 0.05 mmol) and Cs₂CO₃ (909 mg, 2.79 mmol) in 1,4-dioxane (10 mL). The resulting mixture was stirred at 100° C. for 15 h under a nitrogen atmosphere. The reaction mixture was filtered through Celite. The filter cake was washed with EtOAc (3×20 mL) and the filtrate was concentrated under reduced pressure. The residue was purified by preparative TLC (EtOAc:PE, 2:1) to give the title compound (220 mg, 70%) as a yellow oil; MS (ESI) m/z [M+H]⁺ 335.0.

### Intermediate 28

### (1 S,3S)—N¹-(Thiazolo[5,4-b]pyridin-2-yl)cyclopentane-1,3-diamine

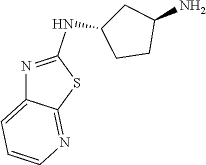 [SMILES: N[C@H]1CC[C@H](Nc2nc3cccnc3s2)C1]

tert-Butyl ((1 S,3S)-3-(thiazolo[5,4-b]pyridin-2-ylamino)cyclopentyl)carbamate Intermediate 27 (200 mg, 0.60 mmol) was added to TFA (2 mL) and DCM (6 mL). The resulting mixture was stirred at 25° C. for 3 h. The reaction mixture was concentrated at reduced pressure to give the TFA salt of the crude title compound (370 mg, 90%) as a brown oil; MS (ESI) m/z [M+H]⁺ 235.0.

### Intermediate 29

### tert-Butyl ((1S,3S)-3-((5-(6-oxopyridazin-1(6H)-yl)pyridin-2-yl)amino)cyclopentyl)carbamate

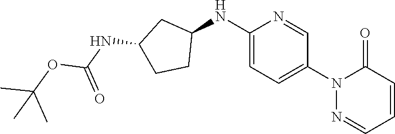 [SMILES: CC(C)(C)OC(=O)N[C@H]1CC[C@H](Nc2ccc(-n3ncccc3=O)cn2)C1]

Cs₂CO₃ (606 mg, 1.86 mmol) was added to tert-butyl ((1 S,3S)-3-((5-iodopyridin-2-yl)amino)cyclopentyl)carbamate Intermediate 1 (250 mg, 0.62 mmol), pyridazin-3(2H)-one (119 mg, 1.24 mmol), Cu(I)I (59 mg, 0.31 mmol) and N¹,N²-dimethylethane-1,2-diamine (27.3 mg, 0.31 mmol) in 1,4-dioxane (5 mL) at 25° C. The resulting suspension was stirred at 100° C. for 16 h under a nitrogen atmosphere. The solvent was removed under reduced pressure. The residue was poured into water (50 mL) and extracted with EtOAc (2×100 mL). The organic layer was dried over Na₂SO₄, filtered and evaporated. The residue was purified by preparative TLC (7 M NH₃ in MeOH:DCM, 1:30) to give the title compound (173 mg, 75%) as a brown solid; MS (ESI) m/z [M+H]⁺ 372.0.

### Intermediate 30

### 2-(6-(((1S,3S)-3-Aminocyclopentyl)amino)pyridin-3-yl)pyridazin-3(2H)-one

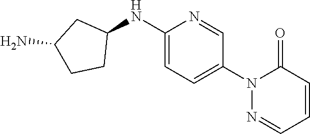 [SMILES: N[C@H]1CC[C@H](Nc2ccc(-n3ncccc3=O)cn2)C1]

4 M HCl in MeOH (5.0 mL, 20.00 mmol) was added to tert-butyl ((1S,3S)-3-((5-(6-oxopyridazin-1(6H)-yl)pyridin-2-yl)amino)cyclopentyl)carbamate Intermediate 29 (168 mg, 0.45 mmol) in MeOH (5 mL) at 25° C. The resulting solution was stirred at 60° C. for 3 h. The solvent was removed under reduced pressure to give the HCl salt of the crude title compound (156 mg, 100%) as a brown solid; MS (ESI) m/z [M+H]⁺ 272.0.

### Intermediate 31

### tert-Butyl ((1S,3S)-3-((5-(6-oxopyrimidin-1(6H)-yl)pyridin-2-yl)amino)cyclopentyl)carbamate

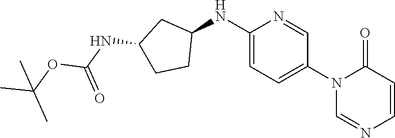 [SMILES: CC(C)(C)OC(=O)N[C@H]1CC[C@H](Nc2ccc(-n3cnccc3=O)cn2)C1]

Cs₂CO₃ (727 mg, 2.23 mmol) was added to tert-butyl ((1S,3S)-3-((5-iodopyridin-2-yl)amino)cyclopentyl)carbamate Intermediate 1 (300 mg, 0.74 mmol), pyrimidin-4(3H)-one (214 mg, 2.23 mmol), CuI (71 mg, 0.37 mmol) and DMCDA (53 mg, 0.37 mmol) in 1,4-dioxane (5 mL) at 15° C. The resulting suspension was stirred at 100° C. for 2 h under a nitrogen atmosphere. The solvent was removed under reduced pressure. The residue was poured into water (100 mL) and extracted with EtOAc (2×50 mL). The organic layer was dried over Na₂SO₄, filtered and concentrated. The residue was purified by preparative TLC (7 M NH₃ in MeOH:DCM, 1:30) and then by flash chromatography on silica (gradient: 7-10% MeOH in DCM) to give the title compound (165 mg, 59%) as a brown solid; MS (ESI) m/z [M+H]⁺ 372.0.

### Intermediate 32

### 3-(6-(((1S,3S)-3-Aminocyclopentyl)amino)pyridin-3-yl)pyrimidin-4(3H)-one

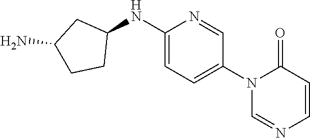 [SMILES: N[C@H]1CC[C@H](Nc2ccc(-n3cnccc3=O)cn2)C1]

tert-Butyl ((1S,3S)-3-((5-(6-oxopyrimidin-1(6H)-yl)pyridin-2-yl)amino)cyclopentyl)carbamate Intermediate 31 (155 mg, 0.42 mmol) was added to TFA (5 mL) at 25° C. The resulting solution was stirred at 60° C. for 2 h. The solvent was removed under reduced pressure to give the TFA salt of the crude title compound (356 mg, 99%) as a brown gum; MS (ESI) m/z [M+H]⁺ 271.9.

### Intermediate 33

### 6′-Chloro-3-fluoro-2,3′-bipyridine

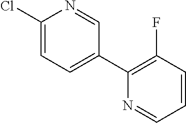 [SMILES: Fc1cccnc1-c1ccc(Cl)nc1]

2-Bromo-3-fluoropyridine (500 mg, 2.84 mmol) was added to (6-chloropyridin-3-yl)boronic acid (581 mg, 3.69 mmol), K₂CO₃ (1.18 g, 8.52 mmol) and Pd(dtbpf)Cl₂ (185 mg, 0.28 mmol) in 1,4-dioxane (4 mL) and water (1 mL) at rt under a nitrogen atmosphere, and the resulting mixture was stirred at 100° C. for 2 h. The reaction mixture was poured into water (100 mL) and extracted with DCM (2×50 mL). The combined organic layer was dried over Na₂SO₄, filtered, and evaporated to afford crude product. The crude product was purified by flash chromatography on silica (gradient: 0-100% EtOAc in PE) to give the crude title compound (750 mg) as a brown solid which was used without further purification; MS (ESI) m/z [M+H]⁺ 209.

### Intermediate 34

### tert-Butyl ((1S,3S)-3-((3-fluoro-[2,3′-bipyridin]-6′-yl)amino)cyclopentyl)carbamate

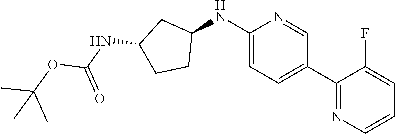 [SMILES: CC(C)(C)OC(=O)N[C@H]1CC[C@H](Nc2ccc(-c3ncccc3F)cn2)C1]

Cs₂CO₃ (937 mg, 2.88 mmol) was added to 6′-chloro-3-fluoro-2,3′-bipyridine Intermediate 33 (200 mg, 0.96 mmol), tert-butyl ((1S,3S)-3-aminocyclopentyl)carbamate (288 mg, 1.44 mmol) and Pd-PEPPSI-IpentCl 2-methylpyridine (64 mg, 0.08 mmol) in 1,4-dioxane (5 mL) at rt and the resulting suspension was stirred at 100° C. for 16 h under a nitrogen atmosphere. The reaction mixture was poured into water (100 mL) and extracted with DCM (2×50 mL). The combined organic layer was washed sequentially with brine (2×25 mL), dried over Na₂SO₄, filtered and evaporated. The crude product was purified by preparative TLC (7 M NH₃ in MeOH:DCM, 1:30), to give the title compound (314 mg, 88%) as a brown gum; MS (ESI) m/z [M+H]⁺=373.

### Intermediate 35

### (1S,3S)—N¹-(3-Fluoro-[2,3′-bipyridin]-6′-yl)cyclopentane-1,3-diamine

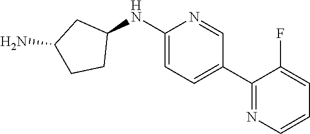 [SMILES: N[C@H]1CC[C@H](Nc2ccc(-c3ncccc3F)cn2)C1]

4 M HCl in MeOH (1.11 mL, 4.44 mmol) was added to tert-butyl ((1S,3S)-3-((3-fluoro-[2,3′-bipyridin]-6′-yl)amino)cyclopentyl)carbamate Intermediate 34 (331 mg, 0.89 mmol) in MeOH (5 mL) at rt and the resulting solution was stirred for 16 h and then at 50° C. for 1 h. The solvent was removed under reduced pressure to give the HCl salt of the title compound (390 mg, 100%) as a yellow gum which was used without further purification: MS (ESI) m/z [M+H]⁺ 273.

### Intermediate 36

### tert-Butyl ((1S,3S)-3-((5-iodopyrimidin-2-yl)amino)cyclopentyl)carbamate

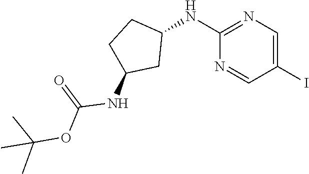 [SMILES: CC(C)(C)OC(=O)N[C@H]1CC[C@H](Nc2ncc(I)cn2)C1]

K₂CO₃ (690 mg, 4.99 mmol) was added to 2-chloro-5-iodopyrimidine (900 mg, 3.74 mmol) and tert-butyl ((1S,3S)-3-aminocyclopentyl)carbamate (500 mg, 2.50 mmol) in DMSO (2 mL) at 20° C. The resulting suspension was stirred at 100° C. for 3 h. The reaction mixture was poured into sat brine (200 mL) and extracted with EtOAc (3×150 mL). The organic layers were combined and washed with sat brine (3×100 mL), dried over Na₂SO₄, filtered and evaporated. The crude product was purified by flash chromatography (gradient: 2-30% EtOAc in PE) to give the title compound (950 mg, 94%) as a pale yellow solid; MS (ESI) m/z [M+H]⁺ 404.9.

### Intermediate 37

### tert-Butyl ((1S,3S)-3-((5-(6-oxopyridazin-1(6H)-yl)pyrimidin-2-yl)amino)cyclopentyl)carbamate

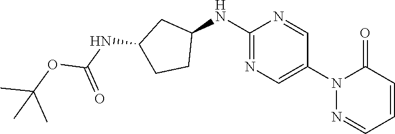 [SMILES: CC(C)(C)OC(=O)N[C@H]1CC[C@H](Nc2ncc(-n3ncccc3=O)cn2)C1]

DMCDA (44.0 mg, 0.31 mmol) was added to tert-butyl ((1S,3S)-3-((5-iodopyrimidin-2-yl)amino)cyclopentyl)carbamate Intermediate 36 (250 mg, 0.62 mmol), pyridazin-3-ol (178 mg, 1.86 mmol), Cu(I)I (59 mg, 0.31 mmol) and Cs₂CO₃ (403 mg, 1.24 mmol) in 1,4-dioxane (15 mL) at 20° C. The resulting suspension was stirred at 100° C. for 15 h under a nitrogen atmosphere. The reaction mixture was poured into sat brine (150 mL) and extracted with EtOAc (3×100 mL). The organic layer was dried over Na₂SO₄, filtered and evaporated. The residue was purified by preparative TLC (EtOAc) to give the title compound (190 mg, 82%) as a pale yellow oil which solidified on standing; MS (ESI) m/z [M+H]⁺ 373.3.

### Intermediate 38

### 2-(2-(((1S,3S)-3-Aminocyclopentyl)amino)pyrimidin-5-yl)pyridazin-3(2H)-one

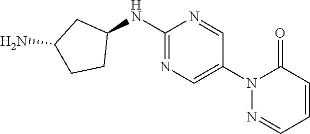 [SMILES: N[C@H]1CC[C@H](Nc2ncc(-n3ncccc3=O)cn2)C1]

4 M HCl in MeOH (0.114 mL, 0.46 mmol) was added to tert-butyl ((1S,3S)-3-((5-(6-oxopyridazin-1(6H)-yl)pyrimidin-2-yl)amino)cyclopentyl)carbamate Intermediate 37 (170 mg, 0.46 mmol) in MeOH (15 mL) at 20° C. The resulting solution was stirred at 60° C. for 6 h. The solvent was removed under reduced pressure to give the HCl salt of the crude title compound (150 mg, 95%) as a yellow gum; MS (ESI) m/z [M+H]⁺ 272.9.

### Intermediate 39

### 2-Chloro-3-tosyl-3H-imidazo[4,5-b]pyridine*

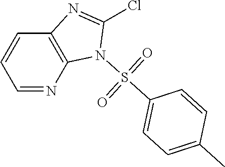 [SMILES: Cc1ccc(S(=O)(=O)n2c(Cl)nc3cccnc32)cc1]

4-Methylbenzenesulfonyl chloride (1.290 g, 6.77 mmol) was added to 2-chloro-3H-imidazo[4,5-b]pyridine (0.990 g, 6.45 mmol), DMAP (0.079 g, 0.64 mmol) and DIPEA (3.38 mL, 19.3 mmol) in DCE (35 mL) at 20° C. The resulting solution was stirred at 60° C. for 15 h. The reaction mixture was poured into sat brine (150 mL) and extracted with EtOAc (3×100 mL). The organic layers were combined and washed with sat brine (3×100 mL), dried over Na₂SO₄, filtered and evaporated. The crude product was purified by flash chromatography (gradient: 5-30% EtOAc in PE) to give the title compound (1.7 g, 86%) as a white solid; MS (ESI) m/z [M+H]⁺ 307.9. (*regiochemistry was not confirmed)

### Intermediate 40

### 2-(2-(((1S,3S)-3-((3-Tosyl-3H-imidazo[4,5-b]pyridin-2-yl)amino)cyclopentyl)amino)pyrimidin-5-yl)pyridazin-3(2H)-one*

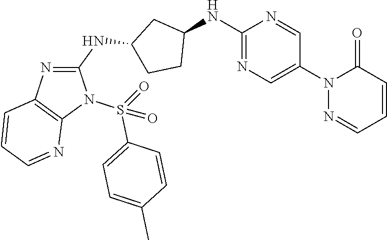 [SMILES: Cc1ccc(S(=O)(=O)n2c(N[C@H]3CC[C@H](Nc4ncc(-n5ncccc5=O)cn4)C3)nc3cccnc32)cc1]

2-(2-(((1S,3S)-3-Aminocyclopentyl)amino)pyrimidin-5-yl)pyridazin-3(2H)-one 2HCl Intermediate 38 (130 mg, 0.38 mmol) was added to 2-chloro-3-tosyl-3H-imidazo[4,5-b]pyridine* Intermediate 39 (232 mg, 0.75 mmol), Pd-PEPPSI-IpentCl 2-methylpyridine (16 mg, 0.02 mmol) and Cs₂CO₃ (613 mg, 1.88 mmol) in 1,4-dioxane (10 mL). The resulting mixture was stirred at 100° C. for 15 h under a nitrogen atmosphere. The reaction mixture was filtered through Celite. The filter cake was washed with EtOAc (3×20 mL). The filtrate was concentrated under reduced pressure. The residue was purified by preparative TLC (MeOH:DCM, 1:10), to give the title compound (110 mg, 53%) as a red solid; MS (ESI) m/z [M+H]⁺ 544.1. (*regiochemistry was not confirmed)

### Intermediate 41

### 2-Chloro-5-(2-(difluoromethoxy)-6-fluorophenyl)pyridine

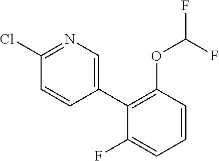 [SMILES: Fc1cccc(OC(F)F)c1-c1ccc(Cl)nc1]

Na₂CO₃ (202 mg, 1.91 mmol) was added to (6-chloropyridin-3-yl)boronic acid (100 mg, 0.64 mmol), 1-(difluoromethoxy)-3-fluoro-2-iodobenzene (366 mg, 1.27 mmol), XPhos Pd G2 (50 mg, 0.06 mmol) and XPhos (30 mg, 0.06 mmol) in 1,4-dioxane (2 mL) and water (2 mL) at rt and the resulting suspension was heated in a microwave reactor at 120° C. for 2 h under a nitrogen atmosphere. The reaction mixture was poured into water (100 mL) and extracted with DCM (2×50 mL). The combined organic layer was dried over Na₂SO₄, filtered and evaporated. The crude product was purified by preparative TLC (EtOAc:PE, 1:4) to give the title compound (119 mg, 68%) as a brown solid; MS (ESI) m/z [M+H]⁺=274.

### Intermediate 42

### tert-Butyl ((1S,3S)-3-((5-(2-(difluoromethoxy)-6-fluorophenyl)pyridin-2-yl)amino)cyclopentyl)carbamate

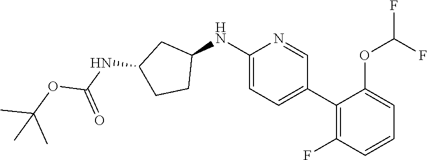 [SMILES: CC(C)(C)OC(=O)N[C@H]1CC[C@H](Nc2ccc(-c3c(F)cccc3OC(F)F)cn2)C1]

Cs₂CO₃ (389 mg, 1.20 mmol) was added to 2-chloro-5-(2-(difluoromethoxy)-6-fluorophenyl)pyridine Intermediate 41 (109 mg, 0.40 mmol), tert-butyl ((1S,3S)-3-aminocyclopentyl)carbamate (120 mg, 0.60 mmol) and Pd-PEPPSI-IpentCl 2-methylpyridine (27 mg, 0.03 mmol) in 1,4-dioxane (3 mL) at rt and the resulting suspension was stirred at 100° C. for 16 h under a nitrogen atmosphere. The reaction mixture was poured into water (100 mL) and extracted with DCM (2×50 mL). The combined organic layer was dried over Na₂SO₄, filtered and evaporated. The crude product was purified by preparative TLC (7 M NH₃ in MeOH:DCM, 1:30), to give the title compound (88 mg, 51%) as a brown solid; MS (ESI) m/z [M+H]⁺=438.

### Intermediate 43

### (1S,3S)—N¹-(5-(2-(Difluoromethoxy)-6-fluorophenyl)pyridin-2-yl)cyclopentane-1,3-diamine

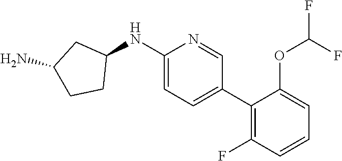 [SMILES: N[C@H]1CC[C@H](Nc2ccc(-c3c(F)cccc3OC(F)F)cn2)C1]

4 M HCl in MeOH (0.24 mL, 0.96 mmol) was added to tert-butyl ((1S,3S)-3-((5-(2-(difluoromethoxy)-6-fluorophenyl)pyridin-2-yl)amino)cyclopentyl)carbamate Intermediate 42 (85 mg, 0.19 mmol) in MeOH (3 mL) at rt and the resulting solution was stirred at 60° C. for 2 h. The reaction mixture was poured into water (50 mL), extracted with 7 M NH₃ in MeOH:DCM (2×25 mL). The organic layers were combined and washed with sat NaHCO₃ (aq, 2×25 mL), dried over Na₂SO₄, filtered and evaporated to give (65 mg, 99%) of the title compound as a brown gum which was used in the next step without further purification; MS (ESI) m/z [M+H]⁺=338.

### Intermediate 44

### 1-(6-Chloropyridin-3-yl)quinolin-2(1H)-one

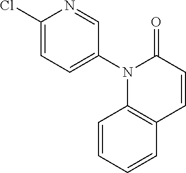 [SMILES: O=c1ccc2ccccc2n1-c1ccc(Cl)nc1]

TMEDA (1.20 g, 10.3 mmol) was added to a mixture of (6-chloropyridin-3-yl)boronic acid (1.63 g, 10.3 mmol), quinolin-2(1H)-one (500 mg, 3.44 mmol), and Cu(OTf)₂ (2.49 g, 6.89 mmol) in 1,4-dioxane (20 mL) and the reaction mixture was stirred at 100° C. for 16 h. The reaction mixture was filtered through Celite and the filtrate was concentrated under reduced pressure. The residue was partitioned between EtOAc (200 mL) and water (50 mL). The aqueous layer was extracted with EtOAc (4×100 mL). The combined organic layer was washed with water (3×300 mL), dried over Na₂SO₄, filtered and concentrated. The crude product was purified by reversed phase flash chromatography on a C18 column (gradient: 5-46% MeCN in water (0.1% NH₃ aq)) to give the title compound (211 mg, 24%) as a green solid; MS (ESI) m/z [M+H]⁺ 256.95.

### Intermediate 45

### tert-Butyl ((1S,3S)-3-((5-(2-oxoquinolin-1(2H)-yl)pyridin-2-yl)amino)cyclopentyl)carbamate

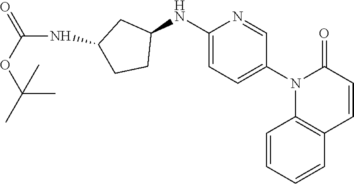 [SMILES: CC(C)(C)OC(=O)N[C@H]1CC[C@H](Nc2ccc(-n3c(=O)ccc4ccccc43)cn2)C1]

Cs₂CO₃ (571 mg, 1.75 mmol) was added to a mixture of 1-(6-chloropyridin-3-yl)quinolin-2(1H)-one Intermediate 44 (90 mg, 0.35 mmol), tert-butyl ((1S,3S)-3-aminocyclopentyl)carbamate (211 mg, 1.05 mmol), and Pd-PEPPSI-IpentCl 2-methylpyridine (29 mg, 0.04 mmol) in 1,4-dioxane (10 mL) and the reaction mixture was stirred at 100° C. for 15 h. The reaction mixture was diluted with EtOAc (200 mL), washed with sat brine (2×10 mL) and water (2×10 mL). The organic layer was dried over Na₂SO₄, filtered, and concentrated. The crude product was purified by preparative TLC (MeOH:DCM, 1:40) to give the title compound (122 mg, 83%) as a white solid; MS (ESI) m/z [M+H]⁺ 421.00.

### Intermediate 46

### 1-(6-(((1S,3S)-3-Aminocyclopentyl)amino)pyridin-3-yl)quinolin-2(1H)-one

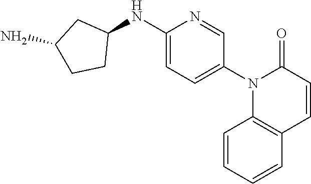 [SMILES: N[C@H]1CC[C@H](Nc2ccc(-n3c(=O)ccc4ccccc43)cn2)C1]

4 M HCl in MeOH (5.0 mL, 20 mmol) was added to a solution of tert-butyl ((1S,3S)-3-((5-(2-oxoquinolin-1(2H)-yl)pyridin-2-yl)amino)cyclopentyl)carbamate Intermediate 45 (110 mg, 0.26 mmol) in MeOH (10 mL) and the reaction mixture was stirred at 80° C. for 2 h. The reaction mixture was concentrated at reduced pressure to give the HCl salt of the title compound (90 mg, 96%) as a brown gum; MS (ESI) m/z [M+H]⁺ 321.00.

### Intermediate 47

### 2-Chloro-3-(4-methoxybenzyl)-3H-imidazo[4,5-b]pyridine and

### 2-Chloro-1-(4-methoxybenzyl)-1H-imidazo[4,5-b]pyridine

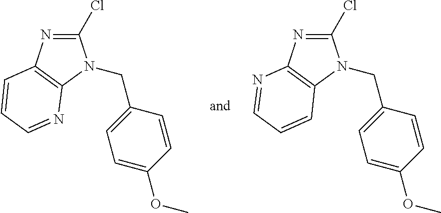 [SMILES: COc1ccc(Cn2c(Cl)nc3cccnc32)cc1 ; COc1ccc(Cn2c(Cl)nc3ncccc32)cc1]

K₂CO₃ (297 mg. 2.15 mmol) was added to a solution of 2-chloro-3H-imidazo[4,5-b]pyridine (220 mg, 1.43 mmol) and 1-(chloromethyl)-4-methoxybenzene (673 mg, 4.30 mmol) in DMF (5 mL) and the reaction mixture was stirred at 60° C. for 16 h. The reaction mixture was diluted with EtOAc (25 mL) and washed with water (3×20 mL). The organic layer was dried over Na₂SO₄, filtered and concentrated. The crude product was purified by preparative TLC (EtOAc:PE, 1:1) to give a mixture of the regioisomeric title compounds (150 mg, 38%) as a yellow solid; MS (ESI) m/z [M+H]⁺ 273.8.

### Intermediate 48

### 1-(6-(((1S,3S)-3-((3-(4-Methoxybenzyl)-3H-imidazo[4,5-b]pyridin-2-yl)amino)cyclopentyl)amino)pyridin-3-yl)quinolin-2(1H)-one and 1-(6-(((1S,3S)-3-((1-(4-methoxybenzyl)-1H-imidazo[4,5-b]pyridin-2-yl)amino)cyclopentyl)amino)pyridin-3-yl)quinolin-2(1H)-one

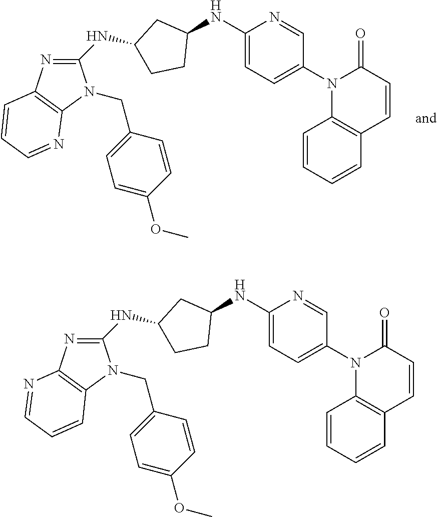 [SMILES: COc1ccc(Cn2c(N[C@H]3CC[C@H](Nc4ccc(-n5c(=O)ccc6ccccc65)cn4)C3)nc3cccnc32)cc1 ; COc1ccc(Cn2c(N[C@H]3CC[C@H](Nc4ccc(-n5c(=O)ccc6ccccc65)cn4)C3)nc3ncccc32)cc1]

tBuBrettPhos G3 (20 mg, 0.02 mmol) was added to a mixture of 2-chloro-3-(4-methoxybenzyl)-3H-imidazo[4,5-b]pyridine and 2-chloro-1-(4-methoxybenzyl)-1H-imidazo[4,5-b]pyridine Intermediate 47 (174 mg, 0.64 mmol), 1-(6-(((1S,3S)-3-aminocyclopentyl)amino)pyridin-3-yl)quinolin-2(1H)-one×2 HCl Intermediate 46 (100 mg, 0.25 mmol) and NaOtBu (122 mg, 1.27 mmol) in DMA (5 mL). The reaction mixture was stirred at 100° C. for 18 h under a nitrogen atmosphere. The reaction mixture was diluted with EtOAc (20 mL) and washed with water (3×25 mL). The organic layer was dried over Na₂SO₄, filtered, and concentrated. The residue was purified by preparative TLC (MeOH:DCM, 1:20), to give a mixture of regioisomeric title compounds (100 mg, 70%) as a yellow solid; MS (ESI) m/z [M+H]⁺ 558.30.

### Intermediate 49

### 1-(6-Chloropyridin-3-yl)-1,8-naphthyridin-2(1H)-one

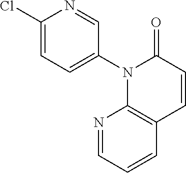 [SMILES: O=c1ccc2cccnc2n1-c1ccc(Cl)nc1]

TMEDA (1.43 g, 12.32 mmol) was added to a solution of (6-chloropyridin-3-yl)boronic acid (1.29 g, 8.21 mmol), 1,8-naphthyridin-2(1H)-one (600 mg, 4.11 mmol) and Cu(OTf)₂ (1.78 g, 4.93 mmol) in 1,4-dioxane (10 mL) at 25° C. The reaction mixture was stirred under a nitrogen atmosphere at 100° C. for 16 h. The mixture was filtered through Celite. The filtrate was concentrated at reduced pressure and the residue was partitioned between EtOAc (200 mL) and water (50 mL). The aqueous layer was extracted with EtOAc (4×100 mL). The combined organic layer was washed with water (3×50 mL). The organic layer was dried over Na₂SO₄, filtered and concentrated. The crude product was purified by reversed phase flash chromatography on a C18 column (gradient: 5-43% MeCN in water (0.1% NH₃ aq)) to give the title compound (252 mg, 23%) as a pale yellow solid; MS (ESI) m/z [M+H]⁺=257.9.

### Intermediate 50

### tert-Butyl ((1S,3S)-3-((5-(2-oxo-1,8-naphthyridin-1(2H)-yl)pyridin-2-yl)amino)cyclopentyl)carbamate

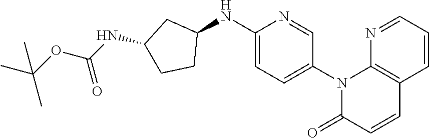 [SMILES: CC(C)(C)OC(=O)N[C@H]1CC[C@H](Nc2ccc(-n3c(=O)ccc4cccnc43)cn2)C1]

Pd-PEPPSI-IpentCl 2-methylpyridine (33 mg, 0.04 mmol) was added to 1-(6-chloropyridin-3-yl)-1,8-naphthyridin-2(1H)-one Intermediate 49 (100 mg, 0.39 mmol), tert-butyl ((1S,3S)-3-aminocyclopentyl)carbamate (117 mg, 0.58 mmol) and Cs₂CO₃ (379 mg, 1.16 mmol) in 1,4-dioxane (5 mL). The reaction mixture was stirred at 100° C. for 18 h under a nitrogen atmosphere. The reaction mixture was diluted with EtOAc (200 mL) and washed with water (3×15 mL). The organic layer was dried over Na₂SO₄, filtered, and concentrated at reduced pressure. The crude product was purified by preparative TLC (MeOH:DCM, 1:30) to give the title compound (140 mg, 86%) as a pale yellow solid; MS m/z (ES⁺), [M+H]⁺ 422.10.

### Intermediate 51

### 1-(6-(((1S,3S)-3-Aminocyclopentyl)amino)pyridin-3-yl)-1,8-naphthyridin-2(1H)-one

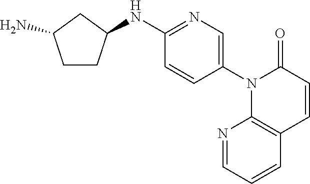 [SMILES: N[C@H]1CC[C@H](Nc2ccc(-n3c(=O)ccc4cccnc43)cn2)C1]

4 M HCl in MeOH (5 mL, 20.0 mmol) was added to tert-butyl ((1S,3S)-3-((5-(2-oxo-1,8-naphthyridin-1(2H)-yl)pyridin-2-yl)amino)cyclopentyl)carbamate Intermediate 50 (130 mg, 0.31 mmol) in MeOH (5 mL). The resulting solution was stirred at 80° C. for 2 h. The solvent was removed under reduced pressure to give the HCl salt of the title compound (105 mg, 95%) as a light brown solid; MS m/z (ES+), [M+H]⁺ 322.0.

### Intermediate 52

### 1-(6-(((1S,3S)-3-((3-(4-Methoxybenzyl)-3H-imidazo[4,5-b]pyridin-2-yl)amino)cyclopentyl)amino)pyridin-3-yl)-1,8-naphthyridin-2(1H)-one and 1-(6-(((1S,3S)-3-((1-(4-methoxybenzyl)-1H-imidazo[4,5-b]pyridin-2-yl)amino)cyclopentyl)amino)pyridin-3-yl)-1,8-naphthyridin-2(1H)-one

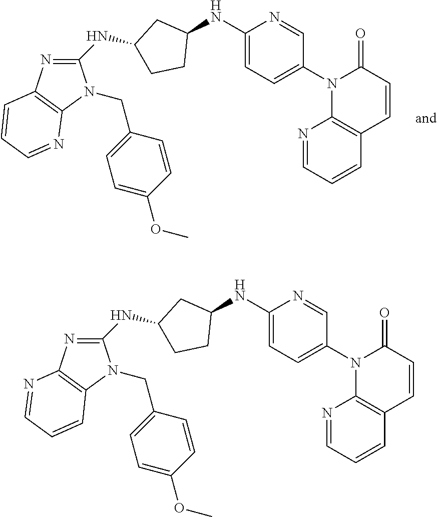 [SMILES: COc1ccc(Cn2c(N[C@H]3CC[C@H](Nc4ccc(-n5c(=O)ccc6cccnc65)cn4)C3)nc3cccnc32)cc1 ; COc1ccc(Cn2c(N[C@H]3CC[C@H](Nc4ccc(-n5c(=O)ccc6cccnc65)cn4)C3)nc3ncccc32)cc1]

Pd-PEPPSI-IpentCl 2 methylpyridine (26 mg, 0.03 mmol) was added to a mixture of 2-chloro-3-(4-methoxybenzyl)-3H-imidazo[4,5-b]pyridine and 2-chloro-1-(4-methoxybenzyl)-1H-imidazo[4,5-b]pyridine Intermediate 47 (111 mg, 0.40 mmol), 1-(6-(((1S,3S)-3-aminocyclopentyl)amino)pyridin-3-yl)-1,8-naphthyridin-2(1H)-one) HCl Intermediate 51 (100 mg, 0.31 mmol) and NaOtBu (60 mg, 0.62 mmol) in 1,4-dioxane (5 mL) and the reaction mixture was stirred at 100° C. for 18 h under a nitrogen atmosphere. The reaction mixture was diluted with EtOAc (20 mL) and washed with water (3×25 mL). The organic layer was dried over Na₂SO₄, filtered, and concentrated at reduced pressure. The crude product was purified by preparative TLC (EtOAc:PE, 1:1) to give a mixture of the regioisomeric title compounds (120 mg, 69%) as a yellow solid; MS m/z (ES+), [M+H]⁺ 559.

### Intermediate 53

### 3-(4-Methoxybenzyl)-2-(methylsulfonyl)-3H-imidazo[4,5-b]pyridine*

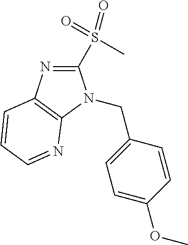 [SMILES: COc1ccc(Cn2c(S(C)(=O)=O)nc3cccnc32)cc1]

1-(Chloromethyl)-4-methoxybenzene (4.45 g, 28.40 mmol) was added to a mixture of 2-(methylsulfonyl)-1H-imidazo[4,5-b]pyridine (3.5 g, 17.8 mmol) and DIPEA (9.3 mL, 53 mmol) in THF (60 mL) and the reaction mixture was stirred at 60° C. for 18 h. The solvent was removed under reduced pressure. The crude product was purified by normal phase flash chromatography on silica (gradient: 1-5% MeOH in DCM) to give the title compound (5.30 g, 94%) as a brown gum and as a single regioisomer; MS (ESI) m/z [M+H]⁺ 317.9. (*regiochemistry was not confirmed)

### Intermediate 54

### tert-Butyl ((1S,3S)-3-((3-(4-methoxybenzyl)-3H-imidazo[4,5-b]pyridin-2-yl)amino)cyclopentyl)carbamate*

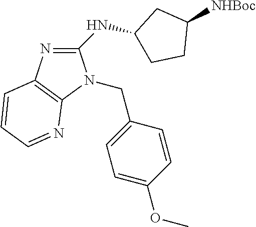

tert-Butyl ((1S,3S)-3-aminocyclopentyl)carbamate (3.03 g, 15.12 mmol) was added to a mixture of 3-(4-methoxybenzyl)-2-(methylsulfonyl)-3H-imidazo[4,5-b]pyridine* Intermediate 53 (4.0 g, 12.60 mmol) in n-BuOH (50 mL) and the reaction mixture was stirred at 120° C. for 18 h. The solvent was removed under reduced pressure. The crude product was purified by flash chromatography on deactivated alumina (gradient: 0-5% MeOH in DCM) to give the title compound (5.20 g, 94%) as a brown gum and as a single regioisomer; MS (ESI) m/z [M+H]⁺ 438.1. (*regiochemistry was not confirmed)

### Intermediate 55

### (1S,3S)—N¹-(3-(4-Methoxybenzyl)-3H-imidazo[4,5-b]pyridin-2-yl)cyclopentane-1,3-diamine*

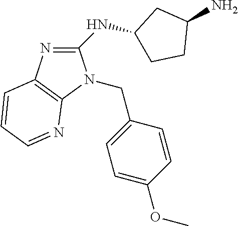 [SMILES: COc1ccc(Cn2c(N[C@H]3CC[C@H](N)C3)nc3cccnc32)cc1]

TFA (15 mL, 194.70 mmol) was added to a solution of tert-butyl ((1S,3S)-3-((3-(4-methoxybenzyl)-3H-imidazo[4,5-b]pyridin-2-yl)amino)cyclopentyl)carbamate* Intermediate 54 (5.2 g, 11.9 mmol) in DCM (50 mL) and the reaction mixture was stirred at 20° C. for 18 h. The solvent was removed under reduced pressure to give the TFA salt of the title compound (6.0 g, 89%) as a brown gum and as a single regioisomer; MS (ESI) m/z [M+H]⁺ 338.05. (*regiochemistry was not confirmed)

### Intermediate 56

### (1S,3S)—N¹-(5-Iodopyridin-2-yl)-N³-(3-(4-methoxybenzyl)-3H-imidazo[4,5-b]pyridin-2-yl)cyclopentane-1,3-diamine*

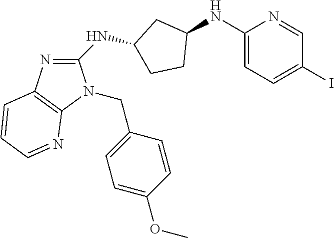 [SMILES: COc1ccc(Cn2c(N[C@H]3CC[C@H](Nc4ccc(I)cn4)C3)nc3cccnc32)cc1]

2-Fluoro-5-iodopyridine (2.162 g, 9.70 mmol) was added to (1S,3S)—N¹-(3-(4-methoxybenzyl)-3H-imidazo[4,5-b]pyridin-2-yl)cyclopentane-1,3-diamine×5 TFA Intermediate 55 (4.0 g, 4.41 mmol) and K₂CO₃ (6.09 g, 44.08 mmol) in DMSO (30 mL) and the reaction mixture was stirred at 120° C. for 18 h. The reaction mixture was poured into sat brine (350 mL), extracted with EtOAc (3×250 mL), and the combined organic layers were washed with sat brine (3×200 mL). The organic layer was dried over Na₂SO₄, filtered, and concentrated. The crude product was purified by normal phase flash chromatography on silica (gradient: 0-1% MeOH (7 M NH₃) in DCM) to give the title compound (2.0 g, 84%) as a yellow gum and as a single regioisomer; MS (ESI) m/z [M+H]⁺ 541. (*regiochemistry was not confirmed)

### Intermediate 57

### 1-(6-(((1S,3S)-3-((3-(4-Methoxybenzyl)-3H-imidazo[4,5-b]pyridin-2-yl)amino)cyclopentyl)amino)pyridin-3-yl)-1,3-dihydro-2H-benzo[d]imidazol-2-one*

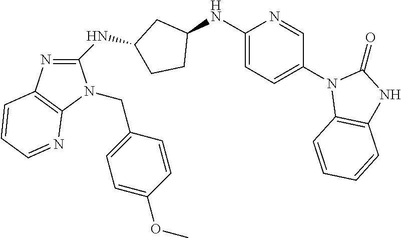 [SMILES: COc1ccc(Cn2c(N[C@H]3CC[C@H](Nc4ccc(-n5c(=O)[nH]c6ccccc65)cn4)C3)nc3cccnc32)cc1]

DMCDA (26 mg, 0.19 mmol) was added to a mixture of 1,3-dihydro-2H-benzo[d]imidazol-2-one (149 mg, 1.11 mmol), (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (200 mg, 0.37 mmol), CuI (35 mg, 0.19 mmol) and Cs₂CO₃ (362 mg, 1.11 mmol) in 1,4-dioxane (5 mL) and the reaction mixture was stirred at 100° C. for 18 h under a nitrogen atmosphere. The reaction mixture was diluted with EtOAc (75 mL) and washed with water (3×35 mL) and sat brine (3×25 mL). The organic layer was dried over Na₂SO₄, filtered, and concentrated. The crude product was purified by preparative TLC (7 M NH₃ in MeOH:DCM, 1:20) to give the title compound (80 mg, 39%) as a brown solid and as a single regioisomer; MS (ESI) m/z [M+H]⁺ 547. (*regiochemistry was not confirmed)

### Intermediate 58

### 1-(6-(((1S,3S)-3-((3-(4-Methoxybenzyl)-3H-imidazo[4,5-b]pyridin-2-yl)amino)cyclopentyl)amino)pyridin-3-yl)-3-methyl-1,3-dihydro-2H-benzo[d]imidazol-2-one*

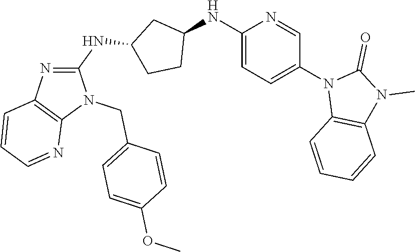 [SMILES: COc1ccc(Cn2c(N[C@H]3CC[C@H](Nc4ccc(-n5c(=O)n(C)c6ccccc65)cn4)C3)nc3cccnc32)cc1]

DMCDA (26 mg, 0.19 mmol) was added to a mixture of 1-methyl-1,3-dihydro-2H-benzo[d]imidazol-2-one (41 mg, 0.28 mmol), (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (50 mg, 0.09 mmol), CuI (35 mg, 0.19 mmol) and Cs₂CO₃ (90 mg, 0.28 mmol) in 1,4-dioxane (3 mL) and the reaction mixture was stirred at 100° C. for 2 h under a nitrogen atmosphere. The reaction mixture was diluted with EtOAc (75 mL) and washed with water (3×25 mL) and sat brine (3×10 mL). The organic layer was dried over Na₂SO₄, filtered, and concentrated. The crude product was purified by preparative TLC (MeOH:EtOAc, 1:10) to give the title compound (40 mg, 77%) as a brown solid and as a single regioisomer; MS (ESI) m/z [M+H]⁺ 441.2. (*regiochemistry was not confirmed)

### Intermediate 59

### 1-(6-(((1S,3S)-3-((3-(4-Methoxybenzyl)-3H-imidazo[4,5-b]pyridin-2-yl)amino)cyclopentyl)amino)pyridin-3-yl)-3-methylimidazolidine-2,4-dione*

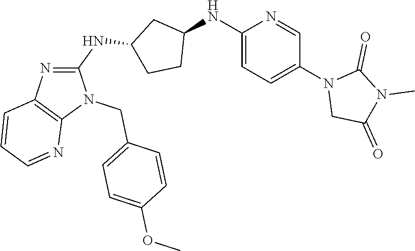 [SMILES: COc1ccc(Cn2c(N[C@H]3CC[C@H](Nc4ccc(N5CC(=O)N(C)C5=O)cn4)C3)nc3cccnc32)cc1]

DMCDA (39 mg, 0.28 mmol) was added to a mixture of (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (300 mg, 0.56 mmol), 3-methylimidazolidine-2,4-dione (127 mg, 1.11 mmol), CuI (53 mg, 0.28 mmol) and Cs₂CO₃ (543 mg, 1.67 mmol) in 1,4-dioxane (5 mL) and the reaction mixture was stirred at 100° C. for 18 h under a nitrogen atmosphere. The reaction mixture was diluted with EtOAc (75 mL) and washed with water (3×25 mL) and sat brine (3×20 mL). The organic layer was dried over Na₂SO₄, filtered, and concentrated. The crude product was purified by preparative TLC (7 M NH₃ in MeOH:DCM, 1:20), to give the title compound (165 mg, 56%) as a brown solid and as a single regioisomer; MS (ESI) m/z [M+H]⁺ 527.30. (*regiochemistry was not confirmed)

### Intermediate 60

### (1S,3S)—N¹-(3-(4-Methoxybenzyl)-3H-imidazo[4,5-b]pyridin-2-yl)cyclopentane-1,3-diamine*

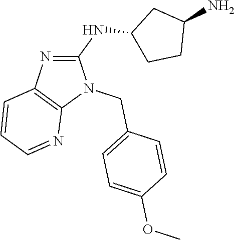 [SMILES: COc1ccc(Cn2c(N[C@H]3CC[C@H](N)C3)nc3cccnc32)cc1]

4 M HCl in MeOH (4 mL, 16.00 mmol) was added to a solution of tert-butyl ((1S,3S)-3-((3-(4-methoxybenzyl)-3H-imidazo[4,5-b]pyridin-2-yl)amino)cyclopentyl)carbamate* Intermediate 54 (1.5 g, 3.43 mmol) in MeOH (10 mL) and the reaction mixture was stirred at 60° C. for 18 h. The solvent was removed under reduced pressure to give the HCl salt of the title compound (1.0 g, 86%) as a brown gum and as a single regioisomer; MS (ESI) m/z [M+H]⁺ 338.05. (*regiochemistry was not confirmed)

### Intermediate 61

### 4-Nitrophenyl (6-chloropyridin-3-yl)carbamate

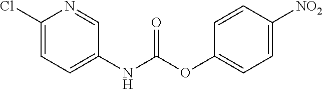 [SMILES: O=C(Nc1ccc(Cl)nc1)Oc1ccc([N+](=O)[O-])cc1]

4-Nitrophenyl carbonochloridate (1.73 g, 8.56 mmol) was added to a solution of 6-chloropyridin-3-amine (1 g, 7.78 mmol) in MeCN (20 mL) and the reaction mixture was stirred at 20° C. for 30 min. The reaction mixture was diluted with MeCN (200 mL), filtered through an organic phase filter, and concentrated to give the crude title compound (2.18 g, 96%) as a purple solid; MS (ESI) m/z [M+H]⁺=293.9.

### Intermediate 62

### 3-(6-Chloropyridin-3-yl)-1-methylimidazolidine-2,4-dione

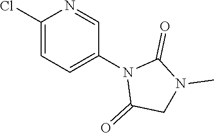

DIPEA (3.90 mL, 22.32 mmol) was added to a suspension of methyl methylglycinate HCl (1.04 g, 7.44 mmol) in MeCN (20 mL) and the reaction mixture was stirred at 20° C. for 15 min. 4-Nitrophenyl (6-chloropyridin-3-yl)carbamate Intermediate 61 (2.18 g, 7.44 mmol) was added and the reaction mixture was stirred at 20° C. for 10 min. The solvent was removed under reduced pressure. The crude product was purified by normal phase flash chromatography on silica (gradient: 65-70% EtOAc in PE) to give the title compound (1.55 g, 92%) as a white solid; MS (ESI) m/z [M+H]⁺=225.8.

### Intermediate 63

### 3-(6-(((1S,3S)-3-((3-(4-Methoxybenzyl)-3H-imidazo[4,5-b]pyridin-2-yl)amino)cyclopentyl)amino)pyridin-3-yl)-1-methylimidazolidine-2,4-dione*

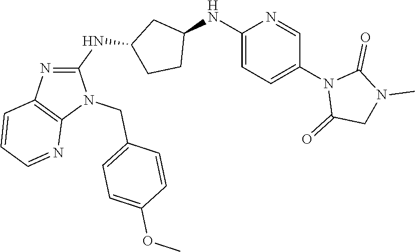 [SMILES: COc1ccc(Cn2c(N[C@H]3CC[C@H](Nc4ccc(N5C(=O)CN(C)C5=O)cn4)C3)nc3cccnc32)cc1]

2.5 M NaOtBu in THF (2.66 mL, 6.65 mmol) was added to a mixture of (1S,3S)—N¹-(3-(4-methoxybenzyl)-3H-imidazo[4,5-b]pyridin-2-yl)cyclopentane-1,3-diamine* HCl Intermediate 60 (829 mg, 2.22 mmol), 3-(6-chloropyridin-3-yl)-1-methylimidazolidine-2,4-dione Intermediate 62 (250 mg, 1.11 mmol) and Pd-PEPPSI-IpentCl 2-methylpyridine (93 mg, 0.11 mmol) in DMA (5.00 mL) at 20° C. The reaction mixture was stirred at 100° C. for 16 h under a nitrogen atmosphere. The reaction mixture was poured into water (50 mL) and extracted with EtOAc (2×100 mL). The combined organic layers were washed with sat brine (3×25 mL), dried over Na₂SO₄, filtered, and concentrated. The crude product was purified by reversed phase flash chromatography on a C18-column (gradient: 40-55% MeCN in water) to give the title compound (243 mg, 42%) as a single regioisomer and as a brown oil which solidified on standing; MS (ESI) m/z [M+H]⁺ 527.2. (*regiochemistry was not confirmed)

### Intermediate 64

### (1S,3S)—N¹-(5-Bromopyrimidin-2-yl)-N³-(3-(4-methoxybenzyl)-3H-imidazo[4,5-b]pyridin-2-yl)cyclopentane-1,3-diamine*

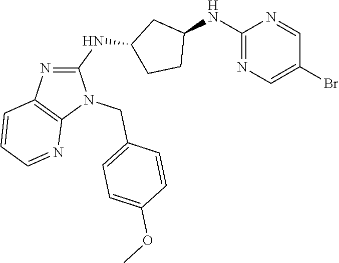 [SMILES: COc1ccc(Cn2c(N[C@H]3CC[C@H](Nc4ncc(Br)cn4)C3)nc3cccnc32)cc1]

(1S,3S)—N¹-(3-(4-Methoxybenzyl)-3H-imidazo[4,5-b]pyridin-2-yl)cyclopentane-1,3-diamine* HCl Intermediate 60 (500 mg, 1.48 mmol) was added to 5-bromo-2-chloropyrimidine (430 mg, 2.22 mmol) and Cs₂CO₃ (966 mg, 2.96 mmol) in DMSO (10 mL) at 20° C. The resulting mixture was stirred at 100° C. for 15 h. The reaction mixture was poured into water (200 mL) and extracted with EtOAc (5×150 mL). The organic layers were combined and washed with sat brine (3×100 mL), dried over Na₂SO₄, filtered and evaporated. The residue was purified by preparative TLC (EtOAc:PE, 3:7) and then by reversed phase flash chromatography on a C18 column (gradient: 0-73% MeCN in water) to give the title compound (237 mg, 32%) as an orange solid; MS (ESI) m/z [M+H]⁺ 494.0 and 496.0 (Br isotope pattern). (*regiochemistry was not confirmed)

### Intermediate 65

### 3-(2-(((1S,3S)-3-((3-(4-Methoxybenzyl)-3H-imidazo[4,5-b]pyridin-2-yl)amino)cyclopentyl)amino)pyrimidin-5-yl)-1-methylpyridin-2(1H)-one*

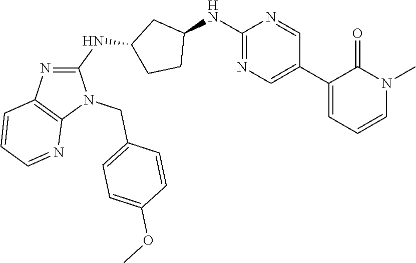 [SMILES: COc1ccc(Cn2c(N[C@H]3CC[C@H](Nc4ncc(-c5cccn(C)c5=O)cn4)C3)nc3cccnc32)cc1]

(1S,3S)—N¹-(5-Bromopyrimidin-2-yl)-N³-(3-(4-methoxybenzyl)-3H-imidazo[4,5-b]pyridin-2-yl)cyclopentane-1,3-diamine* Intermediate 64 (200 mg, 0.40 mmol) was added to (1-methyl-2-oxo-1,2-dihydropyridin-3-yl)boronic acid (390 mg, 2.55 mmol), K₂CO₃ (168 mg, 1.21 mmol) and Pd(dtbpf)Cl₂ (39.5 mg, 0.06 mmol) in 1,4-dioxane (4 mL) and water (1 mL) at 20° C. The resulting mixture was stirred at 100° C. for 15 h under a nitrogen atmosphere. The reaction mixture was poured into water (150 mL) and extracted with EtOAc (4×125 mL). The organic layer was dried over Na₂SO₄, filtered and evaporated. The residue was purified by preparative TLC (MeOH:DCM, 1:15) and then by reversed phase flash chromatography on a C18 column (gradient: 0-51% MeOH in water) to give the title compound (76 mg, 35%) as a brown solid; MS (ESI) m/z [M+H]⁺ 523.3. (*regiochemistry was not confirmed)

### Intermediate 66

### 6′-(((1S,3S)-3-((3-(4-Methoxybenzyl)-3H-imidazo[4,5-b]pyridin-2-yl)amino)cyclopentyl)amino)-3-methyl-2H-[1,3′-bipyridin]-2-one*

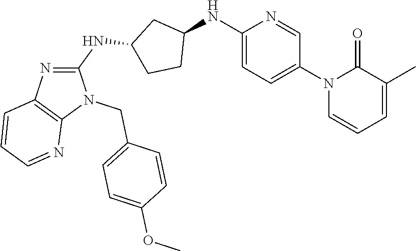 [SMILES: COc1ccc(Cn2c(N[C@H]3CC[C@H](Nc4ccc(-n5cccc(C)c5=O)cn4)C3)nc3cccnc32)cc1]

K₃PO₄ (236 mg, 1.11 mmol) was added to a suspension of (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (200 mg, 0.37 mmol), 3-methylpyridin-2-ol (121 mg, 1.11 mmol), CuI (35 mg, 0.19 mmol) and DMCDA (26 mg, 0.19 mmol) in 1,4-dioxane (5.0 mL) and the reaction mixture was heated at 100° C. for 16 h under a nitrogen atmosphere. The solvent was removed under reduced pressure. The residue was poured into water (100 mL), extracted with EtOAc (2×50 mL), and the combined organic layer was dried over Na₂SO₄, filtered, and concentrated at reduced pressure The residue was purified by preparative TLC (EtOAc:PE, 1:3) to give the title compound (101 mg, 52%) as a brown solid and as a single regioisomer; MS (ESI) m/z [M+H]⁺ 522.45. (*regiochemistry was not confirmed)

### Intermediate 67

### 3-Methoxy-6′-(((1S,3S)-3-((3-(4-methoxybenzyl)-3H-imidazo[4,5-b]pyridin-2-yl)amino)cyclopentyl)amino)-2H-[1,3′-bipyridin]-2-one*

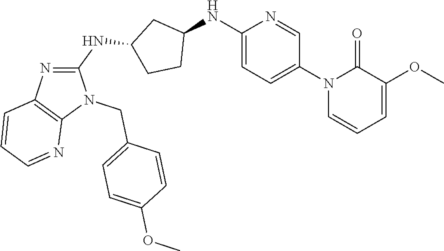 [SMILES: COc1ccc(Cn2c(N[C@H]3CC[C@H](Nc4ccc(-n5cccc(OC)c5=O)cn4)C3)nc3cccnc32)cc1]

Cs₂CO₃ (543 mg, 1.67 mmol) was added to a suspension of (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (300 mg, 0.56 mmol), 3-methoxypyridin-2-ol (208 mg, 1.67 mmol), CuI (53 mg, 0.28 mmol) and DMCDA (39 mg, 0.28 mmol) in 1,4-dioxane (5.0 mL) and the reaction mixture was stirred at 100° C. for 16 h. The solvent was removed under reduced pressure. The residue was poured into water (100 mL), extracted with EtOAc (2×100 mL), and the combined organic layer was dried over Na₂SO₄, filtered, and concentrated. The residue was purified by preparative TLC (7 M NH₃ in MeOH:DCM, 1:20) to give the title compound (86 mg, 29%) as a brown solid and as a single regioisomer; MS (ESI) m/z [M+H]⁺ 538.20. (*regiochemistry was not confirmed)

### Intermediate 68

### 3-Chloro-6′-(((1S,3S)-3-((3-(4-methoxybenzyl)-3H-imidazo[4,5-b]pyridin-2-yl)amino)cyclopentyl)amino)-2H-[1,3′-bipyridin]-2-one*

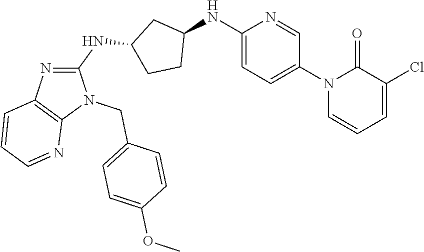 [SMILES: COc1ccc(Cn2c(N[C@H]3CC[C@H](Nc4ccc(-n5cccc(Cl)c5=O)cn4)C3)nc3cccnc32)cc1]

DMCDA (26 mg, 0.19 mmol) was added to a mixture of (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (200 mg, 0.37 mmol), 3-chloropyridin-2-ol (96 mg, 0.74 mmol), Cs₂CO₃ (362 mg, 1.11 mmol) and CuI (35 mg, 0.19 mmol) in 1,4-dioxane (8 mL) and the reaction mixture was stirred at 100° C. for 18 h under a nitrogen atmosphere. The reaction mixture was diluted with EtOAc (75 mL) and washed with water (3×25 mL) and sat brine (3×15 mL). The organic layer was dried over Na₂SO₄, filtered, and concentrated. The residue was purified by preparative TLC (MeOH:DCM, 15:1) to give the title compound (95 mg, 47%) as a brown solid and as a single regioisomer; MS (ESI) m/z [M+H]⁺ 541. (*regiochemistry was not confirmed)

### Intermediate 69

### 6′-(((1S,3S)-3-((3-(4-Methoxybenzyl)-3H-imidazo[4,5-b]pyridin-2-yl)amino)cyclopentyl)amino)-2-oxo-2H-[1,3′-bipyridine]-5-carbonitrile*

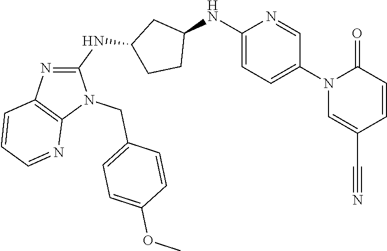 [SMILES: COc1ccc(Cn2c(N[C@H]3CC[C@H](Nc4ccc(-n5cc(C#N)ccc5=O)cn4)C3)nc3cccnc32)cc1]

Cs₂CO₃ (2258 mg, 6.93 mmol) was added to 6′-chloro-2-oxo-2H-[1,3′-bipyridine]-5-carbonitrile Intermediate 18 (321 mg, 1.39 mmol), (1S,3S)—N¹-(3-(4-methoxybenzyl)-3H-imidazo[4,5-b]pyridin-2-yl)cyclopentane-1,3-diamine* HCl Intermediate 60 (674 mg, 1.80 mmol) and Pd-PEPPSI-IpentCl 2-methylpyridine (117 mg, 0.14 mmol) in DMA (5.0 mL) and the reaction mixture was stirred at 100° C. for 16 h under a nitrogen atmosphere. The reaction mixture was poured into water (100 mL) and extracted with EtOAc (2×50 mL). The combined organic layer was washed with sat brine (3×25 mL), dried over Na₂SO₄, filtered, and concentrated. The crude product was purified by reversed phase flash chromatography on a C18 column (gradient: 40-55% MeCN in water). Pure fractions were evaporated to dryness and the product was purified by preparative HPLC, prepMethod K (gradient: 25-35%) to give the title compound (57 mg, 8%) as a beige solid and as a single regioisomer; MS (ESI) m/z [M+H]⁺ 533.35. (*regiochemistry was not confirmed)

### Intermediate 70

### Methyl 6′-(((1S,3S)-3-((3-(4-methoxybenzyl)-3H-imidazo[4,5-b]pyridin-2-yl)amino)cyclopentyl)amino)-2-oxo-2H-[1,3′-bipyridine]-5-carboxylate*

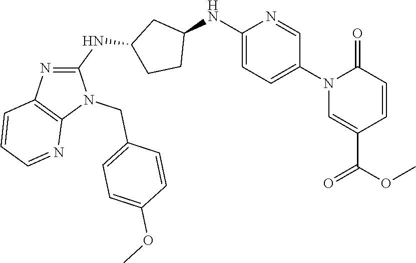 [SMILES: COC(=O)c1ccc(=O)n(-c2ccc(N[C@H]3CC[C@H](Nc4nc5cccnc5n4Cc4ccc(OC)cc4)C3)nc2)c1]

Cs₂CO₃ (724 mg, 2.22 mmol) was added to a mixture of (1S,3S)—N¹-(3-(4-methoxybenzyl)-3H-imidazo[4,5-b]pyridin-2-yl)cyclopentane-1,3-diamine* TFA Intermediate 55 (500 mg, 0.74 mmol), methyl 6′-chloro-2-oxo-2H-[1,3′-bipyridine]-5-carboxylate Intermediate 15 (392 mg, 1.48 mmol) and Pd-PEPPSI-IpentCl 2-methylpyridine (50 mg, 0.06 mmol) in 1,4-dioxane (10 mL) and the reaction mixture was stirred at 100° C. for 16 h under a nitrogen atmosphere. The solvent was removed under reduced pressure. The residue was poured into water (100 mL), extracted with EtOAc (2×100 mL), and the combined organic layer was dried over Na₂SO₄, filtered, and concentrated at reduced pressure. The residue was purified by preparative TLC (EtOAc:PE, 2:1), and then by reversed phase flash chromatography on a C18 column (gradient: 30-50% MeCN in water) to give the title compound (86 mg, 21%) as a brown solid and as a single regioisomer; MS (ESI) m/z [M+H]⁺ 566.40. (*regiochemistry was not confirmed)

### Intermediate 71

### Methyl 6′-(((1S,3S)-3-((3H-imidazo[4,5-b]pyridin-2-yl)amino)cyclopentyl)amino)-2-oxo-2H-[1,3′-bipyridine]-5-carboxylate

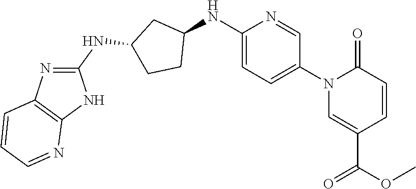 [SMILES: COC(=O)c1ccc(=O)n(-c2ccc(N[C@H]3CC[C@H](Nc4nc5cccnc5[nH]4)C3)nc2)c1]

A mixture of methyl 6′-(((1S,3S)-3-((3-(4-methoxybenzyl)-3H-imidazo[4,5-b]pyridin-2-yl)amino)cyclopentyl)amino)-2-oxo-2H-[1,3′-bipyridine]-5-carboxylate* Intermediate 70 (81 mg, 0.14 mmol) in TFA (5.0 mL) was stirred at 80° C. for 16 h under a nitrogen atmosphere. The solvent was removed under reduced pressure, to give the TFA salt of the title compound (104 mg, 99%) as a yellow solid. The product was used in the next step directly without further purification. (*regiochemistry was not confirmed)

### Intermediate 72

### 3-(6-(((1S,3S)-3-((3-(4-Methoxybenzyl)-3H-imidazo[4,5-b]pyridin-2-yl)amino)cyclopentyl)amino)pyridin-3-yl)-1-methyl-1,3-dihydro-2H-imidazo[4,5-b]pyridin-2-one*

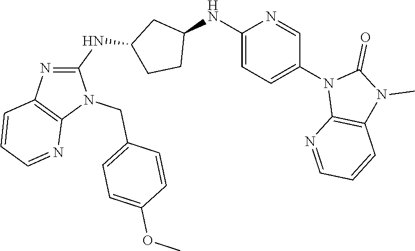 [SMILES: COc1ccc(Cn2c(N[C@H]3CC[C@H](Nc4ccc(-n5c(=O)n(C)c6cccnc65)cn4)C3)nc3cccnc32)cc1]

Sodium 2,2-dimethylpropan-1-olate (121 mg, 1.09 mmol) was added to a mixture of 3-(6-chloropyridin-3-yl)-1-methyl-1,3-dihydro-2H-imidazo[4,5-b]pyridin-2-one Intermediate 19 (47 mg, 0.18 mmol), (1S,3S)—N¹-(3-(4-methoxybenzyl)-3H-imidazo[4,5-b]pyridin-2-yl)cyclopentane-1,3-diamine* HCl Intermediate 60 (80 mg, 0.24 mmol) and Pd-PEPPSI-IpentCl 2-methylpyridine (12 mg, 0.01 mmol) in DMA (3 mL) and the reaction mixture was stirred at 100° C. for 18 h under a nitrogen atmosphere. The reaction mixture was filtered through Celite and the filtrate was concentrated. The crude product was purified by reversed phase flash chromatography on a C18 column (gradient: 0-67% MeCN in water) to give the title compound (65 mg, 64%) as a brown solid and as a single regioisomer; MS (ESI) m/z [M+H]⁺ 562. (*regiochemistry was not confirmed)

### Intermediate 73

### 6′-(((1S,3S)-3-((3-(4-Methoxybenzyl)-3H-imidazo[4,5-b]pyridin-2-yl)amino)cyclopentyl)amino)-2H-[1,3′-bipyridin]-2-one and 6′-(((1S,3S)-3-((1-(4-Methoxybenzyl)-1H-imidazo[4,5-b]pyridin-2-yl)amino)cyclopentyl)amino)-2H-[1,3′-bipyridin]-2-one

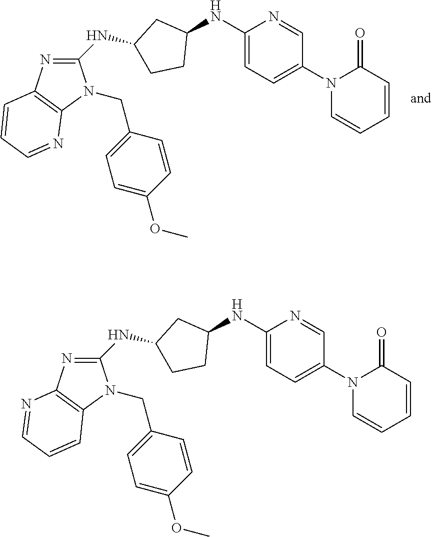 [SMILES: COc1ccc(Cn2c(N[C@H]3CC[C@H](Nc4ccc(-n5ccccc5=O)cn4)C3)nc3cccnc32)cc1 ; COc1ccc(Cn2c(N[C@H]3CC[C@H](Nc4ccc(-n5ccccc5=O)cn4)C3)nc3ncccc32)cc1]

1-(Chloromethyl)-4-methoxybenzene (117 mg, 0.75 mmol) was added to 2-chloro-1H-imidazo[4,5-b]pyridine (125 mg, 0.81 mmol) and MTBD (300 mg, 1.96 mmol) in DMA (4 mL) at 20° C. and the resulting solution was stirred at 20° C. for 18 h under a nitrogen atmosphere. Pd-PEPPSI-IpentCl 2-methylpyridine (27.4 mg, 0.03 mmol) and 6′-(((1S,3S)-3-aminocyclopentyl)amino)-2H-[1,3′-bipyridin]-2-one×HCl Intermediate 3 (100 mg, 0.33 mmol) were added and the resulting suspension was stirred at 100° C. for 16 h. The crude reaction mixture was directly purified by reversed phase flash chromatography on a C18 column (gradient: 5-30% of MeCN in water) to give a mixture of the regioisomeric title compounds (120 mg, 73%) as a yellow gum; MS (ESI) m/z [M+H]⁺ 508.35.

### Intermediate 74

### 2-Chloro-6-fluoro-3H-imidazo[4,5-b]pyridine

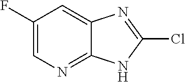 [SMILES: Fc1cnc2[nH]c(Cl)nc2c1]

POCl₃ (6.0 mL, 64 mmol) was added to 6-fluoro-1,3-dihydro-2H-imidazo[4,5-b]pyridin-2-one (130 mg, 0.85 mmol) in an oven-dried microwave vial. Pyridine (0.68 mL, 8.5 mmol) was slowly added at rt to the reaction mixture and the reaction mixture was then heated at 120° C. for 90 min. Tetrabutylammonium chloride (1.18 g, 4.25 mmol) was added and the reaction mixture was heated at 120° C. for 5 h. The volatiles were removed in vacuo, the obtained oil was taken up in EtOAc, and the mixture was sonicated with 7.5% NaHCO₃ (aq). The phases were separated, and the aqueous layer was extracted with EtOAc (×2). The combined organic layers were dried over Na₂SO₄, filtered, and concentrated under reduced pressure. The obtained material was purified by flash chromatography on silica (gradient: 15-100% EtOAc in heptane) to afford 2-chloro-6-fluoro-3H-imidazo[4,5-b]pyridine (89 mg, 61%) as a white solid; MS (ESI) m/z [M+H]⁺ 172.0 and 174.0 (C₁ isotope pattern); ¹H NMR (500 MHz, DMSO-d₆) δ 7.98 (1H, dd), 8.37 (1H, dd).

### Intermediate 75

### 6-Bromo-3-(4-methoxybenzyl)-3H-imidazo[4,5-b]pyridine and 6-Bromo-1-(4-methoxybenzyl)-1H-imidazo[4,5-b]pyridine

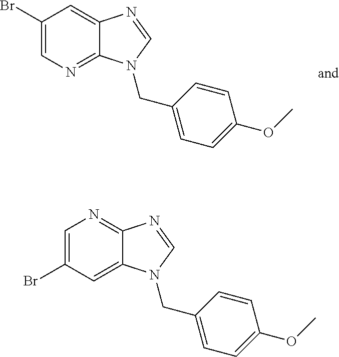 [SMILES: COc1ccc(Cn2cnc3cc(Br)cnc32)cc1 ; COc1ccc(Cn2cnc3ncc(Br)cc32)cc1]

NaH (1.17 g, 29.3 mmol) was added to an ice-cold solution of 6-bromo-3H-imidazo[4,5-b]pyridine (3.87 g, 19.5 mmol) in DMF (95 mL) and the reaction mixture was stirred at 0° C. for 10 min. 1-(Chloromethyl)-4-methoxybenzene (2.78 ml, 20.52 mmol) was added and the reaction mixture allowed to warm to rt and stirred for 17 h. The reaction was quenched with 10% NH₄Cl (aq), diluted with water and extracted with EtOAc (×2). The combined organic layers were washed with brine, dried over Na₂SO₄, filtered, and concentrated under reduced pressure. The residue was purified by flash chromatography on silica (gradient: 40-100% of EtOAc in heptane) to afford a mixture of 6-bromo-3-(4-methoxybenzyl)-3H-imidazo[4,5-b]pyridine and 6-bromo-1-(4-methoxybenzyl)-1H-imidazo[4,5-b]pyridine (5.21 g, 84%) as a yellow oil; MS (ESI) m/z [M+H]⁺ 318.0 and 320.11 (Br isotope pattern).

### Intermediate 76

### 6-Bromo-2-chloro-3-(4-methoxybenzyl)-3H-imidazo[4,5-b]pyridine and 6-Bromo-2-chloro-1-(4-methoxybenzyl)-1H-imidazo[4,5-b]pyridine

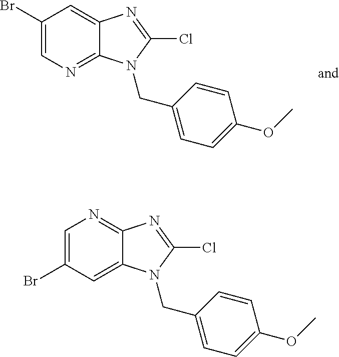 [SMILES: COc1ccc(Cn2c(Cl)nc3cc(Br)cnc32)cc1 ; COc1ccc(Cn2c(Cl)nc3ncc(Br)cc32)cc1]

A mixture of 6-bromo-3-(4-methoxybenzyl)-3H-imidazo[4,5-b]pyridine and 6-bromo-1-(4-methoxybenzyl)-1H-imidazo[4,5-b]pyridine Intermediate 75 (1.00 g, 3.14 mmol) was dissolved in THF (17 mL) under an argon atmosphere, and the solution was cooled to −78° C. 1 M Lithium bis(trimethylsilyl)amide in THF (4.09 mL, 4.09 mmol) was added and the reaction mixture was stirred at −78° C. for 2 h. A solution of 1-chloropyrrolidine-2,5-dione (0.839 g, 6.29 mmol) in THF (17 mL) was added to the reaction mixture and the reaction mixture was cooled to warm to rt over 15 min. The reaction was quenched with 10% NH₄Cl (aq). The mixture was extracted with EtOAc (×2) and the combined organic layers were washed with brine, dried over Na₂SO₄, filtered, and concentrated at reduced pressure. The residue was purified by flash chromatography on silica (gradient: 20-50% EtOAc in heptane) to afford a mixture of 6-bromo-2-chloro-3-(4-methoxybenzyl)-3H-imidazo[4,5-b]pyridine and 6-bromo-2-chloro-1-(4-methoxybenzyl)-1H-imidazo[4,5-b]pyridine (0.376 g, 34%) as a white solid; MS (ESI) m/z [M+H]⁺ 352.1 and 354.1 and 355.9 (Br/Cl isotope pattern); ¹H NMR (500 MHz, DMSO-d₆) δ 3.71 (3H, d), 5.39-5.49 (2H, m), 6.87-6.94 (2H, m), 7.21-7.27 (2H, m), 8.38-8.56 (2H, m).

### Intermediate 77

### 5-(6-Methoxypyridin-3-yl)-2-methylpyrimidine

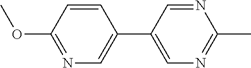

5-Bromo-2-methylpyrimidine (257 mg, 1.48 mmol), (6-methoxypyridin-3-yl)boronic acid (227 mg, 1.48 mmol) and Pd(PPh₃)₄ (86 mg, 0.07 mmol) were mixed in a sealed reactor, and the reactor was evacuated and backfilled with Ar(g). DME (5.94 mL) and 2 M Na₂CO₃ (aq, 1.48 mL, 2.97 mmol) were added and the reaction mixture was stirred at 80° C. for 1h 30 min. The reaction mixture was cooled to rt, EtOAc was added and the mixture was washed with 10% NaHCO₃ and brine. The organic layer was dried over Na₂SO₄, filtered, and concentrated at reduced pressure. The residue was purified by normal phase flash chromatography on silica (gradient: 30-60% of EtOAc in heptane) to give the title compound (217 mg, 73%) as a pale yellow solid; MS (ESI) m/z [M+H]⁺ 202.18.

### Intermediate 78

### 5-(2-Methylpyrimidin-5-yl)pyridin-2-ol

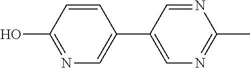

A solution of 5-(6-methoxypyridin-3-yl)-2-methylpyrimidine Intermediate 77 (217 mg, 1.08 mmol) in EtOH (2.5 mL) and 48% HBr in water (5 mL, 45 mmol) was stirred at 80° C. for 6 h, then at rt for 64 h, and then at 80° C. for 6 h. The reaction mixture was cooled to rt and then concentrated at reduced pressure. The residue was precipitated from THF and the beige precipitate was filtered, washed with THF and dried to give the HBr salt of the title compound (277 mg, 74%); MS (ESI) m/z [M+H]⁺ 188.0.

### Intermediate 79

### tert-Butyl ((1S,3S)-3-((5-(2-methylpyrimidin-5-yl)-2-oxo-2H-[1,3′-bipyridin]-6′-yl)amino)cyclopentyl)carbamate

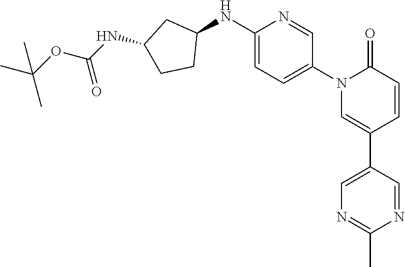 [SMILES: Cc1ncc(-c2ccc(=O)n(-c3ccc(N[C@H]4CC[C@H](NC(=O)OC(C)(C)C)C4)nc3)c2)cn1]

A suspension of 5-(2-methylpyrimidin-5-yl)pyridin-2-ol Intermediate 78 (217 mg, 0.62 mmol) and DIPEA (344 μL, 1.97 mmol) in 1,4-dioxane (2 mL) in a sealed tube was stirred for 5 min under Ar(g) atmosphere. tert-Butyl ((1S,3S)-3-((5-iodopyridin-2-yl)amino)cyclopentyl)carbamate Intermediate 1 (199 mg, 0.49 mmol), CuI (19 mg, 0.10 mmol), K₂CO₃ (273 mg, 1.97 mmol) and DMCDA (16 μL, 0.10 mmol) were added and the reaction vessel was evacuated and back filled with Ar(g) (×3) and then stirred at 90° C. for 4 h. DMSO (696 μL) was added and the reaction mixture was stirred at 90° C. for 23 h. The reaction mixture was cooled to rt. EtOAc was added and the mixture was washed with 10% NH₄Cl (aq), 10% NaHCO₃ (aq) and brine. The organic layer was dried over Na₂SO₄, filtered, and concentrated at reduced pressure. The residue was triturated with heptane:EtOAc (4:1) and the formed beige solid was filtered off, washed with heptane:EtOAc (4:1) and dried to give the title compound (176 mg, 77%); MS (ESI) m/z [M+H]⁺ 463.4.

### Intermediate 80

### 6′-(((1S,3S)-3-Aminocyclopentyl)amino)-5-(2-methylpyrimidin-5-yl)-2H-[1,3′-bipyridin]-2-one

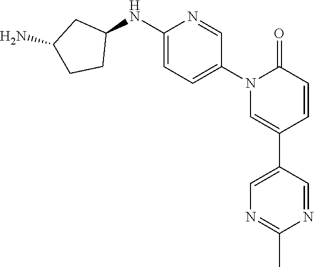 [SMILES: Cc1ncc(-c2ccc(=O)n(-c3ccc(N[C@H]4CC[C@H](N)C4)nc3)c2)cn1]

4 M HCl in 1,4-dioxane (3.79 mL, 15.2 mmol) was added to a suspension of tert-butyl ((1S,3S)-3-((5-(2-methylpyrimidin-5-yl)-2-oxo-2H-[1,3′-bipyridin]-6′-yl)amino)cyclopentyl)carbamate Intermediate 79 (175 mg, 0.38 mmol) in MeOH (1 mL) and the reaction mixture was stirred at rt for 30 min. The solvent was removed to give the HCl salt of the title compound (264 mg, crude) as a brown solid; MS (ESI) m/z [M+H]⁺ 363.3.

### Intermediate 81

### 6′-(((1S,3S)-3-((6-Bromo-3-(4-methoxybenzyl)-3H-imidazo[4,5-b]pyridin-2-yl)amino)cyclopentyl)amino)-5-(2-methylpyrimidin-5-yl)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-5-(2-methylpyrimidin-5-yl)-2H-[1,3′-bipyridin]-2-one

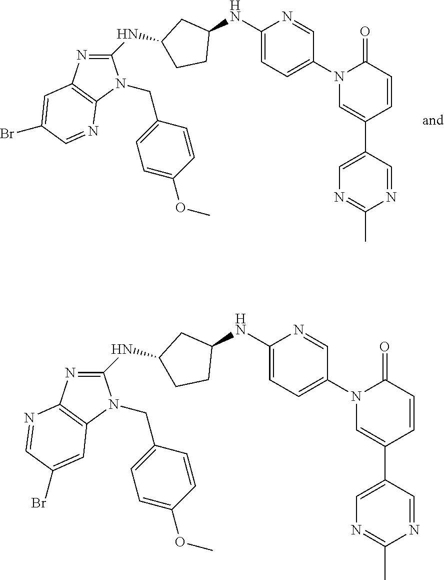 [SMILES: COc1ccc(Cn2c(N[C@H]3CC[C@H](Nc4ccc(-n5cc(-c6cnc(C)nc6)ccc5=O)cn4)C3)nc3cc(Br)cnc32)cc1 ; COc1ccc(Cn2c(N[C@H]3CC[C@H](Nc4ccc(-n5cc(-c6cnc(C)nc6)ccc5=O)cn4)C3)nc3ncc(Br)cc32)cc1]

A mixture of 6-bromo-2-chloro-3-(4-methoxybenzyl)-3H-imidazo[4,5-b]pyridine and 6-bromo-2-chloro-1-(4-methoxybenzyl)-1H-imidazo[4,5-b]pyridine Intermediate 76 (120 mg, 0.34 mmol), 6′-(((1S,3S)-3-aminocyclopentyl)amino)-5-(2-methylpyrimidin-5-yl)-2H-[1,3′-bipyridin]-2-one HCl Intermediate 80 (68.9 mg, 0.19 mmol) and DIPEA (331 μL, 1.90 mmol) in NMP (1.57 mL), in a sealed vial, was stirred at 130° C. for 24 h. KF (22 mg, 0.38 mmol) was added and the reaction mixture was stirred at 130° C. for 18 h. The reaction mixture was concentrated at reduced pressure and the residue was purified by normal phase flash chromatography on silica (gradient: 0-20% MeOH in DCM). The fractions containing the two regioisomers were combined and concentrated to give the title compounds (95 mg, 74%) as a brown solid; MS (ESI) m/z [M+H]⁺ 678 and 680.3 (Br isotope pattern).

### Intermediate 82

### 6′-(((1S,3S)-3-((6-Bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-Bromo-1-(4-methoxybenzyl)-1H-imidazo[4,5-b]pyridin-2-yl)amino)cyclopentyl)amino)-2H-[1,3′-bipyridin]-2-one

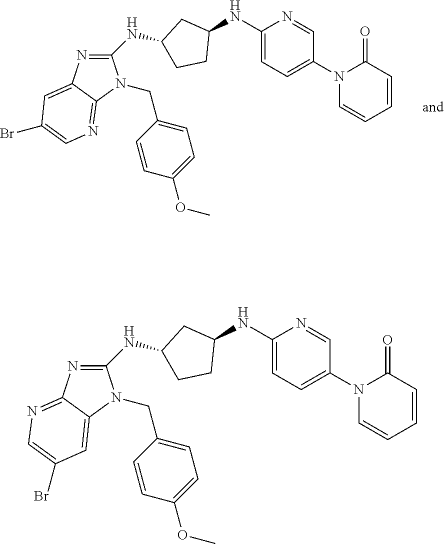 [SMILES: COc1ccc(Cn2c(N[C@H]3CC[C@H](Nc4ccc(-n5ccccc5=O)cn4)C3)nc3cc(Br)cnc32)cc1 ; COc1ccc(Cn2c(N[C@H]3CC[C@H](Nc4ccc(-n5ccccc5=O)cn4)C3)nc3ncc(Br)cc32)cc1]

A solution of 6′-(((1S,3S)-3-aminocyclopentyl)amino)-2H-[1,3′-bipyridin]-2-one×HCl Intermediate 3 (109 mg, 0.40 mmol) in NMP (2.5 mL) was added to a sealed reactor containing a mixture of 6-bromo-2-chloro-3-(4-methoxybenzyl)-3H-imidazo[4,5-b]pyridine and 6-bromo-2-chloro-1-(4-methoxybenzyl)-1H-imidazo[4,5-b]pyridine Intermediate 76 (157 mg, 0.45 mmol) in NMP (2.5 mL) at rt. DIPEA (262 mg, 2.02 mmol) was added and the reaction mixture was stirred at 120° C. for 22 h. The mixture was concentrated under reduced pressure and the residue was purified by flash chromatography on silica (gradient: 0-20% of MeOH in DCM) to give a mixture of the regioisomeric title compounds (78 mg, 33%) as a brown solid; MS (ESI) m/z [M+H]⁺ 586.3 and 588.3 (Br isotope pattern). ¹H NMR (500 MHz, DMSO-d₆) δ 1.44-1.72 (2H, m), 1.85-2.12 (3H, m), 2.12-2.29 (3H, m), 3.69 (3H, d), 4.37 (1H, s), 4.44-4.54 (1H, m), 5.25 (2H, s), 6.27 (1H, t), 6.41-6.47 (1H, m), 6.47-6.56 (1H, m), 6.84-6.93 (2H, m), 6.94-7.02 (1H, m), 7.13-7.2 (2H, m), 7.25-7.43 (2H, m), 7.44-7.51 (1H, m), 7.58-7.69 (2H, m), 7.9-7.95 (1H, m), 8.01 (1H, d).

### Intermediate 83

### 6′-(((1S,3S)-3-((6-Hydroxy-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-Hydroxy-1-(4-methoxybenzyl)-1H-imidazo[4,5-b]pyridin-2-yl)amino)cyclopentyl)amino)-2H-[1,3′-bipyridin]-2-one

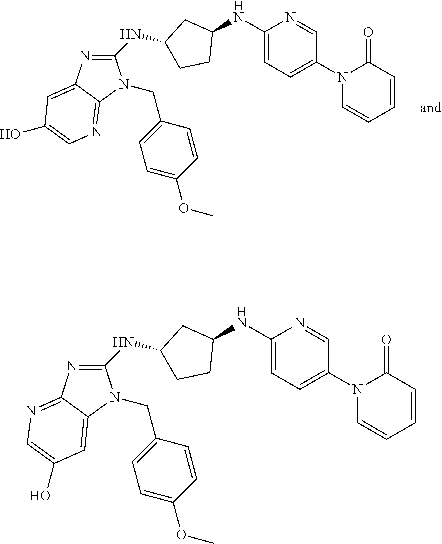 [SMILES: COc1ccc(Cn2c(N[C@H]3CC[C@H](Nc4ccc(-n5ccccc5=O)cn4)C3)nc3cc(O)cnc32)cc1 ; COc1ccc(Cn2c(N[C@H]3CC[C@H](Nc4ccc(-n5ccccc5=O)cn4)C3)nc3ncc(O)cc32)cc1]

A sealed vial with 4,4,4′,4′,5,5,5′,5′-octamethyl-2,2′-bi(1,3,2-dioxaborolane) (57 mg, 0.22 mmol), Pd(dppf)Cl₂·DCM (6.1 mg, 7.4 μmol) and KOAc (29 mg, 0.30 mmol) was evacuated and backfilled with Ar(g). A solution of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (87 mg, 0.15 mmol) in dioxane (1.6 mL) was added and the reaction mixture was stirred at 80° C. for 17 h. The mixture was cooled to rt, diluted with EtOAc and the organic layer was washed with 10% NaHCO₃ (aq) and brine. The organic layer was dried over Na₂SO₄, filtered, and concentrated under reduced pressure. The obtained residue was dissolved in THF (0.4 mL), treated with AcOH (0.043 mL, 0.74 mmol) and 30% H₂O₂ (0.34 mL, 0.30 mmol) and stirred at rt for 1.5 h. The solvent was removed under reduced pressure and the residue was purified by flash chromatography on silica (gradient: 0-20% MeOH in DCM) to give a mixture of the regioisomeric title compounds (20 mg, 26%) as a brown solid; MS (ESI) m/z [M+H]⁺ 524.51.

### Intermediate 84

### 6′-(((1S,3S)-3-((3-(4-Methoxybenzyl)-6-((methoxymethoxy)methyl)-3H-imidazo[4,5-b]pyridin-2-yl)amino)cyclopentyl)amino)-2H-[1,3′-bipyridin]-2-one and 6′-(((1S,3S)-3-((1-(4-Methoxybenzyl)-6-((methoxymethoxy)methyl)-1H-imidazo[4,5-b]pyridin-2-yl)amino)cyclopentyl)amino)-2H-[1,3′-bipyridin]-2-one

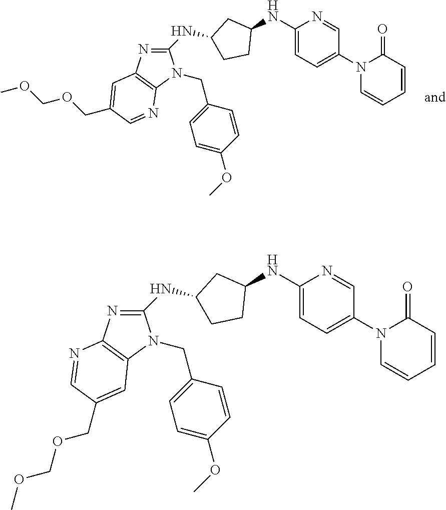 [SMILES: COCOCc1cnc2c(c1)nc(N[C@H]1CC[C@H](Nc3ccc(-n4ccccc4=O)cn3)C1)n2Cc1ccc(OC)cc1 ; COCOCc1cnc2nc(N[C@H]3CC[C@H](Nc4ccc(-n5ccccc5=O)cn4)C3)n(Cc3ccc(OC)cc3)c2c1]

Tributyl((methoxymethoxy)methyl)stannane (148 mg, 0.41 mmol) and tetrakis(triphenylphosphine)palladium(0) (4.7 mg, 4.1 μmol) were added to a mixture of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (48 mg, 0.08 mmol) in DMF (0.8 mL) in a sealed reactor. The reaction mixture was evacuated and backfilled with Ar(g) and then stirred at 110° C. for 16 h. The mixture was cooled to rt, diluted with EtOAc and the organic layer was washed with 10% NaHCO₃ (aq) and brine. The organic layer was dried over Na₂SO₄, filtered, and concentrated under reduced pressure and the residue was purified by flash chromatography on silica (gradient: 0-10% MeOH in DCM) to give a mixture of the regioisomeric title compounds (5.2 mg, 11%) as a brown solid; MS (ESI) m/z [M+H]⁺ 582.3.

### Intermediate 85

### 3-(4-Methoxybenzyl)-6-(trifluoromethyl)-3H-imidazo[4,5-b]pyridine*

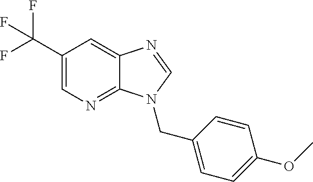 [SMILES: COc1ccc(Cn2cnc3cc(C(F)(F)F)cnc32)cc1]

NaH (70.5 mg, 1.76 mmol) was added to a solution of 6-(trifluoromethyl)-3H-imidazo[4,5-b]pyridine (220 mg, 1.18 mmol) in DMF (4.5 mL), and the mixture stirred at rt for 5 min. 1-(Bromomethyl)-4-methoxybenzene (0.169 mL, 1.18 mmol) was added and the reaction mixture was stirred at rt for 2 h. The reaction was quenched with 10% NH₄Cl (aq) and diluted with water. The mixture was extracted with EtOAc (×3) and the combined organic layers were washed with brine, dried over Na₂SO₄, filtered, and concentrated under reduced pressure. The residue was purified by flash chromatography on silica (gradient: 20-50% of EtOAc in heptane) to afford the title compound (97 mg, 27%) as a beige solid. Only one of the two formed regioisomers was isolated, the second one was lost during purification; MS (ESI) m/z [M+H]⁺ 308.0. (*regiochemistry was not confirmed)

### Intermediate 86

### 2-Chloro-3-(4-methoxybenzyl)-6-(trifluoromethyl)-3H-imidazo[4,5-b]pyridine*

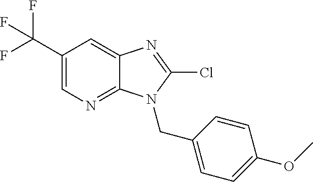 [SMILES: COc1ccc(Cn2c(Cl)nc3cc(C(F)(F)F)cnc32)cc1]

3-(4-Methoxybenzyl)-6-(trifluoromethyl)-3H-imidazo[4,5-b]pyridine* Intermediate 85 (97 mg, 0.31 mmol) was dissolved in THF (2 mL) under an argon atmosphere and the solution was cooled to −78° C. Lithium bis(trimethylsilyl)amide (0.41 mL, 0.41 mmol) was added and the reaction mixture was stirred at −78° C. for 1.5 h. A solution of 1-chloropyrrolidine-2,5-dione (84 mg, 0.63 mmol) in THF (2 mL) was added and the reaction mixture was stirred at −78° C. for 1 h, and then allowed to warm to rt. The reaction was quenched with 10% NH₄Cl (aq) and the mixture was extracted with EtOAc (×2). The combined organic layers were washed with brine, dried over Na₂SO₄, filtered, and concentrated at reduced pressure. The residue was purified by flash chromatography on silica (gradient: 0-30% of EtOAc in heptane) to afford the title compound (63 mg, 59%) as a white solid and as a single regioisomer; MS (ESI) m/z [M+H]⁺ 342.1. ¹H NMR (500 MHz, DMSO-d₆) δ 3.71 (3H, d), 5.49 (2H, s), 6.87-6.93 (2H, m), 7.24-7.3 (2H, m), 8.57 (1H, d), 8.82-8.86 (1H, m). (*regiochemistry was not confirmed)

### Intermediate 87

### 6′-(((1S,3S)-3-((3-(4-Methoxybenzyl)-6-(trifluoromethyl)-3H-imidazo[4,5-b]pyridin-2-yl)amino)cyclopentyl)amino)-2H-[1,3′-bipyridin]-2-one*

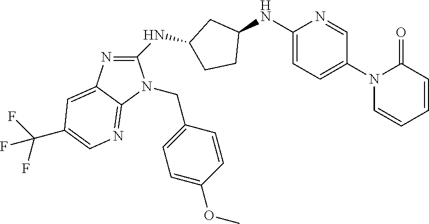 [SMILES: COc1ccc(Cn2c(N[C@H]3CC[C@H](Nc4ccc(-n5ccccc5=O)cn4)C3)nc3cc(C(F)(F)F)cnc32)cc1]

DIPEA (92 mg, 0.71 mmol was added to a solution of 6′-(((1S,3S)-3-aminocyclopentyl)amino)-2H-[1,3′-bipyridin]-2-one×HCl Intermediate 3 (64 mg, 0.24 mmol) and 2-chloro-3-(4-methoxybenzyl)-6-(trifluoromethyl)-3H-imidazo[4,5-b]pyridine* Intermediate 86 (63 mg, 0.19 mmol) in NMP (1 mL) in a sealed reactor at rt, and the reaction mixture was subsequently stirred at 120° C. for 17 h. The mixture was concentrated under reduced pressure and the residue was purified by preparative HPLC (PrepMethod B, gradient: 45-95%). Fractions containing the desired product were combined and concentrated under reduced pressure. The obtained aqueous layer was extracted twice with EtOAc, the combined organic layers were washed with brine, dried over Na₂SO₄, filtered and concentrated under reduced pressure to afford the title compound (42 mg, 31%) as a yellow oil; MS (ESI) m/z [M+H]⁺ 576.5. (*regiochemistry was not confirmed)

### Intermediate 88

### 6′-(((1S,3S)-3-((3-(4-Methoxybenzyl)-3H-imidazo[4,5-b]pyridin-2-yl)amino)cyclopentyl)amino)-3-(trifluoromethoxy)-2H-[1,3′-bipyridin]-2-one*

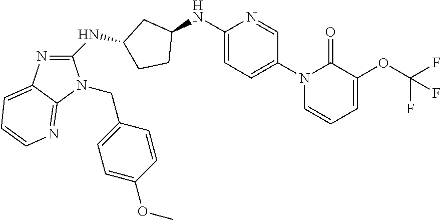 [SMILES: COc1ccc(Cn2c(N[C@H]3CC[C@H](Nc4ccc(-n5cccc(OC(F)(F)F)c5=O)cn4)C3)nc3cccnc32)cc1]

DMCDA (17 mg, 0.12 mmol) was added to a mixture of (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (130 mg, 0.24 mmol), 3-(trifluoromethoxy)pyridin-2-ol (129 mg, 0.72 mmol), Cu(I)I (23 mg, 0.12 mmol) and Cs₂CO₃ (235 mg, 0.72 mmol) in 1,4-dioxane (5 mL) at rt and the reaction mixture was stirred at 100° C. for 18 h under a nitrogen atmosphere. The reaction mixture was diluted with EtOAc (75 mL) and washed with water (3×25 mL) and sat brine (3×25 mL). The organic layer was dried over Na₂SO₄, filtered, and concentrated. The crude product was purified by preparative TLC (7 M NH₃ in MeOH:DCM, 1:20) to give the title compound containing residual impurities (413 mg, 55% purity) as a brown solid and as a single regioisomer; MS (ESI) m/z [M-C₆H₄F₃NO₂+H]⁺ 415. (*regiochemistry was not confirmed)

### Intermediate 89

### 6′-(((1S,3S)-3-((3-(4-Methoxybenzyl)-3H-imidazo[4,5-b]pyridin-2-yl)amino)cyclopentyl)amino)-3-(trifluoromethyl)-2H-[1,3′-bipyridin]-2-one*

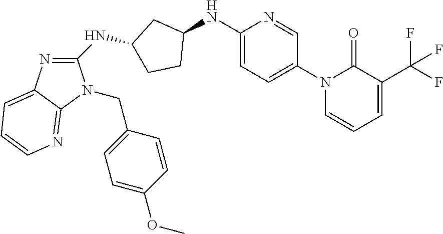 [SMILES: COc1ccc(Cn2c(N[C@H]3CC[C@H](Nc4ccc(-n5cccc(C(F)(F)F)c5=O)cn4)C3)nc3cccnc32)cc1]

DMCDA (68 mg, 0.48 mmol) was added to a mixture of (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (130 mg, 0.24 mmol), 3-(trifluoromethyl)pyridin-2-ol (157 mg, 0.96 mmol), Cu(I)I (92 mg, 0.48 mmol) and Cs₂CO₃ (235 mg, 0.72 mmol) in 1,4-dioxane (3 mL) at rt and the reaction mixture was stirred at 100° C. for 18 h under a nitrogen atmosphere. The reaction mixture was diluted with EtOAc (75 mL) and washed with water (3×25 mL) and sat brine (3×20 mL). The organic layer was dried over Na₂SO₄, filtered and concentrated under reduced pressure and the residue purified by flash chromatography on silica (gradient: 0-18% of MeOH in DCM) to give the title compound (97 mg, 70%) as a brown gum and as a single regioisomer; MS (ESI) m/z [M+H]⁺ 576; ¹H NMR (300 MHz, DMSO-d₆) δ 1.44-1.65 (2H, m), 1.85-2.04 (2H, m), 2.11-2.21 (2H, m), 3.70 (3H, s), 4.28-4.45 (2H, m), 5.48 (2H, s), 6.41 (1H, t), 6.55 (1H, d), 6.73-6.79 (1H, m), 6.87-6.91 (2H, m), 7.02 (1H, d), 7.25-7.27 (1H, m), 7.37-7.46 (4H, m), 7.61-7.65 (1H, m), 7.95-8.01 (3H, m); ¹⁹F NMR (282 MHz, DMSO-d₆) δ −74.07 (3F, s). (*regiochemistry was not confirmed)

### Intermediate 90

### 6-Bromo-2-(methylthio)thiazolo[5,4-b]pyridine

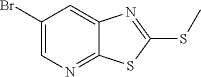

tert-Butyl nitrite (0.807 g, 7.82 mmol) was added dropwise to 6-bromothiazolo[5,4-b]pyridin-2-amine (CAS Reg. No. 1160791-13-8) (0.30 g, 1.30 mmol) and 1,2-dimethyldisulfane (1.16 mL, 13.0 mmol) in MeCN (30 mL) at 20° C. The resulting solution was stirred at 20° C. for 15 h. The reaction mixture was poured into sat brine (150 mL) and extracted with EtOAc (3×100 mL). The organic layer was dried over Na₂SO₄, filtered and evaporated. The residue was purified by preparative TLC (EtOAc:PE, 1:5) to give the title compound (0.3 g, 88%) as a pale-yellow solid. MS (ESI): m/z [M+H]⁺ 260.8 and 262.8. (Br isotope pattern).

### Intermediate 91

### 6-Bromo-2-(methylsulfonyl)thiazolo[5,4-b]pyridine

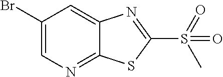

mCPBA (80 wt %, 816 mg, 3.78 mmol) was added dropwise to 6-bromo-2-(methylthio)thiazolo[5,4-b]pyridine Intermediate 90 (395 mg, 1.51 mmol) in DCM (8 mL) at 0° C. The resulting mixture was stirred at 20° C. for 15 h. The reaction mixture was diluted with DCM (100 mL) and washed sequentially with water (3×100 mL). The organic layer was dried over Na₂SO₄, filtered and evaporated. The residue was purified by preparative TLC (PE:EtOAc, 2:1) to give the title compound (440 mg, 99%) as a yellow solid. MS (ESI): m/z [M+H]⁺ 294.8 (Br isotope pattern).

### Intermediate 92

### tert-Butyl ((1S,3S)-3-((5-(7-oxothieno[2,3-c]pyridin-6(7H)-yl)pyridin-2-yl)amino)cyclopentyl)carbamate

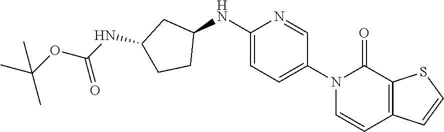 [SMILES: CC(C)(C)OC(=O)N[C@H]1CC[C@H](Nc2ccc(-n3ccc4ccsc4c3=O)cn2)C1]

DMCDA (71 mg, 0.50 mmol) was added to tert-butyl ((1S,3S)-3-((5-iodopyridin-2-yl)amino)cyclopentyl)carbamate Intermediate 1 (200 mg, 0.50 mmol), thieno[2,3-c]pyridin-7(6H)-one (CAS Reg. No. 28981-13-7) (225 mg, 1.49 mmol), CuI (94 mg, 0.50 mmol) and CS₂CO₃ (485 mg, 1.49 mmol) in 1,4-dioxane (10 mL) at 20° C. The resulting suspension was stirred at 100° C. for 15 h under a nitrogen atmosphere. The reaction mixture was diluted with water (50 mL) and extracted with EtOAc (3×100 mL). The organic layers were combined and washed sequentially with water (2×150 mL), dried over Na₂SO₄, filtered and evaporated. The residue was purified by preparative TLC (EtOAc) and then by C18-flash chromatography (gradient: 10-100% MeCN in water) to give the title compound (176 mg, 83%) as a pale-yellow solid. MS (ESI): m/z [M+H]⁺ 426.9.

### Intermediate 93

### 6-(6-(((1S,3S)-3-Aminocyclopentyl)amino)pyridin-3-yl)thieno[2,3-c]pyridin-7(6H)-one

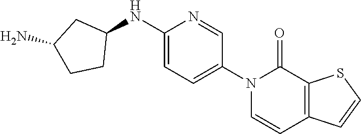 [SMILES: N[C@H]1CC[C@H](Nc2ccc(-n3ccc4ccsc4c3=O)cn2)C1]

4 M HCl in MeOH (2 mL, 8.00 mmol) was added to tert-butyl ((1S,3S)-3-((5-(7-oxothieno[2,3-c]pyridin-6(7H)-yl)pyridin-2-yl)amino)cyclopentyl)carbamate Intermediate 92 (160 mg, 0.38 mmol) in MeOH (5 mL) at 20° C. The resulting solution was stirred at 80° C. for 2 h. The solvent was removed under reduced pressure to give an unspecified HCl salt of the crude title compound (160 mg, 98%) as a pale-yellow solid. MS (ESI): m/z [M+H]⁺ 327.0.

### Intermediate 94

### 6-(6-Chloropyridin-3-yl)thieno[2,3-c]pyridin-7(6H)-one

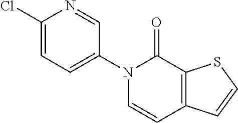 [SMILES: O=c1c2sccc2ccn1-c1ccc(Cl)nc1]

Dimethylglycine HCl (175 mg, 1.25 mmol) was added to 2-chloro-5-iodopyridine (CAS Reg. No. 69045-79-0) (300 mg, 1.25 mmol), thieno[2,3-c]pyridin-7(6H)-one (CAS Reg. No. 28981-13-7) (379 mg, 2.51 mmol), CuI (239 mg, 1.25 mmol) and K₂CO₃ (519 mg, 3.76 mmol) in DMF (5 mL) at 30° C. The resulting suspension was stirred at 100° C. for 12 h under a nitrogen atmosphere. The reaction mixture was diluted with EtOAc (75 mL) and washed sequentially with sat brine (3×250 mL). The organic layer was dried over Na₂SO₄, filtered and evaporated. The residue was purified by preparative TLC (MeOH:DCM=1:20) to give the title compound (214 mg, 64%) as a white solid; MS (ESI): m/z [M+H]⁺ 262.9.

### Intermediate 95

### 1-(6-(((1S,3S)-3-((3-(4-Methoxybenzyl)-3H-imidazo[4,5-b]pyridin-2-yl)amino)cyclopentyl)amino)pyridin-3-yl)imidazolidine-2,4-dione*

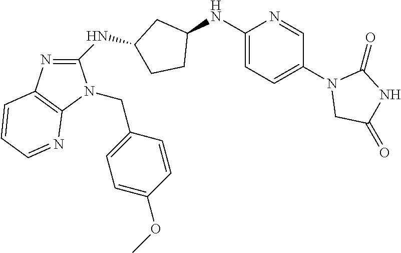 [SMILES: COc1ccc(Cn2c(N[C@H]3CC[C@H](Nc4ccc(N5CC(=O)NC5=O)cn4)C3)nc3cccnc32)cc1]

tBuBrettphos Pd G3 (60.6 mg, 0.07 mmol) was added to 1-(6-chloropyridin-3-yl)imidazolidine-2,4-dione Intermediate 96 (150 mg, 0.71 mmol), (1S,3S)—N1-(1-(4-methoxybenzyl)-1H-imidazo[4,5-b]pyridin-2-yl)cyclopentane-1,3-diamine x HCl Intermediate 55 (345 mg, 0.92 mmol) and sodium tert-pentoxide in THF (2.5 M) (1.70 mL, 4.25 mmol) in DMA (8 mL) at rt and the resulting suspension was stirred at 100° C. for 16 h under nitrogen. The crude product was purified by PrepMethod A (gradient 21-41%) to afford (58 mg, 16%) of the title compound as a pale-yellow gum; MS (ESI) m/z [M+H]⁺ 513.4. (*regiochemistry was not confirmed)

### Intermediate 96

### 1-(6-Chloropyridin-3-yl)imidazolidine-2,4-dione

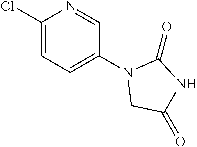 [SMILES: O=C1CN(c2ccc(Cl)nc2)C(=O)N1]

2-Chloroacetyl isocyanate (0.93 g, 7.78 mmol) was added dropwise to 6-chloropyridin-3-amine (1.0 g, 7.78 mmol) in 1,4-dioxane (30 mL) cooled to 0° C. under nitrogen. The resulting solution was stirred at rt for 2 h. DBU (2.93 mL, 19.5 mmol) was added and the resulting solution was stirred at rt for 15 h. The reaction mixture was poured into 0.1 M HCl (200 mL), extracted with EtOAc (3×125 mL), the organic layer was dried over Na₂SO₄, filtered and evaporated to afford the crude product as an orange solid. The crude solid was triturated with petroleum ether:EtOAc (1:1, 30 mL) to give a solid which was collected by filtration and dried under vacuum to give (1.1 g, 67%) of the title compound as an orange solid; MS (ESI) m/z [M+H]⁺ 211.8.

### Intermediate 97

### (1S,3S)—N¹-(5-(2-Methoxyphenyl)pyridin-2-yl)-N3-(3-tosyl-3H-imidazo[4,5-b]pyridin-2-yl)cyclopentane-1,3-diamine*

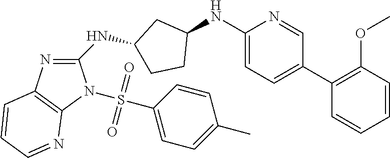 [SMILES: COc1ccccc1-c1ccc(N[C@H]2CC[C@H](Nc3nc4cccnc4n3S(=O)(=O)c3ccc(C)cc3)C2)nc1]

(1S,3S)—N¹-(5-(2-Methoxyphenyl)pyridin-2-yl)cyclopentane-1,3-diamine×3 HCl Intermediate 98 (170 mg, 0.43 mmol) was added to 2-chloro-3-tosyl-3H-imidazo[4,5-b]pyridine* Intermediate 39 (266 mg, 0.87 mmol), Pd-PEPPSI-IpentCl 2-methylpyridine (18 mg, 0.02 mmol) and Cs₂CO₃ (705 mg, 2.16 mmol) in 1,4-dioxane (10 mL) and the mixture was stirred at 100° C. for 16 h under nitrogen. The reaction mixture was filtered through celite. The filter cake was washed with EtOAc (3×20 mL) and the filtrate was concentrated under reduced pressure to afford the crude product which was purified by preparative TLC (MeOH:DCM=1:10) to afford (160 mg, 67%) of the title compound as a red solid; MS (ESI) m/z [M+H]⁺ 555.0. (*regiochemistry was not confirmed)

### Intermediate 98

### (1S,3S)—N¹-(5-(2-Methoxyphenyl)pyridin-2-yl)cyclopentane-1,3-diamine×3 HCl

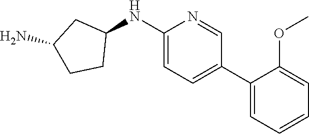 [SMILES: COc1ccccc1-c1ccc(N[C@H]2CC[C@H](N)C2)nc1]

tert-Butyl ((1S,3S)-3-((5-(2-methoxyphenyl)pyridin-2-yl)amino)cyclopentyl)carbamate Intermediate 99 (180 mg, 0.47 mmol) was added to 4 MHCl in MeOH (5 ml, 20.00 mmol). The resulting mixture was stirred at 60° C. for 2 hours. The organic solvents were removed by evaporation to afford (180 mg, 98%) of the title compound as a brown solid which was used in the next step directly without further purification; MS (ESI) m/z [M+H]⁺ 284.1.

### Intermediate 99

### tert-Butyl ((1S,3S)-3-((5-(2-methoxyphenyl)pyridin-2-yl)amino)cyclopentyl)carbamate

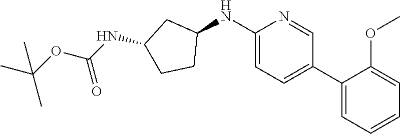 [SMILES: COc1ccccc1-c1ccc(N[C@H]2CC[C@H](NC(=O)OC(C)(C)C)C2)nc1]

tert-Butyl ((1S,3S)-3-((5-iodopyridin-2-yl)amino)cyclopentyl)carbamate Intermediate 1 (250 mg, 0.62 mmol) was added to (2-methoxyphenyl)boronic acid (CAS Reg. No. 5720-06-9) (283 mg, 1.86 mmol), Pd(dppf)Cl₂·DCM (51 mg, 0.06 mmol) and K₃PO₄ (395 mg, 1.86 mmol) in water (2 mL) and 1,4-dioxane (6.00 mL). The resulting mixture was stirred at 80° C. for 3 h under nitrogen. The reaction mixture was quenched with water (150 mL), extracted with EtOAc (3×100 mL), the organic layer was dried over Na₂SO₄, filtered and evaporated to afford crude product which was purified by preparative TLC (EtOAc:petroleum ether=2:1) to afford (190 mg, 80%) of the title compound as a yellow solid; MS (ESI) m/z [M+H]⁺ 384.2.

### Intermediate 100

### (1S,3S)—N¹-(3H-Imidazo[4,5-b]pyridin-2-yl)-N³-(5-iodopyridin-2-yl)cyclopentane-1,3-diamine

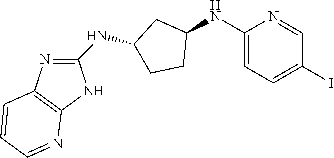 [SMILES: Ic1ccc(N[C@H]2CC[C@H](Nc3nc4cccnc4[nH]3)C2)nc1]

A microwave vial was charged with 2-(methylsulfonyl)-3H-imidazo[4,5-b]pyridine (CAS Reg. No. 57159-81-6) (250 mg, 1.27 mmol), (1S,3S)—N¹-(5-iodopyridin-2-yl)cyclopentane-1,3-diamine (CAS Reg. No. 2272129-95-8) (730 mg, 2.41 mmol) and n-butanol (4 mL). The vial was capped and heated to 160° C. for 2 h in the microwave reactor. The solvent was removed under reduced pressure and the crude product was purified by PrepMethod R (gradient 20-45%) to afford (70 mg, 13%) of the title compound as a colourless gum; MS (ESI) m/z [M+H]⁺ 420.9.

### Intermediate 101

### (1S,3S)—N¹-(5-(2,6-Difluorophenyl)pyridin-2-yl)-N³-(3-tosyl-3H-imidazo[4,5-b]pyridin-2-yl)cyclopentane-1,3-diamine*

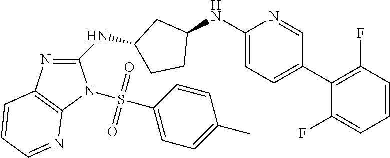 [SMILES: Cc1ccc(S(=O)(=O)n2c(N[C@H]3CC[C@H](Nc4ccc(-c5c(F)cccc5F)cn4)C3)nc3cccnc32)cc1]

(1S,3S)—N¹-(5-(2,6-Difluorophenyl)pyridin-2-yl)cyclopentane-1,3-diamine×3 HCl Intermediate 102 (140 mg, 0.35 mmol) was added to 2-chloro-3-tosyl-3H-imidazo[4,5-b]pyridine* Intermediate 39 (216 mg, 0.70 mmol), Pd-PEPPSI-IpentCl 2-methylpyridine (15 mg, 0.02 mmol) and Cs₂CO₃ (572 mg, 1.76 mmol) in 1,4-dioxane (10 mL). The resulting mixture was stirred at 100° C. for 16 hours under nitrogen. The reaction mixture was filtered through celite. The filter cake was washed with EtOAc (3×20 mL) and the filtrate was concentrated under reduced pressure to afford the crude product which was purified by preparative TLC (MeOH:DCM=1:10) to afford (154 mg, 78%) of the title compound as a red solid; MS (ESI) m/z [M+H]⁺ 561.0. (*regiochemistry was not confirmed)

### Intermediate 102

### (1S,3S)—N¹-(5-(2,6-Difluorophenyl)pyridin-2-yl)cyclopentane-1,3-diamine×3 HCl

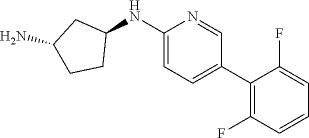 [SMILES: N[C@H]1CC[C@H](Nc2ccc(-c3c(F)cccc3F)cn2)C1]

tert-Butyl ((1S,3S)-3-((5-(2,6-difluorophenyl)pyridin-2-yl)amino)cyclopentyl)carbamate Intermediate 103 (160 mg, 0.41 mmol) was added to 4 M HCl in MeOH (6 mL, 24 mmol). The resulting mixture was stirred at 60° C. for 2 h. The reaction mixture was evaporated to dryness afford (160 mg, 98%) of the title compound as a red oil which was used without further purification; MS (ESI) m/z [M+H]⁺ 290.0.

### Intermediate 103

### tert-Butyl ((1S,3S)-3-((5-(2,6-difluorophenyl)pyridin-2-yl)amino)cyclopentyl)carbamate

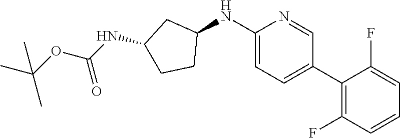 [SMILES: CC(C)(C)OC(=O)N[C@H]1CC[C@H](Nc2ccc(-c3c(F)cccc3F)cn2)C1]

tert-Butyl ((1S,3S)-3-((5-iodopyridin-2-yl)amino)cyclopentyl)carbamate Intermediate 1 (200 mg, 0.50 mmol) was added to (2,6-difluorophenyl)boronic acid (235 mg, 1.49 mmol), K₃PO₄ (316 mg, 1.49 mmol) and Pd(dppf)Cl₂·DCM (40 mg, 0.05 mmol) in water (2 mL) and 1,4-dioxane (6 mL). The resulting mixture was stirred at 80° C. for 3 h under nitrogen. The reaction mixture was quenched with water (100 mL), extracted with EtOAc (3×75 mL). The organic layer was dried over Na₂SO₄, filtered and evaporated to afford crude product which was purified by preparative TLC (MeOH:DCM=1:10) to afford (187 mg, 97%) of the title compound as a white solid; MS (ESI) m/z [M+H]⁺ 390.2.

### Intermediate 104

### (1S,3S)—N¹-(5-(2-Fluorophenyl)pyridin-2-yl)cyclopentane-1,3-diamine×3.5 HCl

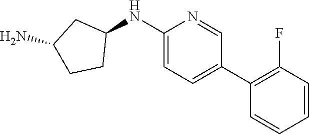 [SMILES: N[C@H]1CC[C@H](Nc2ccc(-c3ccccc3F)cn2)C1]

4 M HCl in MeOH (0.555 mL, 2.22 mmol) was added to tert-butyl ((1S,3S)-3-((5-(2-fluorophenyl)pyridin-2-yl)amino)cyclopentyl)carbamate Intermediate 105 (165 mg, 0.44 mmol) in MeOH (1.0 mL) at rt. The resulting solution was stirred at 60° C. for 3 h. The solvent was removed under reduced pressure to afford (177 mg, 100%) of the title compound as a brown gum which was used in the next step directly without further purification; MS (ESI) m/z [M+H]⁺ 272.0.

### Intermediate 105

### tert-Butyl ((1S,3S)-3-((5-iodopyridin-2-yl)amino)cyclopentyl)carbamate

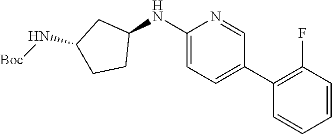 [SMILES: CC(C)(C)OC(=O)N[C@H]1CC[C@H](Nc2ccc(-c3ccccc3F)cn2)C1]

Cs₂CO₃ (485 mg, 1.49 mmol) was added to tert-butyl ((1S,3S)-3-((5-iodopyridin-2-yl)amino)cyclopentyl)carbamate Intermediate 1 (200 mg, 0.50 mmol), 2-fluorophenylboronic acid, pinacol ester (110 mg, 0.50 mmol) and Pd(dtbpf)Cl₂ (32 mg, 0.05 mmol) in 1,4-dioxane (5 mL) at rt. The resulting suspension was stirred at 100° C. for 2 h under nitrogen. The solvent was removed under reduced pressure. The residue was poured into water (100 mL), extracted with EtOAc (2×50 mL), the organic layer was dried over Na₂SO₄, filtered and evaporated to afford crude product. The residue was purified by preparative TLC (EtOAc:PE=1:2) to afford (168 mg, 91%) of the title compound as a brown solid; MS (ESI) m/z [M+H]⁺ 372.0.

### Intermediate 106

### (1S,3S)—N¹-(5-(2-Fluoro-6-(trifluoromethoxy)phenyl)pyridin-2-yl)cyclopentane-1,3-diamine×HCl

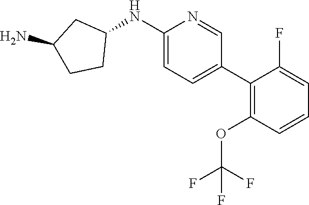 [SMILES: N[C@@H]1CC[C@@H](Nc2ccc(-c3c(F)cccc3OC(F)(F)F)cn2)C1]

4 M HCl in MeOH (1.0 mL, 4.0 mmol) was added to tert-butyl ((1S,3S)-3-((5-(2-fluoro-6-(trifluoromethoxy)phenyl)pyridin-2-yl)amino)cyclopentyl)carbamate Intermediate 107 (80 mg, 0.18 mmol) in MeOH (2.5 mL) at rt and the resulting mixture was stirred at 60° C. for 2 h. The resulting mixture was evaporated to dryness to afford crude product which was used without further purification; MS (ESI) m/z [M+H]⁺ 356.0.

### Intermediate 107

### tert-Butyl ((1S,3S)-3-((5-(2-fluoro-6-(trifluoromethoxy)phenyl)pyridin-2-yl)amino)cyclopentyl)carbamate

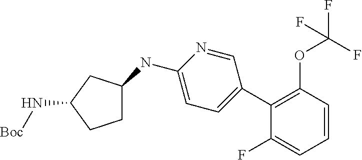 [SMILES: CC(C)(C)OC(=O)N[C@H]1CC[C@H](Nc2ccc(-c3c(F)cccc3OC(F)(F)F)cn2)C1]

CataCXium A Pd G3 (56 mg, 0.08 mmol) was added to tert-butyl ((1S,3S)-3-((5-iodopyridin-2-yl)amino)cyclopentyl)carbamate Intermediate 1 (310 mg, 0.77 mmol), (2-fluoro-6-(trifluoromethoxy)phenyl)boronic acid (310 mg, 1.38 mmol) and CsF (350 mg, 2.31 mmol) in 1,4-dioxane (16 mL) and water (4 mL) at rt and the resulting solution was stirred at 100° C. for 15 h under nitrogen. The reaction mixture was filtered through a Celite pad. The filter cake was washed with DCM (4×100 mL). The resulting filtrate was evaporated to dryness to afford crude product. The residue was purified by preparative TLC (EtOAc:petroleum ether=1:2) to afford (87 mg, 25%) of the title compound as a pale-yellow oil; MS (ESI) m/z [M+H]⁺ 456.0.

### Intermediate 108

### 5-Chloro-6′-(((1S,3S)-3-((1-(4-methoxybenzyl)-1H-imidazo[4,5-b]pyridin-2-yl)amino)cyclopentyl)amino)-2H-[1,3′-bipyridin]-2-one*

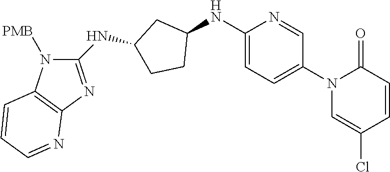

DMCDA (26 mg, 0.19 mmol) was added to 5-chloropyridin-2-ol (CAS Reg. No. 4214-79-3) (144 mg, 1.11 mmol), (1S,3S)—N¹-(5-iodopyridin-2-yl)-N³-(3-(4-methoxybenzyl)-3H-imidazo[4,5-b]pyridin-2-yl)cyclopentane-1,3-diamine* Intermediate 56 (200 mg, 0.37 mmol), CuI (35 mg, 0.19 mmol) and Cs₂CO₃ (362 mg, 1.11 mmol) in 1,4-dioxane (5 mL) at rt under nitrogen. The resulting solution was stirred at 100° C. for 20 h. The reaction mixture was diluted with EtOAc (75 mL) and washed sequentially with water (3×25 mL) and saturated brine (3×20 mL). The organic layer was dried over Na₂SO₄, filtered and evaporated to afford crude product. The residue was purified by preparative TLC (MeOH:DCM=1:20) to afford (188 mg, 94%) of the title compound as a brownish green solid; MS (ESI) m/z [M+H]⁺ 542. (*regiochemistry of the PMB group was not confirmed)

### Intermediate 109

### 5-Methoxy-6′-(((1S,3S)-3-((1-(4-methoxybenzyl)-1H-imidazo[4,5-b]pyridin-2-yl)amino)cyclopentyl)amino)-2H-[1,3′-bipyridin]-2-one*

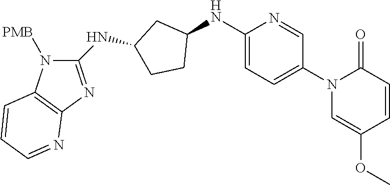

DMCDA (26 mg, 0.19 mmol) was added to 5-methoxypyridin-2(1H)-one (CAS Reg. No. 61941-79-5) (139 mg, 1.11 mmol), (1S,3S)—N¹-(5-iodopyridin-2-yl)-N³-(1-(4-methoxybenzyl)-1H-imidazo[4,5-b]pyridin-2-yl)cyclopentane-1,3-diamine* Intermediate 56 (200 mg, 0.37 mmol), CuI (35 mg, 0.19 mmol) and Cs₂CO₃ (362 mg, 1.11 mmol) in 1,4-dioxane (5 mL) at rt and the resulting solution was stirred at 100° C. for 18 h under nitrogen. The reaction mixture was diluted with EtOAc (75 mL) and washed sequentially with water (3×25 mL) and saturated brine (2×20 mL). The organic layer was dried over Na₂SO₄, filtered and evaporated to afford crude product. The crude product was purified by preparative TLC (NH3 in MeOH:DCM=1:20) to afford (70 mg, 35%) of the title compound as a white solid; MS (ESI) m/z [M+H]⁺ 418. (*regiochemistry of the PMB group was not confirmed)

### Intermediate 110

### 1-(4-(((1S,3S)-3-((3-Tosyl-3H-imidazo[4,5-b]pyridin-2-yl)amino)cyclopentyl)amino)phenyl)pyridin-2(1H)-one*

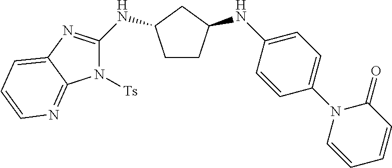 [SMILES: Cc1ccc(S(=O)(=O)n2c(N[C@H]3CC[C@H](Nc4ccc(-n5ccccc5=O)cc4)C3)nc3cccnc32)cc1]

1-(4-(((1S,3S)-3-Aminocyclopentyl)amino)phenyl)pyridin-2(1H)-one×2 HCl Intermediate 111 (100 mg, 0.29 mmol) was added to Cs₂CO₃ (286 mg, 0.88 mmol), Pd-PEPPSI-IpentCl 2-methylpyridine (12 mg, 0.01 mmol) and 2-chloro-3-tosyl-3H-imidazo[4,5-b]pyridine* Intermediate 39 (117 mg, 0.38 mmol) in 1,4-dioxane (10 mL). The resulting mixture was stirred at 100° C. for 15 h under nitrogen. The reaction mixture was filtered through celite. The filter cake was washed with EtOAc (3×20 mL) and the filtrate was concentrated under reduced pressure to afford the crude product which was purified by preparative TLC (MeOH:DCM=1:10) to afford (80 mg, 51%) of the title compound as a brown solid; MS (ESI) m/z [M+H]⁺ 541.2. (*regiochemistry was not confirmed)

### Intermediate 111

### 1-(4-(((1S,3S)-3-Aminocyclopentyl)amino)phenyl)pyridin-2(1H)-one×HCl

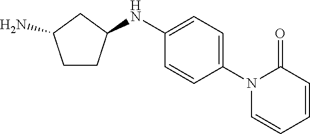 [SMILES: N[C@H]1CC[C@H](Nc2ccc(-n3ccccc3=O)cc2)C1]

4 M HCl in MeOH (2.0 mL, 8.0 mmol) was added to tert-butyl ((1S,3S)-3-((4-(2-oxopyridin-1(2H)-yl)phenyl)amino)cyclopentyl)carbamate Intermediate 112 (200 mg, 0.54 mmol) in MeOH (10 mL) at rt and the resulting solution was stirred at 60° C. for 15 h. The solvent was removed under reduced pressure to afford (180 mg, 97%) of the title compound as a pale-yellow oil which solidified on standing. The product was used in the next step directly without further purification; MS (ESI) m/z [M+H]⁺ 270.1.

### Intermediate 112

### tert-Butyl ((1S,3S)-3-((4-(2-oxopyridin-1(2H)-yl)phenyl)amino)cyclopentyl)carbamate

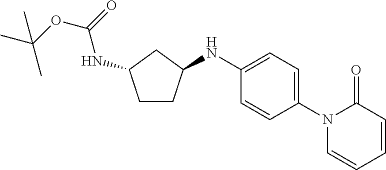 [SMILES: CC(C)(C)OC(=O)N[C@H]1CC[C@H](Nc2ccc(-n3ccccc3=O)cc2)C1]

Pd-PEPPSI-IpentCl 2-methylpyridine (30 mg, 0.04 mmol) was added to 1-(4-iodophenyl)pyridin-2(1H)-one (CAS Reg. No. 766556-75-6) (300 mg, 1.01 mmol), tert-butyl ((1S,3S)-3-aminocyclopentyl)carbamate (CAS Reg. No. 655400-44-8) (202 mg, 1.01 mmol) and Cs₂CO₃ (494 mg, 1.51 mmol) in 1,4-dioxane (15 mL) at rt and the resulting suspension was stirred at 100° C. for 15 h under nitrogen. The reaction mixture was filtered through silica. The solvent was removed under reduced pressure and the residue was purified by preparative TLC (EtOAc:petroleum ether=2:1) to afford (220 mg, 59%) of the title compound as a pale-yellow oil which solidified on standing; MS (ESI) m/z [M+H]⁺ 370.2.

### Intermediate 113

### 3-(5-(((1S,3S)-3-Aminocyclopentyl)amino)pyrazin-2-yl)-1-methylpyridin-2(1H)-one×HCl

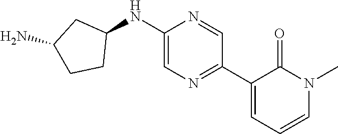 [SMILES: Cn1cccc(-c2cnc(N[C@H]3CC[C@H](N)C3)cn2)c1=O]

4 M HCl in MeOH (5.0 mL, 20 mmol) was added to a stirred solution of tert-butyl ((1S,3S)-3-((5-(1-methyl-2-oxo-1,2-dihydropyridin-3-yl)pyrazin-2-yl)amino)cyclopentyl)carbamate Intermediate 114 (70 mg, 0.18 mmol) in MeOH (5 mL) at rt and the resulting solution was stirred at 60° C. for 2 h. The solvent was removed under reduced pressure to afford (68 mg, 98%) of the title compound as a yellow solid which was used in the next step directly without further purification; MS (ESI) m/z [M+H]⁺ 286.0.

### Intermediate 114

### tert-Butyl ((1S,3S)-3-((5-(1-methyl-2-oxo-1,2-dihydropyridin-3-yl)pyrazin-2-yl)amino)cyclopentyl)carbamate

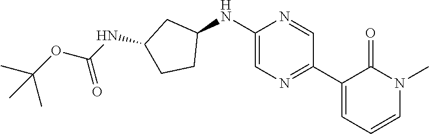 [SMILES: Cn1cccc(-c2cnc(N[C@H]3CC[C@H](NC(=O)OC(C)(C)C)C3)cn2)c1=O]

Cs₂CO₃ (147 mg, 0.45 mmol) was added to 3-(5-bromopyrazin-2-yl)-1-methylpyridin-2(1H)-one Intermediate 115 (60 mg, 0.23 mmol), tert-butyl ((1S,3S)-3-aminocyclopentyl)carbamate (CAS Reg. No. 655400-44-8) (68 mg, 0.34 mmol) and Pd-PEPPSI-IpentCl 2-methylpyridine (10 mg, 0.01 mmol) in 1,4-dioxane (3 mL) at rt. The resulting suspension was stirred at 100° C. for 15 h under nitrogen. The reaction mixture was diluted with EtOAc (200 mL) and washed sequentially with saturated brine (2×15 mL) and water (2×15 mL). The organic layer was dried over Na₂SO₄, filtered and evaporated to afford crude product. The residue was purified by preparative TLC (EtOAc) to afford (82 mg, 94%) of the title compound as a yellow solid; MS (ESI) m/z [M+H]⁺ 386.0.

### Intermediate 115

### 3-(5-Bromopyrazin-2-yl)-1-methylpyridin-2(1H)-one

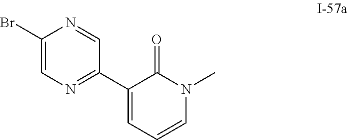

2-Bromo-5-iodopyrazine (CAS Reg. No. 622392-04-5) (1.68 g, 5.88 mmol) was added to (1-methyl-2-oxo-1,2-dihydropyridin-3-yl)boronic acid (CAS Reg. No. 1454558-46-3) (300 mg, 1.96 mmol), KOAc (578 mg, 5.88 mmol) and Pd(dppf)Cl₂·DCM (160 mg, 0.20 mmol) in 1,4-dioxane (16 mL) and water (4 mL) at rt and the resulting mixture was stirred at 100° C. for 2 h under nitrogen. The reaction mixture was poured into water (150 mL) and extracted with EtOAc (4×100 mL). The combined organic layers were dried (Na₂SO₄), filtered and evaporated and the obtained material was purified by flash chromatography on silica (gradient: 0-100% EtOAc in n-heptane) to afford (360 mg, 69%) of the title compound as a brown solid; MS (ESI): m/z [M+H]⁺ 265.8/267.8 (Br isotope pattern).

### Intermediate 116

### tert-Butyl ((1S,3S)-3-((5-(3-methyl-2,6-dioxo-3,6-dihydropyrimidin-1(2H)-yl)pyridin-2-yl)amino)cyclopentyl)carbamate

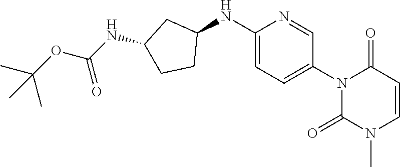 [SMILES: Cn1ccc(=O)n(-c2ccc(N[C@H]3CC[C@H](NC(=O)OC(C)(C)C)C3)nc2)c1=O]

tert-Butyl ((1S,3S)-3-((5-iodopyridin-2-yl)amino)cyclopentyl)carbamate Intermediate 1 (600 mg, 1.49 mmol) was added to a mixture of 1-methyluracil (CAS Reg. No. 615-77-0) (563 mg, 4.46 mmol), K₂CO₃ (617 mg, 4.46 mmol), Cu(I)I (576 mg, 2.98 mmol) and rel-(1R,2R)—N¹,N²-dimethylcyclohexane-1,2-diamine (423 mg, 2.98 mmol) in DMF (15 mL) and the mixture subsequently stirred at 100° C. for 15 h. The reaction mixture was cooled to rt and diluted with sat brine (150 mL). It was extracted with EtOAc (3×100 mL), the combined organic layers were dried (Na₂SO₄), filtered and evaporated and the obtained material was purified by preparative TLC (MeOH:DCM=1:15) to give the title compound (262 mg, 44%) as a yellow solid; MS (ESI): m/z [M+H]⁺ 402.2.

### Intermediate 117

### 3-(6-(((1S,3S)-3-Aminocyclopentyl)amino)pyridin-3-yl)-1-methylpyrimidine-2,4(1H,3H)-dione

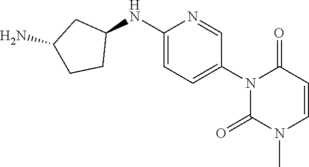 [SMILES: Cn1ccc(=O)n(-c2ccc(N[C@H]3CC[C@H](N)C3)nc2)c1=O]

tert-Butyl ((1S,3S)-3-((5-(3-methyl-2,6-dioxo-3,6-dihydropyrimidin-1(2H)-yl)pyridin-2-yl)amino)cyclopentyl)carbamate Intermediate 116 (80 mg, 0.20 mmol) was treated with TFA (1 mL) in DCM (3 mL) at 20° C. for 2 h. The reaction mixture was concentrated under reduced pressure to afford an unspecified TFA salt of the title compound (120 mg, 94%) as a brown gum; MS (ESI): m/z [M+H]⁺ 302.2.

### Intermediate 118

### tert-Butyl ((1S,3S)-3-((5-(difluoromethoxy)-2-oxo-2H-[1,3′-bipyridin]-6′-yl)amino)cyclopentyl)carbamate

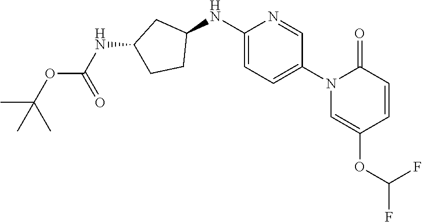 [SMILES: CC(C)(C)OC(=O)N[C@H]1CC[C@H](Nc2ccc(-n3cc(OC(F)F)ccc3=O)cn2)C1]

tert-Butyl ((1S,3S)-3-((5-iodopyridin-2-yl)amino)cyclopentyl)carbamate Intermediate 1 (600 mg, 1.49 mmol) was added to a mixture of 5-(difluoromethoxy)-2(1H)-pyridinone (CAS Reg. No. 130318-75-1) (360 mg, 2.23 mmol), Cs₂CO₃ (1.45 g, 4.46 mmol), Cu(I)I (283 mg, 1.49 mmol) and rel-(1R,2R)—N¹,N²-dimethylcyclohexane-1,2-diamine (212 mg, 1.49 mmol) in 1,4-dioxane (15 mL) at rt and the mixture subsequently stirred at 100° C. for 18 h under nitrogen. The reaction mixture was cooled to rt, combined with another crude reaction mixture of comparable size (starting from 500 mg Intermediate 1) and diluted with EtOAc (150 mL). The organic layer was washed with water (3×125 mL), dried (Na₂SO₄), filtered and evaporated and the obtained material was purified by flash chromatography on silica (gradient: 0-100% EtOAc in PE) to give the title compound (887 mg, 74%) as a brown gum; MS (ESI): m/z [M+H]⁺ 437; ¹⁹F NMR (282 MHz, DMSO-d₆) 5-82.30.

### Intermediate 119

### 6′-(((1S,3S)-3-Aminocyclopentyl)amino)-5-(difluoromethoxy)-2H-[1,3′-bipyridin]-2-one

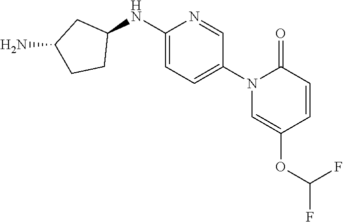 [SMILES: N[C@H]1CC[C@H](Nc2ccc(-n3cc(OC(F)F)ccc3=O)cn2)C1]

tert-Butyl ((1S,3S)-3-((5-(difluoromethoxy)-2-oxo-2H-[1,3′-bipyridin]-6′-yl)amino)cyclopentyl)carbamate Intermediate 118 (867 mg, 1.99 mmol) was treated with TFA (0.5 mL, 6.5 mmol) in DCM (20 mL) at 40° C. for 1 h. The reaction mixture was concentrated under reduced pressure to afford an unspecified TFA salt of the title compound (1.43 g, 91%) as a brown gum; MS (ESI): m/z [M+H]⁺ 337.1; ¹⁹F NMR (282 MHz, DMSO-d₆) δ −82.3.

### Intermediate 120

### tert-Butyl ((1S,3S)-3-((3-(difluoromethoxy)-2-oxo-2H-[1,3′-bipyridin]-6′-yl)amino)cyclopentyl)carbamate

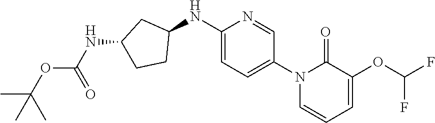 [SMILES: CC(C)(C)OC(=O)N[C@H]1CC[C@H](Nc2ccc(-n3cccc(OC(F)F)c3=O)cn2)C1]

rel-(1R,2R)—N¹,N²-Dimethylcyclohexane-1,2-diamine (88 mg, 0.62 mmol) was added to a mixture of tert-butyl ((1S,3S)-3-((5-iodopyridin-2-yl)amino)cyclopentyl)carbamate Intermediate 1 (250 mg, 0.62 mmol), 3-(difluoromethoxy)-2(1H)-pyridinone (CAS Reg. No. 1241752-48-6) (150 mg, 0.93 mmol), Cs₂CO₃ (606 mg, 1.86 mmol) and Cu(I)I (118 mg, 0.62 mmol) in 1,4-dioxane (15 mL) at rt and the mixture subsequently stirred at 100° C. for 18 h under nitrogen. The reaction mixture was cooled to rt, combined with another crude reaction mixture of identical size (starting from 250 mg Intermediate 1) and diluted with water (125 mL). It was extracted with EtOAc (4×100 mL), and the combined organic layers were dried (Na₂SO₄), filtered and evaporated. The obtained material was purified by preparative TLC (7 M NH₃ in MeOH:DCM, 1:20) to give the title compound (428 mg, 79%) as a brown solid; MS (ESI): m/z [M+H]⁺ 437.2; ¹⁹F NMR (282 MHz, DMSO-d₆) δ −82.30.

### Intermediate 121

### 6′-(((1S,3S)-3-Aminocyclopentyl)amino)-3-(difluoromethoxy)-2H-[1,3′-bipyridin]-2-one

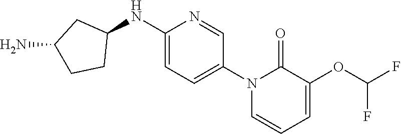 [SMILES: N[C@H]1CC[C@H](Nc2ccc(-n3cccc(OC(F)F)c3=O)cn2)C1]

A solution of tert-butyl ((1S,3S)-3-((3-(difluoromethoxy)-2-oxo-2H-[1,3′-bipyridin]-6′-yl)amino)cyclopentyl)carbamate Intermediate 120 (410 mg, 0.94 mmol) in MeOH (8 mL) was treated with 4 M HCl in MeOH (2.0 mL, 8.0 mmol) and stirred at 60° C. for 2 h. The reaction mixture was concentrated under reduced pressure to afford an unspecified HCl salt of the title compound (398 mg, 100%) as a yellow solid; MS (ESI): m/z [M+H]⁺ 337; ¹⁹F NMR (282 MHz, DMSO-d₆) δ −82.3.

### Intermediate 122

### tert-Butyl ((1S,3S)-3-((5-(2,4-dioxo-1,4-dihydrothieno[3,2-d]pyrimidin-3(2H)-yl)pyridin-2-yl)amino)cyclopentyl)carbamate

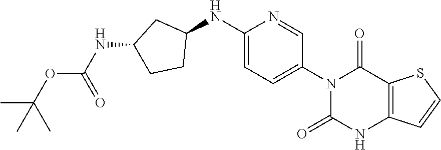 [SMILES: CC(C)(C)OC(=O)N[C@H]1CC[C@H](Nc2ccc(-n3c(=O)[nH]c4ccsc4c3=O)cn2)C1]

tert-Butyl ((1S,3S)-3-((5-iodopyridin-2-yl)amino)cyclopentyl)carbamate Intermediate 1 (300 mg, 0.74 mmol) was added to a mixture of rel-(1R,2R)—N¹,N²-dimethylcyclohexane-1,2-diamine (53 mg, 0.37 mmol), thieno[3,2-d]pyrimidine-2,4(1H,3H)-dione (CAS Reg. No. 16233-51-5) (375 mg, 2.23 mmol), K₂CO₃ (308 mg, 2.23 mmol) and Cu(I)I (71 mg, 0.37 mmol) in DMF (15 mL) at rt and the mixture subsequently stirred at 100° C. for 15 h under nitrogen. The reaction mixture was cooled to rt and diluted with sat brine (125 mL). It was extracted with EtOAc (4×75 mL), and the combined organic layers were dried (Na₂SO₄), filtered and evaporated. The obtained material was purified by preparative TLC (MeOH:DCM=1:10) to give the title compound (230 mg, 70%) as a yellow solid; MS (ESI): m/z [M+H]⁺ 443.9.

### Intermediate 123

### 3-(6-(((1S,3S)-3-Aminocyclopentyl)amino)pyridin-3-yl)thieno[3,2-d]pyrimidine-2,4(1H,3H)-dione

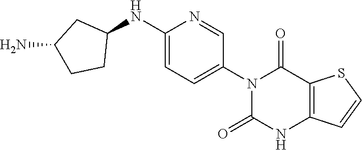 [SMILES: N[C@H]1CC[C@H](Nc2ccc(-n3c(=O)[nH]c4ccsc4c3=O)cn2)C1]

tert-Butyl ((1S,3S)-3-((5-(2,4-dioxo-1,4-dihydrothieno[3,2-d]pyrimidin-3(2H)-yl)pyridin-2-yl)amino)cyclopentyl)carbamate Intermediate 122 (190 mg, 0.43 mmol) was treated with TFA (2 mL) in DCM (6 mL) at 20° C. for 3 h. The reaction mixture was concentrated under reduced pressure to afford an unspecified TFA salt of the title compound (263 mg, 90%) as a brown oil; MS (ESI): m/z [M+H]⁺ 343.9.

### Intermediate 124

### 6′-(((1S,3S)-3-((5-Bromobenzo[d]thiazol-2-yl)amino)cyclopentyl)amino)-2H-[1,3′-bipyridin]-2-one

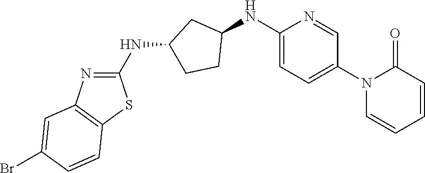 [SMILES: O=c1ccccn1-c1ccc(N[C@H]2CC[C@H](Nc3nc4cc(Br)ccc4s3)C2)nc1]

6′-(((1S,3S)-3-Aminocyclopentyl)amino)-2H-[1,3′-bipyridin]-2-one×HCl Intermediate 3 (408 mg, 1.51 mmol) was added to a mixture of 5-bromo-2-chlorobenzothiazole (CAS Reg. No. 824403-26-1) (250 mg, 1.01 mmol) and K₂CO₃ (417 mg, 3.02 mmol) in DMSO (10 mL) at rt and the resulting mixture was stirred at 120° C. for 18 h. The reaction mixture was diluted with water (75 mL) and extracted with EtOAc (5×100 mL). The combined organic layers were dried (Na₂SO₄), filtered and evaporated and the obtained material was purified by preparative TLC (EtOAc) to give the title compound (359 mg, 74%) as a yellow solid; MS (ESI): m/z [M+H]⁺ 481.8/483.8 (Br isotope pattern).

### Intermediate 125

### tert-Butyl ((1S,3S)-3-((6-bromobenzo[d]oxazol-2-yl)amino)cyclopentyl)carbamate

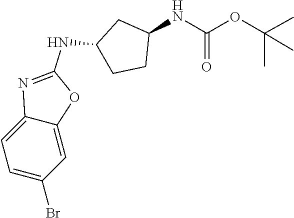 [SMILES: CC(C)(C)OC(=O)N[C@H]1CC[C@H](Nc2nc3ccc(Br)cc3o2)C1]

Na₂CO₃ (318 mg, 3.00 mmol) was added to a mixture of tert-butyl ((1S,3S)-3-aminocyclo-pentyl)carbamate (200 mg, 1.00 mmol) and 6-bromo-2-chlorobenzoxazole (CAS Reg. No. 1260643-15-9) (464 mg, 2.00 mmol) in DMSO (3 mL) at rt and the resulting mixture was stirred at 100° C. for 3 h. The reaction mixture was poured into water (100 mL) and extracted with EtOAc (2×100 mL). The combined organic layers were dried over Na₂SO₄, filtered and evaporated and the obtained material was purified by preparative TLC (7 M NH₃ in MeOH:DCM, 1:30) to give the title compound (383 mg, 97%) as a brown solid; MS (ESI): m/z [M+H]⁺ 395.9/397.9 (Br isotope pattern).

### Intermediate 126

### tert-Butyl ((1S,3S)-3-((6-(4,4,5,5-tetramethyl-1,3,2-dioxaborolan-2-yl)benzo[d]oxazol-2-yl)amino)cyclopentyl)carbamate

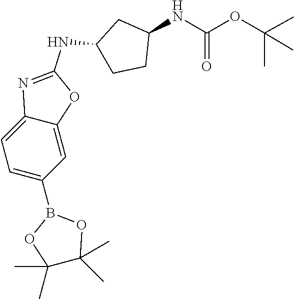 [SMILES: CC(C)(C)OC(=O)N[C@H]1CC[C@H](Nc2nc3ccc(B4OC(C)(C)C(C)(C)O4)cc3o2)C1]

KOAc (223 mg, 2.27 mmol) was added to a mixture of tert-butyl ((1S,3S)-3-((6-bromoben-[d]oxazol-2-yl)amino)cyclopentyl)carbamate Intermediate 125 (300 mg, 0.76 mmol), bis(pinacolato)diboron (CAS Reg. No. 73183-34-3) (384 mg, 1.51 mmol) and Pd(dppf)Cl₂·DCM (40 mg, 0.05 mmol) in 1,4-dioxane (1 mL) at 20° C. and the resulting mixture was stirred at 100° C. for 16 h under nitrogen. The reaction mixture was poured into water (100 mL) and extracted with EtOAc (2×100 mL). The combined organic layers were dried over Na₂SO₄, filtered and evaporated to afford crude product which was purified by preparative TLC (7 M NH₃ in MeOH:DCM, 1:20) to afford the title compound (360 mg, 100%) as a brown solid; MS (ESI) m/z [M+H]⁺ 444.1.

### Intermediate 127

### tert-Butyl ((1S,3S)-3-((6-hydroxybenzo[d]oxazol-2-yl)amino)cyclopentyl)carbamate

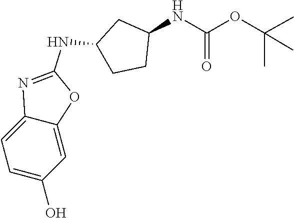 [SMILES: CC(C)(C)OC(=O)N[C@H]1CC[C@H](Nc2nc3ccc(O)cc3o2)C1]

Sodium perborate tetrahydrate (CAS Reg. No. 10486-00-7) was added to tert-butyl ((1S,3S)-3-((6-(4,4,5,5-tetramethyl-1,3,2-dioxaborolan-2-yl)benzo[d]oxazol-2-yl)amino)cyclopentyl)carbamate Intermediate 126 (350 mg, 0.77 mmol) in a mixture of THF (6 mL) and water (3 mL) at 30° C. and the resulting mixture was stirred at this temperature for 16 h. The reaction mixture was poured into water (100 mL) and extracted with EtOAc (2×100 mL). The combined organic layers were dried over Na₂SO₄, filtered and evaporated to afford crude product which was purified by preparative TLC (7 M NH₃ in MeOH:DCM, 1:20) to afford the title compound (168 mg, 64%) as a pale-yellow solid; MS (ESI) m/z [M+H]⁺ 334.

### Intermediate 128

### tert-Butyl ((1S,3S)-3-((6-(difluoromethoxy)benzo[d]oxazol-2-yl)amino)cyclopentyl)carbamate

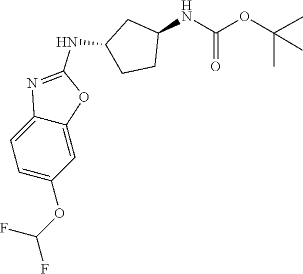 [SMILES: CC(C)(C)OC(=O)N[C@H]1CC[C@H](Nc2nc3ccc(OC(F)F)cc3o2)C1]

KOH (6 M in water) was added to a mixture of tert-butyl ((1S,3S)-3-((6-hydroxybenzo[d]oxazol-2-yl)amino)cyclopentyl)carbamate Intermediate 127 (153 mg, 0.46 mmol) and diethyl (bromodifluoromethyl)phosphonate (CAS Reg. diethyl (bromodifluoromethyl)phosphonate (CAS Reg. No. 65094-22-6) (367 mg, 1.37 mmol) in MeCN (2 mL) at 30° C. and the resulting mixture was stirred at 50° C. for 1 h. The reaction mixture was poured into water (100 mL) and extracted with EtOAc (2×100 mL). The combined organic layers were dried over Na₂SO₄, filtered and evaporated to afford crude product which was purified by preparative TLC (7 M NH₃ in MeOH:DCM, 1:30) to afford the title compound (52 mg, 30%) as a brown gum; MS (ESI) m/z [M+H]⁺ 383.8.

### Intermediate 129

### (1S,3S)—N¹-(6-(Difluoromethoxy)benzo[d]oxazol-2-yl)cyclopentane-1,3-diamine

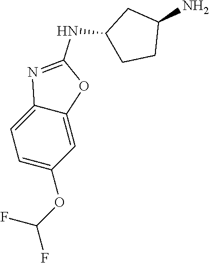 [SMILES: N[C@H]1CC[C@H](Nc2nc3ccc(OC(F)F)cc3o2)C1]

tert-Butyl ((1S,3S)-3-((6-(difluoromethoxy)benzo[d]oxazol-2-yl)amino)cyclopentyl)carbamate Intermediate 128 (42 mg, 0.11 mmol) was treated with TFA (1 mL) in DCM (1 mL) at 20° C. for 16 h. The reaction mixture was concentrated under reduced pressure to afford an unspecified TFA salt of the title compound (51 mg, 100%); MS (ESI): m/z [M+H]⁺ 283.9; ¹⁹F NMR (282 MHz, DMSO-d₆) δ −81.46.

### Intermediate 130

### 6′-Chloro-5-(1H-tetrazol-5-yl)-2H-[1,3′-bipyridin]-2-one

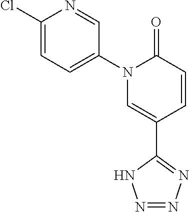 [SMILES: O=c1ccc(-c2nnn[nH]2)cn1-c1ccc(Cl)nc1]

Bis(4-nitrophenyl) phosphorazidate (CAS Reg. No. 51250-91-0) (880 mg, 2.4 mmol) was added to a solution of 6′-chloro-2-oxo-2H-[1,3′-bipyridine]-5-carboxamide Intermediate 17 (300 mg, 1.20 mmol) in pyridine (5 mL) under air. The resulting mixture was stirred at 90° C. for 16 h, subsequently cooled to rt and concentrated under reduced pressure. The obtained residue was purified by C18-flash chromatography (gradient: 0-20% MeCN in water) to give the title compound (210 mg, 64%) as a white solid. MS (ESI): m/z [M+H]⁺ 275.0.

### Intermediate 131

### 6′-Chloro-5-(1-(4-methoxybenzyl)-1H-tetrazol-5-yl)-2H-[1,3′-bipyridin]-2-one

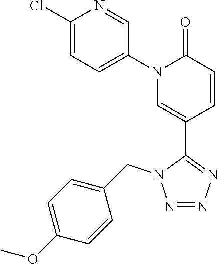 [SMILES: COc1ccc(Cn2nnnc2-c2ccc(=O)n(-c3ccc(Cl)nc3)c2)cc1]

4-Methoxybenzyl chloride (CAS Reg. No. 824-94-2) (690 mg, 4.4 mmol) was added to a mixture of 6′-chloro-5-(1H-tetrazol-5-yl)-2H-[1,3′-bipyridin]-2-one Intermediate 130 (810 mg, 2.95 mmol) and K₂CO₃ (815 mg, 5.90 mmol) in DMF (15 mL) and the resulting suspension was stirred at 80° C. for 3 h. The reaction mixture was diluted with water (100 mL) and extracted with EtOAc (3×150 mL). The combined organic layers were dried over Na₂SO₄, filtered and evaporated and the crude material was purified by C18-flash chromatography (gradient: 30-40% MeCN in water) to give the title compound (1000 mg, 86%) as a white solid. MS (ESI): m/z [M+H]⁺ 395.1.

### Intermediate 132

### tert-Butyl ((1S,3S)-3-((5-(1-(4-methoxybenzyl)-1H-tetrazol-5-yl)-2-oxo-2H-[1,3′-bipyridin]-6′-yl)amino)cyclopentyl)carbamate

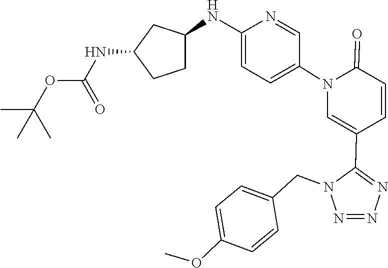 [SMILES: COc1ccc(Cn2nnnc2-c2ccc(=O)n(-c3ccc(N[C@H]4CC[C@H](NC(=O)OC(C)(C)C)C4)nc3)c2)cc1]

Pd-PEPPSI-IPentCl 2-methylpyridine (0.179 g, 0.21 mmol) was added to a mixture of 6′-chloro-5-(1-(4-methoxybenzyl)-1H-tetrazol-5-yl)-2H-[1,3′-bipyridin]-2-one Intermediate 131 (0.84 g, 2.13 mmol), Cs₂CO₃ (1.37 g, 4.26 mmol) and tert-butyl ((1S,3S)-3-aminocyclopentyl)carbamate (CAS Reg. No 645400-44-8) (0.43 g, 2.13 mmol) in DMF (13 mL) and it was stirred under an atmosphere of nitrogen at 100° C. for 18 h. The reaction mixture was filtered through Celite and the filter cake washed with EtOAc (20 mL). The combined filtrates were concentrated under reduced pressure and the residue was purified by flash chromatography on silica (gradient: 0-10% MeOH in DCM) to afford (0.70 g, 59%) of the title compound as a yellow solid. MS (ESI): m/z [M+H]⁺ 559.3.

### Intermediate 133

### 6′-(((1S,3S)-3-Aminocyclopentyl)amino)-5-(1-(4-methoxybenzyl)-1H-tetrazol-5-yl)-2H-[1,3′-bipyridin]-2-one

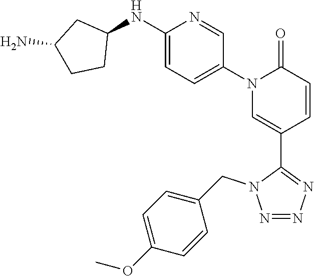 [SMILES: COc1ccc(Cn2nnnc2-c2ccc(=O)n(-c3ccc(N[C@H]4CC[C@H](N)C4)nc3)c2)cc1]

TFA (3 mL, 39 mmol) was added to tert-butyl ((1S,3S)-3-((5-(1-(4-methoxybenzyl)-1H-tetrazol-5-yl)-2-oxo-2H-[1,3′-bipyridin]-6′-yl)amino)cyclopentyl)carbamate Intermediate 132 (690 mg, 1.24 mmol) in DCM (10 mL) at rt and it was stirred under an atmosphere of nitrogen at rt for 2 h. The solvent was removed under reduced pressure to give an unspecified TFA salt of the title compound (600 mg, 85%) as an orange gum which was used without further purification. MS (ESI): m/z [M+H]⁺ 459.0.

### Intermediate 134

### 5-(1-(4-Methoxybenzyl)-1H-tetrazol-5-yl)-6′-(((1S,3S)-3-((3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((1-(4-Methoxybenzyl)-1H-imidazo[4,5-b]pyridin-2-yl)amino)cyclopentyl)amino)-5-(1-(4-methoxybenzyl)-1H-tetrazol-5-yl)-2H-[1,3′-bipyridin]-2-one

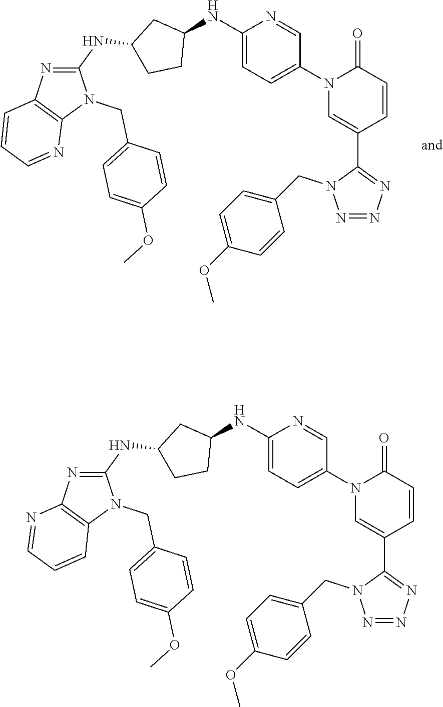 [SMILES: COc1ccc(Cn2nnnc2-c2ccc(=O)n(-c3ccc(N[C@H]4CC[C@H](Nc5nc6cccnc6n5Cc5ccc(OC)cc5)C4)nc3)c2)cc1 ; COc1ccc(Cn2nnnc2-c2ccc(=O)n(-c3ccc(N[C@H]4CC[C@H](Nc5nc6ncccc6n5Cc5ccc(OC)cc5)C4)nc3)c2)cc1]

1-(Chloromethyl)-4-methoxybenzene (27 mg, 0.17 mmol) was added to 2-chloro-1H-imidazo[4,5-b]pyridine (20 mg, 0.13 mmol) and K₂CO₃ (54 mg, 0.39 mmol) in DMA (3 mL) and the resulting mixture was stirred at 80° C. for 3 h under a nitrogen atmosphere. Pd-PEPPSI-IpentCl 2-methylpyridine (11 mg, 0.01 mmol) and 6′-(((1S,3S)-3-aminocyclopentyl)amino)-5-(1-(4-methoxybenzyl)-1H-tetrazol-5-yl)-2H-[1,3′-bipyridin]-2-one Intermediate 133 (60 mg, 0.13 mmol) were added at 20° C. and the resulting mixture was stirred at 100° C. for 18 h. The reaction mixture was filtered through a pad of Celite and the filter cake was washed with EtOAc (50 mL). The combined filtrates were concentrated under reduced pressure and the obtained material was purified by reversed phase flash chromatography on a C18 column (gradient: 30-40% of MeCN in water) to give a crude mixture of the regioisomeric title compounds (80 mg, 88%) as a brown gum; MS (ESI) m/z [M+H]⁺ 696.3.

### Intermediate 135

### 6′-Fluoro-2H-[1,3′-bipyridin]-2-one

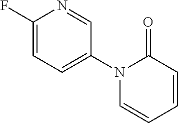 [SMILES: O=c1ccccn1-c1ccc(F)nc1]

rel-(1R,2R)—N¹,N²-Dimethylcyclohexane-1,2-diamine (1.50 g, 10.5 mmol) was added to a mixture of pyridin-2(1H)-one (CAS Reg. No. 142-08-5) (5.0 g, 52.6 mmol), 2-fluoro-5-iodopyridine (CAS Reg. No. 171197-80-1) (17.6 g, 78.9 mmol), Cu(I)I (4.01 g, 21.0 mmol) and K₂CO₃ (14.53 g, 105.2 mmol) in 1,4-dioxane (50 mL) and it was stirred at 90° C. for 18 h under a nitrogen atmosphere. The solvent was removed under reduced pressure and the residue purified by flash chromatography on silica (gradient: 0-50% EtOAc in PE) to give the title compound (11.6 g, 116%) as a brown solid. MS (ESI): m/z [M+H]⁺ 191.1. ¹H NMR (300 MHz, DMSO-d₆) δ ppm 6.36 (1H, dt), 6.52 (1H, d), 7.36 (1H, dd), 7.54 (1H, ddd), 7.73 (1H, dd), 8.14 (1H, ddd), 8.35 (1H, dd).

### EXAMPLES

### Example 1

### 6′-(((1S,3S)-3-(Benzo[d]oxazol-2-ylamino)cyclopentyl)amino)-2H-[1,3′-bipyridin]-2-one—Compound 1

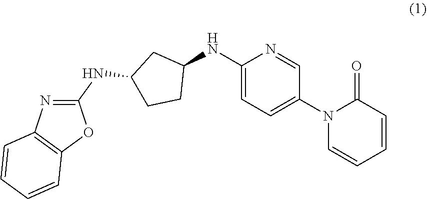 [SMILES: O=c1ccccn1-c1ccc(N[C@H]2CC[C@H](Nc3nc4ccccc4o3)C2)nc1]

2-Chlorobenzo[d]oxazole (100 mg, 0.65 mmol) was added to 6′-(((1S,3S)-3-aminocyclopentyl)amino)-2H-[1,3′-bipyridin]-2-one×HCl Intermediate 3 (100 mg, 0.33 mmol) and Na₂CO₃ (104 mg, 0.98 mmol) in DMSO (10 mL) at rt and the resulting solution was stirred at 100° C. for 5 h. The reaction mixture was diluted with EtOAc (50 mL), and washed with brine (3×125 mL). The organic layer was dried (Na₂SO₄), filtered and evaporated to afford a crude product. The crude product was purified by preparative HPLC, PrepMethod F (gradient: 20-50%), to give the title compound (31 mg, 25%) as a white solid; HRMS (ESI) m/z [M+H]⁺ calcd for C₂₂H₂₂N₅O₂: 388.1768, found: 388.1782; ¹H NMR (300 MHz, DMSO-d₆) δ 1.37-1.77 (2H, m), 1.84-2.11 (2H, m), 2.11-2.36 (2H, m), 4.30 (2H, dp), 6.28 (1H, td), 6.38-6.50 (1H, m), 6.56 (1H, d), 6.85-7.06 (2H, m), 7.11 (1H, td), 7.25 (1H, dd), 7.33 (1H, dd), 7.39-7.52 (2H, m), 7.61 (1H, dd), 7.94 (1H, d), 8.03 (1H, d).

### Example 2

### 6′-(((1S,3S)-3-((6-Methylbenzo[d]oxazol-2-yl)amino)cyclopentyl)amino)-2H-[1,3′-bipyridin]-2-one—Compound 2

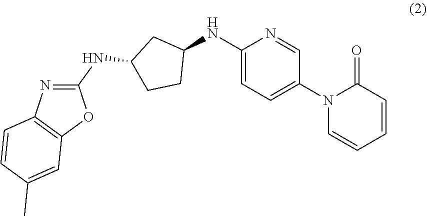 [SMILES: Cc1ccc2nc(N[C@H]3CC[C@H](Nc4ccc(-n5ccccc5=O)cn4)C3)oc2c1]

2-Chloro-6-methylbenzo[d]oxazole (43.7 mg, 0.26 mmol) was added to 6′-(((1S,3S)-3-aminocyclopentyl)amino)-2H-[1,3′-bipyridin]-2-one×HCl Intermediate 3 (100 mg, 0.33 mmol) and Na₂CO₃ (104 mg, 0.98 mmol) in DMSO (10 mL) at rt and the resulting solution was stirred at 100° C. for 5 h. The reaction mixture was diluted with EtOAc (100 mL), and washed sequentially with brine (3×35 mL). The organic layer was dried (Na₂SO₄), filtered and evaporated. The crude product was purified by preparative HPLC, PrepMethod F (gradient: 22-52%), to give the title compound (60 mg, 46%) as a white solid. ¹H-NMR (300 MHz, DMSO-d₆) δ 1.42-1.75 (2H, m), 1.81-2.05 (2H, m), 2.16 (2H, ddd), 2.31 (3H, s), 4.27 (2H, dq), 6.25 (1H, td), 6.43 (1H, ddd), 6.52 (1H, dd), 6.86-7.00 (2H, m), 7.04-7.17 (2H, m), 7.30-7.51 (2H, m), 7.59 (1H, ddd), 7.80-7.98 (2H, m). HRMS (ESI) m/z [M+H]⁺ calcd for C₂₃H₂₄N₅O₂: 402.1924, found: 402.1936.

### Example 3

### 6′-(((1S,3S)-3-((6-Bromobenzo[d]oxazol-2-yl)amino)cyclopentyl)amino)-2H-[1,3′-bipyridin]-2-one—Compound 3

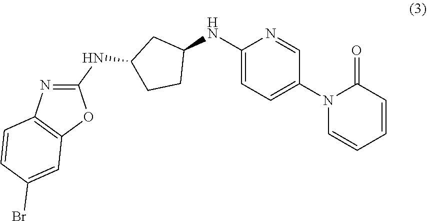 [SMILES: O=c1ccccn1-c1ccc(N[C@H]2CC[C@H](Nc3nc4ccc(Br)cc4o3)C2)nc1]

6-Bromo-2-chlorobenzo[d]oxazole (68 mg, 0.29 mmol) was added to 6′-(((1S,3S)-3-aminocyclopentyl)amino)-2H-[1,3′-bipyridin]-2-one×HCl Intermediate 3 (100 mg, 0.33 mmol) and Na₂CO₃ (104 mg, 0.98 mmol) in DMSO (5 mL) at rt and the resulting solution was stirred at 100° C. for 5 h. The reaction mixture was diluted with EtOAc (100 mL) and washed sequentially with sat brine (3×35 mL). The organic layer was dried (Na₂SO₄), filtered and evaporated. The crude product was purified by preparative HPLC, PrepMethod H (gradient: 22-59%), to give the title compound (65 mg, 43%) as a white solid. HRMS (ESI) m/z [M+H]⁺ calcd for C₂₂H₂₁BrN₅O₂: 466.0874, found: 466.0884. ¹H NMR (300 MHz, DMSO-d₆) δ 1.47-1.69 (2H, m), 1.88-2.25 (4H, m), 4.19-4.41 (2H, m), 6.27 (1H, td), 6.44 (1H, d), 6.54 (1H, d), 6.97 (1H, d), 7.19 (1H, d), 7.27 (1H, dd), 7.41 (1H, dd), 7.48 (1H, ddd), 7.59-7.64 (2H, m), 7.94 (1H, d), 8.23 (1H, d).

### Example 4

### 6′-(((1S,3S)-3-((6-(Hydroxymethyl)benzo[d]oxazol-2-yl)amino)cyclopentyl)amino)-2H-[1,3′-bipyridin]-2-one—Compound 4

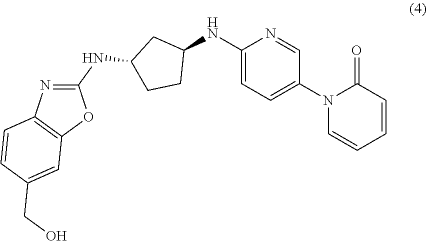 [SMILES: O=c1ccccn1-c1ccc(N[C@H]2CC[C@H](Nc3nc4ccc(CO)cc4o3)C2)nc1]

(Tributylstannyl)methanol (207 mg, 0.64 mmol) was added to 6′-(((1S,3S)-3-((6-bromobenzo[d]oxazol-2-yl)amino)cyclopentyl)amino)-2H-[1,3′-bipyridin]-2-one Example 3 (200 mg, 0.43 mmol) and Pd(PPh₃)₄ (25 mg, 0.02 mmol) in 1,4-dioxane (3 mL) at rt, and the resulting suspension was stirred at 80° C. for 16 h under a nitrogen atmosphere. The reaction mixture was filtered through celite. The filter cake was washed with DCM (2×5 mL). The filtrate was concentrated under reduced pressure. The crude product was purified by preparative HPLC, PrepMethod K (gradient: 2-19%), to give (8 mg, 4%) of the title compound as a white solid; HRMS (ESI) m/z [M+H]⁺ calcd for C₂₃H₂₄N₅O₃: 418.1874, found: 418.1880; ¹H NMR (300 MHz, CD₃OD) δ 1.57-1.82 (2H, m), 1.98-2.23 (2H, m), 2.23-2.42 (2H, m), 4.24-4.48 (2H, m), 4.64 (2H, s), 6.48 (1H, td), 6.64 (2H, ddd), 7.08-7.4 (3H, m), 7.47 (1H, dd), 7.55-7.69 (2H, m), 7.97 (1H, d).

### Example 5

### 2-(((1S,3S)-3-((2-Oxo-2H-[1,3′-bipyridin]-6′-yl)amino)cyclopentyl)amino)benzo[d]oxazole-6-carbonitrile—Compound 5

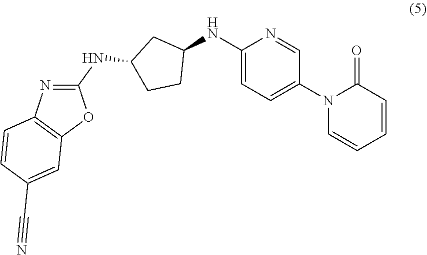 [SMILES: N#Cc1ccc2nc(N[C@H]3CC[C@H](Nc4ccc(-n5ccccc5=O)cn4)C3)oc2c1]

Zn(s) (26 mg, 0.40 mmol) was added to 6′-(((1S,3S)-3-((6-bromobenzo[d]oxazol-2-yl)amino)cyclopentyl)amino)-2H-[1,3′-bipyridin]-2-one Example 3 (318 mg, 0.40 mmol), Zn(CN)₂ (95 mg, 0.81 mmol), dppf (45 mg, 0.08 mmol) and Pd₂(dba)₃ (37 mg, 0.04 mmol) in DMF (5 mL) at rt and the resulting suspension was stirred at 100° C. for 16 h under a nitrogen atmosphere. The reaction mixture was poured into water (100 mL), extracted with EtOAc (2×50 mL). The organic layer was dried (Na₂SO₄), filtered and evaporated. The crude product was purified by reversed phase flash chromatography on a C18 column (gradient: 50-75% MeCN in water) to give (30 mg, 18%) of the title compound as a light grey solid; HRMS (ESI) m/z [M+H]⁺ calcd for C₂₃H₂₁N₆O₂: 413.1720, found: 413.1716; ¹H NMR (300 MHz, DMSO-d₆) δ 1.44-1.71 (2H, m), 1.87-2.11 (2H, m), 2.14-2.26 (2H, m), 4.23-4.43 (2H, m), 6.28 (1H, td), 6.45 (1H, dd), 6.54 (1H, d), 7.00 (1H, d), 7.27-7.54 (3H, m), 7.54-7.75 (2H, m), 7.88-7.97 (2H, m), 8.66 (1H, d).

### Example 6

### 6′-(((1S,3S)-3-((6-Bromobenzo[d]thiazol-2-yl)amino)cyclopentyl)amino)-2H-[1,3′-bipyridin]-2-one—Compound 6

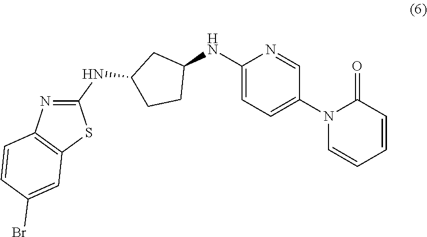 [SMILES: O=c1ccccn1-c1ccc(N[C@H]2CC[C@H](Nc3nc4ccc(Br)cc4s3)C2)nc1]

6-Bromo-2-chlorobenzo[d]thiazole (81 mg, 0.33 mmol) was added to 6′-(((1S,3S)-3-aminocyclopentyl)amino)-2H-[1,3′-bipyridin]-2-one×HCl Intermediate 3 (100 mg, 0.33 mmol) and Na₂CO₃ (104 mg, 0.98 mmol) in DMSO (10 mL) at rt and the resulting solution was stirred at 100° C. for 5 h. The reaction mixture was diluted with EtOAc (50 mL), and washed sequentially with brine (3×125 mL). The organic layer was dried over Na₂SO₄, filtered and evaporated. The crude product was purified by preparative HPLC, PrepMethod A (gradient: 38-68%), to give the title compound (65 mg, 41%) as a white solid; HRMS (ESI) m/z [M+H]⁺ calcd for C₂₂H₂₁BrN₅OS: 482.0644, found: 482.0694; ¹H NMR (300 MHz, DMSO-d₆) δ 1.48-1.65 (2H, m), 1.88-2.04 (2H, m), 2.11-2.27 (2H, m), 4.35 (2H, br d), 6.28 (1H, t), 6.45 (1H, d), 6.54 (1H, d), 6.98 (1H, d), 7.28-7.51 (4H, m), 7.61 (1H, dd), 7.92 (2H, dd), 8.28 (1H, d).

### Example 7

### 6′-(((1S,3S)-3-(Benzo[d]thiazol-2-ylamino)cyclopentyl)amino)-2H-[1,3′-bipyridin]-2-one—Compound 7

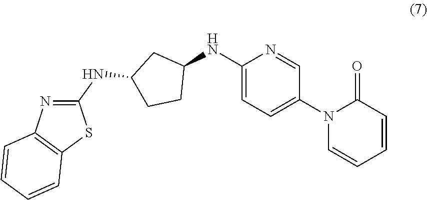 [SMILES: O=c1ccccn1-c1ccc(N[C@H]2CC[C@H](Nc3nc4ccccc4s3)C2)nc1]

2-Chlorobenzo[d]thiazole (55 mg, 0.33 mmol) was added to 6′-(((1S,3S)-3-aminocyclopentyl)amino)-2H-[1,3′-bipyridin]-2-one×HCl Intermediate 3 (100 mg, 0.33 mmol) and Na₂CO₃ (104 mg, 0.98 mmol) in DMSO (10 mL) at rt and the resulting solution was stirred at 100° C. for 5 h. The reaction mixture was diluted with EtOAc (100 mL) and washed sequentially with brine (3×25 mL). The organic layer was dried over Na₂SO₄, filtered and evaporated to afford a crude product which was purified by preparative HPLC, PrepMethod F (gradient: 25-55%), to give the title compound (51 mg, 39%) as a white solid; MS (ESI) m/z [M+H]⁺ 404 ¹H NMR (300 MHz, DMSO-d₆) δ 1.48-1.65 (2H, m), 1.89-2.03 (2H, m), 2.08-2.28 (2H, m), 4.30-4.41 (2H, m), 6.27 (1H, td), 6.45 (1H, d), 6.54 (1H, d), 6.96-7.04 (2H, m), 7.21 (1H, td), 7.36-7.51 (3H, m), 7.61 (1H, dd), 7.66 (1H, dd), 7.94 (1H, d), 8.13 (1H, d).

### Example 8

### 6′-(((1S,3S)-3-((6-Methylbenzo[d]thiazol-2-yl)amino)cyclopentyl)amino)-2H-[1,3′-bipyridin]-2-one—Compound 8

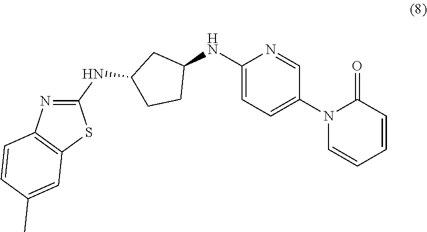 [SMILES: Cc1ccc2nc(N[C@H]3CC[C@H](Nc4ccc(-n5ccccc5=O)cn4)C3)sc2c1]

2-Chloro-6-methylbenzo[d]thiazole (100 mg, 0.54 mmol) was added to 6′-(((1S,3S)-3-aminocyclopentyl)amino)-2H-[1,3′-bipyridin]-2-one×HCl Intermediate 3 (251 mg, 0.82 mmol) and Na₂CO₃ (173 mg, 1.63 mmol) in DMSO (10 mL) at rt and the resulting solution was stirred at 100° C. for 5 h. The reaction mixture was diluted with EtOAc (100 mL), and washed sequentially with brine (3×35 mL). The organic layer was dried over Na₂SO₄, filtered and evaporated. The crude product was purified by preparative HPLC, PrepMethod A (gradient; 33-63%); to give the title compound (52 mg, 23%) as a white solid; HRMS (ESI) m/z [M+H]⁺ calcd for C₂₃H₂₄N₅OS: 418.1696, found: 418.1704; ¹H NMR (300 MHz, DMSO-d₆) δ 1.42-1.69 (2H, m), 1.83-2.04 (2H, m), 2.08-2.25 (2H, m), 2.29 (3H, s), 4.32 (2H, hept), 6.25 (1H, td), 6.43 (1H, dt), 6.48-6.59 (1H, m), 6.87-7.05 (2H, m), 7.25 (1H, d), 7.34-7.52 (3H, m), 7.59 (1H, ddd), 7.92 (1H, d), 7.99 (1H, d).

### Example 9

### 2-(((1S,3S)-3-((2-Oxo-2H-[1,3′-bipyridin]-6′-yl)amino)cyclopentyl)amino)benzo[d]thiazole-5-carbonitrile—Compound 9

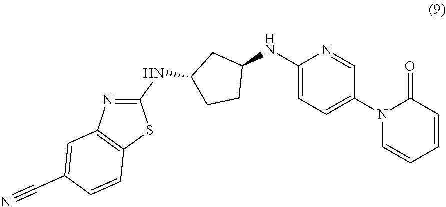 [SMILES: N#Cc1ccc2sc(N[C@H]3CC[C@H](Nc4ccc(-n5ccccc5=O)cn4)C3)nc2c1]

6′-(((1S,3S)-3-Aminocyclopentyl)amino)-2H-[1,3′-bipyridin]-2-one×HCl Intermediate 3 (100 mg, 0.33 mmol) was added to 2-chlorobenzo[d]thiazole-5-carbonitrile (63 mg, 0.33 mmol) and Na₂CO₃ (104 mg, 0.98 mmol) in DMSO (4 mL) at rt and the resulting solution was stirred at 100° C. for 2 h. The reaction mixture was diluted with EtOAc (100 mL) and washed sequentially with brine (3×25 mL). The organic layer was dried (Na₂SO₄), filtered and evaporated to afford a crude product. The crude product was purified by preparative HPLC, PrepMethod A (gradient: 29-59%), to give the title compound (69 mg, 49%) as a white solid; HRMS (ESI) m/z [M+H]⁺ calcd for C₂₃H₂₁N₆OS: 429.1492, found: 429.1504; ¹H-NMR (300 MHz, DMSO-d₆) δ 1.50-1.66 (2H, m), 1.90-2.04 (2H, m), 2.14-2.29 (2H, m), 4.32-4.44 (2H, m), 6.28 (1H, td), 6.45 (1H, d), 6.54 (1H, d), 7.00 (1H, d), 7.38-7.52 (3H, m), 7.61 (1H, d), 7.77 (1H, d), 7.90 (1H, d), 7.94 (1H, d), 8.52 (1H, d).

### Example 10

### 6′-(((1S,3S)-3-((1H-Benzo[d]imidazol-2-yl)amino)cyclopentyl)amino)-2H-[1,3′-bipyridin]-2-one-compound 10

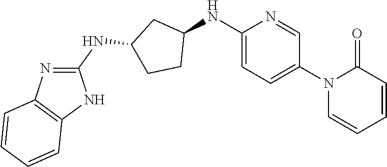 [SMILES: O=c1ccccn1-c1ccc(N[C@H]2CC[C@H](Nc3nc4ccccc4[nH]3)C2)nc1]

tBuBrettPhos G3 (4.3 mg, 5.00 μmol) and 6′-(((1S,3S)-3-aminocyclopentyl)amino)-2H-[1,3′-bipyridin]-2-one×3 HCl Intermediate 3 (19 mg, 0.05 mmol) were added as a stock solutions in NMP (180 μL) to a vial containing 2-bromo-1H-benzo[d]imidazole (9.9 mg, 0.05 mmol) in a glovebox, followed by MTBD (32 μL, 0.22 mmol). The vial was sealed, and the reaction mixture was stirred at rt for 16 h. SiliaMetS Thiol (SH) Metal Scavenger was added and the mixture was stirred for 3 h at rt. The reaction mixture was diluted with MeOH and loaded on an SCX-2 column (500 mg/6 mL) and eluted with 2 M NH₃ in MeOH (5 mL). The volatiles were removed in vacuo and the sample was diluted with DMSO and purified by preparative HPLC, PrepMethod E (gradient: 5-95%), to give (3.6 mg, 18%) of the title compound; HRMS (ESI) m/z [M+H]⁺ calcd for C₂₂H₂₃N₆O: 387.1928, found: 387.1526; ¹H NMR (600 MHz, DMSO-d₆) δ 1.47-1.61 (2H, m), 1.88-2.01 (2H, m), 2.18 (2H, qt), 4.26 (1H, h), 4.35 (1H, q), 6.27 (1H, td), 6.45 (1H, dt), 6.54 (1H, dd), 6.68 (1H, d), 6.82 (1H, td), 6.88 (1H, td), 6.96 (1H, d), 7.12 (2H, dd), 7.41 (1H, dd), 7.48 (1H, ddd), 7.61 (1H, ddd), 7.93 (1H, d), 10.61 (1H, s).

### Example 11

### 6′-(((1S,3S)-3-(Oxazolo[5,4-b]pyridin-2-ylamino)cyclopentyl)amino)-2H-[1,3′-bipyridin]-2-one—Compound 11

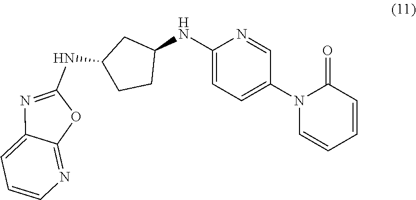 [SMILES: O=c1ccccn1-c1ccc(N[C@H]2CC[C@H](Nc3nc4cccnc4o3)C2)nc1]

2-Chlorooxazolo[5,4-b]pyridine (17.15 mg, 0.11 mmol), 6′-(((1S,3S)-3-aminocyclopentyl)amino)-2H-[1,3′-bipyridin]-2-one×HCl Intermediate 3 (30 mg, 0.11 mmol) and NMP (0.5 mL) were added to a vial, followed by DIPEA (0.097 mL, 0.55 mmol. The vial was sealed and heated at 130° C. for 20 h. The reaction mixture was cooled to rt, diluted with DMSO (1.5 mL) and purified by preparative HPLC, PrepMethod C (gradient: 15-55%), to give the title compound (6 mg, 13%) as a white solid; HRMS (ESI) m/z [M+H]⁺ calcd for C₂₁H₂₁N₆O₂: 389.1720, found: 389.1706; ¹H NMR (500 MHz, CD₃OD) δ 1.57-1.86 (2H, m), 1.99-2.1 (1H, m), 2.1-2.22 (1H, m), 2.22-2.45 (2H, m), 4.23-4.48 (2H, m), 6.47 (1H, td), 6.58-6.69 (2H, m), 7.21 (1H, dd), 7.45 (1H, dd), 7.56-7.67 (3H, m), 7.87 (1H, dd), 7.95 (1H, d).

### Example 12

### 3-Methoxy-6′-(((1S,3S)-3-(oxazolo[5,4-b]pyridin-2-ylamino)cyclopentyl)amino)-2H-[1,3′-bipyridin]-2-one—Compound 12

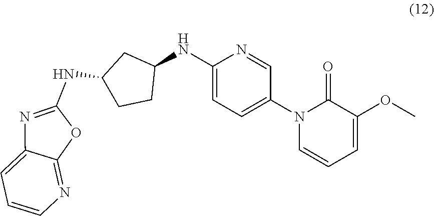 [SMILES: COc1cccn(-c2ccc(N[C@H]3CC[C@H](Nc4nc5cccnc5o4)C3)nc2)c1=O]

2-Chlorooxazolo[5,4-b]pyridine (100 mg, 0.65 mmol) was added to 6′-(((1S,3S)-3-aminocyclopentyl)amino)-3-methoxy-2H-[1,3′-bipyridin]-2-one×4.2 TFA Intermediate 5 (756 mg, 0.97 mmol), Pd-PEPPSI-IpentCl 2-methylpyridine (27 mg, 0.03 mmol) and Cs₂CO₃ (1054 mg, 3.24 mmol) in 1,4-dioxane (10 mL) at 25° C. The resulting mixture was stirred at 100° C. for 15 h under a nitrogen atmosphere. The reaction mixture was poured into sat brine (150 mL) and extracted with EtOAc (4×100 mL). The organic layers were combined and washed with sat brine (3×100 mL), dried over Na₂SO₄, filtered and evaporated. The residue was purified by preparative TLC (7 M NH₃ in MeOH:DCM, 1:15) and then by preparative HPLC, PrepMethod F (gradient: 10-40%), to give the title compound (135 mg, 49%) as a white solid; HRMS (ESI) m/z [M+H]⁺ calcd for C₂₂H₂₃N₆O₃: 419.1826, found: 419.1832; ¹H NMR (300 MHz, DMSO-d₆) δ 1.45-1.71 (2H, m), 1.95 (2H, m), 2.17 (2H, m), 3.70 (3H, s), 4.30 (2H, m), 6.17 (1H, m), 6.53 (1H, d), 6.90 (2H, m), 7.14 (2H, m), 7.38 (1H, m), 7.56 (1H, d), 7.86 (2H, m), 8.40 (1H, d).

### Example 13

### 5-Chloro-6′-(((1S,3S)-3-(oxazolo[5,4-b]pyridin-2-ylamino)cyclopentyl)amino)-2H-[1,3′-bipyridin]-2-one—Compound 13

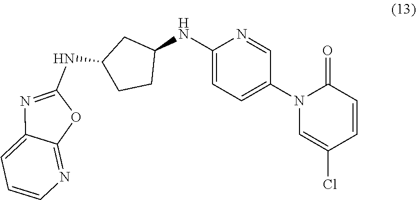 [SMILES: O=c1ccc(Cl)cn1-c1ccc(N[C@H]2CC[C@H](Nc3nc4cccnc4o3)C2)nc1]

6′-(((1S,3S)-3-Aminocyclopentyl)amino)-5-chloro-2H-[1,3′-bipyridin]-2-one×5 TFA Intermediate 7 (396 mg, 0.45 mmol) was added to 2-chlorooxazolo[5,4-b]pyridine (100 mg, 0.65 mmol), Pd-PEPPSI-IpentCl 2-methylpyridine (27 mg, 0.03 mmol) and Cs₂CO₃ (1.05 g, 3.24 mmol) in 1,4-dioxane (10 mL) at 25° C. The resulting suspension was stirred at 100° C. for 18 h under a nitrogen atmosphere. The reaction mixture was concentrated, diluted with EtOAc (125 mL) and washed sequentially with water (3×50 mL) and sat brine (3×35 mL). The organic layer was dried over Na₂SO₄, filtered and evaporated. The residue was purified by preparative TLC (7 M NH₃ in MeOH:DCM, 1:15) and then by preparative HPLC, PrepMethod F (gradient: 16-46%), to give the title compound (123 mg, 45%) as a white solid; HRMS (ESI) m/z [M+H]⁺ calcd for C₂₁H₂₀ClN₆O₂: 423.1330, found: 423.1370; ¹H NMR (300 MHz, DMSO-d₆) δ ppm 1.45-1.74 (2H, m) 1.87-2.07 (2H, m) 2.11-2.28 (2H, m) 4.20-4.45 (2H, m) 6.51 (2H, dd) 7.02 (1H, d) 7.17 (1H, dd) 7.43 (1H, dd) 7.50-7.64 (2H, m) 7.84 (1H, dd) 7.94 (2H, dd) 8.37-8.47 (1H, m).

### Example 14

### 5-Methoxy-6′-(((1S,3S)-3-(oxazolo[5,4-b]pyridin-2-ylamino)cyclopentyl)amino)-2H-[1,3′-bipyridin]-2-one—Compound 14

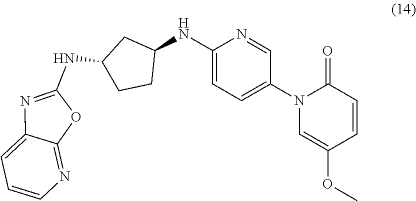 [SMILES: COc1ccc(=O)n(-c2ccc(N[C@H]3CC[C@H](Nc4nc5cccnc5o4)C3)nc2)c1]

6′-(((1S,3S)-3-Aminocyclopentyl)amino)-5-methoxy-2H-[1,3′-bipyridin]-2-one×7 TFA Intermediate 9 (498 mg, 0.45 mmol) was added to 2-chlorooxazolo[5,4-b]pyridine (100 mg, 0.65 mmol), Pd-PEPPSI-IpentCl 2-methylpyridine (27.2 mg, 0.03 mmol) and Cs₂CO₃ (1054 mg, 3.24 mmol) in 1,4-dioxane (5 mL) at 25° C. The resulting suspension was stirred at 100° C. for 18 h under a nitrogen atmosphere. The reaction mixture was concentrated, diluted with EtOAc (125 mL) and washed sequentially with water (3×50 mL) and sat brine (3×35 mL). The organic layer was dried over Na₂SO₄, filtered and evaporated. The residue was purified by preparative TLC (7 M NH₃ in MeOH:DCM, 1:15) and then by preparative HPLC, PrepMethod F (gradient: 11-41%), to give the title compound (180 mg, 66%) as a white solid; HRMS (ESI) m/z [M+H]⁺ calcd for C₂₂H₂₃N₆O₃: 419.1826, found: 419.1806; ¹H NMR (300 MHz, DMSO-d₆) δ ppm 1.46-1.74 (2H, m) 1.88-2.07 (2H, m) 2.11-2.31 (2H, m) 3.64 (3H, s) 4.19-4.47 (2H, m) 6.43 (1H, d) 6.55 (1H, d) 6.96 (1H, d) 7.11-7.26 (2H, m) 7.36 (1H, dd) 7.44 (1H, dd) 7.58 (1H, dd) 7.84 (1H, dd) 7.99 (1H, d) 8.42 (1H, d).

### Example 15

### 6′-(((1S,3S)-3-(Oxazolo[5,4-b]pyridin-2-ylamino)cyclopentyl)amino)-2-oxo-2H-[1,3′-bipyridine]-3-carbonitrile—Compound 15

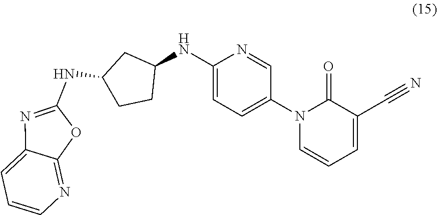 [SMILES: N#Cc1cccn(-c2ccc(N[C@H]3CC[C@H](Nc4nc5cccnc5o4)C3)nc2)c1=O]

Cs₂CO₃ (517 mg, 1.59 mmol) was added to 6′-(((1S,3S)-3-aminocyclopentyl)amino)-2-oxo-2H-[1,3′-bipyridine]-3-carbonitrile×4 HCl Intermediate 12 (140 mg, 0.32 mmol), 2-chlorooxazolo[5,4-b]pyridine (49.0 mg, 0.32 mmol) and Pd-PEPPSI-IpentCl 2-methylpyridine (13.35 mg, 0.02 mmol) in DMF (5 mL) at 20° C. The resulting mixture was stirred at 100° C. for 5 h under a nitrogen atmosphere. The reaction mixture was diluted with water (50 mL) and extracted with EtOAc (4×100 mL). The organic layers were combined and washed with sat brine (5×150 mL). The organic layer was dried over Na₂SO₄, filtered and evaporated. The residue was purified by preparative TLC (EtOAc) and then by preparative HPLC, PrepMethod A (gradient: 20-50%), to give the title compound (50 mg, 38%) as a pale yellow solid; HRMS (ESI) m/z [M+H]⁺ calcd for C₂₂H₂₀N₇O₂: 414.1672, found: 414.1642; ¹H NMR (300 MHz, DMSO-d₆) δ 1.45-1.72 (2H, m), 1.98 (2H, m), 2.18 (2H, tt), 4.31 (2H, m), 6.47 (1H, m), 6.56 (1H, d), 7.11 (1H, d), 7.17 (1H, m), 7.48 (1H, m), 7.59 (1H, m), 7.84 (1H, m), 8.00 (1H, d), 8.06 (1H, m), 8.23 (1H, m), 8.42 (1H, d).

### Example 16

### 3-Chloro-6′-(((1S,3S)-3-(oxazolo[5,4-b]pyridin-2-ylamino)cyclopentyl)amino)-2H-[1,3′-bipyridin]-2-one—Compound 16

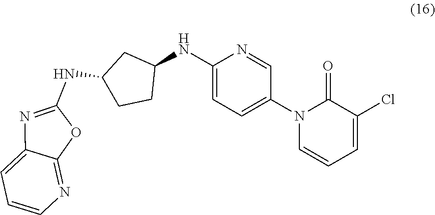 [SMILES: O=c1c(Cl)cccn1-c1ccc(N[C@H]2CC[C@H](Nc3nc4cccnc4o3)C2)nc1]

2-Chlorooxazolo[5,4-b]pyridine (108 mg, 0.70 mmol) was added to 6′-(((1S,3S)-3-aminocyclopentyl)amino)-3-chloro-2H-[1,3′-bipyridin]-2-one Intermediate 26 (178 mg, 0.58 mmol), Pd-PEPPSI-IpentCl 2-methylpyridine (24.56 mg, 0.03 mmol) and Cs₂CO₃ (951 mg, 2.92 mmol) in 1,4-dioxane (20 mL) at 30° C. The resulting solution was stirred at 100° C. for 15 h. The reaction mixture was concentrated, diluted with EtOAc (250 mL) and washed with sat brine (3×75 mL). The organic layer was dried over Na₂SO₄, filtered and evaporated. The residue was purified by preparative TLC (MeOH:DCM, 1:15) and then by preparative HPLC, PrepMethod A (gradient: 27-47%), to give the title compound (49.5 mg, 20%) as a white solid; HRMS (ESI) m/z [M+H]⁺ calcd for C₂₁H₂₀ClN₆O₂: 423.1330, found: 423.1324; ¹H NMR (300 MHz, DMSO-d₆) δ 1.60 (2H, m), 1.99 (2H, m), 2.18 (2H, m), 4.32 (2H, m), 6.31 (1H, m), 6.55 (1H, d), 7.05 (1H, d), 7.17 (1H, m), 7.45 (1H, m), 7.59 (1H, m), 7.67 (1H, m), 7.79-7.86 (2H, m), 7.98 (1H, d), 8.42 (1H, d).

### Example 17

### 6′-(((1S,3S)-3-(Oxazolo[5,4-b]pyridin-2-ylamino)cyclopentyl)amino)-2-oxo-2H-[1,3′-bipyridine]-5-carbonitrile—Compound 17

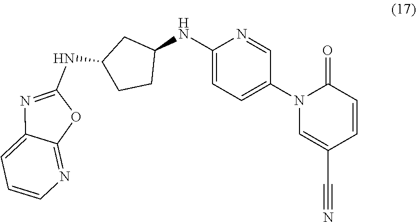 [SMILES: N#Cc1ccc(=O)n(-c2ccc(N[C@H]3CC[C@H](Nc4nc5cccnc5o4)C3)nc2)c1]

(1S,3S)—N¹-(Oxazolo[5,4-b]pyridin-2-yl)cyclopentane-1,3-diamine×3 TFA Intermediate 14 (194 mg, 0.35 mmol) was added to 6′-chloro-2-oxo-2H-[1,3′-bipyridine]-5-carbonitrile Intermediate 18 (80 mg, 0.35 mmol), Cs₂CO₃ (338 mg, 1.04 mmol) and Pd-PEPPSI-IpentCl 2-methylpyridine (14.5 mg, 0.02 mmol) in 1,4-dioxane (10 mL). The resulting mixture was stirred at 100° C. for 15 h under a nitrogen atmosphere. The reaction mixture was filtered through Celite. The filter cake was washed with EtOAc (3×20 mL). The filtrate was concentrated under reduced pressure and the crude product was purified by preparative TLC (MeOH:DCM, 1:10) and then by preparative HPLC, PrepMethod K (gradient: 3-14%), to give the title compound (27 mg, 18%) as a white solid; HRMS (ESI) m/z [M+H]⁺ calcd for C₂₂H₂₀N₇O₂: 414.1672, found: 414.1676; ¹H NMR (300 MHz, CD₃CD) δ ppm 1.58-1.86 (2H, m) 1.99-2.24 (2H, m) 2.24-2.45 (2H, m) 4.25-4.52 (2H, m) 6.57-6.72 (2H, m) 7.23 (1H, dd) 7.48 (1H, dd) 7.61 (1H, dd) 7.71 (1H, dd) 7.89 (1H, dd) 7.97-8.05 (1H, m) 8.37 (1H, dd).

### Example 18

### 1-Methyl-3-(6-(((1S,3S)-3-(oxazolo[5,4-b]pyridin-2-ylamino)cyclopentyl)amino)pyridin-3-yl)-1,3-dihydro-2H-imidazo[4,5-b]pyridin-2-one—Compound 18

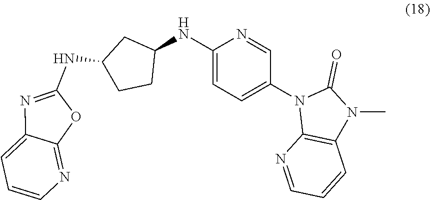 [SMILES: Cn1c(=O)n(-c2ccc(N[C@H]3CC[C@H](Nc4nc5cccnc5o4)C3)nc2)c2ncccc21]

3-(6-(((1S,3S)-3-Aminocyclopentyl)amino)pyridin-3-yl)-1-methyl-1,3-dihydro-2H-imidazo[4,5-b]pyridin-2-one×3 TFA Intermediate 21 (250 mg, 0.38 mmol) was added to 2-chlorooxazolo[5,4-b]pyridine (58.0 mg, 0.38 mmol), Cs₂CO₃ (611 mg, 1.88 mmol) and Pd-PEPPSI-IpentCl 2-methylpyridine (15.78 mg, 0.02 mmol) in 1,4-dioxane (10 mL) at 25° C. The resulting mixture was stirred at 100° C. for 15 h under a nitrogen atmosphere. The reaction mixture was poured into sat brine (150 mL) and extracted with EtOAc (4×100 mL) The organic layers were combined and washed with sat brine (3×100 mL), dried over Na₂SO₄, filtered and evaporated. The residue was purified by preparative TLC (7M NH₃ in MeOH:DCM, 1:15) and then by preparative HPLC, PrepMethod F (gradient: 18-38%), to give the title compound (81 mg, 48%) as a white solid; HRMS (ESI) m/z [M+H]⁺ calcd for C₂₃H₂₃N₈O₂: 443.1938, found: 443.1912; ¹H NMR (300 MHz, DMSO-d₆) δ ppm 1.48-1.74 (2H, m) 1.93-2.11 (2H, m) 2.14-2.36 (2H, m) 3.41 (3H, s partially overlapping with solvent peak) 4.21-4.46 (2H, m) 6.60 (1H, d) 6.97 (1H, d) 7.06-7.25 (2H, m) 7.48-7.64 (3H, m) 7.84 (1H, dd) 7.94 (1H, dd) 8.11 (1H, d) 8.42 (1H, d).

### Example 19

### 6′-(((1S,3S)-3-(Thiazolo[5,4-b]pyridin-2-ylamino)cyclopentyl)amino)-2H-[1,3′-bipyridin]-2-one—Compound 19

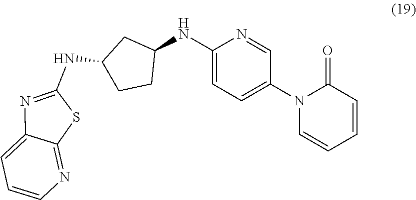 [SMILES: O=c1ccccn1-c1ccc(N[C@H]2CC[C@H](Nc3nc4cccnc4s3)C2)nc1]

2-Chlorothiazolo[5,4-b]pyridine (30 mg, 0.18 mmol), 6′-(((1S,3S)-3-aminocyclopentyl)amino)-2H-[1,3′-bipyridin]-2-one×HCl Intermediate 3 (40 mg, 0.15 mmol) and NMP (0.35 mL) were added to a vial, followed by DIPEA (0.129 mL, 0.74 mmol). The vial was sealed and heated at 130° C. for 20 h. The mixture was diluted with EtOAc and washed with 1 M NaHCO₃. The water phase was separated and extracted with EtOAc (×2). The combined organic layer was dried over Na₂SO₄, filtered and evaporated. The residue was diluted with DMSO and purified by preparative HPLC, PrepMethod D (gradient: 5-50%), to give the title compound (15 mg, 25%) as a yellow solid; HRMS (ESI) m/z [M+H]⁺ calcd for C₂₁H₂₁N₆OS: 405.1492, found: 405.1452; ¹H NMR (500 MHz, CD₃OD) δ 1.57-1.82 (2H, m), 2.01-2.2 (2H, m), 2.23-2.44 (2H, m), 4.32-4.53 (2H, m), 6.41-6.52 (1H, m), 6.58-6.68 (2H, m), 7.29 (1H, dd), 7.45 (1H, dd), 7.54-7.66 (2H, m), 7.70 (1H, dd), 7.96 (1H, d), 8.05-8.15 (1H, m).

### Example 20

### 6′-(((1S,3S)-3-(Thiazolo[4,5-b]pyridin-2-ylamino)cyclopentyl)amino)-2H-[1,3′-bipyridin]-2-one—Compound 20

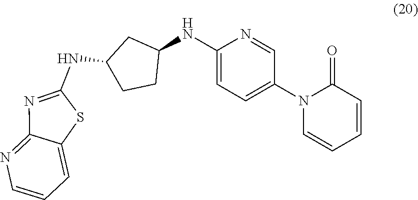 [SMILES: O=c1ccccn1-c1ccc(N[C@H]2CC[C@H](Nc3nc4ncccc4s3)C2)nc1]

2-Chlorothiazolo[4,5-b]pyridine (30 mg, 0.18 mmol), 6′-(((1S,3S)-3-aminocyclopentyl)amino)-2H-[1,3′-bipyridin]-2-one×HCl Intermediate 3 (50 mg, 0.16 mmol) and NMP (0.35 mL) were added to a vial, followed by DIPEA (0.2 mL, 1.15 mmol). The vial was sealed and heated at 130° C. for 24 h. The mixture was diluted with DMSO/MeCN (2:1) to 4 mL and purified by preparative HPLC, PrepMethod D (gradient: 5-50%), to give the title compound (48 mg, 72%) as a beige solid. According to ¹HNMR the sample contained 0.65 mol eq NMP; HRMS (ESI) m/z [M+H]⁺ calcd for C₂₁H₂₁N₆OS: 405.1492, found: 405.1458; ¹H NMR (500 MHz, CD₃OD) δ 1.59-1.78 (2H, m), 2.07-2.17 (2H, m), 2.22-2.35 (2H, m), 4.33-4.44 (1H, m), 4.44-4.56 (1H, m), 6.47 (1H, td), 6.59-6.66 (2H, m), 7.02 (1H, dd), 7.45 (1H, dd), 7.56-7.65 (2H, m), 7.95 (1H, d), 8.00 (1H, dd), 8.22 (1H, dd).

### Example 21

### 3-Methoxy-6′-(((1S,3S)-3-(thiazolo[5,4-b]pyridin-2-ylamino)cyclopentyl)amino)-2H-[1,3′-bipyridin]-2-one—Compound 21

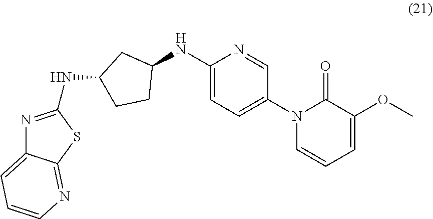 [SMILES: COc1cccn(-c2ccc(N[C@H]3CC[C@H](Nc4nc5cccnc5s4)C3)nc2)c1=O]

6′-(((1S,3S)-3-Aminocyclopentyl)amino)-3-methoxy-2H-[1,3′-bipyridin]-2-one×4 TFA Intermediate 5 (300 mg, 0.40 mmol) was added to 2-bromothiazolo[5,4-b]pyridine (85 mg, 0.40 mmol), Cs₂CO₃ (646 mg, 1.98 mmol) and Pd-PEPPSI-IpentCl 2-methylpyridine (33.4 mg, 0.04 mmol) in 1,4-dioxane (10 mL) at 25° C. The resulting mixture was stirred at 100° C. for 15 h under a nitrogen atmosphere. The reaction mixture was poured into sat brine (150 mL) and extracted with EtOAc (4×100 mL). The organic layers were combined and washed with sat brine (3×100 mL), dried over Na₂SO₄, filtered and evaporated. The residue was purified by preparative TLC (EtOAc:PE, 1:10) and then by preparative HPLC, PrepMethod A (gradient: 19-49%), to give the title compound (40 mg, 23%) as a white solid; HRMS (ESI) m/z [M+H]⁺ calcd for C₂₂H₂₃N₆O₂S: 435.1598, found: 435.1594; ¹H NMR (300 MHz, DMSO-d₆) δ 1.44-1.67 (2H, m), 1.98 (2H, m), 2.10-2.29 (2H, m), 3.72 (3H, s), 4.37 (2H, m), 6.20 (1H, m), 6.54 (1H, d), 6.86 (1H, m), 6.98 (1H, d), 7.17 (1H, m), 7.24 (1H, m), 7.39 (1H, m), 7.60-7.74 (1H, m), 7.92 (1H, d), 8.09 (1H, m), 8.47 (1H, d).

### Example 22

### 3-Methyl-6′-(((1S,3S)-3-(thiazolo[5,4-b]pyridin-2-ylamino)cyclopentyl)amino)-2H-[1,3′-bipyridin]-2-one—Compound 22

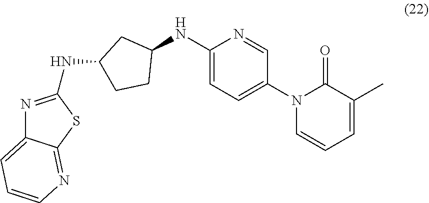 [SMILES: Cc1cccn(-c2ccc(N[C@H]3CC[C@H](Nc4nc5cccnc5s4)C3)nc2)c1=O]

Cs₂CO₃ (1490 mg, 4.57 mmol) was added to 6′-(((1S,3S)-3-aminocyclopentyl)amino)-3-methyl-2H-[1,3′-bipyridin]-2-one Intermediate 23 (260 mg, 0.91 mmol), 2-bromothiazolo[5,4-b]pyridine (236 mg, 1.10 mmol) and Pd-PEPPSI-IpentCl 2-methylpyridine (38.5 mg, 0.05 mmol) in 1,4-dioxane (10 mL) at 25° C. The resulting mixture was stirred at 100° C. for 15 h under a nitrogen atmosphere. The reaction mixture was diluted with EtOAc (100 mL) and washed with sat brine (3×100 mL). The organic layer was dried over Na₂SO₄, filtered and evaporated. The crude product was purified by preparative HPLC, PrepMethod L (gradient: 15-19%), to give the title compound (81 mg, 21%) as a yellow gum; HRMS (ESI) m/z [M+H]⁺ calcd for C₂₂H₂₃N₆OS: 419.1648, found: 419.1632; ¹H NMR (300 MHz, DMSO-d₆) δ ppm 1.57-1.73 (2H, m) 2.04 (3H, s) 2.06-2.13 (2H, m) 2.15-2.36 (2H, m) 4.19-4.65 (3H, m, partially overlapping with solvent) 6.27 (1H, t) 6.97 (1H, d) 7.26 (1H, dd) 7.37-7.57 (2H, m) 7

### Example 23

### 2-Oxo-6′-(((1S,3S)-3-(thiazolo[5,4-b]pyridin-2-ylamino)cyclopentyl)amino)-2H-[1,3′-bipyridine]-3-carbonitrile—Compound 23

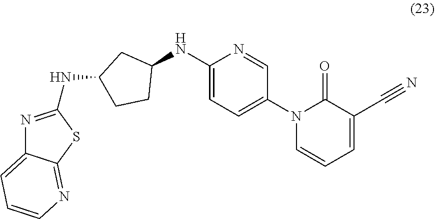 [SMILES: N#Cc1cccn(-c2ccc(N[C@H]3CC[C@H](Nc4nc5cccnc5s4)C3)nc2)c1=O]

Cs₂CO₃ (502 mg, 1.54 mmol) was added to 6′-(((1S,3S)-3-aminocyclopentyl)amino)-2-oxo-2H-[1,3′-bipyridine]-3-carbonitrile×4 HCl Intermediate 12 (136 mg, 0.31 mmol), 2-bromothiazolo[5,4-b]pyridine (133 mg, 0.62 mmol) and Pd-PEPPSI-IpentCl 2-methylpyridine (12.96 mg, 0.02 mmol) in DMF (7 mL) at 20° C. The mixture was stirred at 100° C. for 15 h under a nitrogen atmosphere. The reaction mixture was diluted with water (90 mL) and extracted with EtOAc (5×150 mL). The organic layers were combined, washed with sat brine (5×300 mL), dried over Na₂SO₄, filtered and evaporated. The residue was purified by preparative TLC (EtOAc) and then by preparative HPLC, PrepMethod A (gradient: 29-44%), to give the title compound (35 mg, 26%) as a yellow solid; HRMS (ESI) m/z [M+H]⁺ calcd for C₂₂H₂₀N₇OS: 430.1444, found: 430.1460; ¹H NMR (300 MHz, DMSO-d₆) δ 1.47-1.68 (2H, m), 1.98 (2H, m), 2.19 (2H, d), 4.31-4.48 (2H, m), 6.47 (1H, m), 6.56 (1H, d), 7.11 (1H, d), 7.25 (1H, m), 7.48 (1H, m), 7.67 (1H, m), 8.00 (1H, d), 8.08 (2H, m), 8.23 (1H, m), 8.47 (1H, d).

### Example 24

### 3-Chloro-6′-(((1S,3S)-3-(thiazolo[5,4-b]pyridin-2-ylamino)cyclopentyl)amino)-2H-[1,3′-bipyridin]-2-one—Compound 24

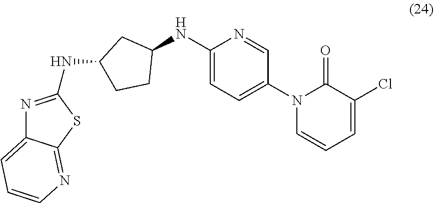 [SMILES: O=c1c(Cl)cccn1-c1ccc(N[C@H]2CC[C@H](Nc3nc4cccnc4s3)C2)nc1]

2-Bromothiazolo[5,4-b]pyridine (96 mg, 0.45 mmol) was added to Cs₂CO₃ (336 mg, 1.03 mmol) and 6′-(((1S,3S)-3-aminocyclopentyl)amino)-3-chloro-2H-[1,3′-bipyridin]-2-one×2 HCl Intermediate 26 (130 mg, 0.34 mmol) in 1,4-dioxane (20 mL) at 30° C. The resulting solution was stirred at 100° C. for 18 h. The reaction mixture was concentrated, diluted with EtOAc (250 mL) and washed with water (3×75 mL). The organic layer was dried over Na₂SO₄, filtered and evaporated. The residue was purified by preparative TLC (EtOAc:PE, 1:10) and then by preparative HPLC, PrepMethod F (gradient: 35-50%), to give the title compound (26 mg, 17%) as a white solid; HRMS (ESI) m/z [M+H]⁺ calcd for C₂₁H₂₀ClN₆OS: 439.1102, found: 439.1106; ¹H NMR (300 MHz, DMSO-d₆) δ 1.55 (2H, m), 1.88-2.02 (2H, m), 2.10-2.30 (2H, m), 4.37 (2H, m), 6.29 (1H, m), 6.53 (1H, d), 7.04 (1H, d), 7.23 (1H, m), 7.43 (1H, m), 7.59-7.72 (2H, m), 7.80 (1H, m), 7.96 (1H, d), 8.07 (1H, m), 8.45 (1H, d).

### Example 25

### 5-Methoxy-6′-(((1S,3S)-3-(thiazolo[5,4-b]pyridin-2-ylamino)cyclopentyl)amino)-2H-[1,3′-bipyridin]-2-one—Compound 25

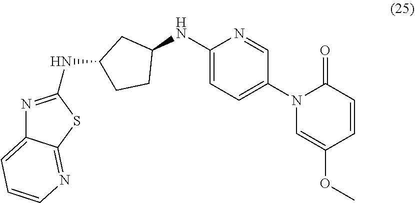 [SMILES: COc1ccc(=O)n(-c2ccc(N[C@H]3CC[C@H](Nc4nc5cccnc5s4)C3)nc2)c1]

6′-(((1S,3S)-3-Aminocyclopentyl)amino)-5-methoxy-2H-[1,3′-bipyridin]-2-one×7 TFA Intermediate 9 (300 mg, 0.27 mmol) was added to 2-bromothiazolo[5,4-b]pyridine (88 mg, 0.41 mmol), Pd-PEPPSI-IpentCl 2-methylpyridine (11.49 mg, 0.01 mmol) and Cs₂CO₃ (623 mg, 1.91 mmol) in 1,4-dioxane (8 mL) at 25° C. The resulting suspension was stirred at 100° C. for 18 h under a nitrogen atmosphere. The reaction mixture was concentrated and diluted with EtOAc (100 mL), washed sequentially with water (3×45 mL) and sat brine (3×35 mL). The organic layer was dried over Na₂SO₄, filtered and evaporated. The residue was purified by preparative TLC (7 M NH₃ MeOH:DCM, 1:15) and then by preparative HPLC, PrepMethod F (gradient: 23-43%), to give the title compound (39 mg, 32%) as a white solid; HRMS (ESI) m/z [M+H]⁺ calcd for C₂₂H₂₃N₆O₂S: 435.1598, found: 435.1560; ¹H NMR (300 MHz, DMSO-d₆) δ 1.43-1.73 (2H, m), 1.89-2.06 (2H, m), 2.1-2.33 (2H, m), 3.64 (3H, s), 4.25-4.52 (2H, m), 6.43 (1H, d), 6.54 (1H, d), 6.97 (1H, d), 7.12-7.31 (2H, m), 7.31-7.51 (2H, m), 7.67 (1H, dd), 7.98 (1H, d), 8.09 (1H, dd), 8.47 (1H, d).

### Example 26

### 2-Oxo-6′-(((1S,3S)-3-(thiazolo[5,4-b]pyridin-2-ylamino)cyclopentyl)amino)-2H-[1,3′-bipyridine]-5-carbonitrile—Compound 26

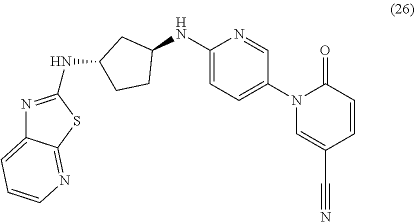 [SMILES: N#Cc1ccc(=O)n(-c2ccc(N[C@H]3CC[C@H](Nc4nc5cccnc5s4)C3)nc2)c1]

(1S,3S)—N¹-(Thiazolo[5,4-b]pyridin-2-yl)cyclopentane-1,3-diamine×4 TFA Intermediate 28 (209 mg, 0.30 mmol) was added to 6′-chloro-2-oxo-2H-[1,3′-bipyridine]-5-carbonitrile Intermediate 18 (70 mg, 0.30 mmol), Cs₂CO₃ (295 mg, 0.91 mmol) and Pd-PEPPSI-IpentCl 2-methylpyridine (12.71 mg, 0.02 mmol) in 1,4-dioxane (10 mL). The resulting mixture was stirred at 100° C. for 15 h under a nitrogen atmosphere. The reaction mixture was filtered through Celite. The filter cake was washed with EtOAc (3×20 mL) and the filtrate was concentrated under reduced pressure. The residue was purified by preparative TLC (EtOAc:PE, 5:1) and then by preparative HPLC, PrepMethod K (gradient: 3-14%), to give the title compound (18.4 mg, 14%) as a white solid; HRMS (ESI) m/z [M+H]⁺ calcd for C₂₂H₂₀N₇OS: 430.1444, found: 430.1418; ¹H NMR (300 MHz, CD₃CD) δ ppm 1.57-1.83 (2H, m) 2.00-2.18 (2H, m) 2.22-2.42 (2H, m) 4.31-4.53 (2H, m) 6.57-6.68 (2H, m) 7.29 (1H, dd) 7.46 (1H, dd) 7.66-7.73 (2H, m) 7.99 (1H, d) 8.10 (1H, dd) 8.35 (1H, dd).

### Example 27

### 6′-(((1S,3S)-3-((7-Methoxy-3H-imidazo[4,5-b]pyridin-2-yl)amino)cyclopentyl)amino)-2H-[1,3′-bipyridin]-2-one—Compound 27

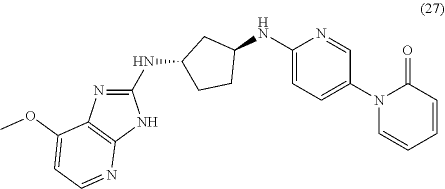 [SMILES: COc1ccnc2[nH]c(N[C@H]3CC[C@H](Nc4ccc(-n5ccccc5=O)cn4)C3)nc12]

TCDI (235 mg, 1.32 mmol) was added to 6′-(((1S,3S)-3-aminocyclopentyl)amino)-2H-[1,3′-bipyridin]-2-one×3 HCl Intermediate 3 (250 mg, 0.66 mmol) and NaOH (158 mg, 3.95 mmol) in DMF (1 mL) at rt and the resulting solution was stirred at 100° C. for 2 h. The reaction cooled to rt before the addition of 4-methoxypyridine-2,3-diamine (101 mg, 0.72 mmol) and EDC (252 mg, 1.32 mmol). The resulting solution was then stirred at 100° C. for 15 h. The mixture was filtered through a Celite pad, the filter cake was washed with DCM (3×5 mL). The filtrate was concentrated and first purified by reversed phase chromatography on a C18-column (gradient: 5-22% MeCN in water/0.1% NH₃) to afford a crude product (300 mg), which was further purified by preparative TLC (7 M NH₃ in MeOH:DCM, 1:20), to afford a crude product (80 mg). Final purification was done by preparative HPLC, PrepMethod F (gradient: 10-30%), to give the title compound (30 mg, 11%) as a white solid; HRMS (ESI) m/z [M+H]⁺ calcd for C₂₂H₂₄N₇O₂: 418.1986, found: 418.1990; ¹H NMR (300 MHz, DMSO-d₆) 1.44-1.63 (2H, m), 1.86-2.02 (2H, m), 2.08-2.24 (2H, m), 3.94 (3H, s), 4.26-4.39 (2H, m), 6.27 (1H, td), 6.44 (1H, d), 6.54 (1H, d), 6.60 (1H, d), 6.71 (1H, br d), 6.94 (1H, d), 7.38-7.51 (2H, m), 7.61 (1H, dd), 7.75 (1H, br s), 7.93 (1H, d), 11.17 (1H, d)

### Example 28

### 2-(6-(((1S,3S)-3-((3H-Imidazo[4,5-b]pyridin-2-yl)amino)cyclopentyl)amino)pyridin-3-yl)pyridazin-3(2H)-one—Compound 28

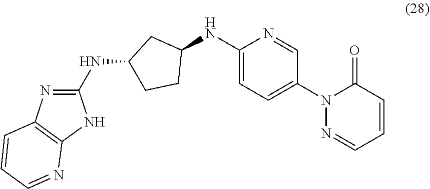 [SMILES: O=c1cccnn1-c1ccc(N[C@H]2CC[C@H](Nc3nc4cccnc4[nH]3)C2)nc1]

TEA (0.344 mL, 2.47 mmol) was added to 2-(6-(((1S,3S)-3-aminocyclopentyl)amino)pyridin-3-yl)pyridazin-3(2F)-one×2.5 HCl Intermediate 30 (179 mg, 0.49 mmol) and TCDI (176 mg, 0.99 mmol) in DMF (5 mL) at 25° C. The resulting suspension was stirred at 100° C. for 2 h under a nitrogen atmosphere. EDC (189 mg, 0.99 mmol) and pyridine-2,3-diamine (54 mg, 0.49 mmol) was added to the mixture at 25° C. The resulting suspension was stirred at 100° C. for 16 h under a nitrogen atmosphere. The crude product was purified by reversed phase flash chromatography on a C18 column (gradient: 45-60% MeCN in water) and then by preparative HPLC, PrepMethod J (gradient: 6-21%), to give the title compound (51 mg, 26%) as a yellow solid; HRMS (ESI) m/z [M+H]⁺ calcd for C₂₀H₂₁N₈O: 389.1832, found: 389.1836; ¹H NMR (300 MHz, DMSO-d₆) δ ppm 1.43-1.73 (2H, m) 1.83-2.09 (2H, m) 2.10-2.34 (2H, m) 4.14-4.51 (2H, m) 6.54 (1H, d) 6.85 (1H, dd) 6.92-7.08 (2H, m) 7.13-7.27 (1H, m) 7.32-7.62 (3H, m) 7.87 (1H, dd) 8.02 (1H, dd) 8.09-8.26 (2H, m).

### Example 29

### 3-(6-(((1S,3S)-3-((3H-Imidazo[4,5-b]pyridin-2-yl)amino)cyclopentyl)amino)pyridin-3-yl)pyrimidin-4(3H)-one—Compound 29

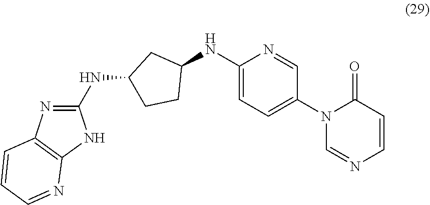 [SMILES: O=c1ccncn1-c1ccc(N[C@H]2CC[C@H](Nc3nc4cccnc4[nH]3)C2)nc1]

TEA (0.279 mL, 2.00 mmol) was added to TCDI (143 mg, 0.80 mmol) and 3-(6-(((1S,3S)-3-aminocyclopentyl)amino)pyridin-3-yl)pyrimidin-4(3H)-one×5.2 TFA Intermediate 32 (346 mg, 0.40 mmol) in DMF (5 mL) at 20° C. The resulting suspension was stirred at 100° C. for 2 h under a nitrogen atmosphere. EDC (153 mg, 0.80 mmol) and pyridine-2,3-diamine (44 mg, 0.40 mmol) was added to the mixture at 20° C. The resulting suspension was stirred at 100° C. for 16 h under a nitrogen atmosphere. The crude product was purified by reversed phase flash chromatography on a C18 column (gradient: 40-50% MeCN in water) and then by preparative HPLC, PrepMethod J (gradient: 2-17%), to give the title compound (26 mg, 16%) as a purple solid; HRMS (ESI) m/z [M+H]⁺ calcd for C₂₀H₂₁N₈O: 389.1832, found: 389.1822; ¹H NMR (300 MHz, DMSO-d₆) δ 1.38-1.67 (2H, m), 1.81-2.06 (2H, m), 2.06-2.24 (2H, m), 4.17-4.43 (2H, m), 6.42-6.6 (2H, m), 6.82 (1H, dd), 7.01-7.18 (2H, m), 7.35 (1H, dd), 7.44 (1H, dd), 7.84 (1H, d), 7.91-8.01 (2H, m), 8.18 (1H, s), 8.39 (1H, s).

### Example 30

### (1S,3S)—N¹-(3-Fluoro-[2,3′-bipyridin]-6′-yl)-N³-(3H-imidazo[4,5-b]pyridin-2-yl)cyclopentane-1,3-diamine—Compound 30

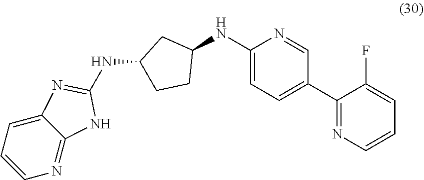 [SMILES: Fc1cccnc1-c1ccc(N[C@H]2CC[C@H](Nc3nc4cccnc4[nH]3)C2)nc1]

2-(Methylsulfonyl)-3H-imidazo[4,5-b]pyridine (134 mg, 0.68 mmol) was added to (1S,3S)—N¹-(3-fluoro-[2,3′-bipyridin]-6′-yl)cyclopentane-1,3-diamine×4.6 HCl Intermediate 35 (300 mg, 0.68 mmol) in n-BuOH (5 mL) in a microwave vial at rt and the resulting solution was stirred at 160° C. for 2 h in a microwave reactor. The crude product was pre-purified by reversed phase flash chromatography on a C18 column (gradient: 40-65% MeCN in water) to afford a crude product (70 mg) which was further purified by preparative HPLC, PrepMethod F (gradient: 44-56%), to give the title compound (10 mg, 4%) as a pale yellow solid; HRMS (ESI) m/z [M+H]⁺ calcd for C₂₁H₂₁FN₇: 390.1836, found: 390.1842; ¹H-NMR (300 MHz, DMSO-d₆) 1.51-1.65 (2H, m), 1.90-2.05 (2H, m), 2.12-2.25 (2H, m), 4.22-4.49 (2H, m), 6.59 (1H, d), 6.81-6.91 (1H, m), 7.09 (1H, d), 7.18 (1H, s), 7.30-7.45 (2H, m), 7.70-7.80 (1H, m), 7.87 (1H, d), 7.93-8.00 (1H, m), 8.45-8.50 (1H, m), 8.59-8.61 (1H, m).

### Example 31

### 2-(2-(((1S,3S)-3-((3H-Imidazo[4,5-b]pyridin-2-yl)amino)cyclopentyl)amino)pyrimidin-5-yl)pyridazin-3(2H)-one—Compound 31

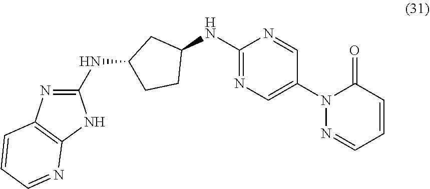 [SMILES: O=c1cccnn1-c1cnc(N[C@H]2CC[C@H](Nc3nc4cccnc4[nH]3)C2)nc1]

2-(2-(((1S,3S)-3-((3-Tosyl-3H-imidazo[4,5-b]pyridin-2-yl)amino)cyclopentyl)amino)pyrimidin-5-yl)pyridazin-3(2H)-one* Intermediate 40 (80 mg, 0.15 mmol) was added to K₂CO₃ (41 mg, 0.29 mmol) in MeOH (5 mL). The resulting mixture was stirred at 60° C. for 3 h. The reaction mixture was filtered through a filter paper. The filter cake was washed with MeOH (3×20 mL) and the filtrate was concentrated under reduced pressure. The crude product was purified by preparative TLC (MeOH:DCM, 1:10) and then by preparative HPLC, PrepMethod F (gradient: 5-30%), to give the title compound (10 mg, 17%) as a white solid; HRMS (ESI) m/z [M+H]⁺ calcd for C₁₉H₂₀N₉O: 390.1786, found: 390.1758; ¹H NMR (300 MHz, DMSO-d₆) δ ppm 1.45-1.74 (2H, m) 1.92-2.05 (2H, m) 2.09-2.28 (2H, m) 4.13-4.59 (2H, m) 6.70-6.93 (1H, m) 7.07 (1H, dd) 7.10-7.28 (1H, m) 7.30-7.42 (1H, m) 7.50 (1H, dd) 7.65-7.76 (1H, m) 7.77-7.96 (1H, m) 7.98-8.14 (1H, m) 8.49 (2H, s) 10.45-11.83 (1H, m). (*regiochemistry was not confirmed)

### Example 32

### (1S,3S)—N¹-(5-(2-(Difluoromethoxy)-6-fluorophenyl)pyridin-2-yl)-N³-(3H-imidazo[4,5-b]pyridin-2-yl)cyclopentane-1,3-diamine—Compound 32

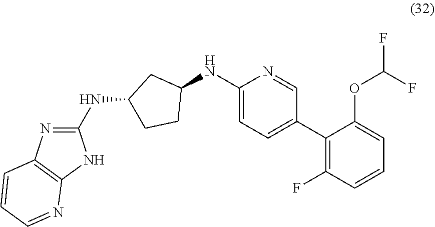 [SMILES: Fc1cccc(OC(F)F)c1-c1ccc(N[C@H]2CC[C@H](Nc3nc4cccnc4[nH]3)C2)nc1]

Cs₂CO₃ (174 mg, 0.53 mmol) was added to (1S,3S)—N¹-(5-(2-(difluoromethoxy)-6-fluorophenyl)pyridin-2-yl)cyclopentane-1,3-diamine Intermediate 43 (60 mg, 0.18 mmol), 2-chloro-3-tosyl-3H-imidazo[4,5-b]pyridine* Intermediate 39 (109 mg, 0.36 mmol) and Pd-PEPPSI-IpentCl 2-methylpyridine (12 mg, 0.01 mmol) in 1,4-dioxane (3 mL) at rt and the resulting suspension was stirred at 100° C. for 16 h under a nitrogen atmosphere. A solution of K₂CO₃ (74 mg, 0.53 mmol) in MeOH (1 mL) was added to the mixture at rt and the resulting suspension was stirred at 60° C. for 1 h. The reaction mixture was poured into water (100 mL), extracted with DCM (2×50 mL), and the organic layers were combined and washed with sat brine (2×50 mL). The organic layer was dried over Na₂SO₄, filtered and evaporated. The residue was first purified by preparative TLC (7 M NH₃ in MeOH:DCM, 1:20) to afford a crude product (51 mg) which was further purified by preparative HPLC, PrepMethod F (gradient: 50-68%), to give the title compound (5 mg, 6%) as a white solid; HRMS (ESI) m/z [M+H]⁺ calcd for C₂₃H₂₂F₃N₆O: 455.1802, found: 455.1814; ¹H-NMR (300 MHz, DMSO-d₆) δ ppm 1.45-1.65 (2H, m), 1.88-2.05 (2H, m), 2.12-2.25 (2H, m), 4.23-4.44 (2H, m), 6.55 (1H, d), 6.75-6.98 (3H, m), 7.12-7.27 (3H, m), 7.33-7.48 (3H, m), 7.78-7.95 (1H, d), 7.97 (1H, s), 11.06 (1H, d); ¹⁹F-NMR (282 MHz, DMSO-d₆) δ ppm −113.761, −81.749. (*regiochemistry was not confirmed)

### Example 33

### 1-(6-(((1S,3S)-3-((3H-Imidazo[4,5-b]pyridin-2-yl)amino)cyclopentyl)amino)pyridin-3-yl)quinolin-2(1H)-one—Compound 33

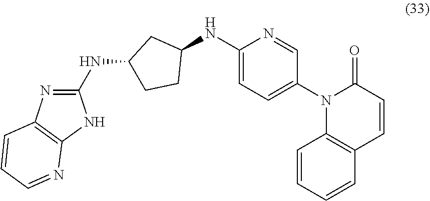 [SMILES: O=c1ccc2ccccc2n1-c1ccc(N[C@H]2CC[C@H](Nc3nc4cccnc4[nH]3)C2)nc1]

A solution of 1-(6-(((1S,3S)-3-((3-(4-methoxybenzyl)-3H-imidazo[4,5-b]pyridin-2-yl)amino)cyclopentyl)amino)pyridin-3-yl)quinolin-2(1H)-one and 1-(6-(((1S,3S)-3-((1-(4-methoxybenzyl)-1H-imidazo[4,5-b]pyridin-2-yl)amino)cyclopentyl)amino)pyridin-3-yl)quinolin-2(1H)-one Intermediate 48 (60 mg, 0.11 mmol) in TFA (2 mL) was stirred at 60° C. for 5 h. The reaction mixture was concentrated and the crude product was purified by preparative HPLC, PrepMethod I (gradient: 5-20%), to give the title compound (15 mg, 32%) as a yellow solid; HRMS (ESI) m/z [M+H]⁺ calcd for C₂₅H₂₄N₇O: 438.2036, found: 438.2056; ¹H NMR (300 MHz, DMSO-d₆) δ 1.58-1.74 (m, 2H), 2.06 (br s, 2H), 2.32 (br s, 2H), 4.42 (d, 2H), 6.70 (ddd, 3H), 7.19-7.29 (m, 3H), 7.34-7.51 (m, 2H), 7.75-7.84 (m, 2H), 7.89 (d, 1H), 8.04 (t, 2H), 8.99 (s, 1H), 12.26 (br s, 1H).

### Example 34

### 1-(6-(((1S,3S)-3-((3H-Imidazo[4,5-b]pyridin-2-yl)amino)cyclopentyl)amino)pyridin-3-yl)-1,8-naphthyridin-2(1H)-one—Compound 34

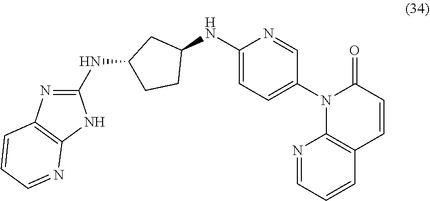 [SMILES: O=c1ccc2cccnc2n1-c1ccc(N[C@H]2CC[C@H](Nc3nc4cccnc4[nH]3)C2)nc1]

TFA (2 mL) was added to a mixture of 1-(6-(((1S,3S)-3-((3-(4-methoxybenzyl)-3H-imidazo[4,5-b]pyridin-2-yl)amino)cyclopentyl)amino)pyridin-3-yl)-1,8-naphthyridin-2(1H)-one and 1-(6-(((1S,3S)-3-((1-(4-methoxybenzyl)-1H-imidazo[4,5-b]pyridin-2-yl)amino)cyclopentyl)amino)pyridin-3-yl)-1,8-naphthyridin-2(1H)-one Intermediate 52 (100 mg, 0.18 mmol) and the reaction mixture was stirred at 60° C. for 5 h under a nitrogen atmosphere. The reaction mixture was filtered, and the filtrate was concentrated. The crude product was purified by preparative HPLC, PrepMethod J (gradient: 6-18%), to give the title compound (19 mg, 24%) as a white solid; MS (ESI) m/z [M+H]⁺ 439.2: ¹H NMR (300 MHz, DMSO-d₆) δ 1.53-1.70 (m, 2H), 1.94-2.14 (m, 2H), 2.15-2.33 (m, 2H), 4.26-4.46 (m, 2H), 6.58 (d, 1H), 6.74 (d, 1H), 6.88 (d, 1H), 7.04 (dd, 1H), 7.23-7.32 (m, 2H), 7.62 (dd, 1H), 7.81 (dd, 1H), 7.96 (dd, 1H), 8.03 (d, 1H), 8.11-8.23 (m, 2H), 8.42 (dd, 1H), 12.79 (br s, 1H).

### Example 35

### 1-(6-(((1S,3S)-3-((3H-Imidazo[4,5-b]pyridin-2-yl)amino)cyclopentyl)amino)pyridin-3-yl)-1,3-dihydro-2H-benzo[d]imidazol-2-one—Compound 35

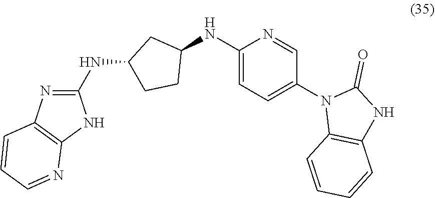 [SMILES: O=c1[nH]c2ccccc2n1-c1ccc(N[C@H]2CC[C@H](Nc3nc4cccnc4[nH]3)C2)nc1]

1-(6-(((1S,3S)-3-((3-(4-Methoxybenzyl)-3H-imidazo[4,5-b]pyridin-2-yl)amino)cyclopentyl)amino)pyridin-3-yl)-1,3-dihydro-2H-benzo[d]imidazol-2-one* Intermediate 57 (75 mg, 0.14 mmol) was suspended in TFA (5 mL) and the reaction mixture was stirred at 80° C. for 4 h. The reaction mixture was filtered through Celite. The filter cake was washed with DCM (2×5 mL) and the combined filtrate was concentrated under reduced pressure. The crude product was purified by preparative HPLC, PrepMethod J (gradient: 12-17%), to give the title compound (25 mg, 41%) as a white solid; HRMS (ESI) m/z [M+H]⁺ calcd for C₂₃H₂₃N₈O: 427.1990, found: 427.1992; ¹H NMR (300 MHz, DMSO-d₆) δ 1.46-1.72 (2H, m), 1.90-2.10 (2H, m), 2.13-2.31 (2H, m), 4.24-4.45 (2H, m), 6.58-6.67 (1H, m), 6.83 (1H, d), 6.88-7.09 (6H, m), 7.42-7.57 (3H, m), 7.91 (1H, dd), 8.05 (1H, d), 8.11-8.17 (1H, m), 11.05 (1H, s). (*regiochemistry was not confirmed)

### Example 36

### 1-(6-(((1S,3S)-3-((3H-Imidazo[4,5-b]pyridin-2-yl)amino)cyclopentyl)amino)pyridin-3-yl)-3-methyl-1,3-dihydro-2H-benzo[d]imidazol-2-one—Compound 36

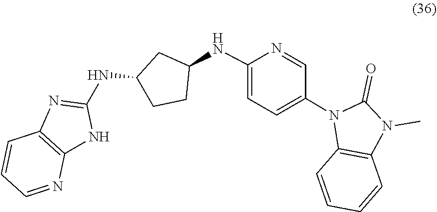 [SMILES: Cn1c(=O)n(-c2ccc(N[C@H]3CC[C@H](Nc4nc5cccnc5[nH]4)C3)nc2)c2ccccc21]

A solution of 1-(6-(((1S,3S)-3-((3-(4-methoxybenzyl)-3H-imidazo[4,5-b]pyridin-2-yl)amino)cyclopentyl)amino)pyridin-3-yl)-3-methyl-1,3-dihydro-2H-benzo[d]imidazol-2-one* Intermediate 58 (30 mg, 0.05 mmol) in TFA (2 mL) was stirred at 80° C. for 16 h. The reaction mixture was filtered through celite. The filter cake was washed with DCM (2×5 mL) and the combined filtrate was concentrated under reduced pressure. The crude product was purified by preparative HPLC, PrepMethod F (gradient: 25-55%), to give the title compound (12 mg, 51%) as a white solid; HRMS (ESI) m/z [M+H]⁺ calcd for C₂₄H₂₅N₈O: 441.2146, found: 441.2170; ¹H NMR (300 MHz, DMSO-d₆) 1.36-1.71 (2H, m), 1.88-2.09 (2H, m), 2.12-2.13 (2H, m), 3.37 (3H, s), 4.22-4.25 (2H, m), 6.60 (1H, d), 6.71-7.27 (7H, m), 7.36 (1H, t), 7.47 (1H, dd), 7.85 (1H, d), 8.04 (1H, d), 10.77 (0.5H, br s), 11.36 (0.4H, br s). Mixture of tautomers approximately 1:1.

### Example 37

### 1-(6-(((1S,3S)-3-((3H-Imidazo[4,5-b]pyridin-2-yl)amino)cyclopentyl)amino)pyridin-3-yl)-3-methylimidazolidine-2,4-dione—Compound 37

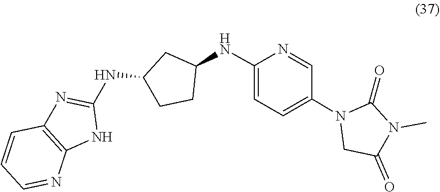 [SMILES: CN1C(=O)CN(c2ccc(N[C@H]3CC[C@H](Nc4nc5cccnc5[nH]4)C3)nc2)C1=O]

1-(6-(((1S,3S)-3-((3-(4-Methoxybenzyl)-3H-imidazo[4,5-b]pyridin-2-yl)amino)cyclopentyl)amino)pyridin-3-yl)-3-methyl imidazolidine-2,4-dione*Intermediate 59 (155 mg, 0.29 mmol) was dissolved in TFA (5 mL) and the reaction mixture was stirred at 80° C. for 4 h. The reaction mixture was filtered through Celite. The filter cake was washed with DCM (2×5 mL) and the combined filtrate was concentrated under reduced pressure. The crude product was purified by preparative HPLC, PrepMethod J (gradient: 8-17%), to give the title compound (74 mg, 62%) as a white solid; HRMS (ESI) m/z [M+H]⁺ calcd for C₂₀H₂₃N₈O₂: 407.1938, found: 407.1940; ¹H NMR (300 MHz, DMSO-d₆) 1.46-1.71 (2H, m), 1.90-2.08 (2H, m), 2.11-2.28 (2H, m), 2.90 (3H, s), 4.25-4.31 (2H, m), 4.37 (2H, s), 6.53 (1H, d), 6.75 (1H, br s), 7.17 (1H, dd), 7.66 (1H, dd), 7.78 (1H, dd), 8.03 (1H, dd), 8.09-8.13 (1H, m), 8.91 (1H, d), 13.21 (1H, br s). (*regiochemistry was not confirmed)

### Example 38

### 3-(6-(((1S,3S)-3-((3H-Imidazo[4,5-b]pyridin-2-yl)amino)cyclopentyl)amino)pyridin-3-yl)-1-methylimidazolidine-2,4-dione—Compound 38

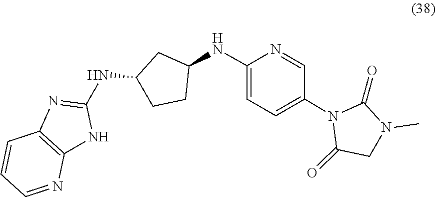 [SMILES: CN1CC(=O)N(c2ccc(N[C@H]3CC[C@H](Nc4nc5cccnc5[nH]4)C3)nc2)C1=O]

A solution of 3-(6-(((1S,3S)-3-((3-(4-methoxybenzyl)-3H-imidazo[4,5-b]pyridin-2-yl)amino)cyclopentyl)amino)pyridin-3-yl)-1-methylimidazolidine-2,4-dione* Intermediate 63 (236 mg, 0.45 mmol) in TFA (5.0 mL) was stirred at 80° C. for 2 h. The reaction mixture was concentrated under reduced pressure and the crude product was purified by preparative HPLC, PrepMethod A (gradient: 8-38%), to give the title compound (26 mg, 14%) as a white solid; HRMS (ESI) m/z [M+H]⁺ calcd for C₂₀H₂₃N₈O₂: 407.1938, found: 407.1914; ¹H NMR (300 MHz, DMSO-d₆) 1.42-1.63 (2H, m), 1.85-2.02 (2H, m), 2.09-2.23 (2H, m), 2.90 (3H, s), 4.06 (2H, s), 4.24-4.34 (2H, m), 6.51 (1H, d), 6.76 (0.5H, dd), 6.85-6.93 (2H, m), 7.16 (0.5H, d), 7.27 (1H, dd), 7.35 (1H, dd), 7.78 (0.5H, d), 7.85 (1H, d), 7.91 (0.5H, d), 10.74 (0.5H, br s), 11.34 (0.5H, br s). Mixture of tautomers approximately 1:1. (*regiochemistry was not confirmed)

### Example 39

### 3-(2-(((1S,3S)-3-((3H-Imidazo[4,5-b]pyridin-2-yl)amino)cyclopentyl)amino)pyrimidin-5-yl)-1-methylpyridin-2(1H)-one—Compound 39

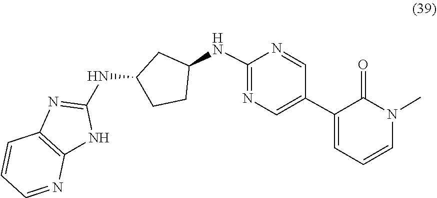 [SMILES: Cn1cccc(-c2cnc(N[C@H]3CC[C@H](Nc4nc5cccnc5[nH]4)C3)nc2)c1=O]

3-(2-(((1S,3S)-3-((3-(4-Methoxybenzyl)-3H-imidazo[4,5-b]pyridin-2-yl)amino)cyclopentyl)amino)pyrimidin-5-yl)-1-methylpyridin-2(1H)-one* Intermediate 65 (69 mg, 0.13 mmol) was added to TFA (5 mL, 64.90 mmol) at 20° C. The resulting mixture was stirred at 80° C. for 3 h. The solvent was removed under reduced pressure. The synthesis procedure was repeated for a second batch with 3-(2-(((1S,3S)-3-((1-(4-methoxybenzyl)-1H-imidazo[4,5-b]pyridin-2-yl)amino)cyclopentyl)amino)pyrimidin-5-yl)-1-methylpyridin-2(1H)-one* Intermediate 65 (40 mg, 0.08 mmol). The two batches were combined and purified by preparative HPLC, PrepMethod A (gradient: 16-31%), to give the title compound (25 mg, 29%) as a white solid; HRMS (ESI) m/z [M+H]+ calcd for C₂₁H₂₃N₈O: 403.1990, found: 403.1986; ¹H NMR (300 MHz, DMSO-d₆) δ 1.48-1.71 (2H, m), 1.89-2.05 (2H, m), 2.1-2.25 (2H, m), 3.50 (3H, s), 4.17-4.59 (2H, m), 6.30 (1H, t), 6.72-6.94 (1H, m), 6.94-7.26 (1H, m), 7.29-7.48 (2H, m), 7.67 (2H, ddd), 7.76-7.98 (1H, m), 8.65 (2H, s), 10.63-11.54 (1H, m). (*regiochemistry was not confirmed)

### Example 40

### 6′-(((1S,3S)-3-((3H-Imidazo[4,5-b]pyridin-2-yl)amino)cyclopentyl)amino)-3-methyl-2H-[1,3′-bipyridin]-2-one—Compound 40

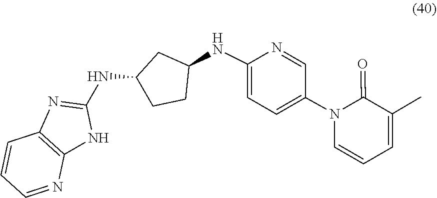 [SMILES: Cc1cccn(-c2ccc(N[C@H]3CC[C@H](Nc4nc5cccnc5[nH]4)C3)nc2)c1=O]

A solution of 6′-(((1S,3S)-3-((3-(4-methoxybenzyl)-3H-imidazo[4,5-b]pyridin-2-yl)amino)cyclopentyl)amino)-3-methyl-2H-[1,3′-bipyridin]-2-one* Intermediate 66 (91 mg, 0.17 mmol) in TFA (5.0 mL) was stirred at 80° C. for 2 h. The solvent was removed under reduced pressure. The crude product was purified by preparative HPLC, PrepMethod A (gradient 15-35%), to give the title compound (18 mg, 25%) as a white solid; HRMS (ESI) m/z [M+H]⁺ calcd for C₂₂H₂₄N₇O: 402.2036, found: 402.2046; ¹H NMR (300 MHz, DMSO-d₆) 1.46-1.66 (2H, m), 1.89-2.06 (5H, m), 2.12-2.25 (2H, m), 4.25-4.39 (2H, m), 6.18 (1H, t), 6.53 (1H, d), 6.90-6.98 (2H, m), 7.34-7.40 (2H, m), 7.44 (1H, dd), 7.49 (1H, dd), 7.69 (1H, br d), 7.89-7.91 (2H, m), 12.09 (1H, br s). (*regiochemistry was not confirmed)

### Example 41

### 6′-(((1S,3S)-3-((3H-Imidazo[4,5-b]pyridin-2-yl)amino)cyclopentyl)amino)-3-methoxy-2H-[1,3′-bipyridin]-2-one—Compound 41

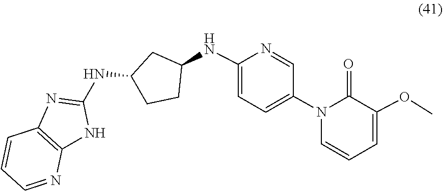 [SMILES: COc1cccn(-c2ccc(N[C@H]3CC[C@H](Nc4nc5cccnc5[nH]4)C3)nc2)c1=O]

A solution of 3-methoxy-6′-(((1S,3S)-3-((3-(4-methoxybenzyl)-3H-imidazo[4,5-b]pyridin-2-yl)amino)cyclopentyl)amino)-2H-[1,3′-bipyridin]-2-one* Intermediate 67 (84 mg, 0.16 mmol) in TFA (5.0 mL) was stirred at 80° C. for 2 h. The solvent was removed under reduced pressure and the crude product was purified by preparative HPLC, PrepMethod K (gradient: 2-20%), to give the title compound (8 mg, 13%) as a brown solid; HRMS (ESI) m/z [M+H]⁺ calcd for C₂₂H₂₄NO₂: 418.1986, found: 418.1986; ¹H NMR (300 MHz, DMSO-d₆) 1.52-1.74 (2H, m), 1.96-2.12 (2H, m), 2.15-2.31 (2H, m), 3.71 (3H, s), 4.27-4.39 (2H, m), 6.21 (1H, t), 6.63 (1H, d), 6.87 (1H, dd), 7.11-7.41 (3H, m), 7.49 (1H, dd), 7.81 (1H, dd), 7.97 (1H, d), 8.04 (1H, d), 9.03 (1H, br s), 12.44 (1H, br s). (*regiochemistry was not confirmed)

### Example 42

### 6′-(((1S,3S)-3-((3H-Imidazo[4,5-b]pyridin-2-yl)amino)cyclopentyl)amino)-3-chloro-2H-[1,3′-bipyridin]-2-one—Compound 42

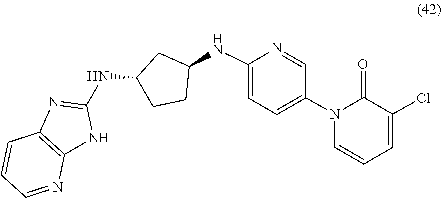 [SMILES: O=c1c(Cl)cccn1-c1ccc(N[C@H]2CC[C@H](Nc3nc4cccnc4[nH]3)C2)nc1]

A mixture of 3-chloro-6′-(((1S,3S)-3-((3-(4-methoxybenzyl)-3H-imidazo[4,5-b]pyridin-2-yl)amino)cyclopentyl)amino)-2H-[1,3′-bipyridin]-2-one* Intermediate 68 (90 mg, 0.17 mmol) in TFA (3 mL) was stirred at 80° C. for 3 h. The reaction mixture was filtered through Celite and the filter cake was washed with DCM (2×5 mL). The filtrate was concentrated under reduced pressure. The crude product was purified by preparative HPLC, PrepMethod H (gradient: 11-41%), to give the title compound (45 mg, 64%) as a white solid; HRMS (ESI) m/z [M+H]⁺ calcd for C₂₁H₂₁ClN₇O: 422.1490, found: 422.1508; ¹H NMR (300 MHz, DMSO-d₆) δ 1.46-1.64 (2H, m), 1.87-2.03 (2H, m), 2.11-2.22 (2H, m), 4.25-4.35 (2H, m), 6.29 (1H, t), 6.54 (1H, d), 6.74-6.78 (0.5H, m), 6.87-7.03 (2H, m), 7.17 (0.5H, d), 7.36 (1H, dd), 7.43 (1H, dd), 7.65 (1H, d), 7.77-7.81 (1.5H, m), 7.91 (0.5H, d), 7.96 (1H, d), 10.73 (0.5H, br s), 11.34 (0.5H, br s). Mixture of tautomers approximately 1:1. (*regiochemistry was not confirmed)

### Example 43

### 6′-(((1S,3S)-3-((3H-Imidazo[4,5-b]pyridin-2-yl)amino)cyclopentyl)amino)-2-oxo-2H-[1,3′-bipyridine]-5-carbonitrile—Compound 43

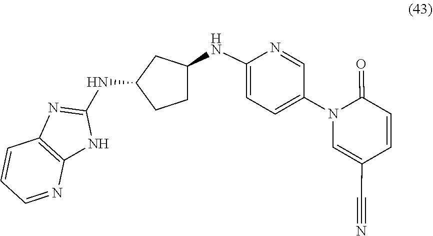 [SMILES: N#Cc1ccc(=O)n(-c2ccc(N[C@H]3CC[C@H](Nc4nc5cccnc5[nH]4)C3)nc2)c1]

A mixture of 6′-(((1S,3S)-3-((3-(4-methoxybenzyl)-3H-imidazo[4,5-b]pyridin-2-yl)amino)cyclopentyl)amino)-2-oxo-2H-[1,3′-bipyridine]-5-carbonitrile* Intermediate 69 (55 mg, 0.10 mmol) in TFA (4.0 mL) was stirred at 80° C. for 2 h. The solvent was removed under reduced pressure. The crude product was purified by preparative HPLC, PrepMethod F (gradient: 7-32%), to give the title compound (5 mg, 12%) as a white solid: HRMS (ESI) m/z [M+H]⁺ calcd for C₂₂H₂₁N₈O: 413.1832, found: 413.1826; ¹H NMR (300 MHz, DMSO-d₆) δ 1.45-1.65 (2H, m), 1.85-2.00 (2H, m), 2.10-2.30 (2H, m), 4.25-4.51 (2H, m), 6.49-6.62 (2H, m), 6.76-7.18 (3H, m), 7.28-7.42 (1H, m), 7.45-7.55 (1H, m), 7.65-7.83 (1H, m), 7.86-7.96 (1H, m), 7.99 (1H, d), 8.57 (1H, s), 10.74 (0.5H, s), 11.35 (0.5H, s). Mixture of tautomers approximately 1:1. (*regiochemistry was not confirmed)

### Example 44

### 6′-(((1S,3S)-3-((3H-Imidazo[4,5-b]pyridin-2-yl)amino)cyclopentyl)amino)-2-oxo-2H-[1,3′-bipyridine]-5-carboxylic acid—Compound 44

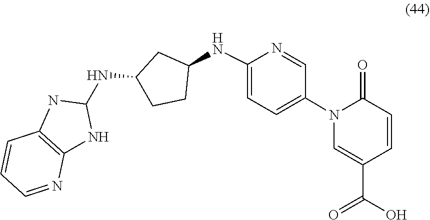 [SMILES: O=C(O)c1ccc(=O)n(-c2ccc(N[C@H]3CC[C@H](NC4Nc5cccnc5N4)C3)nc2)c1]

NaOH (54 mg, 1.36 mmol) was added to a solution of methyl 6′-(((1S,3S)-3-((3H-imidazo[4,5-b]pyridin-2-yl)amino)cyclopentyl)amino)-2-oxo-2H-[1,3′-bipyridine]-5-carboxylate×2.5 TFA Intermediate 71 (99 mg, 0.14 mmol) in MeOH (6 mL) and water (2 mL) and the reaction mixture was stirred at 20° C. for 16 h under a nitrogen atmosphere. The solvent was removed under reduced pressure. The residue was diluted with water (10 mL) and pH was adjusted to 3 with 1 M HCl (aq). The reaction mixture was poured into water (50 mL), extracted with EtOAc (2×100 mL), and the combined organic layer was dried over Na₂SO₄, filtered, and concentrated at reduced pressure. The crude product was purified by preparative HPLC, PrepMethod J (gradient: 6-21%), to give the title compound (16 mg, 27%) as a white solid; HRMS (ESI) m/z [M+H]⁺ calcd for C₂₂H₂₂N₇O₃: 432.1778, found: 432.1778; ¹H NMR (300 MHz, DMSO-d₆) δ ppm 1.42-1.71 (m, 2H) 1.84-2.08 (m, 2H) 2.09-2.36 (m, 2H) 4.18-4.51 (m, 2H) 6.35-6.67 (m, 2H) 6.75-6.93 (m, 1H) 6.94-7.05 (m, 1H) 7.07-7.23 (m, 1H) 7.33-7.41 (m, 1H) 7.45 (dd, 1H) 7.74-7.94 (m, 2H) 7.96-8.04 (m, 1H) 8.09-8.25 (m, 1H).

### Example 45

### 3-(6-(((1S,3S)-3-((3H-Imidazo[4,5-b]pyridin-2-yl)amino)cyclopentyl)amino)pyridin-3-yl)-1-methyl-1,3-dihydro-2H-imidazo[4,5-b]pyridin-2-one—Compound 45

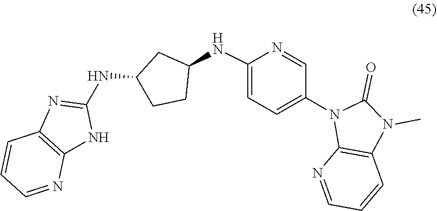 [SMILES: Cn1c(=O)n(-c2ccc(N[C@H]3CC[C@H](Nc4nc5cccnc5[nH]4)C3)nc2)c2ncccc21]

A solution of 3-(6-(((1S,3S)-3-((3-(4-methoxybenzyl)-3H-imidazo[4,5-b]pyridin-2-yl)amino)cyclopentyl)amino)pyridin-3-yl)-1-methyl-1,3-dihydro-2H-imidazo[4,5-b]pyridin-2-one* Intermediate 72 (50 mg, 0.09 mmol) in TFA (1 mL) was stirred at 80° C. for 18 h. The reaction mixture was filtered through Celite and the filter cake was washed with DCM (2×5 mL). The combined filtrate was concentrated under reduced The crude product was purified by preparative HPLC, PrepMethod F (gradient: 15-35%), to give the title compound (10 mg, 25%) as a white solid; HRMS (ESI) m/z [M+H]⁺ calcd for C₂₃H₂₄N₉O: 442.2098, found: 442.2092; ¹H NMR (300 MHz, DMSO-d₆) δ ppm 1.46-1.71 (m, 2H) 1.87-2.10 (m, 2H), 2.12-2.35 (m, 2H) 3.42 (s, 3H) 4.14-4.53 (m, 2H) 6.57-6.65 (m, 1H) 6.81-6.91 (m, 1H) 6.91-6.98 (m, 1H) 7.09-7.19 (m, 1H) 7.40 (dd, 1H) 7.48-7.62 (m, 2H) 7.84-7.91 (m, 1H) 7.94 (dd, 1H) 8.11 (d, 1H), 11.32 (br s, 1H). (*regiochemistry was not confirmed)

### Example 46

### 6′-(((1S,3S)-3-((3H-Imidazo[4,5-b]pyridin-2-yl)amino)cyclopentyl)amino)-2H-[1,3′-bipyridin]-2-one—Compound 46

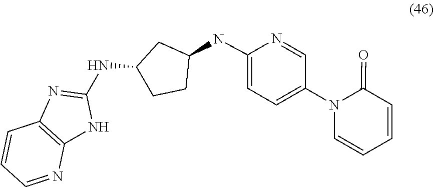

A mixture of 6′-(((1S,3S)-3-((3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((1-(4-methoxybenzyl)-1H-imidazo[4,5-b]pyridin-2-yl)amino)cyclopentyl)amino)-2H-[1,3′-bipyridin]-2-one Intermediate 73 (100 mg, 0.20 mmol) was treated with TFA (3 mL) at 20° C. and the resulting solution was stirred at 80° C. for 18 h under a nitrogen atmosphere. The reaction mixture was cooled to rt, filtered through Celite and the filter cake was washed with DCM (2×5 mL). The combined filtrates were concentrated under reduced pressure and the obtained residue was purified by reversed phase flash chromatography on a C18 column (gradient 0-29% MeCN in water (1% TFA)) followed by preparative HPLC, PrepMethod A gradient: 12-42%), to give the title compound (10 mg, 13%) as a white solid. HRMS (ESI) m/z [M+H]⁺ calcd for C₂₁H₂₂N₇O: 388.1880, found: 388.1896. ¹H NMR (300 MHz, Methanol-d₄) 1.55-1.74 (2H, m), 2.04-2.13 (2H, m), 2.22-2.42 (2H, m), 4.38 (2H, h), 6.46 (1H, td), 6.57-6.67 (2H, m), 6.96 (1H, dd), 7.39-7.54 (2H, m), 7.54-7.68 (2H, m), 7.82-8.00 (2H, m).

### Example 47

### 6′-(((1S,3S)-3-((6-Chloro-3H-imidazo[4,5-b]pyridin-2-yl)amino)cyclopentyl)amino)-2H-[1,3′-bipyridin]-2-one—Compound 47

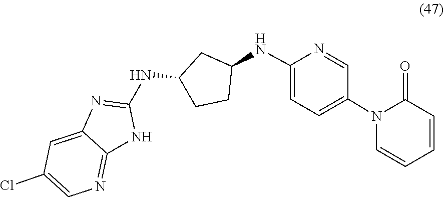 [SMILES: O=c1ccccn1-c1ccc(N[C@H]2CC[C@H](Nc3nc4cc(Cl)cnc4[nH]3)C2)nc1]

2,6-Dichloro-3H-imidazo[4,5-b]pyridine (23.9 mg, 0.13 mmol) and 6′-(((1S,3S)-3-aminocyclopentyl)amino)-2H-[1,3′-bipyridin]-2-one Intermediate 3 (31 mg, 0.12 mmol) were charged in a closed vial, followed by addition of NMP (0.4 mL) and TEA (32 μL, 0.23 mmol). The mixture was heated at 110° C. under air atmosphere for 24 h. Another amount of TEA (32 μL, 0.23 mmol) was added and the reaction mixture was stirred at 110° C. for another 20 h. The volatiles were removed under reduced pressure and the residue was purified by preparative HPLC (PrepMethod B, gradient: 15-55%) to give the title compound (18 mg, 37%) as an off-white solid; HRMS (ESI) m/z [M+H]⁺ calcd for C₂₁H₂₁ClN₇O: 422.1490, found: 422.1502; ¹H NMR (500 MHz, DMSO-d₆) δ 1.46-1.64 (2H, m), 1.95 (2H, dt), 2.17 (2H, s), 4.31 (2H, dq), 6.27 (1H, td), 6.44 (1H, dt), 6.53 (1H, d), 6.96 (1H, dd), 7.25-7.49 (4H, m), 7.60 (1H, d), 7.77-7.95 (2H, m), 11.27 (1H, d).

### Example 48-Example 50

The following examples were synthesized and purified in analogy with the description for Example 47 starting from 6′-(((1S,3S)-3-aminocyclopentyl)amino)-2H-[1,3′-bipyridin]-2-one Intermediate 3 and commercially available starting material if not otherwise stated. The reaction time was 20 h.

### Example 48-50

| | | Inter- | | |
|---|---|---|---|---|
| Ex | | me- | | |
| No | Structure/Name | diate | Yield | Analytical Data |
| 48 | 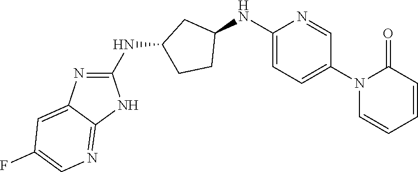 [SMILES: O=c1ccccn1-c1ccc(N[C@H]2CC[C@H](Nc3nc4cc(F)cnc4[nH]3)C2)nc1] | 74 | 20 mg, 33% | HRMS (ESI) m/z [M + H]⁺ calcd for C₂₁H₂₁FN₇O: 406.1786, found: 406.1782. ¹H NMR (500 MHz, DMSO-d₆) δ 1.45- 1.64 (2 H, m), 1.85-2.01 (4 H, m), 4.31 (2 H, dq), 6.27 (1 H, td), 6.44 (1 H, dt), 6.53 (1 H, dd), 6.95 (1 H, d), 7.29 (2 H, dd), 7.40 (1 H, dd), 7.47 (1 H, ddd), 7.60 (1 H, ddd), 7.82 (1 H, d), 7.91-7.95 (1 H, m), 11.25 (1 H, d). |
| | 6′-(((1S,3S)-3-((6-Fluoro-3H- | | | |
| | imidazo[4,5-b]pyridin-2- | | | |
| | yl)amino)cyclopentyl)amino)- | | | |
| | 2H-[1,3′-bipyridin]-2-one- | | | |
| | compound 48 | | | |
| 49 | 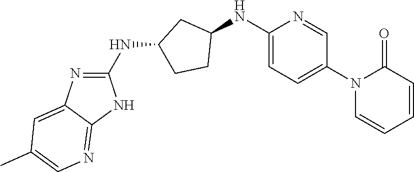 [SMILES: Cc1cnc2[nH]c(N[C@H]3CC[C@H](Nc4ccc(-n5ccccc5=O)cn4)C3)nc2c1] | | 8.9 mg, 17% | HRMS (ESI) m/z [M + H]⁺ calcd for C₂₂H₂₄N₇O: 402.2036, found: 402.2036. ¹H NMR (500 MHz, DMSO-d₆) δ 1.45- 1.62 (2 H, m), 1.87-2.01 (2 H, m), 2.12- 2.21 (2 H, m), 2.27 (3 H, s), 4.27 (1 H, q), 4.33 (1 H, q), 6.26 (1 H, td), 6.44 (1 H, dt), 6.53 (1 H, d), 6.95 (1 H, d), 7.01- 7.32 (2 H, m), 7.40 (1 H, dd), 7.47 (1 H, ddd), 7.61 (2 H, dd), 7.92 (1 H, d), 10.52-11.44 (1 H, m). |
| | 6′-(((1S,3S)-3-((6-Methyl-3H- | | | |
| | imidazo[4,5-b]pyridin-2- | | | |
| | yl)amino)cyclopentyl)amino)- | | | |
| | 2H-[1,3′-bipyridin]-2-one- | | | |
| | compound 49 | | | |
| 50 | 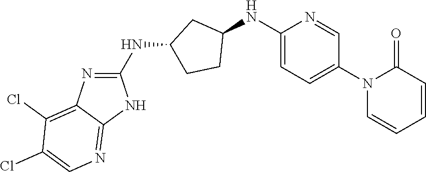 [SMILES: O=c1ccccn1-c1ccc(N[C@H]2CC[C@H](Nc3nc4c(Cl)c(Cl)cnc4[nH]3)C2)nc1] | | 11 mg, 28% | HRMS (ESI) m/z [M + H]⁺ calcd for C₂₁H₂₀Cl₂N₇O: 456.1100, found: 456.1116. ¹H NMR (500 MHz, DMSO-d₆) δ 1.45- 1.65 (2 H, m), 1.87-2.03 (2 H, m), 2.13- 2.24 (2 H, m), 4.32 (2 H, dq), 6.27 (1 H, td), 6.44 (1 H, dt), 6.53 (1 H, d), 6.68 (1 H, s), 6.96 (1 H, d), 7.28 (1 H, s), 7.40 (1 H, dd), 7.47 (1 H, ddd), 7.60 (1 H, dd), 7.93 (2 H, d). |
| | 6′-(((1S,3S)-3-((6,7-Dichloro- | | | |
| | 3H-imidazo[4,5-b]pyridin-2- | | | |
| | yl)amino)cyclopentyl)amino)- | | | |
| | 2H-[1,3′-bipyridin]-2-one- | | | |
| | compound 50 | | | |

### Example 51

### 6′-(((1S,3S)-3-((6-(Trifluoromethoxy)-3H-imidazo[4,5-b]pyridin-2-yl)amino)cyclopentyl)amino)-2H-[1,3′-bipyridin]-2-one—Compound 51

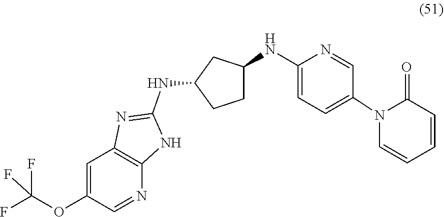 [SMILES: O=c1ccccn1-c1ccc(N[C@H]2CC[C@H](Nc3nc4cc(OC(F)(F)F)cnc4[nH]3)C2)nc1]

1,1′-Thiocarbonyldiimidazole (55 mg, 0.31 mmol) was added to a solution of 6′-(((1S,3S)-3-aminocyclopentyl)amino)-2H-[1,3′-bipyridin]-2-one×HCl Intermediate 3 (42 mg, 0.16 mmol) in DMF (1 mL) and the reaction mixture was stirred at 100° C. for 1 h. The reaction mixture was cooled to rt and a solution of 5-(trifluoromethoxy)pyridine-2,3-diamine (32 mg, 0.16 mmol) in DMF (0.5 mL), TEA (0.043 mL, 0.31 mmol) and EDC (60 mg, 0.31 mmol) were added. The reaction mixture was stirred at 100° C. for 17 h, cooled to rt and poured into water. The aqueous layer was extracted with EtOAc (×2) and the combined organic layers were washed with brine, dried over Na₂SO₄, filtered and concentrated under reduced pressure. The residue was purified by preparative HPLC (PrepMethod B, gradient: 10-50%), and fractions containing the desired product were combined and concentrated under reduced pressure. The obtained aqueous layer was extracted with EtOAc (×2), the combined organic layers were washed with brine, dried over Na₂SO₄, filtered and concentrated under reduced pressure to afford the title compound (14 mg, 19%) as a yellow solid; HRMS (ESI) m/z [M+H]⁺ calcd for C₂₂H₂₁F₃N₇O₂: 472.1704, found: 472.1692; ¹H NMR (500 MHz, DMSO-d₆) δ 1.43-1.66 (2H, m), 1.85-2.06 (2H, m), 2.1-2.27 (2H, m), 4.22-4.43 (2H, m), 6.23-6.3 (1H, m), 6.44 (1H, d), 6.53 (1H, dd), 6.95 (1H, dd), 7.29-7.51 (4H, m), 7.56-7.63 (1H, m), 7.79-7.98 (2H, m), 10.72-11.89 (1H, m).

### Example 52

### 6′-(((1S,3S)-3-((6-Bromo-3H-imidazo[4,5-b]pyridin-2-yl)amino)cyclopentyl)amino)-5-(2-methylpyrimidin-5-yl)-2H-[1,3′-bipyridin]-2-one—Compound 52

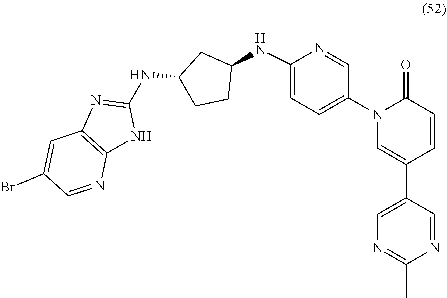 [SMILES: Cc1ncc(-c2ccc(=O)n(-c3ccc(N[C@H]4CC[C@H](Nc5nc6cc(Br)cnc6[nH]5)C4)nc3)c2)cn1]

A mixture of 6′-(((1S,3S)-3-((6-bromo-3-(4-methoxybenzyl)-3H-imidazo[4,5-b]pyridin-2-yl)amino)cyclopentyl)amino)-5-(2-methylpyrimidin-5-yl)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-5-(2-methylpyrimidin-5-yl)-2H-[1,3′-bipyridin]-2-one Intermediate 81 (95 mg, 0.14 mmol) in TFA (2 mL) was stirred in a sealed reactor at 100° C. for 1 h. The reaction mixture was concentrated at reduced pressure and the residue was purified by preparative HPLC, PrepMethod B (gradient: 10-50%). The compound containing fractions were combined and the organic solvent was removed at reduced pressure. The aqueous layer was extracted twice with a mixture of EtOAc:MeOH (9:1). The combined organic layer was washed with brine, dried over Na₂SO₄, filtered, and concentrated to give the title compound (31 mg, 39%); HRMS (ESI) m/z [M+H]⁺ calcd for C₂₆H₂₅BrN₉O: 558.1360, found: 558.1362; ¹H NMR (500 MHz, DMSO-d₆) δ 1.46-1.69 (2H, m), 1.87-2.07 (2H, m), 2.18 (2H, s), 2.62 (3H, s), 4.24-4.42 (2H, m), 6.56 (1H, d), 6.60 (1H, d), 6.99 (1H, s), 7.18-7.45 (1H, m), 7.46-7.6 (2H, m), 7.8-8.01 (2H, m), 8.04 (1H, d), 8.19 (1H, d), 8.97 (2H, s), 10.87 (0.5H, s), 11.61 (0.5H, s). Mixture of tautomers approximately 1:1.

### Example 53

### 6′-(((1S,3S)-3-((3H-Imidazo[4,5-b]pyridin-2-yl)amino)cyclopentyl)amino)-5-(2-methylpyrimidin-5-yl)-2H-[1,3′-bipyridin]-2-one—Compound 53

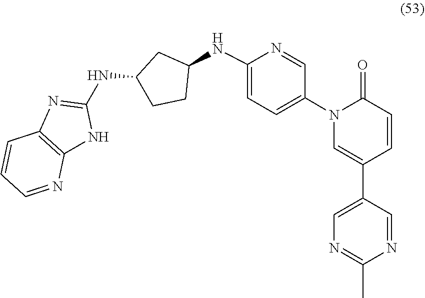 [SMILES: Cc1ncc(-c2ccc(=O)n(-c3ccc(N[C@H]4CC[C@H](Nc5nc6cccnc6[nH]5)C4)nc3)c2)cn1]

10% Pd/C (4.4 mg, 4.12 μmol) was added to a solution of 6′-(((1S,3S)-3-((6-bromo-3H-imidazo[4,5-b]pyridin-2-yl)amino)cyclopentyl)amino)-5-(2-methylpyrimidin-5-yl)-2H-[1,3′-bipyridin]-2-one Example 52 (23 mg, 0.04 mmol) in MeOH (0.41 mL) and the reaction mixture was hydrogenated (2 bar) at rt for 6 days. The reaction mixture was filtered through a plug of Celite and the filtrate was concentrated at reduced pressure. The residue was purified by preparative HPLC, PrepMethod B (gradient: 5-40%), to give the title compound (4 mg, 20%) as a white solid; HRMS (ESI) m/z [M+H]⁺ calcd for C₂₆H₂₆N₉O: 480.2254, found: 480.2262; ¹H NMR (500 MHz, DMSO-d₆) δ 1.48-1.64 (2H, m), 1.88-2.03 (2H, m), 2.14-2.25 (2H, m), 2.62 (3H, s), 4.23-4.43 (2H, m), 6.56 (1H, dd), 6.60 (1H, d), 6.74-6.93 (1H, m), 6.97 (1H, dd), 7-7.22 (1H, m), 7.32-7.41 (1H, m), 7.49 (1H, dd), 7.75-7.94 (1H, m), 7.98 (1H, dd), 8.04 (1H, d), 8.20 (1H, dd), 8.97 (2H, s), 10.74 (0.5H, s), 11.36 (0.5H, s). Mixture of tautomers approximately 1:1.

### Example 54

### 6′-(((1S,3S)-3-((6-Bromo-3H-imidazo[4,5-b]pyridin-2-yl)amino)cyclopentyl)amino)-2H-[1,3′-bipyridin]-2-one—Compound 54

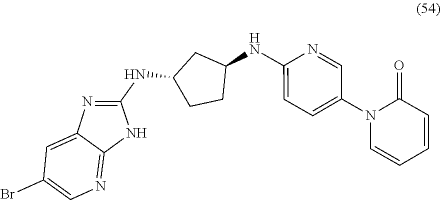 [SMILES: O=c1ccccn1-c1ccc(N[C@H]2CC[C@H](Nc3nc4cc(Br)cnc4[nH]3)C2)nc1]

In a sealed reactor, a mixture of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (16 mg, 0.03 mmol) was dissolved in TFA (500 μL) and the reaction mixture was stirred at 100° C. for 1 h. Excess TFA was removed under reduced pressure and the residue was purified by preparative HPLC (PrepMethod B, gradient: 10-50%) to afford the title compound (5.1 mg, 40%) as a white solid; HRMS (ESI) m/z [M+H]⁺ calcd for C₂₁H₂₁BrN₇O: 466.0986, found: 466.1000; ¹H NMR (500 MHz, DMSO-d₆, 25° C.) δ 1.43-1.65 (2H, m), 1.83-2.06 (2H, m), 2.17 (2H, d), 4.21-4.42 (2H, m), 6.23-6.3 (1H, m), 6.44 (1H, d), 6.53 (1H, dd), 6.96 (1H, dd), 7.2-7.67 (5H, m), 7.81-8.02 (2H, m), 10.63-11.86 (1H, m).

### Example 55

### 6′-(((1S,3S)-3-((6-Cyclopropyl-3H-imidazo[4,5-b]pyridin-2-yl)amino)cyclopentyl)amino)-2H-[1,3′-bipyridin]-2-one—Compound 55

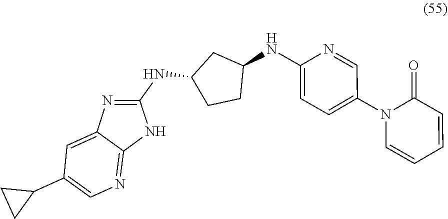 [SMILES: O=c1ccccn1-c1ccc(N[C@H]2CC[C@H](Nc3nc4cc(C5CC5)cnc4[nH]3)C2)nc1]

A mixture of cyclopropylboronic acid (22 mg, 0.26 mmol), diacetoxypalladium (II) (1.2 mg, 5.2 μmol), CataCXium A (3.7 mg, 10 μmol) and CsF (39 mg, 0.26 mmol) in a sealed vial was evacuated and backfilled with Ar(g) (×3) and a solution of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (76 mg, 0.13 mmol) in DME (1 mL) was added at rt. The reaction mixture was stirred at 120° C. for 19 h, cooled to rt and diluted with EtOAc. The organic layer was washed with 10% NaHCO₃ (aq) and brine, dried over Na₂SO₄, filtered, and concentrated under reduced pressure. The residue was dissolved in TFA (1 mL) in a sealed reactor and stirred at 100° C. for 30 min. Excess TFA was removed under reduced pressure and the obtained residue purified by preparative HPLC, PrepMethod B (gradient: 5-40%), to give the title compound (7.4 mg, 13% over two steps) as a white solid; HRMS (ESI) m/z [M+H]⁺ calcd for C₂₄H₂₆N₇O: 428.2194, found: 428.2214; ¹H NMR (500 MHz, DMSO-d₆) δ 0.62 (2H, dd), 0.89 (2H, s), 1.41-1.65 (2H, m), 1.84-2.04 (3H, m), 2.16 (2H, d), 4.17-4.42 (2H, m), 6.26 (1H, t), 6.44 (1H, d), 6.53 (1H, d), 6.85-7.11 (3H, m), 7.40 (1H, dd), 7.47 (1H, ddd), 7.60 (1H, s), 7.65-7.83 (1H, m), 7.92 (1H, d), 10.5-11.31 (1H, m).

### Example 56

### 6′-(((1S,3S)-3-((6-Ethyl-3H-imidazo[4,5-b]pyridin-2-yl)amino)cyclopentyl)amino)-2H-[1,3′-bipyridin]-2-one—Compound 56

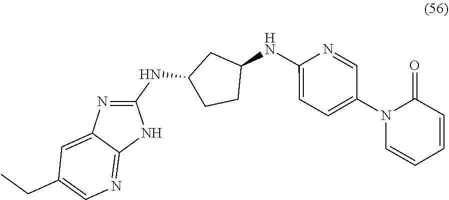 [SMILES: CCc1cnc2[nH]c(N[C@H]3CC[C@H](Nc4ccc(-n5ccccc5=O)cn4)C3)nc2c1]

A mixture of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (45 mg, 0.08 mmol), K₂CO₃ (33 mg, 0.15 mmol), Pd(OAc)₂ (1.2 mg, 5.3 μmol) and CataCXium A (3.9 mg, 11 μmol) in a sealed vial, were suspended in toluene (325 μL) and water (32.5 μL). The reaction mixture was evacuated and backfilled with Ar(g) (×3) and then 1 M triethylborane in hexane (31 μL, 0.03 mmol) was added. The reaction mixture was stirred at 120° C. for 1 h, cooled to rt and diluted with EtOAc. The organic layer was washed with water and brine, dried over Na₂SO₄, filtered, and concentrated under reduced pressure. The obtained residue was dissolved in TFA (1 mL) in a sealed vial and the reaction mixture was stirred at 100° C. for 15 min. Excess TFA was removed under reduced pressure and the residue was purified by preparative HPLC, PrepMethod B (gradient: 10-50%). Fractions containing the desired product were combined and concentrated under reduced pressure. The obtained aqueous layer was extracted with EtOAc (×2), the combined organic layers were washed with brine, dried over Na₂SO₄, filtered and concentrated under reduced pressure to afford the title compound (14.7 mg, 46% over two steps) as a beige solid; HRMS (ESI) m/z [M+H]⁺ calcd for C₂₃H₂₆N₇O: 416.2194, found: 416.2192; H NMR (500 MHz, DMSO-d₆) δ 1.18 (3H, td), 1.43-1.66 (2H, m), 1.85-2.06 (2H, m), 2.09-2.27 (2H, m), 2.59 (2H, q), 4.19-4.4 (2H, m), 6.23-6.3 (1H, m), 6.44 (1H, d), 6.53 (1H, dd), 6.81-7.07 (2H, m), 7.16-7.29 (1H, m), 7.40 (1H, dd), 7.47 (1H, ddd), 7.58-7.62 (1H, m), 7.64-7.83 (1H, m), 7.92 (1H, d), 10.51-11.34 (1H, m).

### Example 57

### 6′-(((1S,3S)-3-((6-Methoxy-3H-imidazo[4,5-b]pyridin-2-yl)amino)cyclopentyl)amino)-2H-[1,3′-bipyridin]-2-one—Compound 57

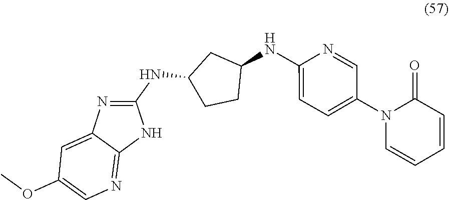 [SMILES: COc1cnc2[nH]c(N[C@H]3CC[C@H](Nc4ccc(-n5ccccc5=O)cn4)C3)nc2c1]

A mixture of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (47 mg, 0.08 mmol), tBuBrettPhos (2.4 mg, 5.0 μmol) and NaOtBu (11 mg, 0.11 mmol) in a sealed vial was evacuated and backfilled with Ar(g) (×3) and then MeOH (32 μL, 0.80 mmol) was added to the reaction mixture. In another sealed vial, Pd₂(dba)₃ (0.7 mg, 0.8 μmol) was dissolved in 1,4-dioxane (150 μL) under an argon atmosphere and the solution was transferred into the first vial. The reaction mixture was stirred at 80° C. for 20 h, cooled to rt and diluted with EtOAc. The organic layer was washed with 10% NaHCO₃ (aq) and brine, dried over Na₂SO₄, filtered, and concentrated under reduced pressure. The obtained residue was dissolved in DCM (1 mL) and TFA (1 mL) and the reaction mixture was stirred at rt for 2.5 h and then at 50° C. for 30 min. The solvent was removed under reduced pressure and the residue was purified by preparative HPLC, PrepMethod B (gradient: 10-50%), to give the title compound (6.2 mg, 19% over two steps) as a beige solid. HRMS (ESI) m/z [M+H]⁺ calcd for C₂₂H₂₄N₇O₂: 418.1986, found: 418.1982. ¹H NMR (500 MHz, DMSO-d₆) δ 1.4-1.67 (2H, m), 1.87-2.04 (2H, m), 2.11-2.26 (2H, m), 3.76 (3H, s), 4.18-4.4 (2H, m), 6.26 (1H, td), 6.44 (1H, dt), 6.53 (1H, d), 6.94 (3H, d), 7.40 (1H, dd), 7.47 (1H, ddd), 7.51-7.7 (2H, m), 7.92 (1H, d), 10.53-11.31 (1H, m).

### Example 58

### 6′-(((1S,3S)-3-((6-(Dimethylphosphoryl)-3H-imidazo[4,5-b]pyridin-2-yl)amino)cyclopentyl)amino)-2H-[1,3′-bipyridin]-2-one—Compound 58

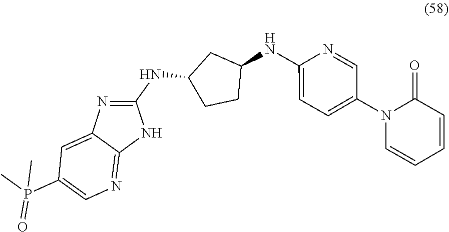 [SMILES: CP(C)(=O)c1cnc2[nH]c(N[C@H]3CC[C@H](Nc4ccc(-n5ccccc5=O)cn4)C3)nc2c1]

A mixture of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (30 mg, 0.05 mmol) and K₃PO₄ (12 mg, 0.06 mmol) in a sealed vial was evacuated and backfilled with Ar(g) (×3) and then dimethylphosphine oxide (40 mg, 0.51 mmol) was added to the vial. In another sealed reactor, Pd₂(dba)₃ (1.4 mg, 1.5 μmol) and XantPhos (1.8 mg, 3.1 μmol) were mixed under an argon atmosphere and dissolved in dioxane (112 μL). The mixture was stirred at rt for 10 min and then added to the first vial. The reaction mixture was stirred at 80° C. for 40 h, cooled to rt and diluted with EtOAc. The organic layer was washed with 10% NaHCO₃ (aq) and brine, dried over Na₂SO₄, filtered, and concentrated under reduced pressure. The obtained residue was dissolved in TFA (1 mL) and the reaction mixture was stirred at 100° C. for 15 min. Excess TFA was removed under reduced pressure and the residue was purified by preparative HPLC, PrepMethod B (gradient: 0-30%) to give the title compound (1 mg, 4% over two steps) as a beige solid; HRMS (ESI) m/z [M+H]⁺ calcd for C₂₃H₂₇N₇O₂P: 464.1958, found: 464.1970; ¹H NMR (500 MHz, DMSO-d₆) δ 1.45-2.27 (12H, m), 4.24-4.45 (2H, m), 6.27 (1H, td), 6.44 (1H, d), 6.51-6.56 (1H, m), 6.98 (1H, d), 7.41 (1H, dd), 7.47 (1H, ddd), 7.61 (1H, d), 7.93 (1H, d), 8.26 (1H, s), 8.22-8.25 (1H, m), 10.96-11.76 (1H, m).

### Example 59

### 6′-(((1S,3S)-3-((6-(Methylthio)-3H-imidazo[4,5-b]pyridin-2-yl)amino)cyclopentyl)amino)-2H-[1,3′-bipyridin]-2-one—Compound 59

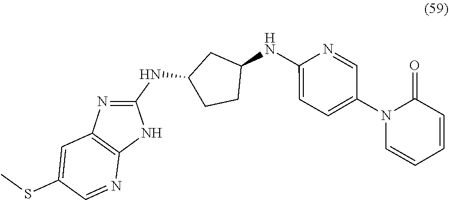 [SMILES: CSc1cnc2[nH]c(N[C@H]3CC[C@H](Nc4ccc(-n5ccccc5=O)cn4)C3)nc2c1]

A mixture of 6′-(((1S,3S)-3-((6-bromo-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-bromo-1-(4-methoxybenzyl)-1H-imidazo[4,5-b]pyridin-2-yl)amino)cyclopentyl)amino)-2H-[1,3′-bipyridin]-2-one Intermediate 82 (43 mg, 0.07 mmol), Cu(I)I (17 mg, 0.09 mmol) and 1,4-diazabicyclo[2.2.2]octane (16.5 mg, 0.15 mmol) in a sealed vial was evacuated and backfilled with Ar(g) (×3) and then DMSO (350 μL) was added. The reaction mixture was stirred at 130° C. for 70 h, cooled to rt and diluted with EtOAc. The organic layer was washed with 10% NaHCO₃ (aq) and brine, dried over Na₂SO₄, filtered, concentrated under reduced pressure to give a crude residue. The aqueous layer was filtered, and the solids were washed with water and combined with the crude residue of the organic layer. The crude product was dissolved in TFA (1 mL) in a sealed reactor and the reaction mixture was stirred at 100° C. for 1 h. Excess TFA was removed under reduced pressure and the residue was purified by preparative HPLC, PrepMethod B (gradient: 10-50%), to give the title compound (4.3 mg, 14% over two steps) as a white solid; HRMS (ESI) m/z [M+H]⁺ calcd for C₂₂H₂₄N₇OS: 434.1758, found: 434.1758; ¹H NMR (500 MHz, DMSO-d₆) δ 1.43-1.68 (2H, m), 1.84-2.07 (2H, m), 2.1-2.27 (2H, m), 2.4-2.46 (3H, m), 4.24-4.4 (2H, m), 6.27 (1H, td), 6.44 (1H, dd), 6.53 (1H, d), 6.92-6.99 (1H, m), 7.06-7.35 (1H, m), 7.35-7.53 (3H, m), 7.61 (1H, d), 7.73-7.98 (2H, m), 10.64-11.61 (1H, m).

### Example 60

### 6′-(((1S,3S)-3-((6-(Difluoromethoxy)-3H-imidazo[4,5-b]pyridin-2-yl)amino)cyclopentyl)amino)-2H-[1,3′-bipyridin]-2-one—Compound 60

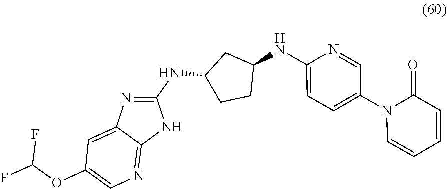 [SMILES: O=c1ccccn1-c1ccc(N[C@H]2CC[C@H](Nc3nc4cc(OC(F)F)cnc4[nH]3)C2)nc1]

3.6 M NaOH (aq, 110 μL, 0.39 mmol) was added to a suspension of 6′-(((1S,3S)-3-((6-hydroxy-3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((6-hydroxy-1-(4-methoxybenzyl)-1H-imidazo[4,5-b]pyridin-2-yl)amino)cyclopentyl)amino)-2H-[1,3′-bipyridin]-2-one Intermediate 83 (21 mg, 0.04 mmol) in MeCN (110 μL). Difluoromethyl trifluoromethanesulfonate (15 μL, 0.12 mmol) was added and the reaction mixture was stirred at rt for 1 h. Another amount of difluoromethyl trifluoromethanesulfonate (15 μL, 0.12 mmol) was added and the reaction mixture was stirred at rt for 1 h. The reaction mixture was diluted with EtOAc, the organic layer was washed with 10% NaHCO₃ (aq) and brine, dried over Na₂SO₄, filtered, and concentrated under reduced pressure. The obtained residue was dissolved in TFA (1 mL) in a sealed vial and the reaction mixture was stirred at 90° C. for 20 min. Excess TFA was removed under reduced pressure and the residue was purified by preparative HPLC, PrepMethod B (gradient: 10-50%), to give the title compound (1.3 mg, 7% over two steps) as a white solid; HRMS (ESI) m/z [M+H]⁺ calcd for C₂₂H₂₂F₂N₇O₂: 454.1798, found: 454.1804; ¹H NMR (500 MHz, DMSO-d₆) δ 1.45-2.28 (6H, m), 4.19-4.42 (2H, m), 5.8-5.99 (1H, m), 6.26 (1H, t), 6.44 (1H, d), 6.5-6.57 (1H, m), 6.89-7.02 (1H, m), 7.19-7.32 (1H, m), 7.40 (1H, dd), 7.47 (1H, ddd), 7.61 (1H, d), 7.92 (1H, d).

### Example 61

### 6′-(((1S,3S)-3-((6-(Hydroxymethyl)-3H-imidazo[4,5-b]pyridin-2-yl)amino)cyclopentyl)amino)-2H-[1,3′-bipyridin]-2-one—Compound 61

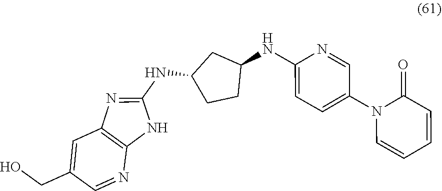 [SMILES: O=c1ccccn1-c1ccc(N[C@H]2CC[C@H](Nc3nc4cc(CO)cnc4[nH]3)C2)nc1]

A mixture of 6′-(((1S,3S)-3-((3-(4-methoxybenzyl)-6-((methoxymethoxy)methyl)-3H-imidazo[4,5-b]pyridin-2-yl)amino)cyclopentyl)amino)-2H-[1,3′-bipyridin]-2-one and 6′-(((1S,3S)-3-((1-(4-methoxybenzyl)-6-((methoxymethoxy)methyl)-1H-imidazo[4,5-b]pyridin-2-yl)amino)cyclopentyl)amino)-2H-[1,3′-bipyridin]-2-one Intermediate 84 (5.2 mg, 8.9 μmol) was dissolved in TFA (300 μL) in a sealed reactor and the reaction mixture was stirred at 100° C. for 15 min. Excess TFA was removed under reduced pressure and the residue was purified by preparative HPLC, PrepMethod B (gradient: 0-30%) to give the title compound (1.1 mg, 30%) as a white solid; HRMS (ESI) m/z [M+H]⁺ calcd for C₂₂H₂₄N₇O₂: 418.1986, found: 418.1990; ¹H NMR (500 MHz, CD₃OD) δ 1.59-1.76 (2H, m), 2.01-2.14 (2H, m), 2.25-2.4 (2H, m), 4.3-4.45 (2H, m), 4.63 (2H, s), 6.47 (1H, td), 6.58-6.65 (2H, m), 7.45 (1H, dd), 7.51 (1H, s), 7.56-7.65 (2H, m), 7.88-7.99 (2H, m).

### Example 62

### 6′-(((1S,3S)-3-((6-(Trifluoromethyl)-3H-imidazo[4,5-b]pyridin-2-yl)amino)cyclopentyl)amino)-2H-[1,3′-bipyridin]-2-one—Compound 62

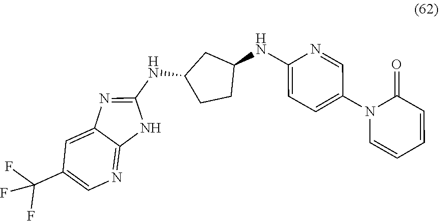 [SMILES: O=c1ccccn1-c1ccc(N[C@H]2CC[C@H](Nc3nc4cc(C(F)(F)F)cnc4[nH]3)C2)nc1]

The title compound was prepared using the same procedure as for Example 54 starting from 6′-(((1S,3S)-3-((3-(4-methoxybenzyl)-6-(trifluoromethyl)-3H-imidazo[4,5-b]pyridin-2-yl)amino)cyclopentyl)amino)-2H-[1,3′-bipyridin]-2-one* Intermediate 87 (42 mg, 0.07 mmol) and TFA (0.735 mL) to give upon purification by preparative HPLC PrepMethod B (gradient: 15-55%) the title compound (15.7 mg, 47%) as a white solid; HRMS (ESI) m/z [M+H]⁺ calcd for C₂₂H₂₁F₃N₇O: 456.1754, found: 456.1766; ¹H NMR (500 MHz, DMSO-d₆) δ 1.44-1.67 (2H, m), 1.86-2.06 (2H, m), 2.19 (2H, td), 4.34 (2H, dq), 6.27 (1H, td), 6.44 (1H, dt), 6.54 (1H, d), 6.96 (1H, d), 7.41 (1H, dd), 7.47 (1H, ddd), 7.51-7.73 (3H, m), 7.93 (1H, d), 8.24 (1H, s), 11.12 (1H, s). (*regiochemistry was not confirmed)

### Example 63

### 6′-(((1S,3S)-3-((3H-Imidazo[4,5-b]pyridin-2-yl)amino)cyclopentyl)amino)-3-(trifluoromethoxy)-2H-[1,3′-bipyridin]-2-one—Compound 63

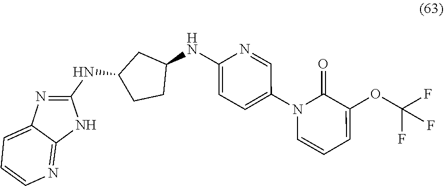 [SMILES: O=c1c(OC(F)(F)F)cccn1-c1ccc(N[C@H]2CC[C@H](Nc3nc4cccnc4[nH]3)C2)nc1]

6′-(((1S,3S)-3-((3-(4-Methoxybenzyl)-3H-imidazo[4,5-b]pyridin-2-yl)amino)cyclopentyl)amino)-3-(trifluoromethoxy)-2H-[1,3′-bipyridin]-2-one* Intermediate 88 (405 mg, 0.23 mmol) was added to TFA (5 mL) and the reaction mixture was stirred at 80° C. for 18 h. The reaction mixture was filtered through a pad of Celite, and the filter cake washed with DCM (2×5 mL). The combined filtrates were concentrated under reduced pressure and the residue was purified by preparative HPLC, PrepMethod H (gradient: 15-45%) to give the title compound (34 mg, 32%) as a white solid; HRMS (ESI) m/z [M+H]⁺ calcd for C₂₂H₂₁F₃N₇O₂: 472.1704, found: 472.1710; ¹H NMR (300 MHz, DMSO-d₆) δ 1.44-1.64 (2H, m), 1.86-2.02 (2H, m), 2.11-2.24 (2H, m), 4.25-4.37 (2H, m), 6.31 (1H, t), 6.54 (1H, d), 6.78-7.17 (3H, m), 7.35 (1H, d), 7.44 (1H, dd), 7.67-7.89 (3H, m), 7.97 (1H, d), 10.74-11.35 (1H, m); ¹⁹F NMR (282 MHz, DMSO-d₆) δ −57.35 (3F, s). (*regiochemistry was not confirmed)

### Example 64

### 6′-(((1S,3S)-3-((3H-Imidazo[4,5-b]pyridin-2-yl)amino)cyclopentyl)amino)-3-(trifluoromethyl)-2H-[1,3′-bipyridin]-2-one—Compound 64

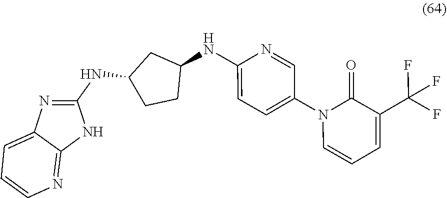 [SMILES: O=c1c(C(F)(F)F)cccn1-c1ccc(N[C@H]2CC[C@H](Nc3nc4cccnc4[nH]3)C2)nc1]

6′-(((1S,3S)-3-((3-(4-Methoxybenzyl)-3H-imidazo[4,5-b]pyridin-2-yl)amino)cyclopentyl)amino)-3-(trifluoromethyl)-2H-[1,3′-bipyridin]-2-one* Intermediate 89 (90 mg, 0.16 mmol) was added to TFA (5 mL) and the reaction mixture was stirred at 80° C. for 18 h. The reaction mixture was filtered through a pad of Celite, and the filter cake washed with DCM (2×5 mL). The combined filtrates were concentrated under reduced pressure and the residue was purified by preparative HPLC, PrepMethod F (gradient: 13-43%) to give the title compound (23 mg, 32%) as a white solid; HRMS (ESI) m/z [M+H]⁺ calcd for C₂₂H₂₁F₃N₇O: 456.1754, found: 456.1734; ¹H NMR (300 MHz, DMSO-d₆) δ 1.45-1.64 (2H, m), 1.87-2.01 (2H, m), 2.13-2.22 (2H, m), 4.25-4.38 (2H, m), 6.41 (1H, t), 6.54 (1H, d), 6.77 (0.5H, dd), 6.87-6.94 (0.5H, m), 7.03 (1H, t), 7.17 (0.5H, d), 7.36 (1H, ddd), 7.45 (1H, dd), 7.78 (0.5H, dd), 7.89-8.01 (4H, m), 10.73-11.34 (1H, m); ¹⁹F NMR (282 MHz, DMSO-d₆) δ −64.13 (3F, s). (*regiochemistry was not confirmed)

### Example 65

### 1-(6-(((1S,3S)-3-((3H-Imidazo[4,5-b]pyridin-2-yl)amino)cyclopentyl)amino)pyridin-3-yl)imidazolidine-2,4-dione—Compound 65

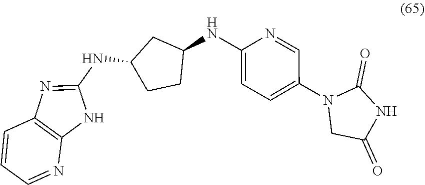 [SMILES: O=C1CN(c2ccc(N[C@H]3CC[C@H](Nc4nc5cccnc5[nH]4)C3)nc2)C(=O)N1]

3-(6-(((1S,3S)-3-((3-(4-Methoxybenzyl)-3H-imidazo[4,5-b]pyridin-2-yl)amino)cyclopentyl)ami-no)pyridin-3-yl)imidazolidine-2,4-dione* Intermediate 95 (50 mg, 0.10 mmol) was added in TFA (5 mL) at rt and the resulting solution was stirred at 80° C. for 18 h. The reaction mixture was filtered through celite. The filter cake was washed with DCM (2×5 mL). The filtrate was concentrated under reduced pressure to afford the crude product. The crude product was purified by preparative HPLC (PrepMethod F, gradient 3-33%) to afford (20 mg, 52%) of the title compound as a white solid. HRMS (ESI) m/z [M+H]⁺ calcd for C₁₉H₂₁N₈O₂: 393.1782, found: 393.1778. ¹H NMR (300 MHz, DMSO-d₆) δ 1.52 (2H, ddt), 1.90 (2H, dp), 2.05-2.26 (2H, m), 4.04-4.54 (4H, m), 6.47 (1H, d), 6.56 (1H, d), 6.7-7.2 (2H, br. m), 7.35 (1H, d), 7.62 (1H, dd), 7.8-8.0 (1H, br s), 8.05 (1H, d), 10.7-11.5 (2H, br m). (*regiochemistry was not confirmed)

### Example 66

### (1S,3S)—N¹-(3H-Imidazo[4,5-b]pyridin-2-yl)-N³-(5-(2-methoxyphenyl)pyridin-2-yl)cyclopentane-1,3-diamine—Compound 66

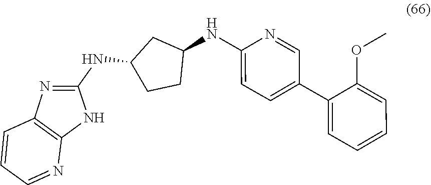 [SMILES: COc1ccccc1-c1ccc(N[C@H]2CC[C@H](Nc3nc4cccnc4[nH]3)C2)nc1]

(1S,3S)—N¹-(5-(2-Methoxyphenyl)pyridin-2-yl)-N³-(3-tosyl-3H-imidazo[4,5-b]pyridin-2-yl)cyclopentane-1,3-diamine* Intermediate 97 (140 mg, 0.25 mmol) was added to K₂CO₃ (70 mg, 0.50 mmol) in MeOH (10 mL) and the resulting mixture was stirred at 60° C. for 3 h. The reaction mixture was filtered through filter paper, the filter cake was washed with MeOH (3×20 mL) and the filtrate was concentrated under reduced pressure to afford the crude product. The crude product was first purified by preparative TLC (MeOH:DCM=1:10) and further purified by preparative HPLC (PrepMethod Q, gradient 66-73%) to afford (22 mg, 22%) of the title compound as a white solid. HRMS (ESI) m/z [M+H]⁺ calcd for C₂₃H₂₅N₆O: 401.2084, found: 401.2064. ¹H NMR (300 MHz, MeOH-d₄) δ 1.51-1.81 (2H, m), 2.06-2.11 (2H, m), 2.24-2.47 (2H, m), 3.80 (3H, s), 4.26-4.44 (2H, m), 6.57 (1H, d), 6.92-7.08 (3H, m), 7.22-7.32 (2H, m), 7.48 (1H, d), 7.61 (1H, dd), 7.92 (1H, d), 8.06 (1H, d). (*regiochemistry was not confirmed)

### Example 67

### (1S,3S)—N¹-(5-(2-Fluoro-6-methoxyphenyl)pyridin-2-yl)-N³-(3H-imidazo[4,5-b]pyridin-2-yl)cyclopentane-1,3-diamine—Compound 67

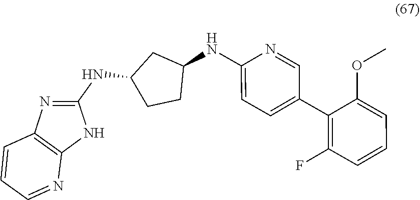 [SMILES: COc1cccc(F)c1-c1ccc(N[C@H]2CC[C@H](Nc3nc4cccnc4[nH]3)C2)nc1]

(1S,3S)—N¹-(3H-imidazo[4,5-b]pyridin-2-yl)-N³-(5-iodopyridin-2-yl)cyclopentane-1,3-diamine Intermediate 100 (50 mg, 0.12 mmol) was added to (2-fluoro-6-methoxyphenyl)boronic acid (CAS Reg. No. 784945-63-3) (101 mg, 0.59 mmol), Pd(dppf)Cl₂·DCM (9.7 mg, 0.01 mmol) and K₃PO₄ (76 mg, 0.36 mmol) in water (1 mL) and 1,4-dioxane (10 mL) and the resulting mixture was stirred at 80° C. for 3 h under nitrogen. The reaction mixture was filtered through celite. The filter cake was washed with EtOAc (3×20 mL) and the filtrate was concentrated under reduced pressure to afford the crude product. The crude product was first purified by preparative TLC (MeOH:DCM=1:10) followed by preparative HPLC (PrepMethod M, gradient 6-21%) to give (23 mg, 45%) of the title compound as a white solid. HRMS (ESI) m/z [M+H]⁺ calcd for C₂₃H₂₄FN₆O: 419.1990, found: 419.1984. ¹H NMR (300 MHz, MeOH-d₄) δ 1.86 (2H, tt), 2.20-2.38 (2H, m), 2.4-2.55 (2H, m), 3.85 (3H, s), 4.3-4.45 (1H, m), 4.45-4.6 (1H, m), 6.87 (1H, t), 6.98 (1H, d), 7.09-7.18 (1H, m), 7.24 (1H, dd), 7.38-7.46 (1H, m), 7.81-7.93 (2H, m), 7.93-8.03 (2H, m). ¹⁹F NMR (282 MHz, CD₃OD) δ −118.150.

### Example 68

### (1S,3S)—N¹-(5-(2,6-Difluorophenyl)pyridin-2-yl)-N³-(3H-imidazo[4,5-b]pyridin-2-yl)cyclopentane-1,3-diamine—Compound 68

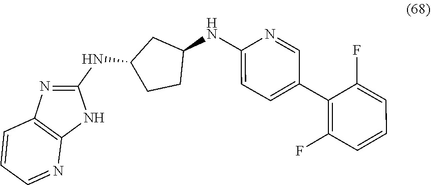 [SMILES: Fc1cccc(F)c1-c1ccc(N[C@H]2CC[C@H](Nc3nc4cccnc4[nH]3)C2)nc1]

(1S,3S)—N¹-(5-(2,6-Difluorophenyl)pyridin-2-yl)-N³-(3-tosyl-3H-imidazo[4,5-b]pyridin-2-yl)cyclopentane-1,3-diamine* Intermediate 101 (120 mg, 0.21 mmol) was added to K₂CO₃ (59 mg, 0.43 mmol) in MeOH (10 mL). The resulting mixture was stirred at 60° C. for 2 h. The reaction mixture was filtered through a filter paper. The filter cake was washed with MeOH (3×20 mL) and the filtrate was concentrated under reduced pressure to afford the crude product. The crude product was first purified by preparative TLC (MeOH:DCM=1:10) followed by preparative HPLC (PrepMethod Q, gradient 66-74%) to afford (26 mg, 30%) of the title compound as a white solid. HRMS (ESI) m/z [M+H]⁺ calcd for C₂₂H₂₁F₂N₆: 407.1790, found: 407.1792. ¹H NMR (300 MHz, MeOH-d₄) δ 1.57-1.78 (2H, m), 2.05-2.15 (2H, m), 2.24-2.43 (2H, m), 4.3-4.45 (2H, m), 6.63 (1H, d), 6.96 (1H, dd), 7.00-7.12 (2H, m), 7.25-7.4 (1H, m), 7.43-7.57 (2H, m), 7.92 (1H, d), 8.03 (1H, d). ¹⁹F NMR (282 MHz, MeOH-d₄) δ −116.942. (*regiochemistry was not confirmed)

### Example 69

### (1S,3S)—N¹-(5-(2-Fluorophenyl)pyridin-2-yl)-N³-(3H-imidazo[4,5-b]pyridin-2-yl)cyclopentane-1,3-diamine—Compound 69

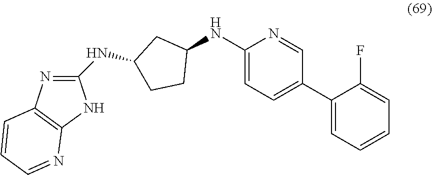 [SMILES: Fc1ccccc1-c1ccc(N[C@H]2CC[C@H](Nc3nc4cccnc4[nH]3)C2)nc1]

Cs₂CO₃ (341 mg, 1.05 mmol) was added to (1S,3S)—N¹-(5-(2-fluorophenyl)pyridin-2-yl)cyclopentane-1,3-diamine×3.5 HCl Intermediate 104 (84 mg, 0.21 mmol), 2-chloro-3-tosyl-3H-imidazo[4,5-b]pyridine* Intermediate 39 (129 mg, 0.42 mmol) and Pd-PEPPSI-IpentCl 2-methylpyridine (14 mg, 0.02 mmol) in 1,4-dioxane (3 mL) at rt. The resulting suspension was stirred at 100° C. for 16 h under nitrogen. The mixture was added into MeOH (3 mL) at rt. The resulting suspension was stirred at 60° C. for 1 hour. The solvent was removed under reduced pressure. The residue was diluted with MeOH (200 mL), filtered through Celite and the filtrate evaporated to dryness. The obtained crude product was purified by preparative HPLC (PrepMethod S, gradient 31-51%) to afford (30 mg, 37%) of the title compound as a white solid. HRMS (ESI) m/z [M+H]⁺ calcd for C₂₂H₂₂FN₆: 389.1884, found: 389.1880. ¹H NMR (300 MHz, DMSO-d₆, two rotamers/tautomers) δ 1.44-1.65 (2H, m), 1.87-2.06 (2H, m), 2.1-2.3 (2H, m), 4.22-4.45 (2H, m), 6.57 (1H, d), 6.74-7.0 (2H, m), 7.17-7.42 (5H, m), 7.48 (1H, dt), 7.59 (1H, dt), 7.8 and 7.92 (1H, d, two rotamers), 8.18 (1H, s), 10.75 and 11.35 (1H, s, two rotamers/tautomers). ¹⁹F NMR (282 MHz, DMSO-d₆) 5-118.453. (*regiochemistry was not confirmed)

### Example 70

### (1S,3S)—N¹-(5-(2-Fluoro-6-(trifluoromethoxy)phenyl)pyridin-2-yl)-N³-(3H-imidazo[4,5-b]pyridin-2-yl)cyclopentane-1,3-diamine—Compound 70

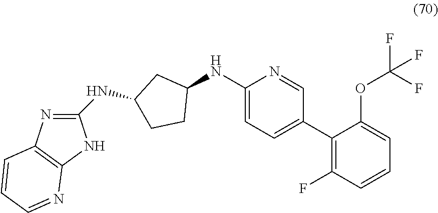 [SMILES: Fc1cccc(OC(F)(F)F)c1-c1ccc(N[C@H]2CC[C@H](Nc3nc4cccnc4[nH]3)C2)nc1]

A microwave vial was charged with the free base (1S,3S)—N¹-(5-(2-fluoro-6-(trifluoromethoxy)phenyl)pyridin-2-yl)cyclopentane-1,3-diamine Intermediate 106 (80 mg, 0.23 mmol), 2-(methylsulfonyl)-3H-imidazo[4,5-b]pyridine (CAS Reg. No. 57159-81-6) (44 mg, 0.23 mmol) and n-butanol (2 mL). The vial was sealed and heated to 180° C. for 2 h in the microwave reactor. The mixture was filtered through a Celite pad. The crude product was first purified by reversed phase flash chromatography on a C18 column (gradient: 5-100% MeCN in water) followed by preparative HPLC (PrepMethod T, gradient 5-22%) to afford (5 mg, 5%) of the title compound as a white solid. HRMS (ESI) m/z [M+H]⁺ calcd for C₂₃H₂₁F₄N₆O: 473.1708, found: 473.1714. ¹⁹F NMR (282 MHz, MeOH-d₄) δ −58.98, −114.71.

### Example 71

### 6′-(((1S,3S)-3-((3H-Imidazo[4,5-b]pyridin-2-yl)amino)cyclopentyl)amino)-5-chloro-2H-[1,3′-bipyridin]-2-one—Compound (71)

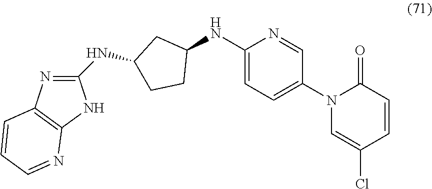 [SMILES: O=c1ccc(Cl)cn1-c1ccc(N[C@H]2CC[C@H](Nc3nc4cccnc4[nH]3)C2)nc1]

5-Chloro-6′-(((1S,3S)-3-((1-(4-methoxybenzyl)-1H-imidazo[4,5-b]pyridin-2-yl)amino)cyclopentyl)amino)-2H-[1,3′-bipyridin]-2-one* Intermediate 108 (180 mg, 0.33 mmol) was added to TFA (3 mL) at rt and the resulting solution was stirred at 80° C. for 16 h. The reaction mixture was filtered through celite. The filter cake was washed with DCM (2×5 mL) and the filtrate was concentrated under reduced pressure to afford the crude product. The crude product first purified by reversed phase flash chromatography on a C18 column (gradient: 0-45% MeCN in water) followed by preparative HPLC (PrepMethod H, gradient 15-45%) to afford (33 mg, 24%) of the title compound as a white solid. HRMS (ESI) m/z [M+H]⁺ calcd for C₂₁H₂₁ClN₇O: 422.1490, found: 422.1510. ¹H NMR (300 MHz, DMSO-d₆) δ 1.40-1.71 (2H, m), 1.88-2.01 (2H, m), 2.1-2.25 (2H, m), 4.25-4.40 (2H, m), 6.45-6.75 (2H, m), 6.68-7.25 (m, 3H), 7.32-7.45 (2H, m), 7.53 (1H, dd), 7.78-7.98 (3H, m), 10.76 and 11.35 (1H, s, rotamers/tautomers). (*regiochemistry was not confirmed)

### Example 72

### 6′-(((1S,3S)-3-((3H-Imidazo[4,5-b]pyridin-2-yl)amino)cyclopentyl)amino)-5-methoxy-2H-[1,3′-bipyridin]-2-one—Compound 72

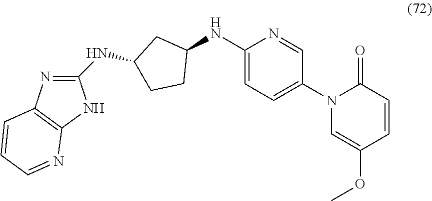 [SMILES: COc1ccc(=O)n(-c2ccc(N[C@H]3CC[C@H](Nc4nc5cccnc5[nH]4)C3)nc2)c1]

5-Methoxy-6′-(((1S,3S)-3-((1-(4-methoxybenzyl)-1H-imidazo[4,5-b]pyridin-2-yl)amino)cyclopentyl)amino)-2H-[1,3′-bipyridin]-2-one* Intermediate 109 (50 mg, 0.09 mmol) was added in TFA (5 mL). The resulting solution was stirred at 80° C. for 4 h. The reaction mixture was filtered through celite. The filter cake was washed with DCM (2×5 mL). The filtrate was concentrated under reduced pressure and the obtained material was purified by preparative HPLC (PrepMethod U, gradient 5-24%) to afford (23 mg, 57%) of the title compound as a white solid. HRMS (ESI) m/z [M+H]⁺ calcd for C₂₂H₂₄N₇O₂: 418.1986, found: 418.1980. ¹H NMR (300 MHz, DMSO-d₆) δ 1.45-1.67 (2H, M), 1.85-2.06 (2H, m), 2.10-2.30 (2H, m), 3.65 (3H, s), 4.25-4.40 (2H, m), 6.41 (1H, d), 6.53 (1H, d), 6.84 (1H, t), 6.92 (1H, d), 7.1-7.2 (2H, m), 7.33-7.46 (3H, m), 7.86 (1H, s), 7.96 (1H, d), 8.14 (1H, s). (*regiochemistry was not confirmed)

### Example 73

### 6′-(((1S,3S)-3-((7-Bromo-3H-imidazo[4,5-b]pyridin-2-yl)amino)cyclopentyl)amino)-2H-[1,3′-bipyridin]-2-one—Compound 73

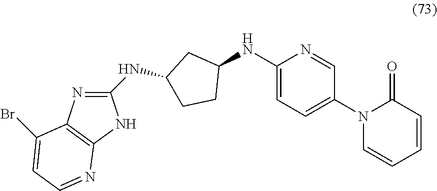 [SMILES: O=c1ccccn1-c1ccc(N[C@H]2CC[C@H](Nc3nc4c(Br)ccnc4[nH]3)C2)nc1]

TCDI (235 mg, 1.32 mmol) was added to 6′-(((1S,3S)-3-aminocyclopentyl)amino)-2H-[1,3′-bipyridin]-2-one×3 HCl Intermediate 3 (250 mg, 0.66 mmol) and NaOH (158 mg, 3.95 mmol) in DMF (5 mL) at rt and the resulting solution was stirred at 100° C. for 2 h. The temperature was decreased to rt and 4-bromopyridine-2,3-diamine (CAS Reg. No. 1232431-75-2) (136 mg, 0.72 mmol) and EDC (252 mg, 1.32 mmol) were added into the reaction mixture at rt and the resulting solution was stirred at 100° C. for 15 h. The mixture was filtered through a Celite pad, the filter cake was washed with DCM (2×5 mL). The filtrate was concentrated and purified directly. The concentrated solution was first purified by reversed phase flash chromatography on a C18 column (gradient: 5-28% MeCN in water/0.1% NH₃) then by preparative TLC (MeOH (7M ammonia solution):DCM=1:20) and finally by preparative HPLC (PrepMethod F, gradient 15-35%) to afford (15 mg, 5%) of the title compound as a white solid. HRMS (ESI) m/z [M+H]⁺ calcd for C₂₁H₂₁BrN₇O: 466.0986, found: 466.0980. ¹H NMR (300 MHz, DMSO-d₆) δ 1.41-1.70 (2H, m), 1.87-2.05 (2H, m), 2.12-2.26 (2H, m), 4.28-4.40 (2H, m), 6.27 (1H, dt), 6.40-6.60 (2H, m), 6.95 (1H, d), 7.13 (1H, s), 7.34-7.53 (3H, m), 7.58-7.73 (2H, m), 7.94 (1H, d), 11.78 (1H, s).

### Example 74

### 1-(4-(((1S,3S)-3-((3H-Imidazo[4,5-b]pyridin-2-yl)amino)cyclopentyl)amino)phenyl)pyridin-2(1H)-one—Compound 74

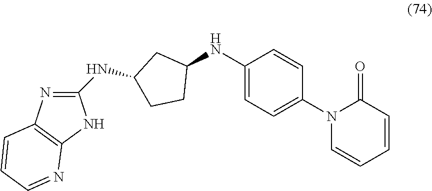 [SMILES: O=c1ccccn1-c1ccc(N[C@H]2CC[C@H](Nc3nc4cccnc4[nH]3)C2)cc1]

1-(4-(((1S,3S)-3-((3-Tosyl-3H-imidazo[4,5-b]pyridin-2-yl)amino)cyclopentyl)amino)phenyl)pyri-din-2(1H)-one* Intermediate 110 (70 mg, 0.13 mmol) was added to K₂CO₃ (36 mg, 0.26 mmol) in MeOH (5 mL) and the resulting mixture was stirred at 60° C. for 3 h. The reaction mixture was filtered through filter paper. The filter cake was washed with MeOH (3×20 mL) and the filtrate was concentrated under reduced pressure to afford the crude product. The crude product was first purified by preparative TLC (MeOH:DCM=1:10) followed by preparative HPLC (PrepMethod F, gradient 39-51%) to afford (10 mg, 20%) of the title compound as a white solid. HRMS (ESI) m/z [M+H]⁺ calcd for C₂₂H₂₃N₆O: 387.1928, found: 387.1902. ¹H NMR (300 MHz, DMSO-d₆) 1.47-1.67 (2H, m), 1.90-2.05 (2H, m), 2.12-2.25 (2H, m), 3.87-3.98 (1H, m), 4.23-4.35 (1H, d), 5.98-6.06 (1H, m), 6.22 (1H, dt), 6.40 (1H, d), 6.61 (2H, d), 6-74-6.95 (1H, m), 7.06 (2H, d), 7.2-7.4 (1H, m), 7.34 (1H, d), 7.4-7.5 (1H, m), 7.55 (1H, br. d), 7.84 (1H, d), 10.86 and 11.48 (1H, s, rotamers/tautomers). (*regiochemistry was not confirmed)

### Example 75

### 3-(5-(((1S,3S)-3-((3H-Imidazo[4,5-b]pyridin-2-yl)amino)cyclopentyl)amino)pyrazin-2-yl)-1-methylpyridin-2(1H)-one—Compound 75

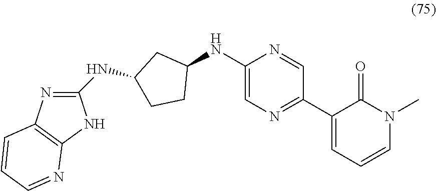 [SMILES: Cn1cccc(-c2cnc(N[C@H]3CC[C@H](Nc4nc5cccnc5[nH]4)C3)cn2)c1=O]

TEA (0.110 mL, 0.79 mmol) was added to 3-(5-(((1S,3S)-3-aminocyclopentyl)amino)pyrazin-2-yl)-1-methylpyridin-2(1H)-one×HCl Intermediate 113 (60 mg, 0.16 mmol) and TCDI (56 mg, 0.31 mmol) in DMF (3 mL) at rt and the resulting solution was stirred at 100° C. for 2 h. The temperature was decreased to room temperature and pyridine-2,3-diamine (CAS Reg. No. 452-58-4) (19 mg, 0.17 mmol) and EDC (60 mg, 0.31 mmol) was added to the solution and the resulting solution was stirred at 100° C. for 15 h. The reaction mixture was filtered, the filter cake was washed with DCM (3×5 mL). The filtrate was concentrated and purified directly. The concentrated solution was first purified by reversed phase flash chromatography on a C18 column (gradient: 0-36% MeCN in water/0.1% NH₃) followed by preparative HPLC (PrepMethod A, gradient 15-35%) to afford (15 mg, 24%) of the title compound as a white solid. HRMS (ESI) m/z [M+H]⁺ calcd for C₂₁H₂₃N₈O: 403.1990, found: 403.1974. ¹H NMR (300 MHz, DMSO-d₆, mixture of rotamers/tautomers) δ 1.45-1.67 (2H, m), 1.91-2.06 (2H, m), 2.13-2.26 (2H, m), 3.52 (3H, s), 4.25-4.43 (2H, m), 6.31-6.40 (1H, m), 6.70-6.94 (1H, m), 7.10 (1H, m), 7.2-7.4 (2H, m), 7.69-8.02 (3H, m), 8.13 (1H, dd), 9.18 (1H, d), 10.78 and 11.41 (1H, ds, mixture of rotamers/tautomers).

### Example 76

### 6-(6-(((1S,3S)-3-(Thiazolo[5,4-b]pyridin-2-ylamino)cyclopentyl)amino)pyridin-3-yl)thieno[2,3-c]pyridin-7(6H)-one—Compound 76

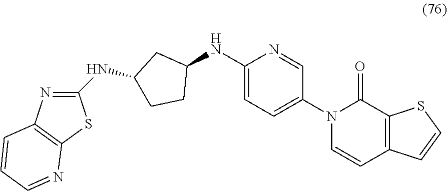 [SMILES: O=c1c2sccc2ccn1-c1ccc(N[C@H]2CC[C@H](Nc3nc4cccnc4s3)C2)nc1]

Cs₂CO₃ (314 mg, 0.96 mmol) was added to 6-(6-(((1S,3S)-3-aminocyclopentyl)amino)pyridin-3-yl)thieno[2,3-c]pyridin-7(6H)-one×3HCl Intermediate 93 (140 mg, 0.32 mmol), 2-chlorothiazolo[5,4-b]pyridine (CAS Reg. No. 91524-96-8) (82 mg, 0.48 mmol) and Pd-PEPPSI-IpentCl 2-methylpyridine (13.51 mg, 0.02 mmol) in 1,4-dioxane (5 mL) at 20° C. The resulting mixture was stirred at 100° C. for 15 h under a nitrogen atmosphere. The reaction mixture was diluted with water (50 mL) and extracted with EtOAc (3×100 mL). The organic layers were combined, dried over Na₂SO₄, filtered and evaporated. The residue was purified by preparative TLC (EtOAc) and then by preparative HPLC, PrepMethod A (gradient: 34-55%) to give the title compound (80 mg, 54%) as a white solid. HRMS (ESI) m/z [M+H]⁺ calcd for C₂₃H₂₁N₆OS₂: 461.1212, found: 461.1220. ¹H NMR (300 MHz, DMSO-d₆) δ 1.46-1.73 (2H, m), 1.89-2.11 (2H, m), 2.14-2.27 (2H, m), 4.29-4.50 (2H, m), 6.57 (1H, d), 6.84 (1H, d), 7.00 (1H, d), 7.25 (1H, dd), 7.41-7.55 (3H, m), 7.67 (1H, dd), 8.01 (1H, d), 8.05-8.14 (2H, m), 8.47 (1H, d).

### Example 77

### 5-(Difluoromethoxy)-6′-(((1S,3S)-3-(thiazolo[5,4-b]pyridin-2-ylamino)cyclopentyl)amino)-2H-[1,3′-bipyridin]-2-one—Compound 77

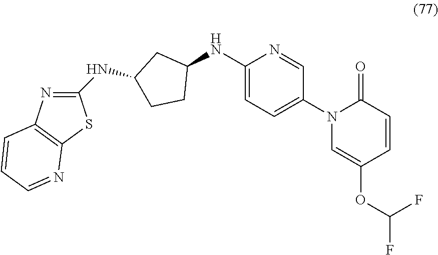 [SMILES: O=c1ccc(OC(F)F)cn1-c1ccc(N[C@H]2CC[C@H](Nc3nc4cccnc4s3)C2)nc1]

6′-(((1S,3S)-3-Aminocyclopentyl)amino)-5-(difluoromethoxy)-2H-[1,3′-bipyridin]-2-one×4TFA Intermediate 119 (836 mg, 1.06 mmol) was added to 2-chlorothiazolo[5,4-b]pyridine (CAS Reg. No. 91524-96-8) (120 mg, 0.70 mmol), Pd-PEPPSI-IpentCl 2-methylpyridine (29.6 mg, 0.04 mmol) and Cs₂CO₃ (687 mg, 2.11 mmol) in 1,4-dioxane (15 mL) at 25° C. The resulting suspension was stirred at 100° C. for 18 h under a nitrogen atmosphere. The reaction mixture was filtered through Celite. The filter cake was washed with DCM (2×5 mL) and the combined filtrates were concentrated under reduced pressure. The residue was purified by preparative TLC (MeOH:EtOAc, 1:40) and then by preparative HPLC, PrepMethod A (gradient: 26-56%) to give the title compound (85 mg, 26%) as a white solid. HRMS (ESI) m/z [M+H]⁺ calcd for C₂₂H₂₁F₂N₆O₂S: 471.1408, found: 471.1404. ¹H NMR (300 MHz, DMSO-d₆) δ 1.46-1.7 (2H, m), 1.86-2.1 (2H, m), 2.1-2.34 (2H, m), 4.25-4.52 (2H, m), 6.52 (2H, dd), 6.73-7.33 (3H, m), 7.44 (1H, dd), 7.52 (1H, dd), 7.6-7.71 (1H, m), 7.71-7.82 (1H, m), 7.92-8.03 (1H, m), 8.03-8.16 (1H, m), 8.46 (1H, d).

### Example 78

### 5-(Difluoromethoxy)-6′-(((1S,3S)-3-(oxazolo[5,4-b]pyridin-2-ylamino)cyclopentyl)amino)-2H-[1,3′-bipyridin]-2-one—Compound 78

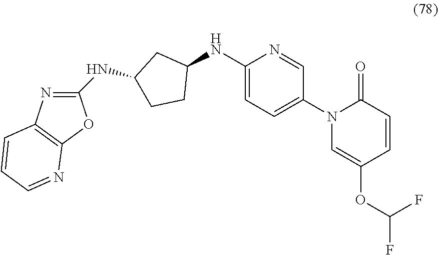 [SMILES: O=c1ccc(OC(F)F)cn1-c1ccc(N[C@H]2CC[C@H](Nc3nc4cccnc4o3)C2)nc1]

6′-(((1S,3S)-3-Aminocyclopentyl)amino)-5-(difluoromethoxy)-2H-[1,3′-bipyridin]-2-one×4TFA Intermediate 119 (923 mg, 1.16 mmol) was added to 2-chlorooxazolo[5,4-b]pyridine (CAS Reg. No. 159870-95-8) (120 mg, 0.78 mmol), Pd-PEPPSI-IpentCl 2-methylpyridine (33 mg, 0.04 mmol) and Cs₂CO₃ (759 mg, 2.33 mmol) in 1,4-dioxane (15 mL) at 25° C. The resulting suspension was stirred at 100° C. for 18 h under a nitrogen atmosphere. The reaction mixture was filtered through celite. The filter cake was washed with DCM (2×5 mL) and the combined filtrates were concentrated under reduced pressure. The residue was purified by preparative TLC (MeOH:EtOAc, 1:40) and then by preparative HPLC, PrepMethod A (gradient: 25-47%) to give the title compound (178 mg, 50%) as a white solid. HRMS (ESI) m/z [M+H]⁺ calcd for C₂₂H₂₁F₂N₆O₃: 455.1638, found: 455.1632. ¹H NMR (300 MHz, DMSO-d₆) δ 1.44-1.76 (2H, m), 1.86-2.07 (2H, m), 2.11-2.27 (2H, m), 4.18-4.48 (2H, m), 6.45-6.6 (2H, m), 6.74-7.32 (3H, m), 7.44 (1H, dd), 7.52 (1H, dd), 7.59 (1H, dd), 7.76 (1H, d), 7.84 (1H, dd), 7.98 (1H, d), 8.41 (1H, d).

### Example 79

### 3-(Difluoromethoxy)-6′-(((1S,3S)-3-(oxazolo[5,4-b]pyridin-2-ylamino)cyclopentyl)amino)-2H-[1,3′-bipyridin]-2-one—Compound 79

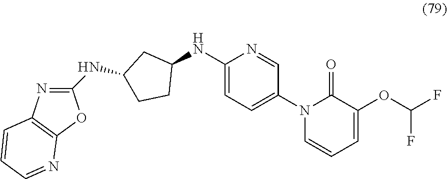 [SMILES: O=c1c(OC(F)F)cccn1-c1ccc(N[C@H]2CC[C@H](Nc3nc4cccnc4o3)C2)nc1]

2-Chlorooxazolo[5,4-b]pyridine (70 mg, 0.45 mmol) was added to 6′-(((1S,3S)-3-aminocyclopentyl)amino)-3-(difluoromethoxy)-2H-[1,3′-bipyridin]-2-one×2.4HCl Intermediate 121 (192 mg, 0.45 mmol), Pd-PEPPSI-IpentCl 2-methylpyridine (19.05 mg, 0.02 mmol) and Cs₂CO₃ (443 mg, 1.36 mmol) in 1,4-dioxane (15 mL) at 20° C. The resulting mixture was stirred at 100° C. for 15 h under a nitrogen atmosphere. The reaction mixture was poured into water (125 mL) and extracted with EtOAc (4×100 mL). The organic layer was dried over Na₂SO₄, filtered and evaporated. The residue was purified by preparative TLC (MeOH:EtOAc, 1:40) and then by preparative HPLC, PrepMethod V (gradient: 10-28%) to give the title compound (70 mg, 33%) as a white solid. HRMS (ESI) m/z [M+H]⁺ calcd for C₂₂H₂₁F₂N₆O₃: 455.1638, found: 455.1622. ¹H NMR (300 MHz, DMSO-d₆) δ 1.4-1.74 (2H, m), 1.84-2.12 (2H, m), 2.12-2.28 (2H, m), 4.15-4.5 (2H, m), 6.30 (1H, t), 6.56 (1H, d), 6.89-7.49 (5H, m), 7.59 (2H, dd), 7.84 (1H, dd), 7.98 (1H, d), 8.41 (1H, d).

### Example 80

### 6′-(((1S,3S)-3-((1H-Imidazo[4,5-b]pyrazin-2-yl)amino)cyclopentyl)amino)-2H-[1,3′-bipyridin]-2-one—Compound 80

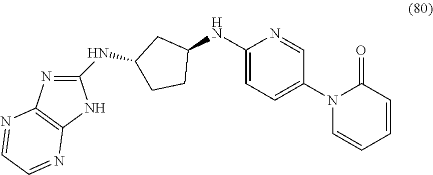 [SMILES: O=c1ccccn1-c1ccc(N[C@H]2CC[C@H](Nc3nc4nccnc4[nH]3)C2)nc1]

The title compound was synthesized using the same procedure as applied for Example 51 starting from 6′-(((1S,3S)-3-aminocyclopentyl)amino)-2H-[1,3′-bipyridin]-2-one Intermediate 3 (55 mg, 0.20 mmol) and pyrazine-2,3-diamine (22 mg, 0.20 mmol) to give upon purification by preparative HPLC, PrepMethod B (gradient: 0-30%), the title compound (4.5 mg, 5.7%) as a brown solid; HRMS (ESI) m/z [M+H]⁺ calcd for C₂₀H₂₁N₈O: 389.1832, found: 389.1836; ¹H NMR (500 MHz, DMSO-d₆) δ 1.45-1.71 (2H, m), 1.88-2.09 (2H, m), 2.11-2.27 (2H, m), 4.28-4.43 (2H, m), 6.22-6.3 (1H, m), 6.44 (1H, d), 6.53 (1H, d), 6.95 (1H, d), 7.41 (1H, d), 7.44-7.51 (1H, m), 7.60 (1H, d), 7.66-7.79 (2H, m), 7.82-7.98 (2H, m), 11.76 (1H, s).

### Example 81

### 3-(Difluoromethoxy)-6′-(((1S,3S)-3-(thiazolo[5,4-b]pyridin-2-ylamino)cyclopentyl)amino)-2H-[1,3′-bipyridin]-2-one—Compound 81

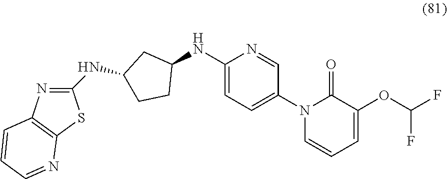 [SMILES: O=c1c(OC(F)F)cccn1-c1ccc(N[C@H]2CC[C@H](Nc3nc4cccnc4s3)C2)nc1]

2-Chlorothiazolo[5,4-b]pyridine (CAS Reg. No. 91524-96-8) (70 mg, 0.41 mmol) was added to 6′-(((1S,3S)-3-aminocyclopentyl)amino)-3-(difluoromethoxy)-2H-[1,3′-bipyridin]-2-one×2.4HCl Intermediate 121 (174 mg, 0.41 mmol), Pd-PEPPSI-IpentCl 2-methylpyridine (17.3 mg, 0.02 mmol) and Cs₂CO₃ (401 mg, 1.23 mmol) in 1,4-dioxane (15 mL) at 20° C. The resulting mixture was stirred at 100° C. for 15 h under a nitrogen atmosphere. The reaction mixture was poured into water (125 mL) and extracted with EtOAc (4×100 mL). The organic layer was dried over Na₂SO₄, filtered and evaporated. The residue was purified by preparative TLC (MeOH:EtOAc, 1:40) and then by preparative HPLC, PrepMethod W (gradient: 23-34%) to give the title compound (10 mg, 5%) as a white solid. HRMS (ESI) m/z [M+H]⁺ calcd for C₂₂H₂₁F₂N₆O₂S: 471.1408, found: 471.1394. ¹H NMR (400 MHz, MeOH-d₄) δ 1.57-1.87 (2H, m), 2-2.24 (2H, m), 2.24-2.45 (2H, m), 4.33-4.54 (2H, m), 6.43 (1H, t), 6.65 (1H, d), 6.75-7.21 (1H, m), 7.31 (1H, dd), 7.42-7.57 (3H, m), 7.72 (1H, dd), 8.00 (1H, d), 8.12 (1H, dd).

### Example 82

### 1-Methyl-3-(6-(((1S,3S)-3-(thiazolo[5,4-b]pyridin-2-ylamino)cyclopentyl)amino)pyridin-3-yl)pyrimidine-2,4(1H,3H)-dione—Compound 82

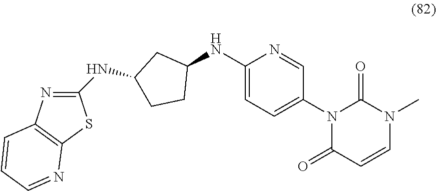 [SMILES: Cn1ccc(=O)n(-c2ccc(N[C@H]3CC[C@H](Nc4nc5cccnc5s4)C3)nc2)c1=O]

3-(6-(((1S,3S)-3-Aminocyclopentyl)amino)pyridin-3-yl)-1-methylpyrimidine-2,4(1H,3H)-dione×3 TFA Intermediate 117 (113 mg, 0.18 mmol) was added to a mixture of 2-chlorothiazolo[5,4-b]pyridine (CAS Reg. No. 91524-96-8) (30 mg, 0.18 mmol), Pd-PEPPSI-IpentCl 2-methylpyridine (7.4 mg, 8.8 μmol) and Cs₂CO₃ (172 mg, 0.53 mmol) in 1,4-dioxane (8 mL) and the resulting suspension was stirred at 100° C. for 15 h under nitrogen. The mixture was filtered through Celite and the filter cake washed with EtOAc (3×20 mL). The combined filtrates were concentrated under reduced pressure and the obtained material was purified by preparative TLC (MeOH:DCM=1:10) followed by preparative HPLC (PrepMethod V, gradient 10-25%) to afford the title compound (8.3 mg, 11%) as a white solid. HRMS (ESI) m/z [M+H]⁺ calcd for C₂₁H₂₂N₇O₂S: 436.1550, found: 436.1562. ¹H NMR (400 MHz, MeOH-d₄) δ 1.61-1.77 (2H, m), 2.03-2.17 (2H, m), 2.25-2.38 (2H, m), 3.40 (3H, s), 4.35 (1H, qn), 4.45 (1H, qn), 5.81 (1H, d), 6.64 (1H, d), 7.27-7.30 (2H, m), 7.63 (1H, d), 7.70 (1H, dd), 7.80 (1H, d), 8.10 (1H, dd).

### Example 83

### 3-(6-(((1S,3S)-3-(Thiazolo[5,4-b]pyridin-2-ylamino)cyclopentyl)amino)pyridin-3-yl)thieno[3,2-d]pyrimidine-2,4(1H,3H)-dione—Compound 83

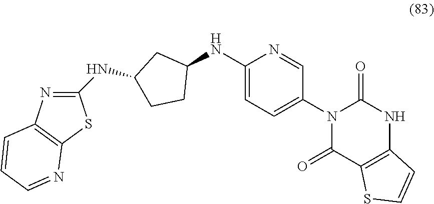 [SMILES: O=c1[nH]c2ccsc2c(=O)n1-c1ccc(N[C@H]2CC[C@H](Nc3nc4cccnc4s3)C2)nc1]

3-(6-(((1S,3S)-3-Aminocyclopentyl)amino)pyridin-3-yl)thieno[3,2-d]pyrimidine-2,4(1H,3H)-dione Intermediate 123 (241 mg, 0.35 mmol) was added to a mixture of 2-chlorothiazolo[5,4-b]pyridine (CAS Reg. No. 91524-96-8) (60 mg, 0.35 mmol), Pd-PEPPSI-IpentCl 2-methylpyridine (15 mg, 0.02 mmol) and Cs₂CO₃ (344 mg, 1.06 mmol) in 1,4-dioxane (10 mL) and the resulting suspension was stirred at 100° C. for 15 h under nitrogen. The mixture was filtered through Celite and the filter cake washed with EtOAc (3×20 mL). The combined filtrates were concentrated under reduced pressure and the obtained material was purified by preparative TLC (MeOH:DCM=1:10) followed by preparative HPLC (PrepMethod V, gradient 10-25%) to afford the title compound (9.2 mg, 5%) as a white solid. HRMS (ESI) m/z [M+H]⁺ calcd for C₂₂H₂₀N₇O₂S₂: 478.1114, found: 478.1124. ¹H NMR (400 MHz, DMSO-d₆) δ 1.50-1.65 (2H, m), 1.91-2.04 (2H, m), 2.13-2.26 (2H, m), 4.31-4.44 (2H, m), 6.53 (1H, d), 6.87 (1H, d), 6.96 (1H, d), 7.24 (1H, dd), 7.28 (1H, dd), 7.67 (1H, dd), 7.83 (1H, d), 8.09 (1H, dd), 8.11 (1H, d), 8.46 (1H, d), 11.95 (1H, brs).

### Example 84

### 6′-(((1S,3S)-3-((5-(Hydroxymethyl)benzo[d]thiazol-2-yl)amino)cyclopentyl)amino)-2H-[1,3′-bipyridin]-2-one—Compound 84

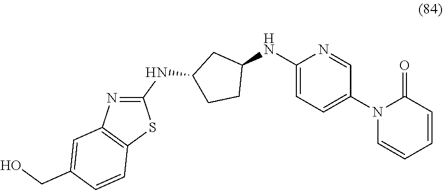 [SMILES: O=c1ccccn1-c1ccc(N[C@H]2CC[C@H](Nc3nc4cc(CO)ccc4s3)C2)nc1]

6′-(((1S,3S)-3-((5-Bromobenzo[d]thiazol-2-yl)amino)cyclopentyl)amino)-2H-[1,3′-bipyridin]-2-one Intermediate 124 (286 mg, 0.59 mmol) was added to a mixture of 1-(tributylstannyl)methanol (CAS Reg. No. 27490-33-1) (286 mg, 0.89 mmol), LiCl (25 mg, 0.59 mmol) and tetrakis(tri-phenylphosphine)palladium(0) (69 mg, 0.06 mmol) in 1,4-dioxane (10 mL) and the resulting suspension was stirred at 100° C. for 18 h under nitrogen. The mixture was poured into KF (6 M in water, 125 mL) and extracted with EtOAc (5×200 mL). The combined organic layers were dried (Na₂SO₄), filtered and evaporated and the obtained material was purified by preparative TLC (EtOAc) followed by preparative HPLC (PrepMethod W, gradient 15-40%) to afford the title compound (36 mg, 14%) as a white solid. HRMS (ESI) m/z [M+H]⁺ calcd for C₂₃H₂₄N₅O₂S: 434.1646, found: 434.1642. ¹H NMR (300 MHz, DMSO-d₆) δ 1.47-1.65 (2H, m), 1.84-2.05 (2H, m), 2.11-2.30 (2H, m), 4.28-4.41 (2H, m), 4.50 (2H, d), 5.13 (1H, t), 6.27 (1H, dt), 6.45 (1H, d), 6.54 (1H, d), 6.97 (2H, d), 7.33 (1H, s), 7.41 (1H, dd), 7.47 (1H, ddd), 7.57-7.62 (2H, m), 7.94 (1H, d), 8.10 (1H, d).

### Example 85

### 6′-(((1S,3S)-3-((6-(Difluoromethoxy)benzo[d]oxazol-2-yl)amino)cyclopentyl)amino)-2H-[1,3′-bipyridin]-2-one—Compound 85

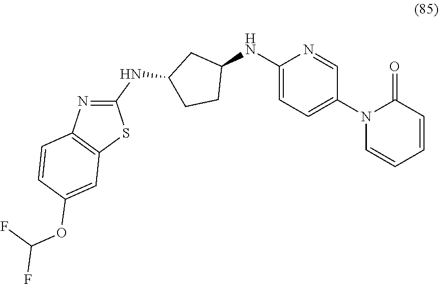 [SMILES: O=c1ccccn1-c1ccc(N[C@H]2CC[C@H](Nc3nc4ccc(OC(F)F)cc4s3)C2)nc1]

Na₂CO₃ (51 mg, 0.48 mmol) was added to a mixture of (1S,3S)—N¹-(6-(difluoromethoxy)benzo[d]oxazol-2-yl)cyclopentane-1,3-diamine Intermediate 129 (45 mg, 0.10 mmol) and 6′-fluoro-2H-[1,3′-bipyridin]-2-one Intermediate 135 (28 mg, 0.14 mmol) in DMSO (3 mL) at 20° C. and the resulting mixture was stirred at 120° C. for 16 h. The mixture was poured into water (100 mL) and extracted with EtOAc (2×100 mL). The combined organic layers were dried (Na₂SO₄), filtered and evaporated and the obtained material was purified by preparative TLC (7 M NH₃ in MeOH:DCM, 1:20) followed by preparative HPLC (PrepMethod X, gradient 25-50%) to afford the title compound (2.3 mg, 5%) as a white solid. HRMS (ESI) m/z [M+H]⁺ calcd for C₂₃H₂₂F₂N₅O₃: 454.1686, found: 454.1656. ¹H NMR (300 MHz, MeOH-d₄) δ 1.58-1.78 (2H, m), 1.99-2.18 (2H, m), 2.24-2.37 (2H, m), 4.25-4.43 (2H, m), 6.46 (1H, dt), 6.60-6.64 (2H, m), 6.72 (1H, t), 6.98 (1H, dd), 7.15 (1H, d), 7.23 (1H, d), 7.45 (1H, dd), 7.57-7.63 (2H, m), 7.95 (1H, d). ¹⁹F NMR (282 MHz, MeOH-d₄) δ −82.89.

### Example 86

### 6′-(((1S,3S)-3-((3H-Imidazo[4,5-b]pyridin-2-yl)amino)cyclopentyl)amino)-5-(2H-tetrazol-5-yl)-2H-[1,3′-bipyridin]-2-one—Compound 86

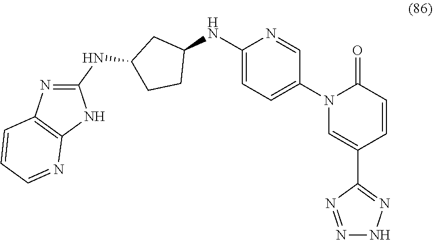 [SMILES: O=c1ccc(-c2nn[nH]n2)cn1-c1ccc(N[C@H]2CC[C@H](Nc3nc4cccnc4[nH]3)C2)nc1]

A mixture of 5-(1-(4-methoxybenzyl)-1H-tetrazol-5-yl)-6′-(((1S,3S)-3-((3-(4-methoxybenzyl)-3H-imidazo[4,5-b]pyridin-2-yl)amino)cyclopentyl)amino)-2H-[1,3′-bipyridin]-2-one and 6′-(((1S,3S)-3-((1-(4-methoxybenzyl)-1H-imidazo[4,5-b]pyridin-2-yl)amino)cyclopentyl)amino)-5-(1-(4-methoxybenzyl)-1H-tetrazol-5-yl)-2H-[1,3′-bipyridin]-2-one Intermediate 134 (80 mg, 0.11 mmol) was dissolved in TFA (3 mL) and the reaction mixture was stirred at 80° C. for 3 h. Excess TFA was removed under reduced pressure and the residue was purified by reversed phase flash chromatography on a C18 column (gradient: 20-25% of MeCN in water) followed by preparative HPLC (PrepMethod S, gradient: 6-26%) to give the title compound (1.7 mg, 3%) as a white solid. HRMS (ESI) m/z [M+H]⁺ calcd for C₂₂H₂₂N₁₁O: 456.2004, found: 456.2014.

¹H NMR (400 MHz, MeOH-d₄) δ 1.63-1.76 (2H, m), 2.06-2.18 (2H, m), 2.30-2.41 (2H, m), 4.37-4.47 (2H, m), 6.67 (1H, d), 6.76 (1H, d), 6.98 (1H, br d), 7.50 (1H, d), 7.56 (1H, dd), 7.95 (1H, d), 8.09 (1H, d), 8.22-8.25 (2H, m).

### Example 87

### 6′-(((1S,3S)-3-((6-Bromothiazolo[5,4-b]pyridin-2-yl)amino)cyclopentyl)amino)-2H-[1,3′-bipyridin]-2-one—Compound 87

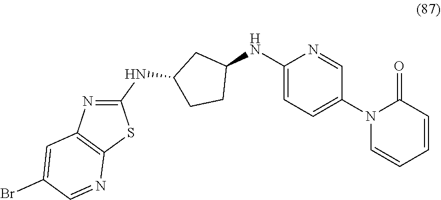 [SMILES: O=c1ccccn1-c1ccc(N[C@H]2CC[C@H](Nc3nc4cc(Br)cnc4s3)C2)nc1]

6-Bromo-2-(methylsulfonyl)thiazolo[5,4-b]pyridine Intermediate 91 (430 mg, 1.47 mmol) was added to 6′-(((1S,3S)-3-aminocyclopentyl)amino)-2H-[1,3′-bipyridin]-2-one×4HCl Intermediate 3 (733 mg, 1.76 mmol) in n-BuOH (8 mL) and the reaction mixture was stirred at 120° C. for 15 h. The solvent was removed under reduced pressure. The crude product was purified by preparative TLC (MeOH:DCM=1:10) followed by preparative HPLC (PrepMethod H, gradient 30-46%) to afford the title compound (24 mg, 3%) as a white solid. HRMS (ESI) m/z [M+H]⁺ calcd for C₂₁H₂₀BrN₆OS: 483.0596, found: 483.0592. ¹H NMR (300 MHz, DMSO-d₆) δ 1.50-1.63 (2H, m), 1.91-2.03 (2H, m), 2.11-2.25 (2H, m), 4.32-4.41 (2H, m), 6.27 (1H, dd), 6.44 (1H, dd), 6.54 (1H, d), 6.98 (1H, d), 7.41 (1H, dd), 7.47 (1H, ddd), 7.60 (1H, dd), 7.90-7.93 (2H, m), 8.18 (1H, d), 8.71 (1H, d).

### Example 88

### 6′-(((1S,3S)-3-((6-Bromothiazolo[5,4-b]pyridin-2-yl)amino)cyclopentyl)amino)-2H-[1,3′-bipyridin]-2-one—Compound 88

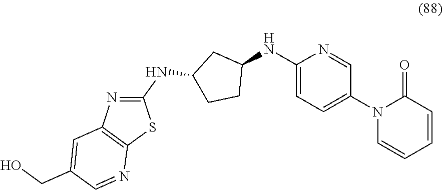 [SMILES: O=c1ccccn1-c1ccc(N[C@H]2CC[C@H](Nc3nc4cc(CO)cnc4s3)C2)nc1]

Pd(dppf)Cl₂·DCM (17 mg, 0.02 mmol) was added to a mixture of 6′-(((1S,3S)-3-((6-bromothiazolo[5,4-b]pyridin-2-yl)amino)cyclopentyl)amino)-2H-[1,3′-bipyridin]-2-one compound 87 (100 mg, 0.21 mmol) and 1-(tributylstannyl)methanol (CAS Reg. No. 27490-33-1) (100 mg, 0.31 mmol) in 1,4-dioxane (5 mL) and it was stirred at 100° C. for 15 h under nitrogen. The reaction mixture was concentrated under reduced pressure and the obtained material was purified by preparative TLC (MeOH:DCM=1:10) followed by preparative HPLC (PrepMethod K, gradient 5-27%) to afford the title compound (22 mg, 23%) as a white solid. HRMS (ESI) m/z [M+H]⁺ calcd for C₂₂H₂₃N₆O₂S: 435.1598, found: 435.1598. ¹H NMR (300 MHz, DMSO-d₆) δ 1.50-1.64 (2H, m), 1.91-2.03 (2H, m), 2.11-2.25 (2H, m), 4.30-4.43 (2H, m), 4.53 (2H, d), 5.26 (1H, t), 6.27 (1H, dt), 6.44 (1H, dd), 6.53 (1H, d), 6.98 (1H, d), 7.41 (1H, dd), 7.47 (1H, ddd), 7.58 (1H, d), 7.60 (1H, dd), 7.94 (1H, d), 8.05 (1H, d), 8.43 (1H, d).

### Example 89

### 6′-(((1S,3S)-3-(Oxazolo[4,5-b]pyridin-2-ylamino)cyclopentyl)amino)-2H-[1,3′-bipyridin]-2-one

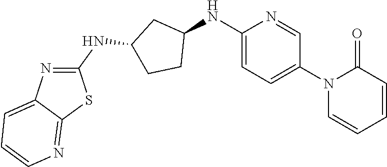 [SMILES: O=c1ccccn1-c1ccc(N[C@H]2CC[C@H](Nc3nc4cccnc4s3)C2)nc1]

2-(Methylthio)oxazolo[4,5-b]pyridine (CAS Reg. No. 169205-95-2) (0.05 g, 0.30 mmol), 6′-(((1S,3S)-3-aminocyclopentyl)amino)-2H-[1,3′-bipyridin]-2-one×HCl Intermediate 3 (92 mg, 0.30 mmol), DIPEA (0.105 mL, 0.60 mmol) and DMF (0.2 mL) were added to a vial. The vial was sealed and the reaction mixture was stirred at 100° C. for 20 h. The reaction solution was cooled to rt, diluted with DMSO (2 mL) and purified by preparative HPLC, PrepMetod C (gradient: 15-55%). The crude product was freeze dried overnight and then re-purified by flash chromatography on silica (100% EtOAc:MeOH, 9:1) to give the title compound (33 mg, 28%) as a white solid after evaporation of solvents, dissolving the residue in CH₃CN:water (1:1) and freeze-drying overnight. HRMS (ESI) m/z [M+H]⁺ calcd for C₂₁H₂₁N₆O₂: 389.1720, found: 389.1732. ¹H NMR (500 MHz, CD₃OD) δ 1.56-1.83 (2H, m), 2-2.21 (2H, m), 2.23-2.4 (2H, m), 4.34-4.47 (2H, m), 6.47 (1H, td), 6.58-6.66 (2H, m), 7.02 (1H, dd), 7.45 (1H, dd), 7.56-7.65 (3H, m), 7.95 (1H, dd), 8.08 (1H, dd).

### Assays and Methods Used

### Assay 1—Biochemical Human PCSK9 Assay

This assay measures binding of compounds to PCSK9 by homogenous time-resolved fluorescence resonance energy transfer (TR-FRET).

To determine the IC₅₀ of inhibitors of the interaction between the human PCSK9 and Alexa647 labelled small molecule, fluorescent probe displacement was monitored by homogenous TR-FRET technology. Upon binding of a terbium (Tb) cryptate conjugated anti-His mouse antibody (mAb Anti-6His Tb cryptate Gold, Cisbio) to PCSK9-TEV-His6, the displacement of the probe from PCSK9 was assessed by reduction of the proximity and FRET signal between the Tb cryptate that serves as a FRET-donor and the Alexa647 probe that serves as acceptor.

Recombinantly expressed and purified PCSK9-TEV-His6 (1 nM) was mixed with a fluorescent probe (5 nM) and anti His-Tb-cryptate antibody (0.2 nM) in assay buffer (10 mM HEPES/NaOH, pH 7.4, 150 mM NaCl, 0.005 (v/v) % Tween 20). 6 μL were subsequently added to an assay-ready plate containing 0.06 μL of controls and test compound 10 dose-response serial dilutions starting at a concentration of 10 mM (with 100 μM top and 3.2 nM lowest final concentration) by using Certus flex dispenser. The plate was sealed, and the reaction was incubated overnight (18-24h) at RT in the dark.

FRET signal quantification was achieved by PHERAstar FSX (BMG) plate reader. The created data file contained the emission of FRET acceptor channel (665 nm, probe), FRET donor channel (620 nm, Tb-cryptate, excitation at 337 nm) and the FRET ratio (665 nm/620 nm signal×10.000) which was used for calculation of a test compound's IC₅₀.

### Assay 2—Biophysical Human PCSK9 Assay

This assay measures binding of compounds to PCSK9 by SPR (“surface plasmon resonance”, a biophysical method).

The SPR binding experiments were performed on a Biacore S200 optical biosensor unit at 30° C. A Series S Sensor Chip SA that is designed to bind biotinylated molecules for interaction analysis in Biacore systems was equilibrated at room temperature prior to use. The running buffer for protein tethering and subsequent ligand binding experiments was 10 mM HEPES pH 7.4, 150 mM NaCl, 0.05% (v/v) Tween 20 pH 7.4.

For the surface tethering of PCSK9, biotinylated human PCSK9 (31-692)-Avi-His6 at a concentration of 0.5 mg/mL was used. Prior to the surface tethering, the surface was exposed to a solution of 50 mM NaOH, 500 mM NaCl via 3 consecutive injections of this solution with a contact time of 60 s and a flowrate of 10 μL min- to remove non-conjugated streptavidin. The PCSK9 protein was diluted to a concentration of 20 μg/mL using running buffer and injected with a contact time of 180-300 s and a flowrate of 10 μL min⁻¹ over a single flow channel (typically flow channel 2 or flow channel 4) with the aim to achieve protein capture levels of >5000 response units (RU). Remaining biotin binding sites were blocked via 2 consecutive injections of a 10 μM D-biotin solution in running buffer with a contact time of 60 s and a flowrate of 10 μL min⁻¹ over all flow-channels. Flow-channels 1 and 3 typically served as a reference surface throughout the subsequent binding experiments.

The binding experiments were all performed at a flow rate of 30 μL min⁻¹ and by employing the method of single-cycle kinetics. This approach involves the sequential injection of a compound concentration series without regeneration steps. A contact time between 90-150 s was selected, which was followed by a 40 min dissociation phase to allow for a proper estimation of the dissociation rate constant. Test compounds were delivered in DMSO at a concentration of 10 mM and a digital dispenser HP D300 was used to set up the compound concentration series using 6 concentrations. The tested concentrations have been 30, 100, 300, 1000, 3000 and 10000 nM. Prior to injecting any compound, the surfaces were equilibrated by injecting running buffer over them in three separate pulses. The data collection rate was set to 10 Hz.

The raw sensorgrams of the compound injections were first subjected to reference subtraction (subtracting the signal from flow channel 1 and/or 3 from the signal from channels 2 and/or 4 respectively) and then blank subtraction (subtracting the signal from injecting DMSO controls from the reference subtracted data). The resulting double-referenced sensorgrams were then fitted using a 1:1 binding interaction model using the manufactures software package to extract kinetic- and affinity data. Active compounds have been defined by creating a detectable binding signal at the highest compound concentration (10 mM) of a ≥3 RUs. Kinetic- and affinity data on active compounds are only provided for those compounds where the binding signal at the highest compound concentration (10 mM) is a ≥50% of the theoretical maximum binding signal for a 1:1 binding interaction (Rₘₐₓ, typically between 15-20 RUs) in order to enable a proper fitting of the data.

### Assay 3—PCSK9 LDL-C Uptake Assay

This assay measures for PCSK9 antagonist activity based on a test compound's capacity to restore LDL-C uptake in HepG2 cells. The assay is based on exogenous PCSK9 and LDL-C complexed with a pH-sensitive dye. Outside the cells, at neutral pH, the pHrodo Red-LDL is dimly fluorescent but upon LDLR mediated endocytosis it fluoresces brightly. PCSK9 traffics the LDLR to intracellular degradation and reduces uptake of LDL-C. Inhibition of PCSK9 reduces LDLR degradation and the increased LDL-C uptake is quantified by fluorescence microscopy.

### Preparation of Assay Reagents

Cell medium: MEM supplemented with 10% FBS, 1× NEAA and Sodium Pyruvate

Assay medium: OptiMem supplemented with penicillin/streptomycin

Cells: HepG2 (ATCC #HB-8065)

PCSK9: Sequence:
MGTVSSRRSW WPLPLLLLLL LLLGPAGARA QEDEDGDYEE
LVLALRSEED GLAEAPEHGT TATFHRCAKD PWRLPGTYVV
VLKEETHLSQ SERTARRLQA QAARRGYLTK ILHVFHGLLP
GFLVKMSGDL LELALKLPHV DYIEEDSSVF AQSIPWNLER
ITPPRYRADE YQPPDGGSLV EVYLLDTSIQ SDHREIEGRV
MVTDFENVPE EDGTRFHRQA SKCDSHGTHL AGVVSGRDAG
VAKGASMRSL RVLNCQGKGT VSGTLIGLEF IRKSQLVQPV
GPLVVLLPLA GGYSRVLNAA CQRLARAGVV LVTAAGNFRD
DACLYSPASA PEVITVGATN AQDQPVTLGT LGTNFGRCVD
LFAPGEDIIG ASSDCSTCFV SQSGTSQAAA HVAGIAAMML
SAEPELTLAE LRQRLIHFSA KDVINEAWFP EDQRVLTPNL
VAALPPSTHG AGWQLFCRTV WSAHSGPTRM ATAIARCAPD
EELLSCSSFS RSGKRRGERM EAQGGKLVCR AHNAFGGEGV
YAIARCCLLP QANCSVHTAP PAEASMGTRV HCHQQGHVLT
GCSSHWEVED LGTHKPPVLR PRGQPNQCVG HREASIHASC
CHAPGLECKV KEHGIPAPQE QVTVACEEGW TLTGCSALPG
TSHVLGAYAV DNTCVVRSRD VSTTGSTSEE AVTAVAICCR
SRHLAQASQE LQENLYFQGH HHHHH

### Step by Step Protocol for Running the Assay:

### Day 1

1. Cryopreserved HepG2 cells were thawed in assay medium and centrifuged for 5 min at 250 g. Supernatant was discarded and the cell pellet resuspended in cell medium and counted with a Nucleocounter. Cells were again centrifuged 5 min at 250 g and the pellet was resuspended to 500000 cells/mL in assay medium.
2. 20 μL of above cell mix was dispensed into black pclear TC treated 384 well plates with Multidrop Combi and left at room temperature for 20 minutes.
3. Plates were incubated at 37° C., 5% CO₂ for 24 h

### Day 2

1. Test compounds were prepared in concentration response in DMSO with a half-log dilution factor in DMSO in Echo 384 LDV plates starting at 10 mM.
2. 30 nL of above test compounds were dispensed with Echo 655 to cells for a top concentration of 10 μM
3. 10 μL PCSK9 in assay medium was dispensed to cells with Multidrop Combi for 125 nM final concentration in the wells.
4. Plates were incubated at 37° C., 5% CO₂ for 24 h

### Day 3

1. 10 μL pHrodo Red-LDL in assay medium was dispensed to cells with Multidrop Combi for 6 μg/mL
2. Plates were incubated at 37° C., 5% CO₂ and imaged with the Incucyte S3 after 4 h and 24 h

Image data was processed using Incucyte 2021, a software to identify cells and red fluorescent intensity. Screener was used to further process data. Data was normalized as % effect of the signal between median values of the on-plate controls of DMSO and 100 nM Evolocumab according to the formula % effect=−100*(x−DMSO)/(Evolocumab−DMSO) where x equals the measured signal. Concentration response data of the normalized values were fitted using a four-parameter logistic fit.

### Assay 4—In Vivo Assessment of Compound Effects on Plasma LDL-C Levels

A liver-specific human PCSK9 knock-in mouse model (hPCSK9-KI) was generated by expressing human PCSK9 under the control of a mouse albumin promoter/enhancer in C57BL/6N mice as previously described in Carreras 2019. For all in vivo studies, heterozygous male hPCSK9-KI mice and their wildtype littermates were housed individually, and heterozygous female hPCSK9-KI mice were house in groups of 4-6 mice per cage. All animals had free access to enrichment and were housed in a temperature-controlled room (22° C.) with a 12:12-h light/dark cycle. They were fed a chow diet and water ad libitum.

For in vivo assessment of test compound activity, mice were randomised into experimental groups of for example 8 mice per group. Mice were ˜12-26 weeks of age at the time of study start. On day −1, blood samples were drawn from the tail vein and baseline levels of plasma LDL-C were assessed using an enzymatic method. Baseline plasma levels of human PCSK9 were assessed by ELISA. Starting on day 0, test compounds were dosed by oral gavage at for example 15 mg/kg BID for up to 21 days. Blood samples were drawn from the tail vein and plasma levels of LDL-C and human PCSK9 were assessed using the afore mentioned methods.

Assay 5—hERG Assay (Human Ether-á-go-go-Related Gene)

This assay (human Ether-á-go-go-Related Gene) measures activity of the compounds at the potassium ion channel hERG (human Ether-á-go-go-Related Gene).

Experiments were performed on the SyncroPatch 384PE high throughput patch clamp platform at room temperature and medium resistance chips with 4 patch holes per site. Chinese hamster ovary K1 (CHO) cell lines over-expressing the ion channel of choice (hERG) were used in assay-ready format and kept in liquid nitrogen or were used from live culture. Cells were either thawed and diluted in HBSS or were detached from flasks and resuspended in HBSS. HBSS comprised 140 mM NaCl, 4 mM KCl, 10 mM HEPES and 5 mM Glucose (pH 7.4). The internal patch clamp solution was KF 120 mM, KCl 20 mM, HEPES 10 mM, EGTA 10 mM, and 25 μM Escin (pH 7.2). After the sealing process was complete, the external solution was exchanged for external patch clamp solution comprising NaCl 80 mM, KCl 4 mM, HEPES 10 mM, CaCl₂ 2 mM, MgCl₂ 1 mM, glucose 5 mM, and NMDG 60 mM (pH 7.4). All solutions were stored at room temperature, except Escin, which was stored at 4° C. All test compounds were dispensed in greiner-bio 384 well plates and tested in a 6 point cumulative assay (final DMSO concentration 0.33%). Only wells that passed previously agreed acceptance criteria for this platform were used in this analysis (30 MegaOhm seal resistance, Z prime>0.4 and current size>0.2 nA).

High Concentration hERG

Experiments were performed on the QPatchII high throughput patch clamp platform at room temperature using single holes QChips. Chinese hamster ovary K1 (CHO) cell lines over-expressing the ion channel of choice (hERG) were used from live culture. All solutions were stored at 4° C. or −20° C. All compounds were dispensed as 10 or 50 mM DMSO stocks, in 96 well plates and diluted to a format that allowed testing in a 6 point cumulative assay (final DMSO concentration 2% or 0.4% DMSO). Only wells that passed previously agreed acceptance criteria for this platform were used in this analysis (500 MegaOhm seal resistance and current size>0.2 nA, with positive controls including Verapamil and DMSO being consistent).

### Assay 6—GSK3b Assay (ThermoFisher Assay)

This assay measures the activity of the compounds at GSK3b (Glycogen synthase kinase-3 beta).

The test compounds were screened in 1% DMSO (final) in the well. For 10-point titrations, 3-fold serial dilutions are conducted from the starting concentration of 10 μM.

### Assay Protocol

Bar-coded Corning, low volume NBS, black 384-well plate
1. 2.5 μL—4× Test Compound or 100 nL 100× plus 2.4 μL kinase buffer
2. 5 μL—2× Peptide/Kinase Mixture
3. 2.5 μL—4× ATP Solution
4. 30-second plate shake
5. 60-minute Kinase Reaction incubation at room temperature
6. 5 μL—Development Reagent Solution
7. 30-second plate shake
8. 60-minute Development Reaction incubation at room temperature
9. Read on fluorescence plate reader and analyse the data

In step 2, the 2× GSK3β (GSK3 beta)/Ser/Thr (Glycogen synthase kinase-3 beta/Serine/Threonine) 09 mixture is prepared in 50 mM HEPES (4-(2-hydroxyethyl)-1-piperazineethanesulfonic acid) pH 7.5, 0.01% BRIJ-35, 10 mM MgCl₂, 1 mM EGTA (egtazic acid). The final 10 μL Kinase Reaction consists of 0.22-0.92 ng GSK3β (GSK3 beta) and 2 μM Ser/Thr 09 in 50 mM HEPES pH 7.5, 0.01% BRIJ-35, 10 mM MgCl₂, 1 mM EGTA.

In step 3 the ATP Solution is diluted to a 4× working concentration in Kinase Buffer (50 mM HEPES pH 7.5, 0.01% BRIJ-35, 10 mM MgCl₂, 1 mM EGTA).

In Step 6 the Development Reagent is diluted 1:512 in Development Buffer (10× Novel PKC Lipid Mix: 2 mg/mL Phosphatidyl Serine, 0.2 mg/mL DAG in 20 mM HEPES, pH 7.4, 0.3% CHAPS).

### Graphing Software

SelectScreen® Kinase Profiling Service uses XLfit from IDBS. The dose response curve is curve fit to model number 205 (sigmoidal dose-response model). If the bottom of the curve does not fit between −20% & 20% inhibition, it is set to 0% inhibition. If the top of the curve does not fit between 70% and 130% inhibition, it is set to 100% inhibition.

TABLE 2
Activity results in Assays 1, 2, 5, 6 and 3 for compounds 1-89

| | | | | Assay 6 | |
|---|---|---|---|---|---|
| | | | Assay 5 | (GSK3B | |
| | Assay 1 | Assay 2 | (hERG Hu | FRET-Z- | Assay 3 |
| | (PCSK9 | (PCSK9 | CHO IF | Lyte CR) | LDL-C |
| | Hu Bind | BIAcore | EPh CR- | GMean | uptake |
| | TRF) | DBA CR) | Syncro) | IC₅₀ (μM) | assay |
| Example | GMean | GMean K_{d} | GMean | ATP Conc: | GMean |
| No | IC₅₀ (nM) | (μM) | IC₅₀ (μM) | 10.000 (μM) | IC₅₀ (μM) |
| 1 | 34.3 | | >40.0 | >10.0 | |
| 2 | 11.8 | | 13.2 | >10.0 | |
| 3 | 8.2 | | 4.2 | >10.0 | |
| 4 | 32.2 | 0.049 | >40.0 | >10.0 | |
| 5 | 8.9 | | 6.0 | 5.52 | 1.715 |
| 6 | 2731 | | 2.64 | >10.0 | |
| 7 | 324 | | 12.6 | >10.0 | |
| 8 | 2081 | | 6.32 | >10.0 | |
| 9 | 17.9 | | 3.56 | 1.51 | |
| 10 | 2471 | 1.55 | 4.0 | >10.0 | |
| 11 | 28 | 0.024 | >40.0 | >10.0 | |
| 12 | 3.5 | 0.003 | >40.0 | >10.0 | 0.158 |
| 13 | 13.3 | | 27.8 | >10.0 | 1.599 |
| 14 | 25.5 | | >40.0 | 0.423 | 0.632 |
| 15 | 3.1 | 0.003 | >40.0 | >10.0 | 0.076 |
| 16 | 1.6 | | 28.4 | >10.0 | 0.081 |
| 17 | 30.5 | | >40.1 | >10.0 | |
| 18 | 2.3 | 0.003 | >40.0 | >10.0 | 0.052 |
| 19 | 4.4 | 0.004 | >40.0 | >10.0 | 0.157 |
| 20 | 603 | 0.672 | >40.1 | >10.0 | |
| 21 | 1.5 | 0.0005 | >40.0 | >10.0 | 0.100 |
| 22 | 1.2 | | 25.7 | >10.0 | 0.054 |
| 23 | 1.2 | | 24.6 | >10.0 | 0.092 |
| 24 | 0.7 | 0.0001 | 13.9 | >10.0 | 0.076 |
| 25 | 5.6 | 0.004 | >40.0 | 0.053 | 0.051 |
| 26 | 6.8 | | 19.7 | >10.0 | 0.168 |
| 27 | 49 | | >40.0 | 2.49 | |
| 28 | 5.7 | | >40.0 | >10.0 | 0.173 |
| 29 | 5.7 | | >40.0 | >10.0 | 0.068 |
| 30 | 34.5 | | 3.15 | >10.0 | |
| 31 | 25.1 | | >40.1 | >10.0 | |
| 32 | 21 | | 2.22 | >10.0 | |
| 33 | 0.9 | 0.0003 | >40.0 | >10.0 | 0.102 |
| 34 | 1.2 | 0.001 | >40.0 | >10.0 | 0.098 |
| 35 | 0.7 | | 27.4 | 7.61 | 0.108 |
| 36 | 0.6 | | 25.8 | >10.0 | 0.060 |
| 37 | 4.4 | 0.003 | >40.0 | >10.0 | 0.127 |
| 38 | 5.7 | | >40.0 | >10.0 | 0.158 |
| 39 | 6.1 | | >40.0 | >10.0 | 0.093 |
| 40 | 1.6 | 0.001 | >40.0 | 8.15 | 0.090 |
| 41 | 3.5 | | >40.0 | 0.066 | 0.186 |
| 42 | 0.9 | | >40.0 | 6.14 | 0.077 |
| 43 | 4.2 | | >40.0 | >10.0 | 0.200 |
| 44 | 4.1 | 0.004 | >40.0 | 7.91 | 0.082 |
| 45 | <0.8 | 0.001 | >40.0 | >10.0 | 0.125 |
| 46 | 4.4 | 0.006 | >40.0 | 0.425 | 0.102 |
| 47 | 1.6 | | >40.0 | 0.331 | 0.064 |
| 48 | 2.1 | | >40.0 | 0.805 | 0.100 |
| 49 | 2.8 | | >40.0 | 5.59 | 0.043 |
| 50 | 10.5 | | 32.0 | 0.024 | 0.190 |
| 51 | 2.5 | | 30.0 | 1.52 | 0.049 |
| 52 | 4.5 | | >40.0 | 0.178 | 0.590 |
| 53 | 19 | | >40.0 | 3.86 | |
| 54 | 0.7 | | >40.1 | 0.306 | 0.041 |
| 55 | 2.6 | | >40.0 | 1.77 | |
| 56 | 3.1 | | >40.0 | 3.13 | 0.395 |
| 57 | 4.3 | | >40.0 | 5.45 | 0.066 |
| 58 | 220 | | >40.0 | | |
| 59 | 3.7 | | >13.5 | 0.794 | 0.106 |
| 60 | 0.6 | 0.001 | 33.0 | | 0.041 |
| 61 | 8.9 | 0.011 | >40.0 | >10.0 | 0.100 |
| 62 | 2.2 | | >40.0 | 0.153 | 0.089 |
| 63 | 2.1 | | >40.0 | >10.0 | 0.051 |
| 64 | 1.7 | | >40.0 | >10.0 | 0.099 |
| 65 | 7.8 | | >40.0 | 8.19 | |
| 66 | 166 | | 1.94 | >10.0 | |
| 67 | 18.1 | 0.057 | 3.98 | >10.0 | |
| 68 | 21.6 | 0.3 | 6.55 | >10.0 | |
| 69 | 83 | | 1.17 | >10.0 | |
| 70 | 24.4 | | | >10.0 | |
| 71 | 2.6 | | >40.0 | 2.83 | 0.123 |
| 72 | 5 | | >40.0 | 0.01 | 0.163 |
| 73 | 4.7 | 0.005 | >40.0 | 0.192 | 0.280 |
| 74 | 173 | | >40.0 | >10.0 | |
| 75 | 16.8 | | >40.0 | >10.0 | |
| 76 | 0.4 | 0.0001 | 2.57 | >10.0 | 0.055 |
| 77 | 4.2 | | 10.4 | 4.72 | 0.204 |
| 78 | 18.9 | | 39.6 | >10.0 | 0.367 |
| 79 | 4.1 | | 4.57 | >10.0 | 0.123 |
| 80 | 5.1 | | >40.0 | 0.305 | 0.158 |
| 81 | 1.5 | | 6.12 | >10.0 | 0.108 |
| 82 | 2.3 | 0.001 | >40.1 | >10.0 | 0.124 |
| 83 | 0.5 | 0.0001 | >40.1 | >10.0 | 0.079 |
| 84 | 239 | | >40.1 | >10.0 | |
| 85 | 14.6 | | | | |
| 86 | 3.5 | | | | 0.187 |
| 87 | 1.4 | | 4.31 | 6.74 | 0.410 |
| 88 | 9.9 | | >40.1 | >10.0 | |
| 89 | 259 | | >40.1 | >10.0 | |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the compound of Formula (I) and the state of the art to which it pertains. Full citations for these references are provided below. The entirety of each of these references is incorporated herein.

| | Full Reference | DOI |
|---|---|---|
| Elbitar 2016 | Elbitar et al., Expert Opin | 10.1080/13543776.2016.1206080 |
| | Therapeutic Patents 2016 26: 1377- | |
| | 1392. | |
| Abifadel 2003 | Abifadel, et al. Nat. Genet. 2003 | 10.1038/ng1161 |
| | 34: 154-6. | |
| Abifadel 2010 | Abifadel, et al. Expert Opin. Ther. | 10.1517/13543776.2010.518615 |
| | Pat. 2010 20: 1547-71. | |
| Maxwell 2003 | Maxwell, et al. J. Lipid Res. 2003 | 10.1194/jlr.M300203-JLR200 |
| | 44: 2109-19. | |
| Persson 2009 | Persson et al. Endocrinology 2009 | 10.1210/en.2008-1281 |
| | 150: 1140-6. | |
| Langhi 2008 | Langhi et al. FEBS Lett. 2008 | 10.1016/j.febslet.2008.02.038 |
| | 582: 949-55. | |
| Robinson | Robinson et al., Artherosclerosis | 10.1016/j.atherosclerosis.2015.10.023 |
| 2015 | 2015 243: 593-597. | |
| Carreras | Carreras, A., et al. (2019). In vivo | 10.1186/s12915-018-0624-2 |
| 2019 | genome and base editing of a | |
| | human PCSK9 knock-in | |
| | hypercholesterolemic mouse model. | |
| | BMC biology 17, 4. | |
| Wuts 2006 | P. G. M. Wuts, T. W. Greene, | ISBN: 978-1118057483 |
| | Greene's Protective Groups in | |
| | Organic Synthesis 4^{th} ed., J. Wiley & | |
| | Sons, 2006. | |
| Angew. | Modern Synthetic Methods for | 10.1002/anie.200300594 |
| Chem. Int. | Copper-Mediated C(aryl)-O, C(aryl)- | |
| Ed. 2003, 42, | N, and C(aryl)-S Bond Formation, | |
| 5400-5449 | Angew. Chem. Int. Ed. 2003, 42, | |
| | 5400-5449 | |
| Chem. Rev. | Palladium-Catalyzed Cross-Coupling | 10.1021/cr00039a007 |
| 1995, 95, | Reactions of Organoboron | |
| 2457-2483 | Compounds, Chem. Rev. 1995, 95, | |
| | 2457-2483 | |
| ACS Catal. | The Stille Reaction, 38 Years Later, | 10.1021/acscatal.5b00448 |
| 2015, 5, | ACS Catal. 2015, 5, 3040-3053 | |
| 3040-3053 | | |
| Adv. Synth. | A Review on the Latest Progress of | 10.1002/adsc.202000495 |
| Catal. 2020, | Chan-Lam Coupling Reaction, Adv. | |
| 362, 3311- | Synth. Catal. 2020, 362, 3311-3331 | |
| 3331 | | |
| Chem. Rev. | Metallaphotoredox: The Merger of | 10.1021/acs.chemrev.1c00383 |
| 2022, 122, | Photoredox and Transition Metal | |
| 1485-1542 | Catalysis | |
| | Chem. Rev. 2022, 122, 1485-1542 | |

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Numbered Statements

1. A compound of Formula (I)

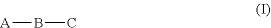

or a pharmaceutically acceptable salt, tautomeric forms or stereoisomers thereof,
wherein A is of the following formula:

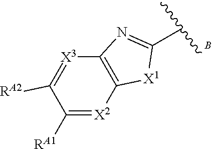

wherein the wavy line indicates the point of attachment to B;
X¹ is selected from O, S or NH;
X² is either N or C—H
X³ is either N or C—R^{A3};
wherein if X¹ is NH and X² is C—H then X³ is C—R^{A3};
when X¹ is NH, R^{A1} is X⁴ and R^{A2} is X⁵;
when X² is N and X¹ is O or S, R^{A1} is X⁴ and R^{A2} is X⁵;
when X² is CH and X¹ is S, R^{A1} is X⁴ and R^{A2} is X⁵;
when X² is CH and X¹ is O, R^{A1} is X⁵ and R^{A2} is X⁴;
X⁴ is selected from the group consisting of:
(i) H;
(ii) halo;
(iii) CN;
(iv) C₁ alkyl optionally substituted by one or more OH, CN, or one or more halo groups; or
(v) C₁ alkoxy, optionally substituted by one or more halo groups;
X⁵ is selected from the group consisting of:
(i) H;
(ii) halo;
(iii) CN;
(iv) C₁₋₆ hydrocarbon, optionally substituted by OH, CN, C₁₋₆ alkyl acyl, C₁₋₆ alkoxy or one or more halo groups;
(v) C₁₋₆ alkoxy, optionally substituted by C₁₋₆ alkyl amido, C₁₋₆ alkyl phosphonyl, or one or more halo groups;
(vi) C₁₋₆ alkylamino;
(vii) C₁₋₆ thioalkyl;
(viii) C₁₋₆ alkyl phosphinyl; or
(ix) C₁₋₆ alkyl phosphonyl;
R^{A3} is selected from the group consisting of:
(i) H;
(ii) halo;
(iii) CN;
(iv) C₁₋₆ hydrocarbon, optionally substituted by OH, CN, C₁₋₆ thioalkyl, C₁₋₆ alkoxy, C₁₋₆ alkyl acyl, C₁₋₆ acyloxy, C(═O)OH, C₁₋₆ alkyl ester, C₁₋₆ alkylamino; —C(═O)NH₂, C₁₋₆ alkyl amido, C₁₋₆ acylamido, C₁₋₆ alkyl sulfinyl, C₁₋₆ alkyl sulfonyl or one or more halo groups;
(v) C₁₋₆ alkoxy, optionally substituted by NH₂, C₄ heterocyclyl and one or more halo groups;
(vi) C₄ heterocycyl;
(vii) C₁₋₆ alkylamino, optionally substituted by CN, OH, C₄ heterocyclyl;
(viii) C₁₋₆ dialkylamino, optionally substituted by —NH₂; or
(ix) C₁₋₆ thioalkyl, optionally substituted by OH or —NH₂;
wherein B is of formula (B-1) or (B-2)
i)

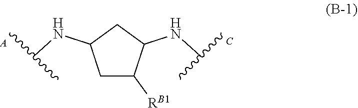

wherein the wavy lines indicate the point of attachment to A and C;
R^{B1} is H, OH, ═CHCH₂—OH, —O—C₁₋₄ alkyl or C₁₋₄ alkyl, wherein the C₁₋₄ alkyl is optionally substituted by OH or OMe;
(ii)

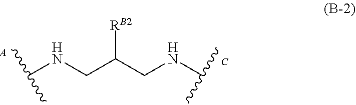

wherein the wavy lines indicate the point of attachment to A and C;
R^{B2} is C₁₋₂ alkyl-OH, CH₂C(═O)NHMe or C₁₋₃ alkyl;
wherein when R^{A1} is H or halo, R^{B2} is C₁₋₂ alkyl-OH or CH₂C(═O)NHMe,
wherein C is selected from the group consisting of C₆₋₁₀ carboaryl, C₅₋₆ heteroaryl or C₅₋₁₀ heterocyclyl, which groups are optionally substituted by:
   (i) C₆₋₁₀ carboaryl, C₄₋₁₀ carbocyclyl, C₅₋₁₀ heteroaryl, C₄₋₁₀ heterocyclyl, or C₅₋₁₀ bridged heterocyclyl, spiro C₆₋₁₂ heterocyclyl or a spiro C₆₋₁₂ carbocyclyl,
which are themselves optionally substituted by one or more of the following groups:
   a) one or two ═O groups;
   b) one or more halo groups;
   c) CN, NH₂, OH;
   d) one or more C₁₋₆ alkyl groups including branched and cyclic and with an optional substituent selected from OH or one or more halo groups;
   e) C₁₋₆ alkoxy with optional substituents of one or more halo groups;
   f) C₁₋₆ alkyl ester;
   g) C₅₋₆ heterocyclyl with an optional methyl, OH or ═O substituent;
   h) C₅₋₆ heteroaryl optionally substituted by C₁₋₆ alkyl;
   i) C₄₋₁₀ carbocyclyl with an optional methyl or ═O substituent;
   j) C₆₋₁₀ carboaryl with optional substituents of one or more halo groups;
   l) P(═O)Me₂;
   m) C(═O)OH or CH₂C(═O)OH; and/or
   n) tetrazolyl, CH₂-tetrazolyl, 5-oxo-4H-1,2,4-oxadiazol-3-yl;
   (ii) one or more groups selected from C(═O)OH, CN, halo, nitro, C₁₋₆ alkyl, C₁₋₆ thioalkyl, C₁₋₆ alkoxy, C₁₋₆ alkyl acyl, C₁₋₆ alkyl amido, di-C₁₋₆ alkyl amido, C₁₋₆ alkyl sulfonamido, and di-C₁₋₆ alkyl sulfonamido.

2. The compound of statement 1, or a pharmaceutically acceptable salt thereof wherein A is selected from one of the following formulae A1-A3:

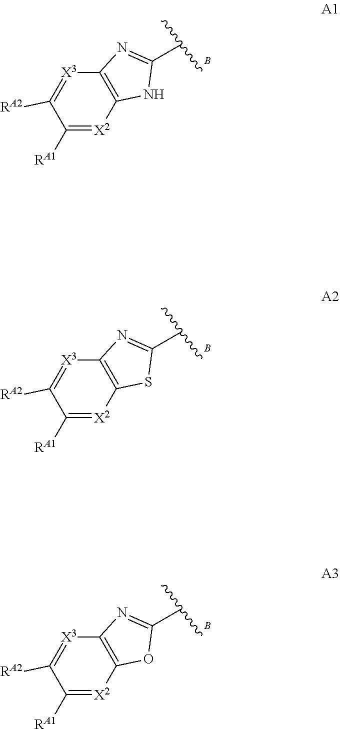

wherein the wavy line indicates the point of attachment to B and X², X³, R^{A1} and R^{A2} are as defined in statement 1.

3. The compound of either statement 1 or statement 2, or a pharmaceutically acceptable salt thereof wherein A is selected from one of the following formulae A4-A14:

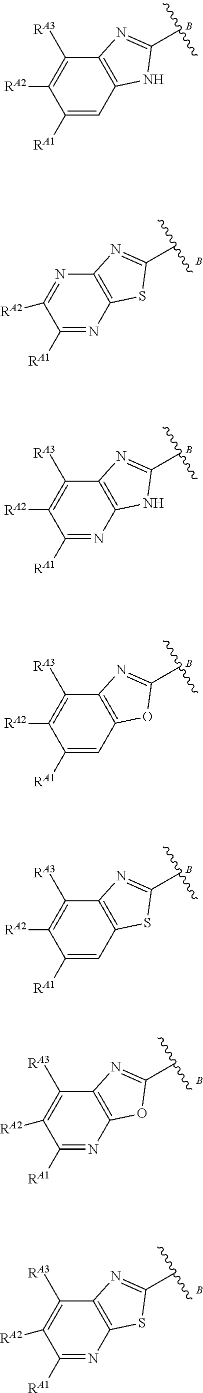

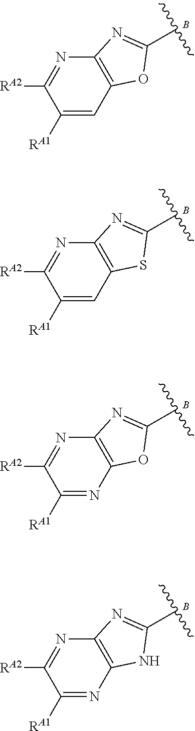

wherein the wavy line indicates the point of attachment to B and R^{A1}, R^{A2} and R^{A3} are as defined in statement 1.

4. The compound of any one of statements 1 to 3, or a pharmaceutically acceptable salt thereof wherein X⁴ is selected from:
(i) H;
(ii) halo;
(iii) CN; or
(iv) C₁ alkyl, optionally substituted by one or more OH groups or one or more halo groups.

5. The compound of any one of statements 1 to 4, or a pharmaceutically acceptable salt thereof wherein X⁴ is selected from:
(i) H;
(ii) CN;
(iii) CH₂OH;
(iv) Br; or
(v) methyl.

6. The compound of any one of statements 1 to 5, or a pharmaceutically acceptable salt thereof wherein X⁵ is selected from:
(i) H;
(ii) halo;
(iii) C₁₋₆ alkyl, optionally substituted by one or more OH or one or more halo groups;
(iv) C₁₋₆ alkoxy, optionally substituted by one or more halo groups;
(v) C₃₋₅ cycloalkyl;
(vi) C₁₋₆ thioalkyl; or
(vii) C₁₋₆ alkyl phosphinyl.

7. The compound of any one of statements 1 to 6, or a pharmaceutically acceptable salt thereof, wherein X⁵ is selected from:
(i) H;
(ii) CH₂OH;
(iii) OCF₂H;
(iv) OCF₃;
(v) CF₃;
(vi) F;
(vii) Cl;
(viii) Br;
(ix) ethyl;
(x) cyclopropyl;
(xii) methyl;
(xiii) P(═O)Me₂;
(xiv) S—CH₃; or
(xv) OMe.

8. The compound according to any one of statements 1 to 7, or a pharmaceutically acceptable salt thereof, wherein R^{A3} is selected from:
(i) H;
(ii) halo;
(iii) CN;
(iv) C₁₋₆ alkyl optionally substituted by OH, or one or more halo groups;
(v) C₂₋₆ alkenyl optionally substituted by OH, or one or more halo groups;
(vi) C₂₋₆ alkynyl optionally substituted by OH, or one or more halo groups; or
(vii) C₁₋₆ alkoxy, optionally substituted by one or more halo groups.

9. The compound according to any one of statements 1 to 8, or a pharmaceutically acceptable salt thereof, wherein R^{A3} is selected from H, Cl, Br, OMe, CH═CH₂, OCF₃, OCF₂H, F, CH₂—CF₂H, CF₃, CF₂H, CN, propargyl, CH₂-cyclopropyl.

10. The compound according to any one of statements 1 to 9, or a pharmaceutically acceptable salt thereof, wherein R^{A3} is selected from H, Cl, Br or OMe.

11. The compound according to any one of statements 1 to 10, or a pharmaceutically acceptable salt thereof, wherein A is of the following formula:

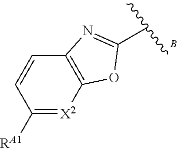

wherein X² is selected from N and C—H; and
R^{A1} is selected from H, CN, CH₂OH, OCHF₂, methyl or Br.

12. The compound according to statement 11, or a pharmaceutically acceptable salt thereof, wherein A is of the following formula:

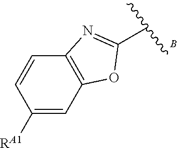

wherein R^{A1} is selected from H, CN, CH₂OH, methyl, OCHF₂, or Br.

13. The compound according to statement 11, or a pharmaceutically acceptable salt thereof, wherein A is of the following formula:

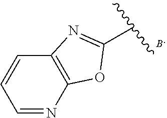

14. The compound according to any one of statements 1 to 10, or a pharmaceutically acceptable salt thereof, wherein A is of the following formula (A2):

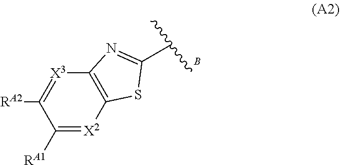

wherein X² is selected from N or C—H;
when X² is N, X³ is either N or C—R^{A3};
when X² is C—H, X³ is C—R^{A3} or N;
R^{A1} is selected from H, methyl or Br;
R^{A2} is H, CN or CH₂OH;
R^{A3} is H.

15. The compound according to any one of statements 1 to 10, or a pharmaceutically acceptable salt thereof, wherein A is of the following formula:

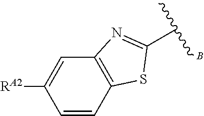

wherein R^{A2} is selected from:
(i) H;
(ii) halo;
(iii) C₁₋₆ alkyl, optionally substituted by one or more OH or one or more halo groups,
(iv) C₁₋₆ alkoxy, optionally substituted by one or more halo groups;
(v) C₃₋₅ cycloalkyl;
(vi) C₁₋₆ thioalkyl;
(vii) C₁₋₆ alkyl phosphinyl; or
(viii) CN.

16. The compound according to any one of statements 1 to 10, or a pharmaceutically acceptable salt thereof, wherein A is of the following formula:

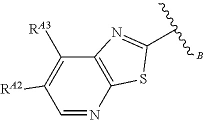

wherein R^{A3} is selected from H, halo or OMe and R^{A2} is selected from H, Br and CH₂OH.

17. The compound according to any one of statements 1 to 10, or a pharmaceutically acceptable salt thereof, wherein A is of the following formula:

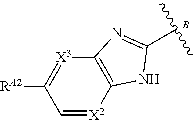

wherein
X² is N or C—H;
when X² is N, X³ is either N or C—R^{A3};
when X² is C—H, X³ is C—R^{A3};
wherein R^{A3} is selected from H, Cl, Br or OMe;
R^{A2} is selected from H, CH₂OH, OCH₃, OCHF₂, OCF₃, CF₃, F, Cl, Br, ethyl, cyclopropyl, methyl, —P(═O)Me₂ or —S—CH₃.

18. The compound according to any one of statements 1 to 10, or a pharmaceutically acceptable salt thereof, wherein A is the following formula:

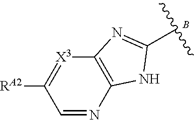

wherein
X³ is N or C—R^{A3};
wherein R^{A3} is selected from H, Cl, Br or OMe;
R^{A2} is selected from H, CH₂OH, OCH₃, OCHF₂, OCF₃, CF₃, F, Cl, Br, ethyl, cyclopropyl, methyl, —P(═O)Me₂ or —S—CH₃.

19. The compound of any one of statements 1-18, or a pharmaceutically acceptable salt thereof, wherein A is selected from the following compounds:

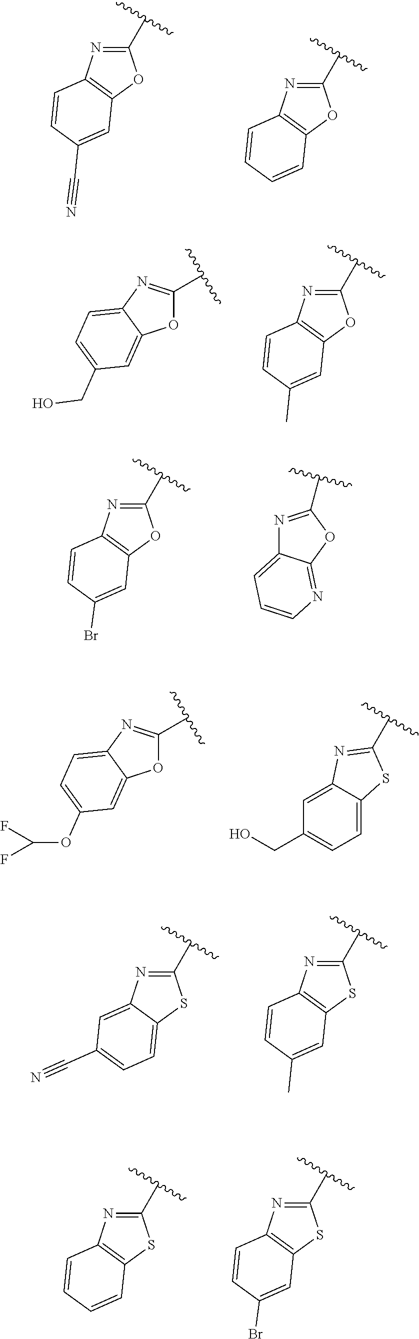

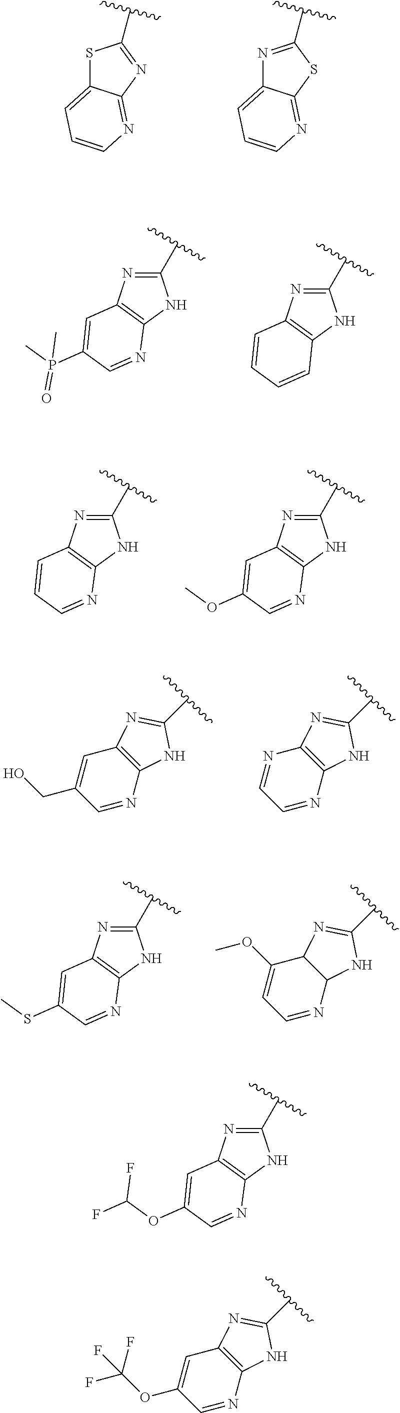

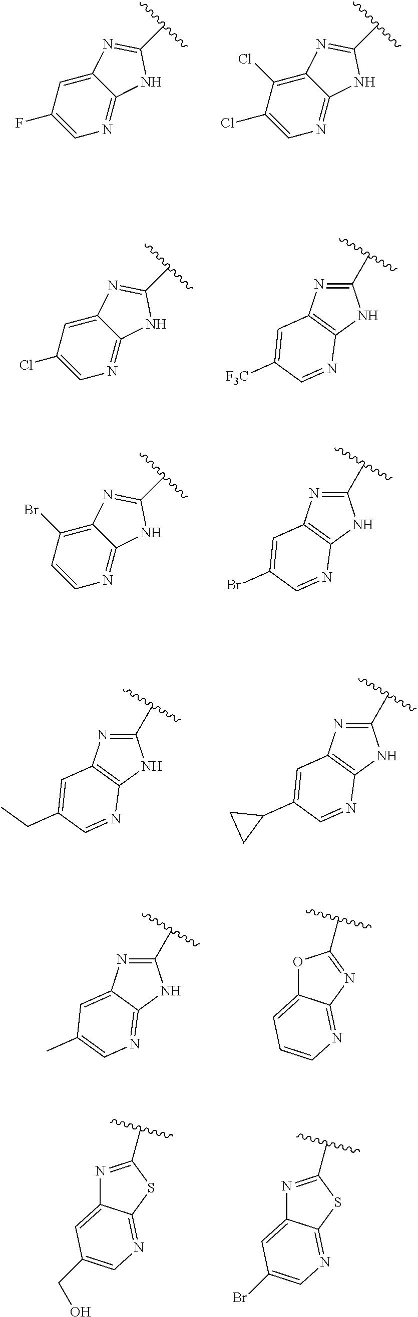

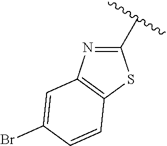 [SMILES: *c1nc2cc(Br)ccc2s1]

wherein the wavy line indicates the point of attachment to B.

20. The compound of any one of statements 1 to 19 or a pharmaceutically acceptable salt thereof, wherein B is of formula (B-1)

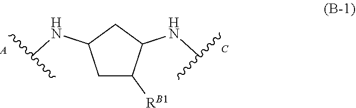

wherein the wavy lines indicate the point of attachment to A and C;
wherein R^{B1} is selected from the group consisting of H, OH, OMe, —O-ethyl, —CH₂OH, —CH₂CH₂OH and ═CHCH₂—OH.

21. The compound of statements 20 or a pharmaceutically acceptable salt thereof, wherein R^{B1} is selected from the group consisting of —CH₂OH, —CH₂CH₂OH and ═CHCH₂—OH.

22. The compound of any one of statements 1 to 20 or a pharmaceutically acceptable salt thereof, wherein B is of the following formula (B-1a):

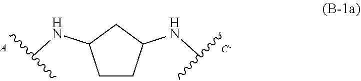

23. The compound of any one of statements 1 to 20 or statement 22 or a pharmaceutically acceptable salt thereof, wherein B is of the following formula (B-1 b):

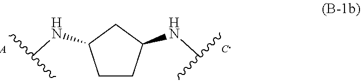

24. The compound of any one of statements 1 to 23 or a pharmaceutically acceptable salt thereof, wherein C is an optionally substituted C₅₋₆ heteroaryl.

25. The compound of any one of statements 1 to 24 or a pharmaceutically acceptable salt thereof, wherein C is an optionally substituted pyridinyl, pyrazinyl or pyrimidinyl, wherein the optional substituents are selected from C₆₋₁₀ carboaryl, C₄₋₁₀ carbocyclyl, C₅₋₁₀ heteroaryl, C₅₋₁₀ heterocyclyl, C₅₋₁₀ bridged heterocyclyl, spiro C₆₋₁₂heterocyclyl or a spiro C₆₋₁₂ carbocyclyl, which are themselves optionally substituted by one or more of the following groups:
a) one or two ═O groups;
b) one or more halo groups;
c) CN, NH₂ or OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆alkyl ester;
g) C₅₋₆ heterocyclyl with an optional methyl, OH or ═O substituent;
h) C₅₋₆ heteroaryl optionally substituted by C₁₋₆ alkyl;
i) C₄₋₁₀ carbocyclyl with an optional methyl or ═O substituent;
j) C₆₋₁₀ carboaryl with optional substituents of one or more halo groups;
l) P(═O)Me₂;
m) C(═O)OH or CH₂C(═O)OH; and/or
n) tetrazolyl, CH₂-tetrazolyl, 5-oxo-4H-1,2,4-oxadiazol-3-yl.

26. The compound of any one of statements 1 to 25 or a pharmaceutically acceptable salt thereof, wherein C is substituted by C₆₋₁₀ carboaryl, C₅₋₁₀ heteroaryl, or C₅₋₁₀ heterocyclyl, which are themselves optionally substituted by:
a) one or two ═O groups;
b) one or more halo groups, CN or NH₂;
c) one or more C₁₋₆ alkyl groups, C₁₋₆ alkoxy, or C₁₋₆ alkyl ester, wherein each group is optionally substituted by one or more halo groups;
d) C₅₋₆ heterocyclyl or C₅₋₆ heteroaryl with an optional methyl substituent;
e) C₆₋₁₀ carboaryl optionally substituted by one or more halo atoms;
f) C(═O)OH or CH₂C(═O)OH; or
g) tetrazolyl, CH₂-tetrazolyl, 5-oxo-4H-1,2,4-oxadiazol-3-yl.

27. The compound of any one of statements 1 to 23 or a pharmaceutically acceptable salt thereof, wherein C is a C₆₋₁₀ carboaryl, C₅₋₆ heteroaryl or C₅₋₁₀ heterocyclyl, wherein each group is substituted by methyl.

28. The compound of statement 27 or a pharmaceutically acceptable salt thereof, wherein the methyl group on C is at the para position.

29. The compound of any one of statements 1 to 23 or a pharmaceutically acceptable salt thereof, wherein C is of the formula (C-1):

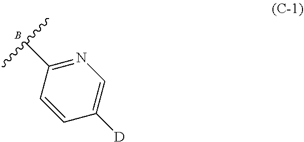

wherein D is C₆₋₁₀ carboaryl, C₅₋₁₀ heteroaryl or C₅₋₁₀ heterocyclyl, each which are themselves optionally substituted by:
i) one or two ═O groups;
ii) one or two C₁₋₄ alkyl groups which can be branched;
iii) OMe;
iv) piperazinyl, or pyrimidinyl optionally substituted by methyl;
v) C(═O)OH;
vi) Cl;
vii) one or more F;
viii) phenyl, optionally substituted by one or more fluoro;
ix) CN;
x) CF₃;
xi) OCF₃;
xii) OCHF₂;
xiii) tetrazolyl, pyrazolyl, triazolyl;
xiv) NH₂;
xv) pyridinyl;
xvi)CH₂OH;
xvii) OH;
xviii) P(═O)Me₂;
xix) CHF₂; or
xx) CH₂CF₃.

30. The compound of statement 29 or a pharmaceutically acceptable salt thereof, wherein D is either an optionally substituted phenyl or an optionally substituted 6 membered heteroaryl which contains 1 or 2 N atoms, one of which is bonded to the pyridyl in (C-1), and which is substituted by ═O at the ortho position, wherein the optional substituents are selected from:
i) methyl;
ii) OMe;
iii) pyrimidinyl substituted by methyl;
iv) C(═O)OH;
v) Cl;
vi) CN;
vii) one or more F groups;
viii) CF₃;
ix) —O—CF₃; or
x) —OCHF₂.

31. The compound of either statement 29 or 30 or a pharmaceutically acceptable salt thereof, wherein D is of the formula (D-1):

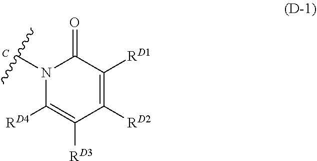

wherein one or two of R^{D1}, R^{D2}, R^{D3} and R^{D4} are selected from:
i) C₁₋₆ alkyl, optionally substituted by one or more halo groups;
ii) C₁₋₆ alkoxy, optionally substituted by one or more halo groups;
iii) C₅₋₆ heterocyclyl or C₅₋₆ heteroaryl with an optional methyl substituent;
iv) C(═O)OH or CH₂C(═O)OH;
v) ═O, halo, NH₂ or CN;
vi) phenyl, optionally substituted by one or more halo atoms;
   and the rest are H; or
   wherein R^{D3} and R^{D4} form an optionally substituted 6 membered carboaromatic, heterocyclic or heteroaromatic ring, wherein the optional substituents are selected from OH, methyl, OMe, halo and C(═O)OH; or
   wherein R^{D1} and R^{D2} form an optionally substituted 5 or 6 membered carboaromatic, heterocyclic or heteroaromatic ring, wherein the optional substituents are selected from OH, methyl, OMe, halo and C(═O)OH; or
   wherein R^{D2} and R^{D3} form an optionally substituted 5 or 6 membered carboaromatic, heterocyclic or heteroaromatic ring, wherein the optional substituents are selected from halo; CN; OH; P(═O)Me₂; C(═O)OH; C₁₋₆ alkoxy optionally substituted by one or more halo groups; or C₁₋₆ alkyl optionally substituted by one or more halo groups; or
   wherein R^{D1}, R^{D2}, R^{D3} and R^{D4} are all H.

32. The compound of statement 31 or a pharmaceutically acceptable salt thereof, wherein one or two of R^{D1}, R^{D2}, R^{D3} and R^{D4} are selected from:
i) methyl;
ii) OMe;
iii) pyrimidinyl substituted by methyl;
iv) tetrazolyl;
v) C(═O)OH;
vi) halo;
vii) CF₃;
viii) CN;
ix) O—CF₃;
x) —OCHF₂;
and the rest of R^{D1}, R^{D2}, R^{D3} and R^{D4} are H.

33. The compound of statement 31 or a pharmaceutically acceptable salt thereof, wherein R^{D1}, R^{D2}, R^{D3} and R^{D4} are H.

34. The compound of statement 31 or a pharmaceutically acceptable salt thereof, wherein R^{D3} is selected from the group consisting of: H; OMe; C(═O)OH; Cl; CN; OCH₂F; tetrazolyl; or pyrimidinyl optionally substituted by methyl; and wherein R^{D1}, R^{D2} and R^{D4} are all H.

35. The compound of statement 31 or a pharmaceutically acceptable salt thereof, wherein R^{D1} is selected from H, methyl, OMe, Cl, OCF₃, CF₃, OCHF₂, and CN, and wherein R^{D2}, R^{D3} and R^{D4} are all H.

36. The compound of statement 31 or a pharmaceutically acceptable salt thereof, wherein R^{D3} and R^{D4} form an unsubstituted phenyl ring or an unsubstituted pyridine ring.

37. The compound of statement 31, or a pharmaceutically acceptable salt thereof, wherein R^{D1} and R^{D2} form an unsubstituted 5 membered heterocycle or heteroaromatic ring.

38. The compound of statement 31 or 37, or a pharmaceutically acceptable salt thereof, wherein R^{D1} and R^{D2} form an unsubstituted thiophene.

39. The compound of statement 29 or a pharmaceutically acceptable salt thereof, wherein C is of the formula (C-1) and D is an optionally substituted phenyl or piperidyl, wherein there are one or two optional substituents selected from F, OCHF₂, OCF₃ or OMe.

40. The compound of statement 29 or a pharmaceutically acceptable salt thereof, wherein D is of the formula (D-2):

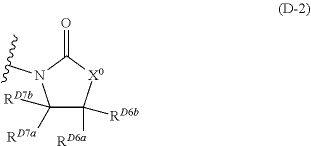

wherein X^{D} is NR^{D5a} or CR^{D5a}R^{D5b};
R^{D5a} is selected from H or methyl;
either R^{D5b} and R^{D6b} are both H or together they are —CH₂—;
R^{D6a} is selected from H, ═O, methyl, CH₂OH or C(═O)OH, wherein when R^{D6a} is ═O, R^{D6b} is absent;
R^{D7a} is selected from H, ═O, methyl, CH₂OH or C(═O)OH;
R^{D7b} is H, wherein when R^{D7a} is ═O, R^{D7b} is absent;
or wherein R^{D6a} and R^{D7a} together form a phenyl ring or a C₆ heteroaromatic ring which is optionally substituted by CN, P(═O)Me₂ or C(═O)OH and R^{D6b} and R^{D7b} are absent.

41. The compound of statement 40 or a pharmaceutically acceptable salt thereof, wherein:
a) when R^{D6a} and R^{D6b} are H, R^{D7a} is selected from ═O, C(═O)OH or CH₂OH, and R^{D7b} is H or when R^{D7a} is ═O, R^{D7b} is absent;
b) when R^{D7a} is H, R^{D7b} is H, R^{D6a} is ═O and R^{D6b} is absent.

42. The compound of statement 40 or a pharmaceutically acceptable salt thereof, wherein R^{D6a} and R^{D7a} together form a phenyl ring or a C₆ heteroaromatic ring which is optionally substituted by CN, C(═O)OH or P(═O)Me₂ and R^{D6b} and R^{D7b} are absent.

43. The compound of statement 40 or a pharmaceutically acceptable salt thereof, wherein R^{D6a} and R^{D7a} form an unsubstituted phenyl ring or an unsubstituted pyridine ring and R^{D6b} and R^{D7b} are absent.

44. The compound of statement 29 or a pharmaceutically acceptable salt thereof, wherein D is of the formula (D-3):

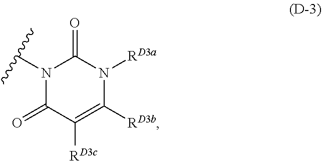

wherein R^{D3a} is selected from H and methyl;
wherein either: R^{D3b} and R^{D3c} are independently selected from H; halo; ON; OH; P(═O)Me₂; C(═O)OH; C₁₋₆ alkoxy optionally substituted by one or more halo groups; or C₁₋₆ alkyl optionally substituted by one or more halo groups; or
R^{D3b} and R^{D3c} together form a C₅₋₆ heteroaryl ring.

45. The compound of statement 29 or a pharmaceutically acceptable salt thereof, wherein D is selected from the following groups:

| Name | Structure |
|---|---|
| 2-oxo-1-pyridyl | 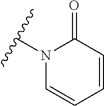 [SMILES: *n1ccccc1=O] |
| 3-methyl-2- oxo-1-pyridyl | 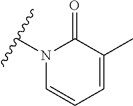 [SMILES: *n1cccc(C)c1=O] |
| 3-methoxy-2- oxo-1-pyridyl | 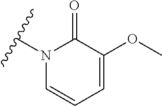 [SMILES: *n1cccc(OC)c1=O] |
| 5-methoxy-2- oxo-1-pyridyl | 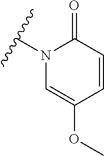 [SMILES: *n1cc(OC)ccc1=O] |
| 5-chloro-2-oxo- 1-pyridyl | 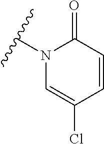 [SMILES: *n1cc(Cl)ccc1=O] |
| 2-oxo-1,8- naphthyridin-1- yl | 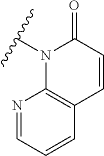 [SMILES: *n1c(=O)ccc2cccnc21] |
| 2-oxo-1-quinolyl | 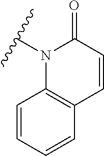 [SMILES: *n1c(=O)ccc2ccccc21] |
| 3-methyl-2- oxo- benzimidazol- 1-yl | 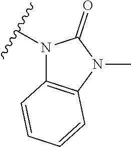 [SMILES: *n1c(=O)n(C)c2ccccc21] |
| 3-methyl-2,4- dioxo- imidazolidin-1-yl | 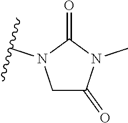 [SMILES: *N1CC(=O)N(C)C1=O] |
| 3-methyl-2,5- dioxo- imidazolin-1- yl | 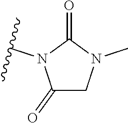 [SMILES: *N1C(=O)CN(C)C1=O] |
| 2,4- dioxoimidazolidin- 1-yl | 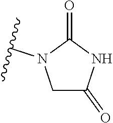 [SMILES: *N1CC(=O)NC1=O] |
| 2,5- dioxoimidazolidin- 1-yl | 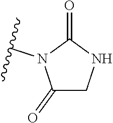 [SMILES: *N1C(=O)CNC1=O] |
| 3-chloro-2-oxo- 1-pyridyl | 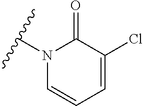 [SMILES: *n1cccc(Cl)c1=O] |
| 3-cyano-2-oxo- 1-pyridyl | 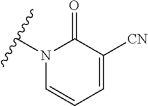 [SMILES: *n1cccc(C#N)c1=O] |
| 5-cyano-2-oxo- 1-pyridyl | 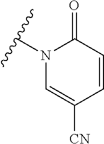 [SMILES: *n1cc(C#N)ccc1=O] |
| 3-fluoro-2- oxopyridin- 1(2H)-yl | 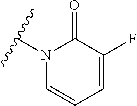 [SMILES: *n1cccc(F)c1=O] |
| 5-methyl-2-oxo- 1-pyridyl | 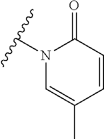 [SMILES: *n1cc(C)ccc1=O] |
| 5-carboxy-2- oxo-1-pyridyl | 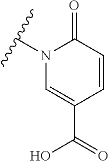 [SMILES: *n1cc(C(=O)O)ccc1=O] |
| 2-oxo-5-(1H- pyrazol-4-yl)-1- pyridyl | 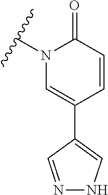 [SMILES: *n1cc(-c2cn[nH]c2)ccc1=O] |
| 2-oxo-5-(1H- triazol-4-yl)-1- pyridyl | 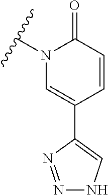 [SMILES: *n1cc(-c2c[nH]nn2)ccc1=O] |
| 5-cyano-3- methyl-2-oxo- benzimidazol-1- yl | 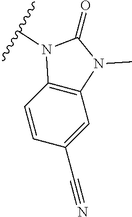 [SMILES: *n1c(=O)n(C)c2cc(C#N)ccc21] |
| 5-carboxy-3- methyl-2-oxo- benzimidazol- 1-yl | 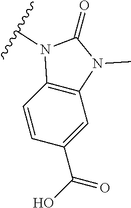 [SMILES: *n1c(=O)n(C)c2cc(C(=O)O)ccc21] |
| 6-carboxy-3- methyl-2-oxo- benzimidazol-1- yl | 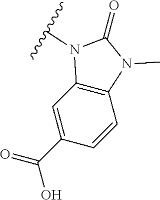 [SMILES: *n1c(=O)n(C)c2ccc(C(=O)O)cc21] |
| (6- dimethylphos- phoryl-3-methyl- 2-oxo- bezimidazol- 1-yl) | 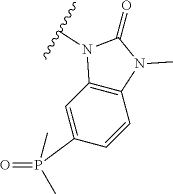 [SMILES: *n1c(=O)n(C)c2ccc(P(C)(C)=O)cc21] |
| 2-oxo-3H- benzimidazol-1- yl | 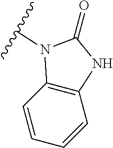 [SMILES: *n1c(=O)[nH]c2ccccc21] |
| 2-oxo-1H- imidazo[4,5- b]pyridin-3-yl | 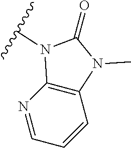 [SMILES: *n1c(=O)n(C)c2cccnc21] |
| 6-oxopyridazin- 1-yl | 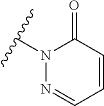 [SMILES: *n1ncccc1=O] |
| 6- oxopyrimidin- 1-yl | 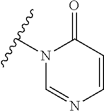 [SMILES: *n1cnccc1=O] |
| 1-methyl-2-oxo- 3-pyridyl | 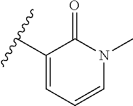 [SMILES: *c1cccn(C)c1=O] |
| 2-oxo-3- (trifluoromethyl)- 1-pyridyl | 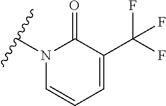 [SMILES: *n1cccc(C(F)(F)F)c1=O] |
| 2-oxo-3- (trifluoromethoxy)- 1-pyridyl | 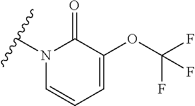 [SMILES: *n1cccc(OC(F)(F)F)c1=O] |
| 2-oxo-5-(1H- tetrazol-5-yl)- 1-pyridyl | 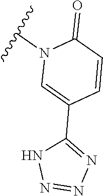 [SMILES: *n1cc(-c2nnn[nH]2)ccc1=O] |
| 2-fluorophenyl | 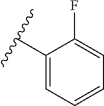 [SMILES: *c1ccccc1F] |
| 5-(2- methylpyrimidin- 5-yl)-2-oxo- 1-pyridyl | 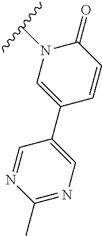 [SMILES: *n1cc(-c2cnc(C)nc2)ccc1=O] |
| 2- (difluoromethoxy)- 6-fluoro- phenyl | 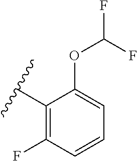 [SMILES: *c1c(F)cccc1OC(F)F] |
| 2- methoxyphenyl | 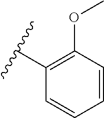 [SMILES: *c1ccccc1OC] |
| 2,6- difluorophenyl | 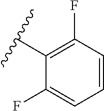 [SMILES: *c1c(F)cccc1F] |
| 2-fluoro-6- methoxy- phenyl | 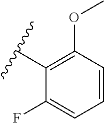 [SMILES: *c1c(F)cccc1OC] |
| 3-fluoro-2- pyridyl | 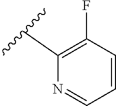 [SMILES: *c1ncccc1F] |
| 7- oxothieno[2,3- c]pyridin-6-yl | 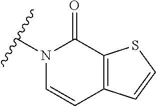 [SMILES: *n1ccc2ccsc2c1=O] |
| 2-fluoro-6- (trifluoromethoxy) phenyl | 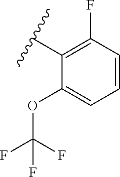 [SMILES: *c1c(F)cccc1OC(F)(F)F] |
| [5- (difluoromethoxy)- 2-oxo-1- pyridyl] | 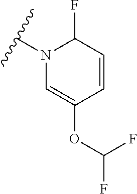 [SMILES: *N1C=C(OC(F)F)C=CC1F] |
| 3- (difluoromethoxy)- 2-oxo-1- pyridyl | 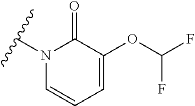 [SMILES: *n1cccc(OC(F)F)c1=O] |
| 3-methyl-2,6- dioxo- pyrimidin-1-yl | 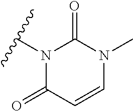 [SMILES: *n1c(=O)ccn(C)c1=O] |
| 2,4-dioxo-1H- thieno[3,2- d]pyrimidin-3-yl | 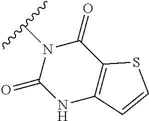 [SMILES: *n1c(=O)[nH]c2ccsc2c1=O] |
| 2-oxo-5-(2H- tetrazol-5-yl)- 1-pyridyl | 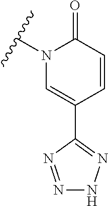 [SMILES: *n1cc(-c2nn[nH]n2)ccc1=O] |

46. The compound of statement 29 or 45, or a pharmaceutically acceptable salt thereof, wherein D is selected from the following groups:

| Name | Structure |
|---|---|
| 2-oxo-1-pyridyl | 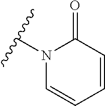 [SMILES: *n1ccccc1=O] |
| 3-methyl-2- oxo-1-pyridyl | 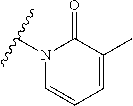 [SMILES: *n1cccc(C)c1=O] |
| 3-methoxy-2- oxo-1-pyridyl | 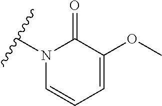 [SMILES: *n1cccc(OC)c1=O] |
| 5-methoxy-2- oxo-1-pyridyl | 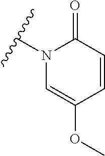 [SMILES: *n1cc(OC)ccc1=O] |
| 5-chloro-2-oxo- 1-pyridyl | 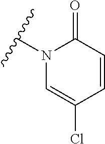 [SMILES: *n1cc(Cl)ccc1=O] |
| 2-oxo-1,8- naphthyridin-1- yl | 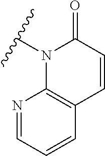 [SMILES: *n1c(=O)ccc2cccnc21] |
| 2-oxo-1-quinolyl | 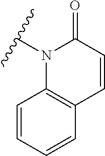 [SMILES: *n1c(=O)ccc2ccccc21] |
| 3-methyl-2- oxo- benzimidazol- 1-yl | 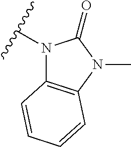 [SMILES: *n1c(=O)n(C)c2ccccc21] |
| 3-methyl-2,4- dioxo- imidazolidin-1-yl | 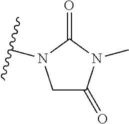 [SMILES: *N1CC(=O)N(C)C1=O] |
| 3-methyl-2,5- dioxo- imidazolin-1- yl | 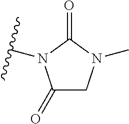 [SMILES: *N1C(=O)CN(C)C1=O] |
| 3-chloro-2-oxo- 1-pyridyl | 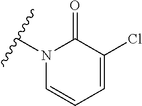 [SMILES: *n1cccc(Cl)c1=O] |
| 3-cyano-2-oxo- 1-pyridyl | 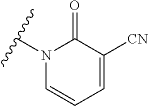 [SMILES: *n1cccc(C#N)c1=O] |
| 5-cyano-2-oxo- 1-pyridyl | 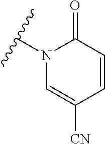 [SMILES: *n1cc(C#N)ccc1=O] |
| 3-fluoro-2- oxopyridin- 1(2H)-yl | 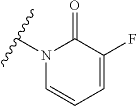 [SMILES: *n1cccc(F)c1=O] |
| 2-fluoro-6- methoxy-phenyl | 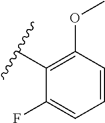 [SMILES: *c1c(F)cccc1OC] |
| 5-carboxy-2- oxo-1-pyridyl | 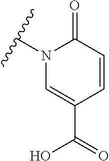 [SMILES: *n1cc(C(=O)O)ccc1=O] |
| 2-oxo-3H- benzimidazol-1- yl | 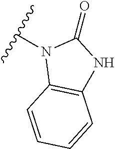 [SMILES: *n1c(=O)[nH]c2ccccc21] |
| 2-oxo-1H- imidazo[4,5- b]pyridin-3-yl | 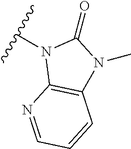 [SMILES: *n1c(=O)n(C)c2cccnc21] |
| 6-oxopyridazin- 1-yl | 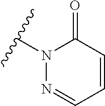 [SMILES: *n1ncccc1=O] |
| 6- oxopyrimidin- 1-yl | 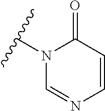 [SMILES: *n1cnccc1=O] |
| 1-methyl-2-oxo- 3-pyridyl | 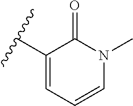 [SMILES: *c1cccn(C)c1=O] |
| 2-oxo-3- (trifluoromethoxy)- 1-pyridyl | 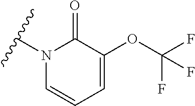 [SMILES: *n1cccc(OC(F)(F)F)c1=O] |
| 2-fluorophenyl | 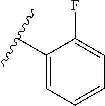 [SMILES: *c1ccccc1F] |
| 5-(2- methylpyrimidin- 5-yl)-2-oxo- 1-pyridyl | 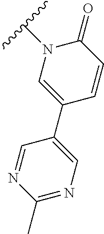 [SMILES: *n1cc(-c2cnc(C)nc2)ccc1=O] |
| 2- (difluoromethoxy)- 6-fluoro- phenyl | 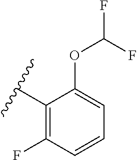 [SMILES: *c1c(F)cccc1OC(F)F] |
| 2- methoxyphenyl | 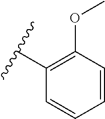 [SMILES: *c1ccccc1OC] |
| 2,6- difluorophenyl | 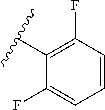 [SMILES: *c1c(F)cccc1F] |
| 3-fluoro-2- pyridyl | 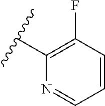 [SMILES: *c1ncccc1F] |
| 7- oxothieno[2,3- c]pyridin-6-yl | 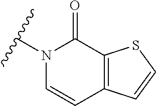 [SMILES: *n1ccc2ccsc2c1=O] |
| 2-oxo-5-(2H- tetrazol-5-yl)- 1-pyridyl | 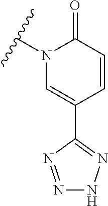 [SMILES: *n1cc(-c2nn[nH]n2)ccc1=O] |
| 2-fluoro-6- (trifluoromethoxy) phenyl | 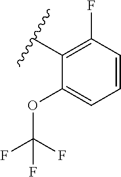 [SMILES: *c1c(F)cccc1OC(F)(F)F] |
| [5- (difluoromethoxy)- 2-oxo-1- pyridyl] | 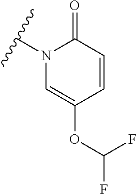 [SMILES: *n1cc(OC(F)F)ccc1=O] |
| 3- (difluoromethoxy)- 2-oxo-1- pyridyl | 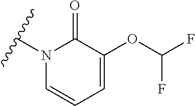 [SMILES: *n1cccc(OC(F)F)c1=O] |
| 3-methyl-2,6- dioxo- pyrimidin-1-yl | 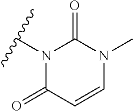 [SMILES: *n1c(=O)ccn(C)c1=O] |
| 2,4-dioxo-1H- thieno[3,2- d]pyrimidin-3-yl | 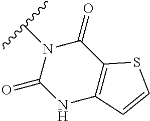 [SMILES: *n1c(=O)[nH]c2ccsc2c1=O] |
| 2-oxo-3- (trifluoromethyl)- 1-pyridyl | 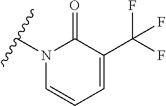 [SMILES: *n1cccc(C(F)(F)F)c1=O] |
| 2,4- dioximidazolidin- 1-yl | 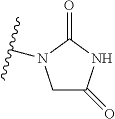 [SMILES: *N1CC(=O)NC1=O] |

47. The compound of any one of statements 1 to 23 or a pharmaceutically acceptable salt thereof, wherein C is of the formula (C-2):

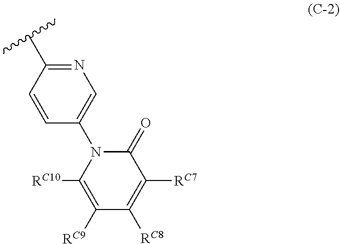

wherein one of R^{C7}, R^{C8}, R^{C9} and R^{C10} are selected from the group consisting of: methyl; OMe; piperazinyl or pyrimidinyl optionally substituted by methyl; C(═O)OH; Cl; F; pyrazolyl; triazolyl; tetrazole; optionally substituted phenyl (wherein the optional substituent is methyl or halo); CN; CF₃; OCHF₂, O—CF₃; and the rest of R^{C7}, R^{C8}, R^{C9} and R^{C10} are H; or
R^{C9} and R^{C10} form a phenyl or 6 membered heteroaromatic ring optionally substituted by methyl, and R^{C7} and R^{C8} are both H; or
wherein R^{C7}, R^{C8}, R^{C9} and R^{C10} are all H.

48. The compound of statement 47 or a pharmaceutically acceptable salt thereof, wherein:
a) all of R^{C7}, R^{C8}, R^{C9} and R^{C10} are H; or
b) R^{C9} is selected from H, CN, OMe, Cl, OCHF₂, tetrazolyl, pyrimidinyl substituted by methyl, C(═O)OH and R^{C7}, R^{C8} and R^{C10} are all H; or
c) R^{C7} is selected from H, methyl, Cl, OMe, CN, CF₃, OCHF₂, OCF₃ and R^{C8}, R^{C9} and R^{C10} are all H.

49. The compound of any one of statements 1 to 23 or a pharmaceutically acceptable salt thereof, wherein C is selected from the group consisting of:

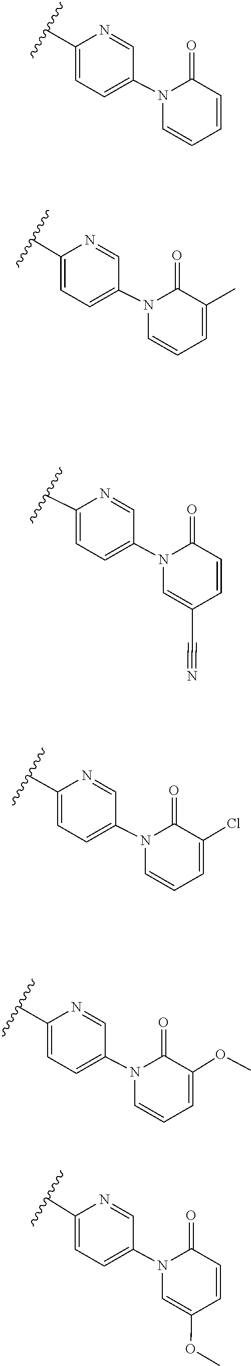 [SMILES: *c1ccc(-n2ccccc2=O)cn1 ; *c1ccc(-n2cccc(C)c2=O)cn1 ; *c1ccc(-n2cc(C#N)ccc2=O)cn1 ; *c1ccc(-n2cccc(Cl)c2=O)cn1 ; *c1ccc(-n2cccc(OC)c2=O)cn1 ; *c1ccc(-n2cc(OC)ccc2=O)cn1]

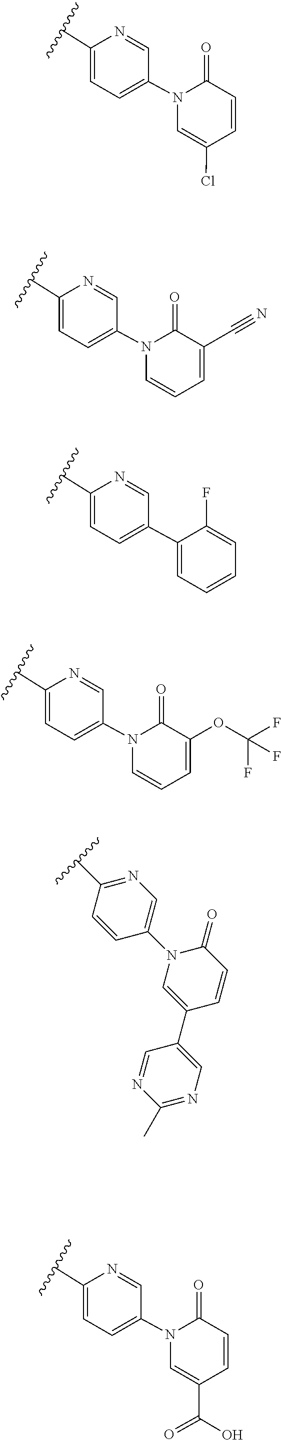 [SMILES: *c1ccc(-n2cc(Cl)ccc2=O)cn1 ; *c1ccc(-n2cccc(C#N)c2=O)cn1 ; *c1ccc(-c2ccccc2F)cn1 ; *c1ccc(-n2cccc(OC(F)(F)F)c2=O)cn1 ; *c1ccc(-n2cc(-c3cnc(C)nc3)ccc2=O)cn1 ; *c1ccc(-n2cc(C(=O)O)ccc2=O)cn1]

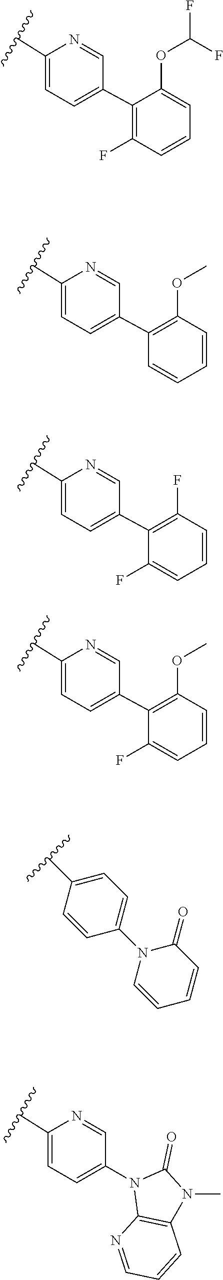 [SMILES: *c1ccc(-c2c(F)cccc2OC(F)F)cn1 ; *c1ccc(-c2ccccc2OC)cn1 ; *c1ccc(-c2c(F)cccc2F)cn1 ; *c1ccc(-c2c(F)cccc2OC)cn1 ; *c1ccc(-n2ccccc2=O)cc1 ; *c1ccc(-n2c(=O)n(C)c3cccnc32)cn1]

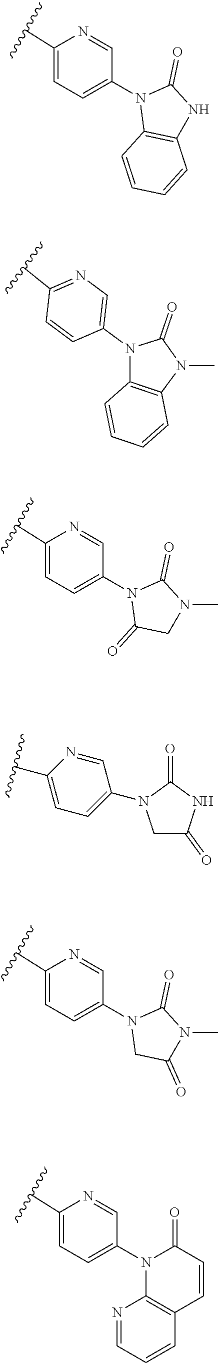 [SMILES: *c1ccc(-n2c(=O)[nH]c3ccccc32)cn1 ; *c1ccc(-n2c(=O)n(C)c3ccccc32)cn1 ; *c1ccc(N2C(=O)CN(C)C2=O)cn1 ; *c1ccc(N2CC(=O)NC2=O)cn1 ; *c1ccc(N2CC(=O)N(C)C2=O)cn1 ; *c1ccc(-n2c(=O)ccc3cccnc32)cn1]

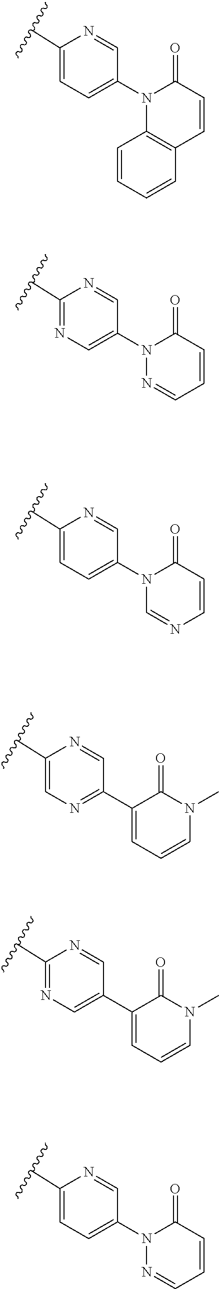 [SMILES: *c1ccc(-n2c(=O)ccc3ccccc32)cn1 ; *c1ncc(-n2ncccc2=O)cn1 ; *c1ccc(-n2cnccc2=O)cn1 ; *c1cnc(-c2cccn(C)c2=O)cn1 ; *c1ncc(-c2cccn(C)c2=O)cn1 ; *c1ccc(-n2ncccc2=O)cn1]

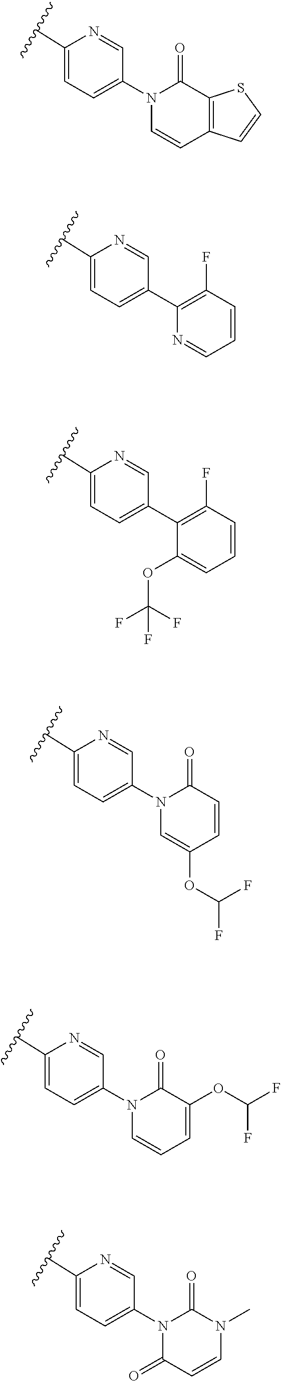 [SMILES: *c1ccc(-n2ccc3ccsc3c2=O)cn1 ; *c1ccc(-c2ncccc2F)cn1 ; *c1ccc(-c2c(F)cccc2OC(F)(F)F)cn1 ; *c1ccc(-n2cc(OC(F)F)ccc2=O)cn1 ; *c1ccc(-n2cccc(OC(F)F)c2=O)cn1 ; *c1ccc(-n2c(=O)ccn(C)c2=O)cn1]

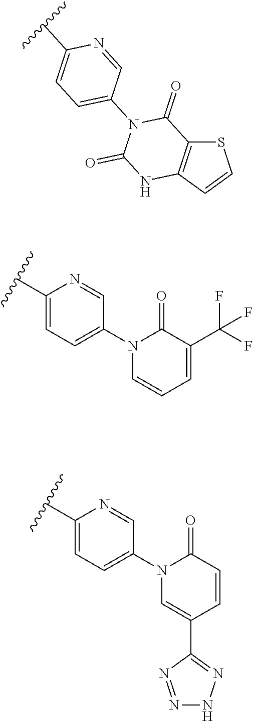 [SMILES: *c1ccc(-n2c(=O)[nH]c3ccsc3c2=O)cn1 ; *c1ccc(-n2cccc(C(F)(F)F)c2=O)cn1 ; *c1ccc(-n2cc(-c3nn[nH]n3)ccc2=O)cn1]

50. The compound of any of the preceding statements or a pharmaceutically acceptable salt thereof, wherein A-B-C is of the formula (I-A), (I-A1), (I-A2), (I-A3), (I-B), (I-B1), (I-B2), (I-B3), (I-C), (I-C1), (I-C₂), (I-C3), (I-D), (I-D1), (I-D2), (I-D3) (I-E), (I-E1), (I-E2) or (I-E3):

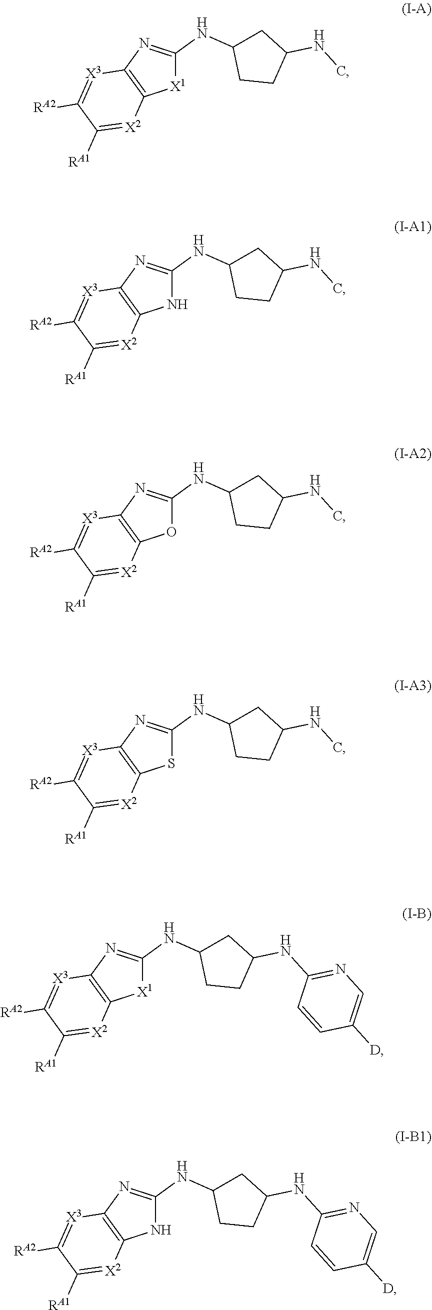

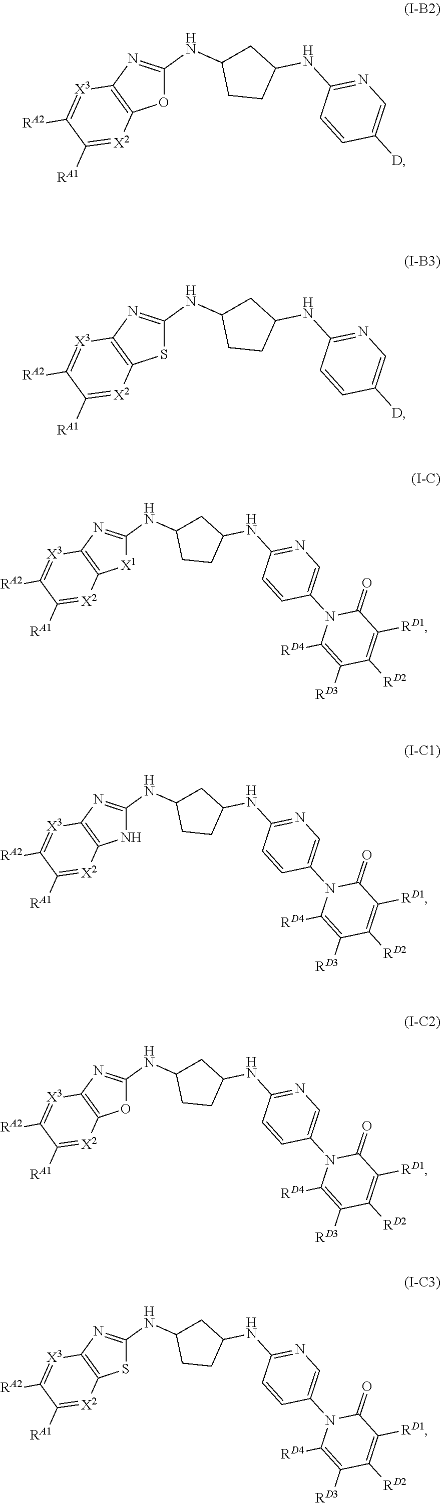

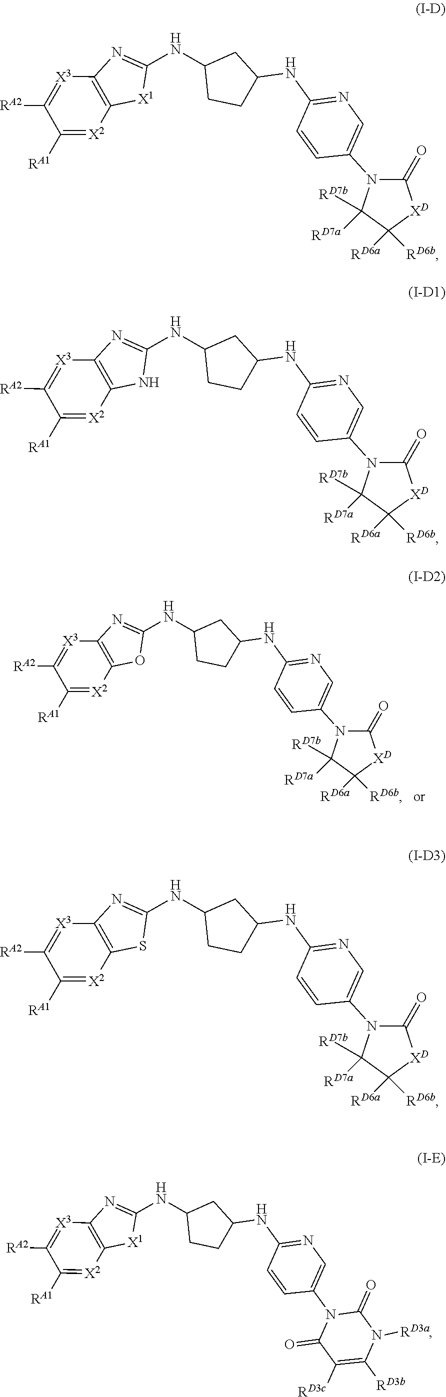

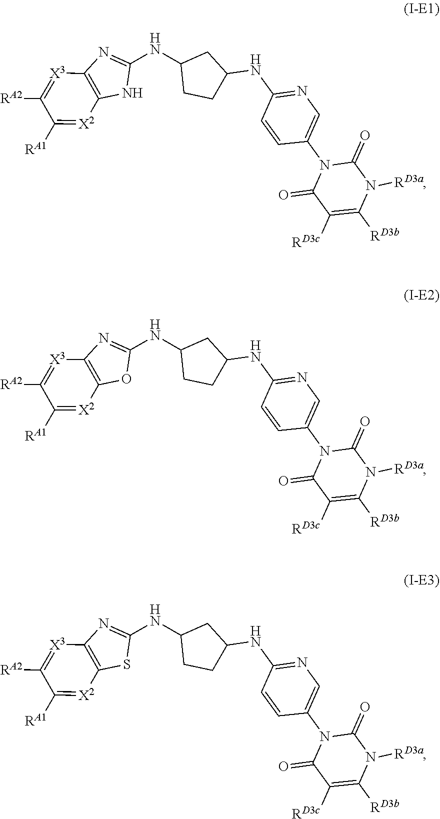

wherein X¹, X², X³, R^{A1}, R^{A2}, R^{A3}, C, D, R^{D1}, R^{D2}, R^{D3}, R^{D4}, R^{D6a}, R^{D6b}, R^{D7a}, R^{D7b}, X^{D}, R^{D3a}, R^{D3b} and R^{D3c} are as defined in any of the preceding statements.

51. The compound of Table 1 or a pharmaceutically acceptable salt thereof.

52. The compound of any one of statements 1 to 51 or a pharmaceutically acceptable salt thereof, for use in therapy.

53. A pharmaceutical composition comprising the compound of any one of statements 1 to 51 or a pharmaceutically acceptable salt thereof, and a pharmaceutically acceptable diluent, carrier or excipient.

54. The compound of any one of statements 1 to 51 or a pharmaceutically acceptable salt thereof, or a pharmaceutical composition according to statement 53 for use in the treatment of a cardiovascular disease.

55. The compound for use according to statement 54 wherein the compound is administered simultaneously, separately or sequentially in combination with an additional active ingredient selected from the group consisting of:
i) a statin;
ii) a cholesterol absorption inhibitor;
iii) a SGLT2 inhibitor;
iv) a P2Y12 inhibitor:
v) a citrate lyase inhibitor; and
vi) anti-hypertensive drugs.

56. The compound use according to statement 54 or 55 when the cardiovascular disease is selected from dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease heart failure or congestive heart failure.

57. Use of a compound of any one of statements 1 to 51 or a pharmaceutically acceptable salt thereof, or a pharmaceutical composition according to statement 53 in a method of medical treatment.

58. A method of medical treatment comprising administering to the patient the pharmaceutical composition of statement 53.

59. Use of a compound of any one of statements 1 to 51 in the manufacture of a medicament for use in therapy.

60. A method of treating PCSK9-mediated disease or disorder in a patient in need thereof comprising administering to the patient a therapeutically effective amount of the compound or pharmaceutically acceptable salt thereof according to any one of statements 1 to 51 or the pharmaceutical composition according to statement 53.

61. The method according to statement 60, wherein the disease or disorder is a cardiovascular disease or disorder.

62. The method according to statement 61, wherein the cardiovascular disease or disorder is selected from dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease heart failure or congestive heart failure.

1. A compound of Formula (I) 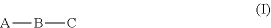
or a pharmaceutically acceptable salt, tautomeric forms or stereoisomers thereof,
wherein A is of the following formula: 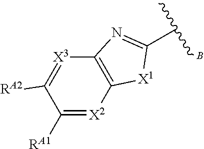
wherein the wavy line indicates the point of attachment to B;
X¹ is selected from O, S or NH;
X² is either N or C—H;
X³ is either N or C—R^{A3};
wherein if X¹ is NH and X² is C—H then X³ is C—R^{A3};
when X¹ is NH, R^{A1} is X⁴ and R^{A2} is X⁵;
when X² is N and X¹ is O or S, R^{A1} is X⁴ and R^{A2} is X⁵;
when X² is CH and X¹ is S, R^{A1} is X⁴ and R^{A2} is X⁵;
when X² is CH and X¹ is O, R^{A1} is X⁵ and R^{A2} is X⁴;
X⁴ is selected from the group consisting of:
(i) H;
(ii) halo;
(iii) CN;
(iv) C₁ alkyl optionally substituted by one or more OH, CN, or one or more halo groups; and
(v) C₁ alkoxy, optionally substituted by one or more halo groups;
X⁵ is selected from the group consisting of:
(i) H;
(ii) halo;
(iii) CN;
(iv) C₁₋₆ hydrocarbon, optionally substituted by OH, CN, C₁₋₆ alkyl acyl, C₁₋₆ alkoxy or one or more halo groups;
(v) C₁₋₆ alkoxy, optionally substituted by C₁₋₆ alkyl amido, C₁₋₆ alkyl phosphonyl, or one or more halo groups;
(vi) C₁₋₆ alkylamino;
(vii) C₁₋₆ thioalkyl;
(viii) C₁₋₆ alkyl phosphinyl; and
(ix) C₁₋₆ alkyl phosphonyl;
R^{A3} is selected from the group consisting of:
(i) H;
(ii) halo;
(iii) CN;
(iv) C₁₋₆ hydrocarbon, optionally substituted by OH, CN, C₁₋₆ thioalkyl, C₁₋₆ alkoxy, C₁₋₆ alkyl acyl, C₁₋₆ acyloxy, C(═O)OH, C₁₋₆ alkyl ester, C₁₋₆ alkylamino; —C(═O)NH₂, C₁₋₆ alkyl amido, C₁₋₆ acylamido, C₁₋₆ alkyl sulfinyl, C₁₋₆ alkyl sulfonyl or one or more halo groups;
(v) C₁₋₆ alkoxy, optionally substituted by NH₂, C₄ heterocyclyl and one or more halo groups;
(vi) C₄ heterocycyl;
(vii) C₁₋₆ alkylamino, optionally substituted by CN, OH, C₄ heterocyclyl;
(viii) C₁₋₆ dialkylamino, optionally substituted by —NH₂; and
(ix) C₁₋₆ thioalkyl, optionally substituted by OH or —NH₂;
wherein B is of formula (B-1) or (B-2) 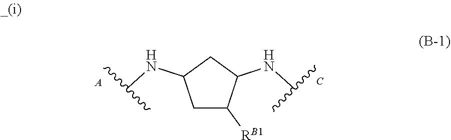
wherein the wavy lines indicate the point of attachment to A and C;
R^{B1} is H, OH, ═CHCH₂—OH, —O—C₁₋₄ alkyl or C₁₋₄ alkyl, wherein the C₁₋₄ alkyl is optionally substituted by OH or OMe; 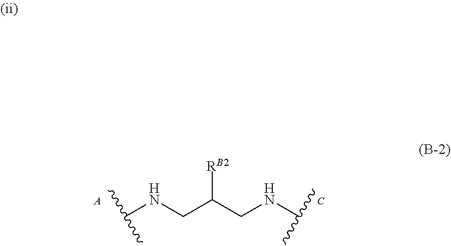
wherein the wavy lines indicate the point of attachment to A and C;
R^{B2} is C₁₋₂ alkyl-OH, CH₂C(═O)NHMe or C₁₋₃ alkyl;
wherein when R^{A1} is H or halo, R^{B2} is C₁₋₂ alkyl-OH or CH₂C(═O)NHMe;
wherein C is selected from the group consisting of C₆₋₁₀ carboaryl, C₅₋₆ heteroaryl and C₅₋₁₀ heterocyclyl, which each group is optionally independently substituted by:
(i) C₆₋₁₀ carboaryl, C₄₋₁₀ carbocyclyl, C₅₋₁₀ heteroaryl, C₄₋₁₀ heterocyclyl, or C₅₋₁₀ bridged heterocyclyl, spiro C₆₋₁₂heterocyclyl or a spiro C₆₋₁₂ carbocyclyl,
which is optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkyl ester;
g) C₅₋₆ heterocyclyl with an optional methyl, OH or ═O substituent;
h) C₅₋₆ heteroaryl optionally substituted by C₁₋₆ alkyl;
i) C₄₋₁₀ carbocyclyl with an optional methyl or ═O substituent;
j) C₆₋₁₀ carboaryl with optional substituents of one or more halo groups;
l) P(═O)Me₂;
m) C(═O)OH or CH₂C(═O)OH;
n) tetrazolyl, CH₂-tetrazolyl, 5-oxo-4H-1,2,4-oxadiazol-3-yl; or
(ii) one or more groups selected from C(═O)OH, CN, halo, nitro, C₁₋₆ alkyl, C₁₋₆ thioalkyl, C₁₋₆ alkoxy, C₁₋₆ alkyl acyl, C₁₋₆ alkyl amido, di-C₁₋₆ alkyl amido, C₁₋₆ alkyl sulfonamido, and di-C₁₋₆ alkyl sulfonamido.

2. The compound of claim 1, or a pharmaceutically acceptable salt thereof,
wherein A is selected from one of the following formulae A1-A3: 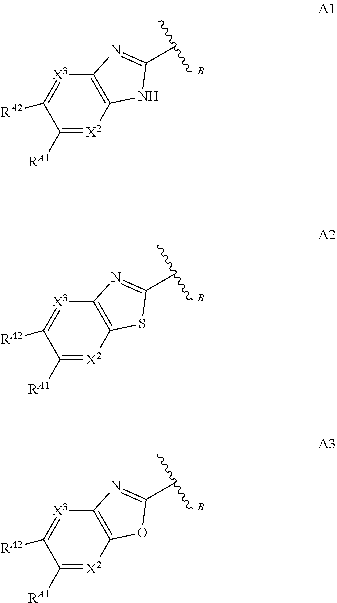
wherein the wavy line indicates the point of attachment to B and X², X³, R^{A1} and R^{A2} are as defined in claim 1, wherein A is optionally selected from one of the following formulae A4-A14: 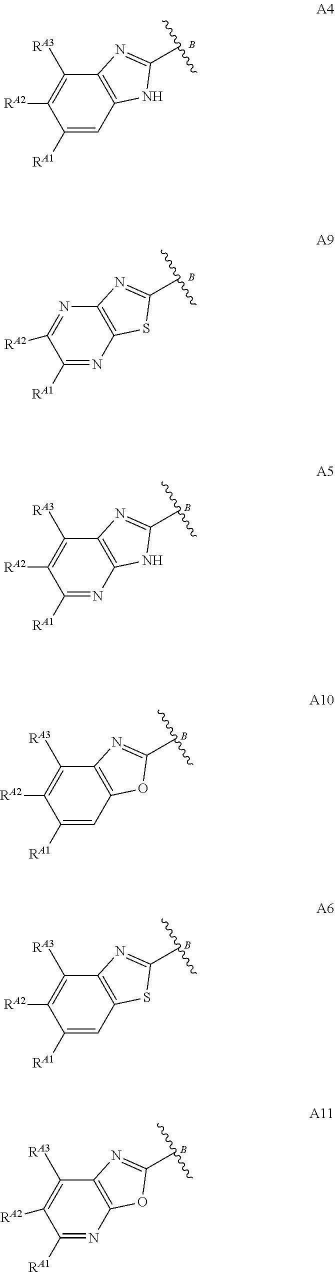 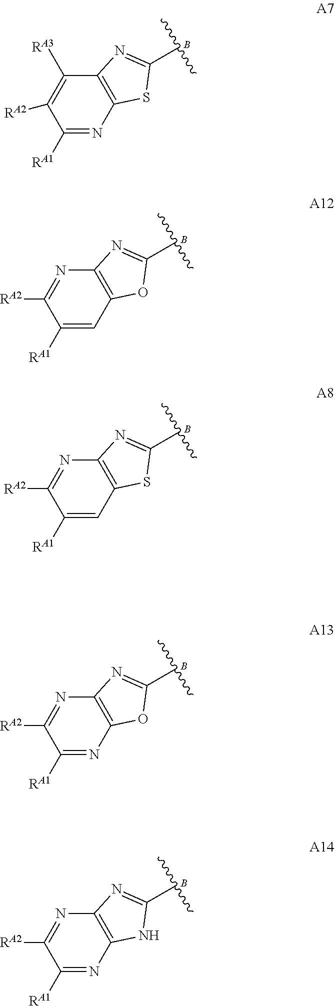
wherein the wavy line indicates the point of attachment to B, and R^{A1}, R^{A2} and R^{A3} are as defined in claim 1.

3. The compound of claim 1, or a pharmaceutically acceptable salt thereof, wherein X⁴ is selected from:
(i) H;
(ii) halo;
(iii) CN; and
(iv) C₁ alkyl, optionally substituted by one or more OH groups or one or more halo groups.

4. The compound of claim 1, or a pharmaceutically acceptable salt thereof, wherein X⁵ is selected from:
(i) H;
(ii) halo;
(iii) C₁₋₆ alkyl, optionally substituted by one or more OH or one or more halo groups;
(iv) C₁₋₆ alkoxy, optionally substituted by one or more halo groups;
(v) C₃₋₅ cycloalkyl;
(vi) C₁₋₆ thioalkyl; and
(vii) C₁₋₆ alkyl phosphinyl.

5. The compound of claim 1, or a pharmaceutically acceptable salt thereof, wherein R^{A3} is selected from:
(i) H;
(ii) halo;
(iii) CN;
(iv) C₁₋₆ alkyl optionally substituted by OH, or one or more halo groups;
(v) C₂₋₆ alkenyl optionally substituted by OH, or one or more halo groups;
(vi) C₂₋₆ alkynyl optionally substituted by OH, or one or more halo groups; and
(vii) C₁₋₆ alkoxy, optionally substituted by one or more halo groups.

6. The compound of claim 1, or a pharmaceutically acceptable salt thereof, wherein A is selected from one of the following formula: 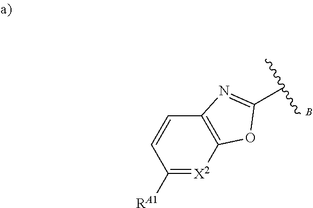
wherein X² is selected from N and C—H; and
R^{A1} is selected from H, CN, CH₂OH, OCHF₂, methyl and Br; 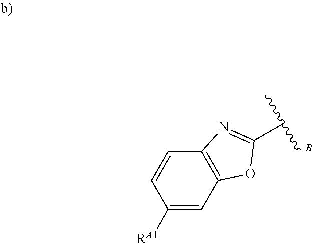
wherein R^{A1} is selected from H, CN, CH₂OH, methyl, OCHF₂, and Br; 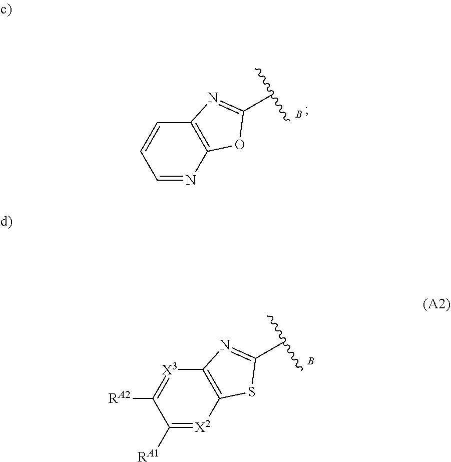
wherein X² is selected from N and C—H;
when X² is N, X³ is either N or C—R^{A3};
when X² is C—H, X³ is C—R^{A3} or N;
R^{A1} is selected from H, methyl and Br;
R^{A2} is H, CN or CH₂OH; and
R^{A3} is H; 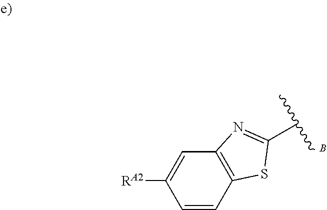
wherein R^{A2} is selected from:
(i) H;
(ii) halo;
(iii) C₁₋₆ alkyl, optionally substituted by one or more OH or one or more halo groups,
(iv) C₁₋₆ alkoxy, optionally substituted by one or more halo groups;
(v) C₃₋₅ cycloalkyl;
(vi) C₁₋₆ thioalkyl;
(vii) C₁₋₆ alkyl phosphinyl; and
(viii) CN; 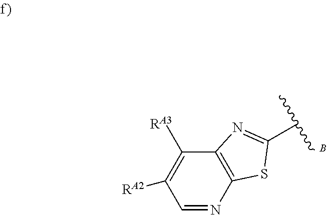
wherein R^{A3} is selected from H, halo and OMe; and
R^{A2} is selected from H, Br and CH₂OH; 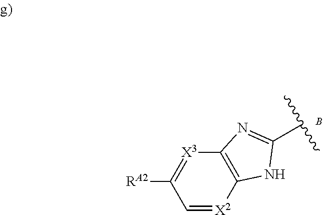
wherein
X² is N or C—H;
when X² is N, X³ is either N or C—R^{A3};
when X² is C—H, X³ is C—R^{A3};
wherein R^{A3} is selected from H, Cl, Br and OMe; and
R^{A2} is selected from H, CH₂OH, OCH₃, OCHF₂, OCF₃, CF₃, F, Cl, Br, ethyl, cyclopropyl, methyl, —P(═O)Me₂ and —S—CH₃; or 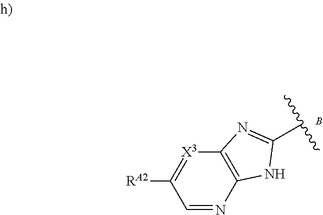
wherein
X³ is N or C—R^{A3};
wherein R^{A3} is selected from H, Cl, Br or OMe;
R^{A2} is selected from H, CH₂OH, OCH₃, OCHF₂, OCF₃, CF₃, F, Cl, Br, ethyl, cyclopropyl, methyl, —P(═O)Me₂ and —S—CH₃.

7. The compound of claim 1, or a pharmaceutically acceptable salt thereof, wherein A is selected from the following compounds: 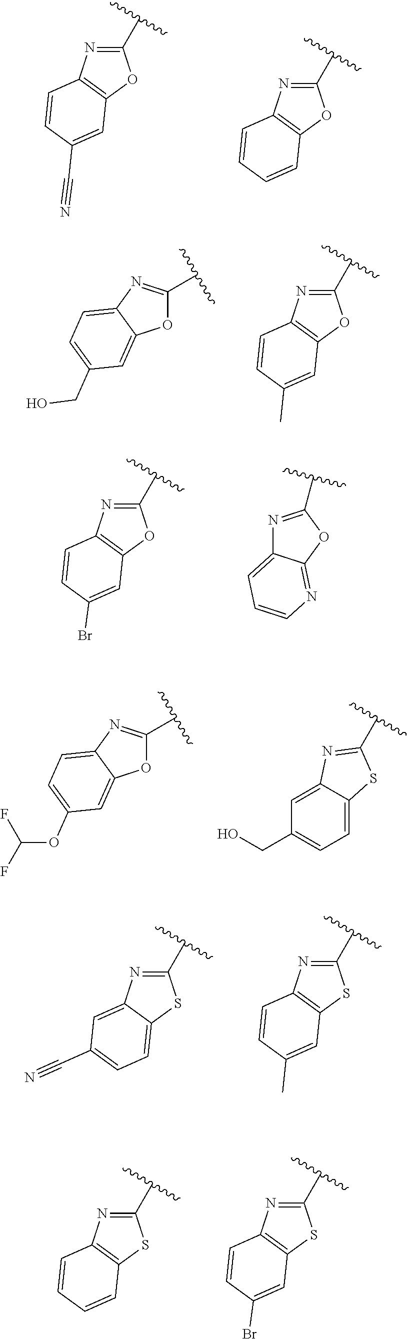 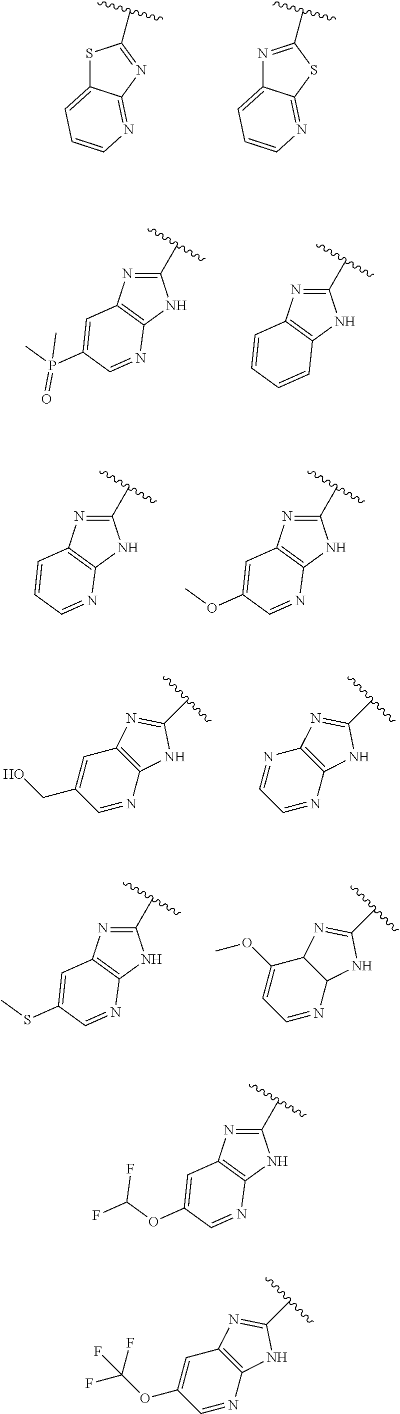 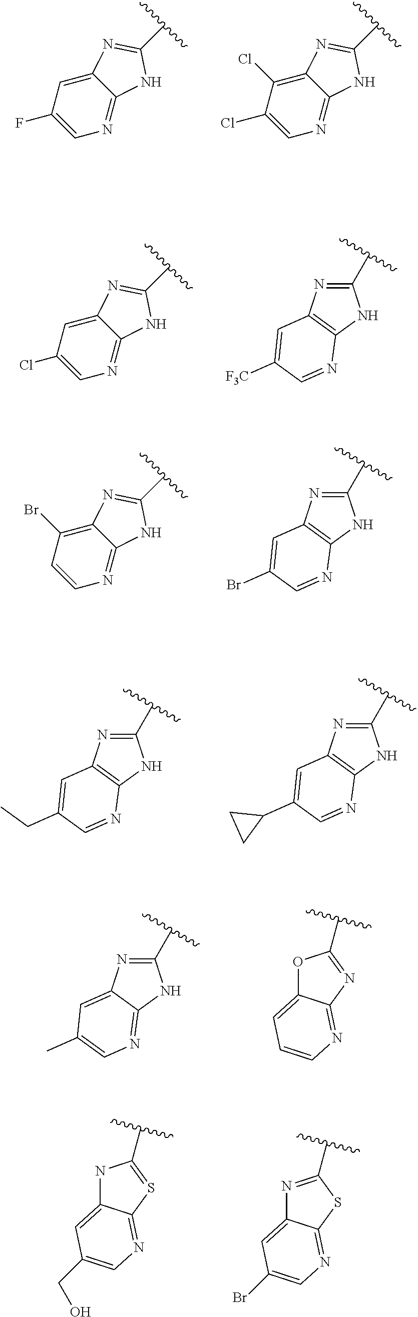 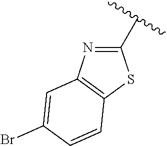 [SMILES: *c1nc2cc(Br)ccc2s1]
wherein the wavy line indicates the point of attachment to B.

8. The compound of claim 1, or a pharmaceutically acceptable salt thereof, wherein B is of the following formula (B-1a):

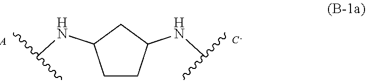

9. The compound of claim 1, or a pharmaceutically acceptable salt thereof, wherein C is an optionally substituted by pyridinyl, pyrazinyl or pyrimidinyl, wherein the optional substituents are selected from C₆₋₁₀ carboaryl, C₄₋₁₀ carbocyclyl, C₅₋₁₀ heteroaryl, C₅₋₁₀ heterocyclyl, C₅₋₁₀ bridged heterocyclyl, spiro C₆₋₁₂heterocyclyl and a spiro C₆₋₁₂ carbocyclyl, optionally substituted by one or more of the following groups:
a) one or two ═O groups;
b) one or more halo groups;
c) CN, NH₂ or OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkyl ester;
g) C₅₋₆ heterocyclyl with an optional methyl, OH or ═O substituent;
h) C₅₋₆ heteroaryl optionally substituted by C₁₋₆ alkyl;
i) C₄₋₁₀ carbocyclyl with an optional methyl or ═O substituent;
j) C₆₋₁₀ carboaryl with optional substituents of one or more halo groups;
l) P(═O)Me₂;
m) C(═O)OH or CH₂C(═O)OH; and/or
n) tetrazolyl, CH₂-tetrazolyl, 5-oxo-4H-1,2,4-oxadiazol-3-yl.

10. The compound of claim 1, or a pharmaceutically acceptable salt thereof, wherein C is of the formula (C-1): 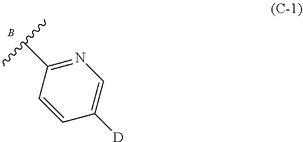
wherein D is C₆₋₁₀ carboaryl, C₅₋₁₀ heteroaryl or C₅₋₁₀ heterocyclyl, optionally substituted by:
i) one or two ═O groups;
ii) one or two C₁₋₄ alkyl groups which can be branched;
iii) OMe;
iv) Piperazinyl or pyrimidinyl optionally substituted by methyl;
v) C(═O)OH;
vi) Cl;
vii) one or more F;
viii) phenyl, optionally substituted by one or more fluoro;
ix) CN;
x) CF₃;
xi) OCF₃;
xii) OCHF₂;
xiii) tetrazolyl, pyrazolyl, triazolyl;
xiv) NH₂;
xv) pyridinyl;
xvi) CH₂OH;
xvii) OH;
xviii) P(═O)Me₂;
xix) CHF₂; or
xx) CH₂CF₃.

11. The compound of claim 10, or a pharmaceutically acceptable salt thereof, wherein D is of the formula (D-1): 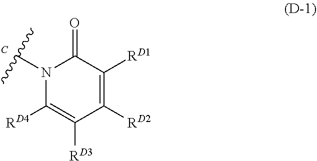
wherein one or two of R^{D1}, R^{D2}, R^{D3} and R^{D4} are selected from:
i) C₁₋₆ alkyl, optionally substituted by one or more halo groups;
ii) C₁₋₆ alkoxy, optionally substituted by one or more halo groups;
iii) C₅₋₆ heterocyclyl or C₅₋₆ heteroaryl, optionally substituted by methyl;
iv) C(═O)OH or CH₂C(═O)OH;
v) ═O, halo, NH₂ or CN;
vi) phenyl, optionally substituted by one or more halo atoms;
and the rest are H; or
wherein R^{D3} and R^{D4} form an optionally substituted 6 membered carboaromatic, heterocyclic or heteroaromatic ring, wherein the optional substituents are selected from OH, methyl, OMe, halo and C(═O)OH; or
wherein R^{D1} and R^{D2} form an optionally substituted 5 or 6 membered carboaromatic, heterocyclic or heteroaromatic ring, wherein the optional substituents are selected from OH, methyl, OMe, halo and C(═O)OH; or
wherein R^{D2} and R^{D3} form an optionally substituted 5 or 6 membered carboaromatic, heterocyclic or heteroaromatic ring, wherein the optional substituents are selected from halo, CN OH, P(═O)Me₂, C(═O)OH, C₁₋₆ alkoxy optionally substituted by one or more halo groups, and C₁₋₆ alkyl optionally substituted by one or more halo groups; or
wherein R^{D1}, R^{D2}, R^{D3} and R^{D4} are all H.

12. The compound of claim 11, or a pharmaceutically acceptable salt thereof, wherein:
a) one or two of R^{D1}, R^{D2}, R^{D3} and R^{D4} are selected from:
i) methyl;
ii) OMe;
iii) pyrimidinyl substituted by methyl;
iv) tetrazolyl;
v) C(═O)OH;
vi) halo;
vii) CF₃;
viii) CN;
ix) O—CF₃; and
x) —OCHF₂;
and the rest of R^{D1}, R^{D2}, R^{D3} and R^{D4} are H;
b) R^{D1}, R^{D2}, R^{D3} and R^{D4} are H;
c) R^{D3} is selected from the group consisting of: H, OMe, C(═O)OH, Cl, CN, OCH₂F, tetrazolyl, and pyrimidinyl optionally substituted by methyl; and wherein R^{D1}, R^{D2} and R^{D4} are all H;
d) R^{D1} is selected from H, methyl, OMe, Cl, OCF₃, CF₃, OCHF₂, and CN, and wherein R^{D2}, R^{D3} and R^{D4} are all H;
e) R^{D3} and R^{D4} form an unsubstituted phenyl ring or an unsubstituted pyridine ring;
f) R^{D1} and R^{D2} form an unsubstituted 5 membered heterocycle or heteroaromatic ring; or
g) R^{D1} and R^{D2} form an unsubstituted thiophene.

13. The compound of claim 10, or a pharmaceutically acceptable salt thereof, wherein D is of the formula (D-2): 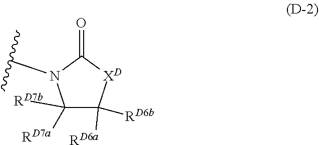
wherein X^{D} is NR^{D5a} or CR^{D5a}R^{D5b};
R^{D5a} is selected from H and methyl;
either R^{D5b} and R^{D6b} are both H or together they are —CH₂—;
R^{D6a} is selected from H, ═O, methyl, CH₂OH and C(═O)OH, wherein when R^{D6a} is ═O, R^{D6b} is absent;
R^{D7a} is selected from H, ═O, methyl, CH₂OH and C(═O)OH; and
R^{D7b} is H, wherein when R^{D7a} is ═O, R^{D7b} is absent;
or wherein R^{D6a} and R^{D7a} together form a phenyl ring or a C₆ heteroaromatic ring which is optionally substituted by CN, P(═O)Me₂ or C(═O)OH and R^{D6b} and R^{D7b} are absent.

14. The compound of claim 10, or a pharmaceutically acceptable salt thereof, wherein D is of the formula (D-3): 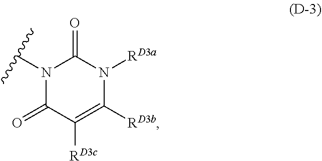
wherein R^{D3a} is selected from H and methyl;
wherein either: R^{D3b} and R^{D3c} are independently selected from H, halo, CN, OH, P(═O)Me₂, C(═O)OH, C₁₋₆ alkoxy optionally substituted by one or more halo groups, C₁₋₆ alkyl optionally substituted by one or more halo groups;
or R^{D3b} and R^{D3c} together form a C₅₋₆ heteroaryl ring.

15. The compound of claim 10, or a pharmaceutically acceptable salt thereof, wherein D is selected from the following groups:

| Name | Structure |
|---|---|
| 2-oxo-1-pyridyl | 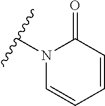 [SMILES: *n1ccccc1=O] |
| 3-methyl-2- oxo-1-pyridyl | 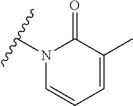 [SMILES: *n1cccc(C)c1=O] |
| 3-methoxy-2- oxo-1-pyridyl | 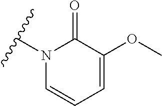 [SMILES: *n1cccc(OC)c1=O] |
| 5-methoxy-2- oxo-1-pyridyl | 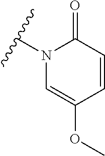 [SMILES: *n1cc(OC)ccc1=O] |
| 5-chloro-2-oxo- 1-pyridyl | 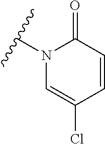 [SMILES: *n1cc(Cl)ccc1=O] |
| 2-oxo-1,8- naphthyridin-1- yl | 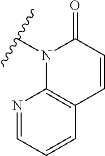 [SMILES: *n1c(=O)ccc2cccnc21] |
| 2-oxo-1-quinolyl | 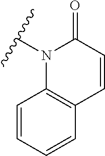 [SMILES: *n1c(=O)ccc2ccccc21] |
| 3-methyl-2- oxo- benzimidazol- 1-yl | 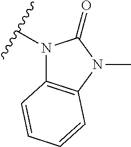 [SMILES: *n1c(=O)n(C)c2ccccc21] |
| 3-methyl-2,4- dioxo- imidazolidin-1-yl | 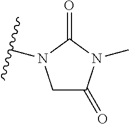 [SMILES: *N1CC(=O)N(C)C1=O] |
| 3-methyl-2,5- dioxo- imidazolin-1- yl | 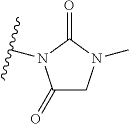 [SMILES: *N1C(=O)CN(C)C1=O] |
| 3-chloro-2-oxo- 1-pyridyl | 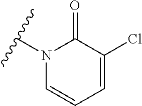 [SMILES: *n1cccc(Cl)c1=O] |
| 3-cyano-2-oxo- 1-pyridyl | 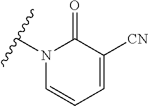 [SMILES: *n1cccc(C#N)c1=O] |
| 5-cyano-2-oxo- 1-pyridyl | 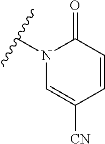 [SMILES: *n1cc(C#N)ccc1=O] |
| 3-fluoro-2- oxopyridin- 1(2H)-yl | 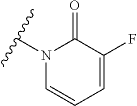 [SMILES: *n1cccc(F)c1=O] |
| 2-fluoro-6- methoxy-phenyl | 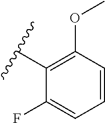 [SMILES: *c1c(F)cccc1OC] |
| 5-carboxy-2- oxo-1-pyridyl | 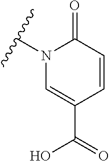 [SMILES: *n1cc(C(=O)O)ccc1=O] |
| 2-oxo-3H- benzimidazol-1- yl | 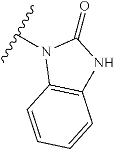 [SMILES: *n1c(=O)[nH]c2ccccc21] |
| 2-oxo-1H- imidazo[4,5- b]pyridin-3-yl | 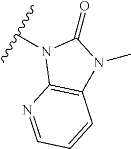 [SMILES: *n1c(=O)n(C)c2cccnc21] |
| 6-oxopyridazin- 1-yl | 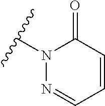 [SMILES: *n1ncccc1=O] |
| 6- oxopyrimidin- 1-yl | 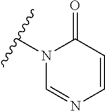 [SMILES: *n1cnccc1=O] |
| 1-methyl-2-oxo- 3-pyridyl | 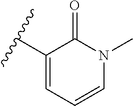 [SMILES: *c1cccn(C)c1=O] |
| 2-oxo-3- (trifluoromethyl)- 1-pyridyl | 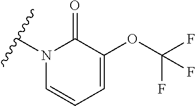 [SMILES: *n1cccc(OC(F)(F)F)c1=O] |
| 2-fluorophenyl | 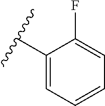 [SMILES: *c1ccccc1F] |
| 5-(2- methylpyrimidin- 5-yl)-2-oxo- 1-pyridyl | 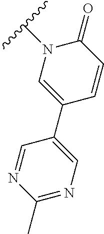 [SMILES: *n1cc(-c2cnc(C)nc2)ccc1=O] |
| 2- (difluoromethoxy)- 6-fluoro- phenyl | 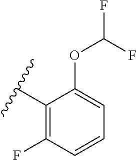 [SMILES: *c1c(F)cccc1OC(F)F] |
| 2- methoxyphenyl | 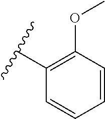 [SMILES: *c1ccccc1OC] |
| 2,6- difluorophenyl | 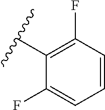 [SMILES: *c1c(F)cccc1F] |
| 3-fluoro-2- pyridyl | 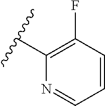 [SMILES: *c1ncccc1F] |
| 7- oxothieno[2,3- c]pyridin-6-yl | 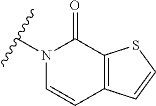 [SMILES: *n1ccc2ccsc2c1=O] |
| 2-oxo-5-(2H- tetrazol-5-yl)- 1-pyridyl | 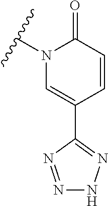 [SMILES: *n1cc(-c2nn[nH]n2)ccc1=O] |
| 2-fluoro-6- (trifluoromethoxy) phenyl | 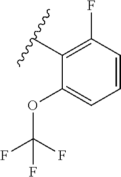 [SMILES: *c1c(F)cccc1OC(F)(F)F] |
| [5- (difluoromethoxy)- 2-oxo-1- pyridyl] | 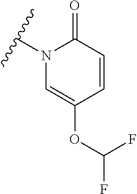 [SMILES: *n1cc(OC(F)F)ccc1=O] |
| 3- (difluoromethoxy)- 2-oxo-1- pyridyl | 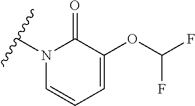 [SMILES: *n1cccc(OC(F)F)c1=O] |
| 3-methyl-2,6- dioxo- pyrimidin-1-yl | 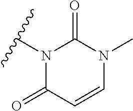 [SMILES: *n1c(=O)ccn(C)c1=O] |
| 2,4-dioxo-1H- thieno[3,2- d]pyrimidin-3-yl | 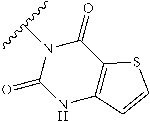 [SMILES: *n1c(=O)[nH]c2ccsc2c1=O] |
| 2-oxo-3- (trifluoromethyl)- 1-pyridyl | 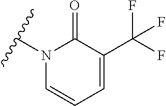 [SMILES: *n1cccc(C(F)(F)F)c1=O] |
| 2,4-dioxo- imidazolidin-1- yl | 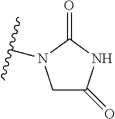 [SMILES: *N1CC(=O)NC1=O] |

16. The compound of claim 1, or a pharmaceutically acceptable salt thereof, wherein C is of the formula (C-2): 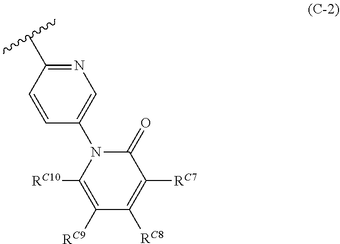
wherein one of R^{C7}, R^{C8}, R^{C9} and R^{C10} are selected from the group consisting of: methyl, OMe, piperazinyl, and pyrimidinyl optionally substituted by methyl, C(═O)OH, Cl, F, pyrazolyl, triazolyl, tetrazole, optionally substituted phenyl wherein the optional substituent is methyl or halo, CN, CF₃, OCHF₂, and O—CF₃; and
R^{C7}, R^{C8}, R^{C9} and R^{C10} are H;
or R^{C9} and R^{C10} form a phenyl or 6 membered heteroaromatic ring optionally substituted by methyl, and
R^{C7} and R^{C8} are both H; or
wherein R^{C7}, R^{C8}, R^{C9} and R^{C10} are all H.

17. The compound of claim 16, or a pharmaceutically acceptable salt thereof, wherein:
a) R^{C7}, R^{C8}, R^{C9} and R^{C10} are H;
or
b) R^{C9} is selected from H, CN, OMe, Cl, OCHF₂, tetrazolyl, pyrimidinyl substituted by methyl, and C(═O)OH; and R^{C7}, R^{C8} and R^{C10} are all H;
or
c) R^{C7} is selected from H, methyl, Cl, OMe, CN, CF₃, OCHF₂, and OCF₃; and
R^{C8}, R^{C9} and R^{C10} are all H.

18. The compound of claim 1, or a pharmaceutically acceptable salt thereof, wherein C is selected from the group consisting of:

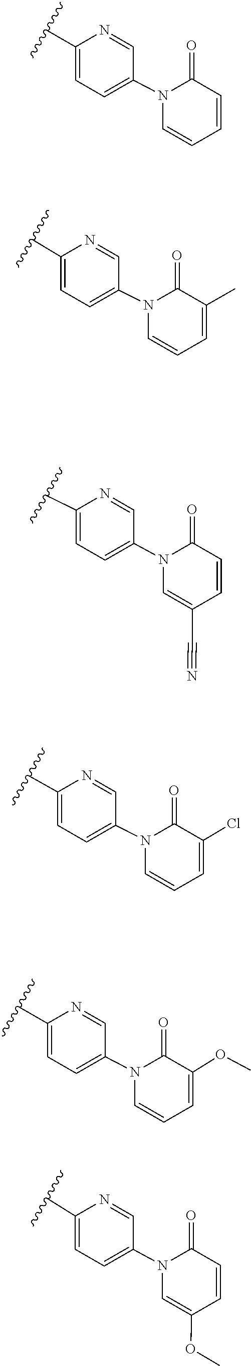 [SMILES: *c1ccc(-n2ccccc2=O)cn1 ; *c1ccc(-n2cccc(C)c2=O)cn1 ; *c1ccc(-n2cc(C#N)ccc2=O)cn1 ; *c1ccc(-n2cccc(Cl)c2=O)cn1 ; *c1ccc(-n2cccc(OC)c2=O)cn1 ; *c1ccc(-n2cc(OC)ccc2=O)cn1]

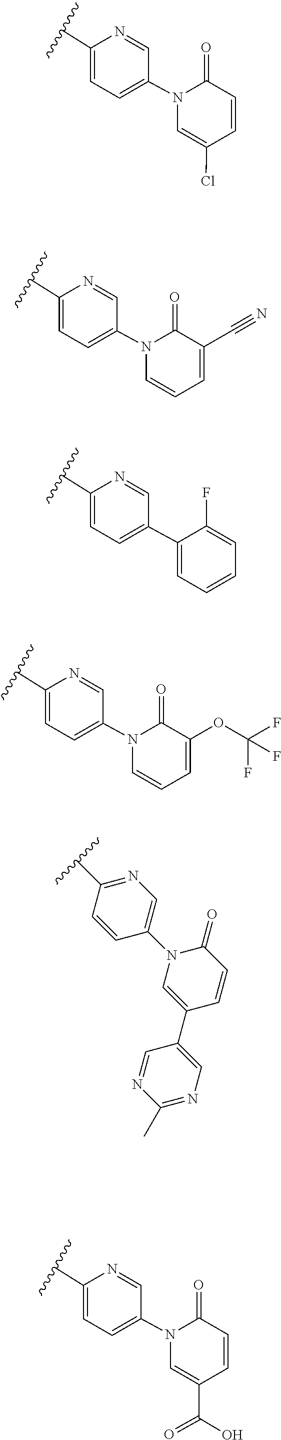 [SMILES: *c1ccc(-n2cc(Cl)ccc2=O)cn1 ; *c1ccc(-n2cccc(C#N)c2=O)cn1 ; *c1ccc(-c2ccccc2F)cn1 ; *c1ccc(-n2cccc(OC(F)(F)F)c2=O)cn1 ; *c1ccc(-n2cc(-c3cnc(C)nc3)ccc2=O)cn1 ; *c1ccc(-n2cc(C(=O)O)ccc2=O)cn1]

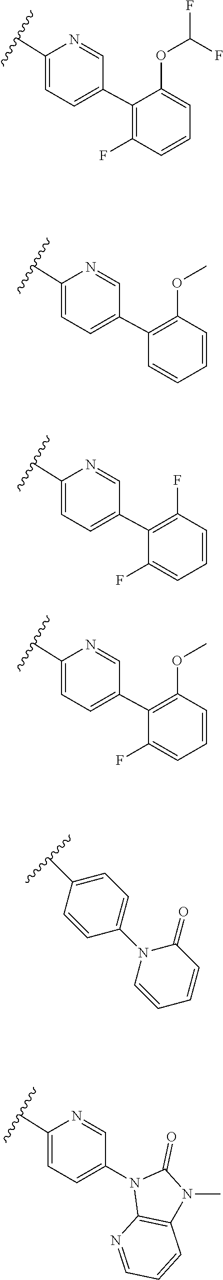 [SMILES: *c1ccc(-c2c(F)cccc2OC(F)F)cn1 ; *c1ccc(-c2ccccc2OC)cn1 ; *c1ccc(-c2c(F)cccc2F)cn1 ; *c1ccc(-c2c(F)cccc2OC)cn1 ; *c1ccc(-n2ccccc2=O)cc1 ; *c1ccc(-n2c(=O)n(C)c3cccnc32)cn1]

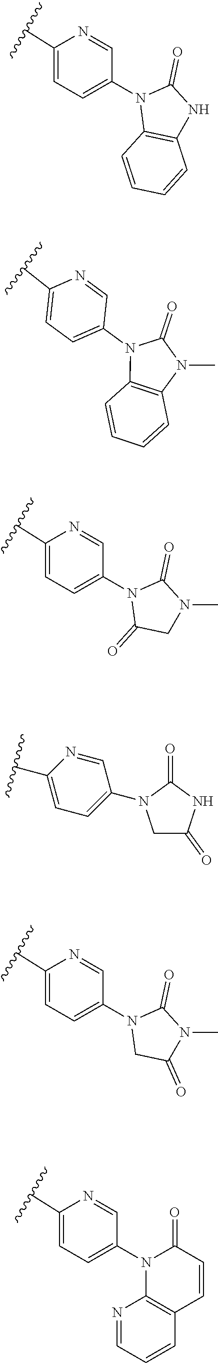 [SMILES: *c1ccc(-n2c(=O)[nH]c3ccccc32)cn1 ; *c1ccc(-n2c(=O)n(C)c3ccccc32)cn1 ; *c1ccc(N2C(=O)CN(C)C2=O)cn1 ; *c1ccc(N2CC(=O)NC2=O)cn1 ; *c1ccc(N2CC(=O)N(C)C2=O)cn1 ; *c1ccc(-n2c(=O)ccc3cccnc32)cn1]

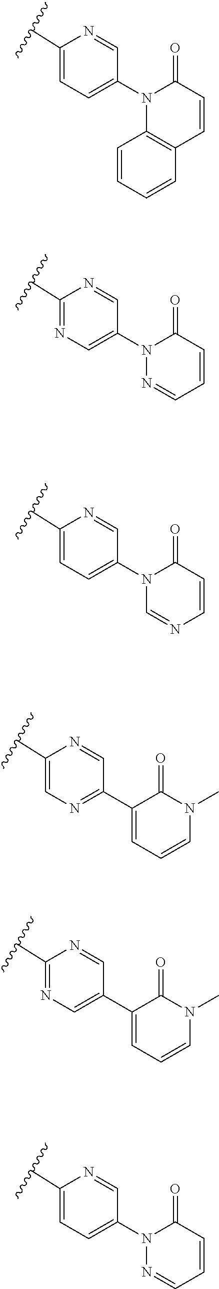 [SMILES: *c1ccc(-n2c(=O)ccc3ccccc32)cn1 ; *c1ncc(-n2ncccc2=O)cn1 ; *c1ccc(-n2cnccc2=O)cn1 ; *c1cnc(-c2cccn(C)c2=O)cn1 ; *c1ncc(-c2cccn(C)c2=O)cn1 ; *c1ccc(-n2ncccc2=O)cn1]

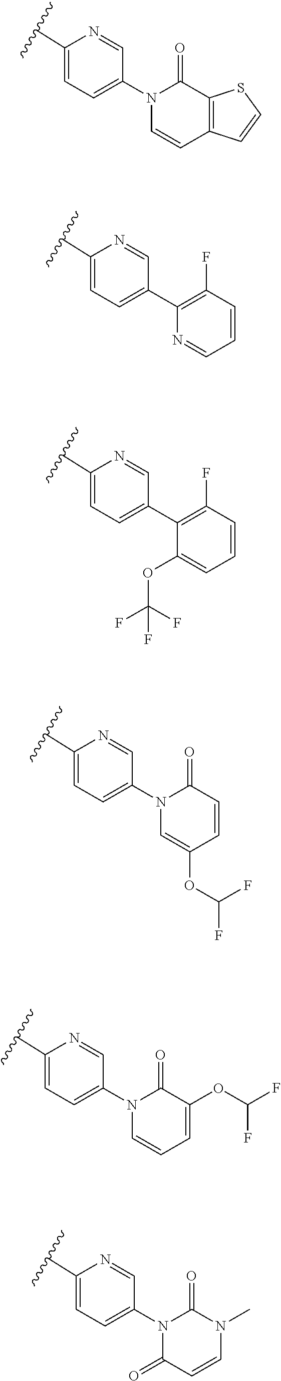 [SMILES: *c1ccc(-n2ccc3ccsc3c2=O)cn1 ; *c1ccc(-c2ncccc2F)cn1 ; *c1ccc(-c2c(F)cccc2OC(F)(F)F)cn1 ; *c1ccc(-n2cc(OC(F)F)ccc2=O)cn1 ; *c1ccc(-n2cccc(OC(F)F)c2=O)cn1 ; *c1ccc(-n2c(=O)ccn(C)c2=O)cn1]

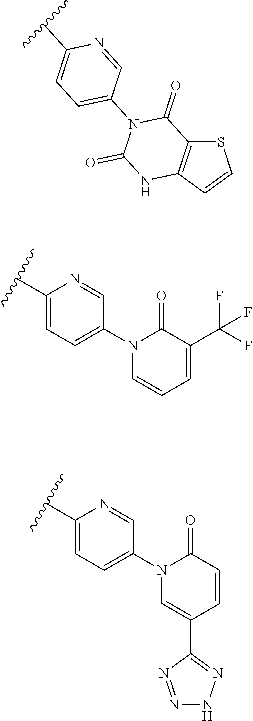 [SMILES: *c1ccc(-n2c(=O)[nH]c3ccsc3c2=O)cn1 ; *c1ccc(-n2cccc(C(F)(F)F)c2=O)cn1 ; *c1ccc(-n2cc(-c3nn[nH]n3)ccc2=O)cn1]

19. The compound of claim 1 or a pharmaceutically acceptable salt thereof, wherein A-B-C is of the formula (I-A), (I-A1), (I-A2), (I-A3), (I-B), (I-B1), (I-B2), (I-B3), (I-C), (I-C1), (I-C₂), (I-C3), (I-D), (I-D1), (I-D2), (I-D3) (I-E), (I-E1), (I-E2) or (I-E3): 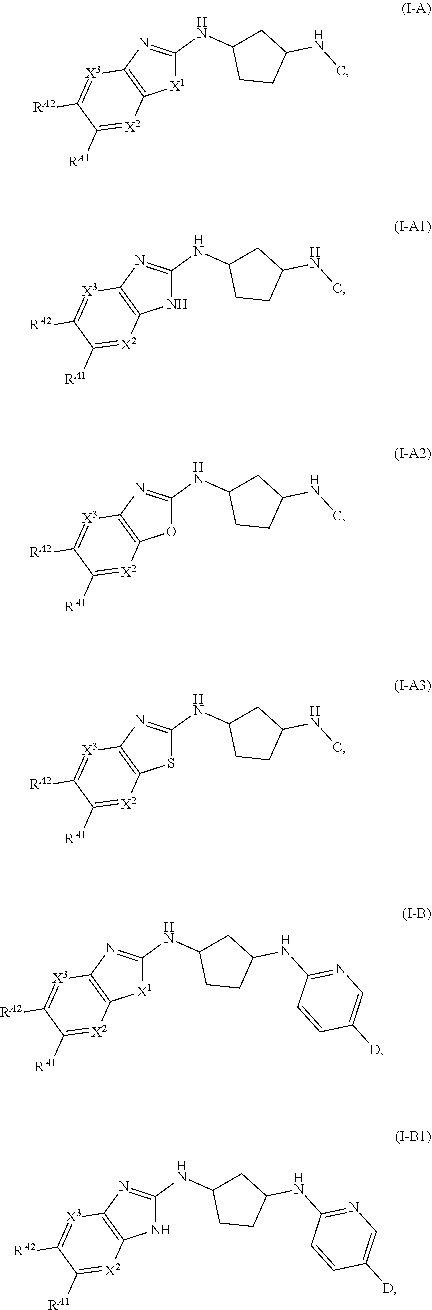 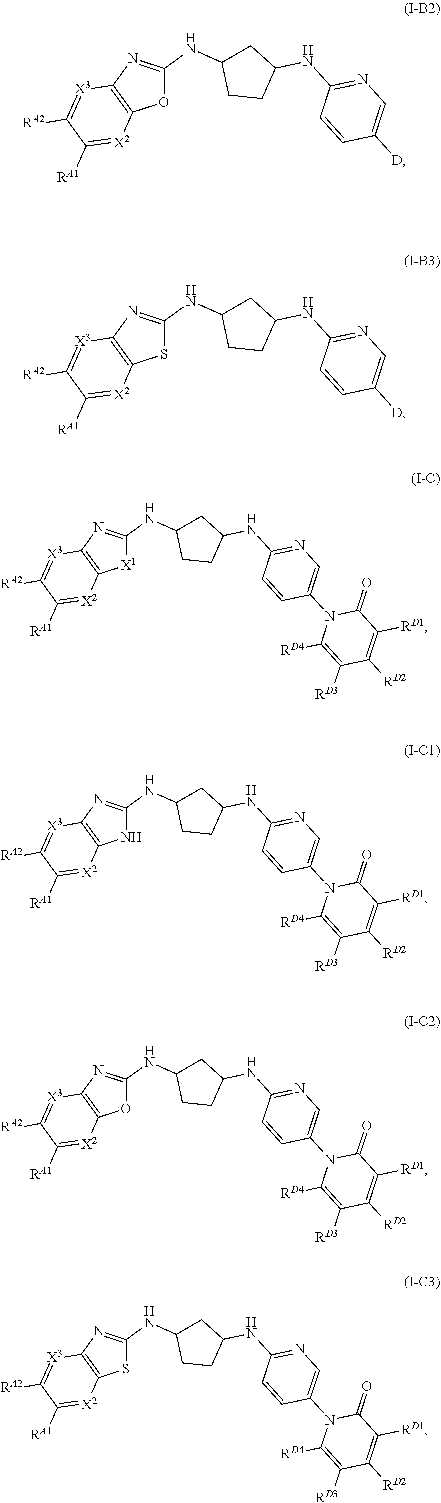 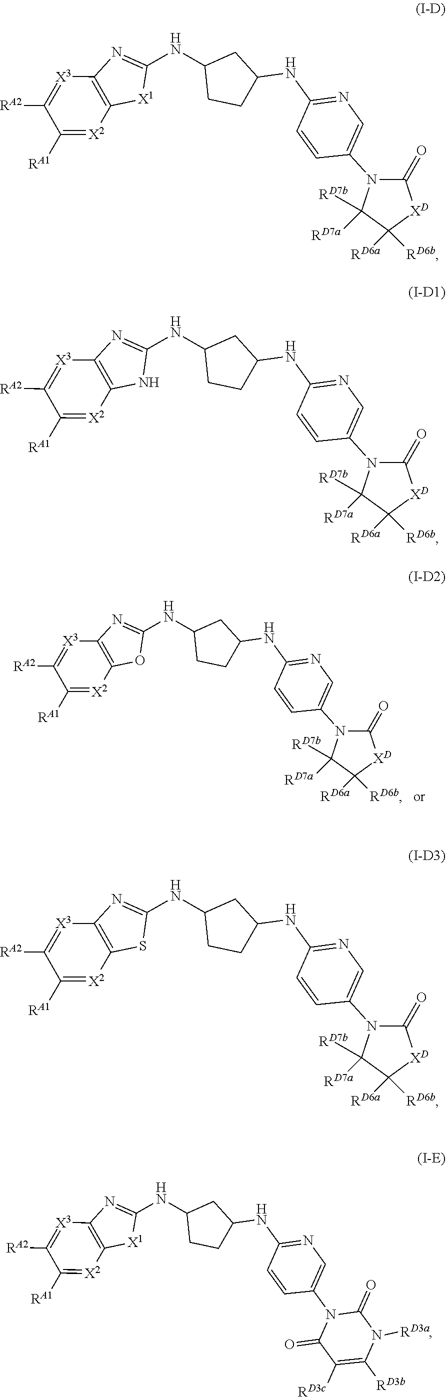 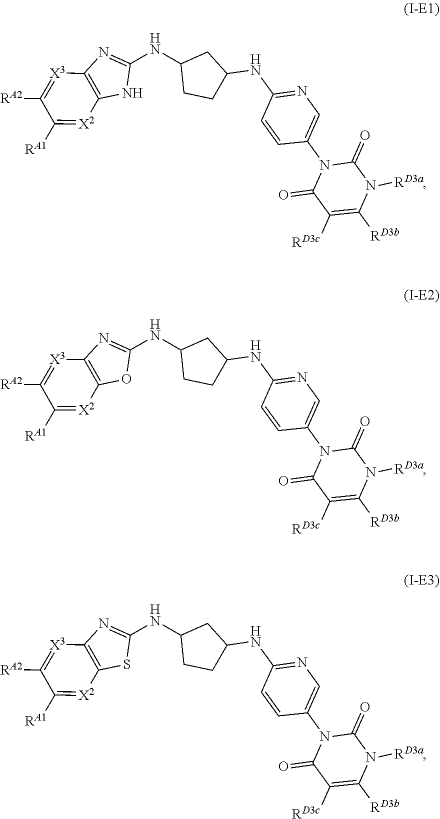
wherein X¹, X², X³, R^{A1}, R^{A2}, R^{A3}, C, D, R^{D1}, R^{D2}, R^{D3}, R^{D4}, R^{D6a}, R^{D6b}, R^{D7a}, R^{D7b}, X^{D} R^{D3a}, R^{D3b} and R^{D3c} are as defined in any of the preceding claims.

20. The compound of claim 1, which is 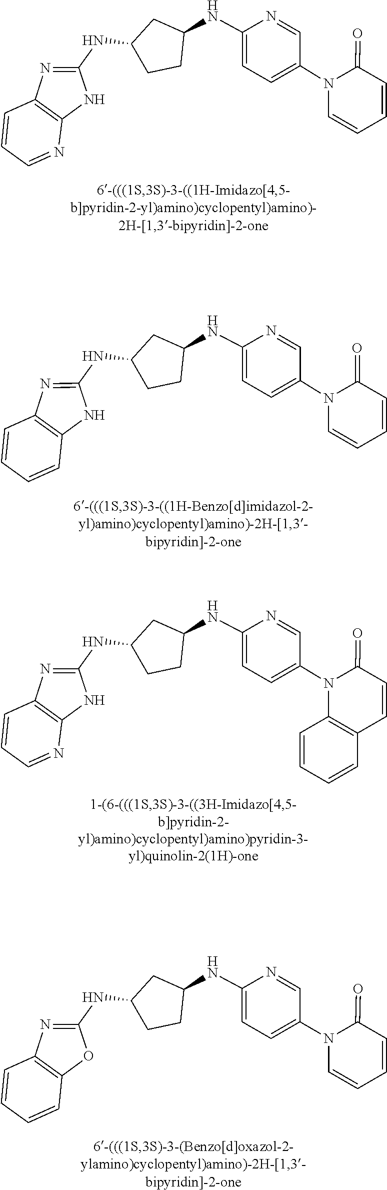 [SMILES: O=c1ccccn1-c1ccc(N[C@H]2CC[C@H](Nc3nc4cccnc4[nH]3)C2)nc1 ; O=c1ccccn1-c1ccc(N[C@H]2CC[C@H](Nc3nc4ccccc4[nH]3)C2)nc1 ; O=c1ccc2ccccc2n1-c1ccc(N[C@H]2CC[C@H](Nc3nc4cccnc4[nH]3)C2)nc1 ; O=c1ccccn1-c1ccc(N[C@H]2CC[C@H](Nc3nc4ccccc4o3)C2)nc1] 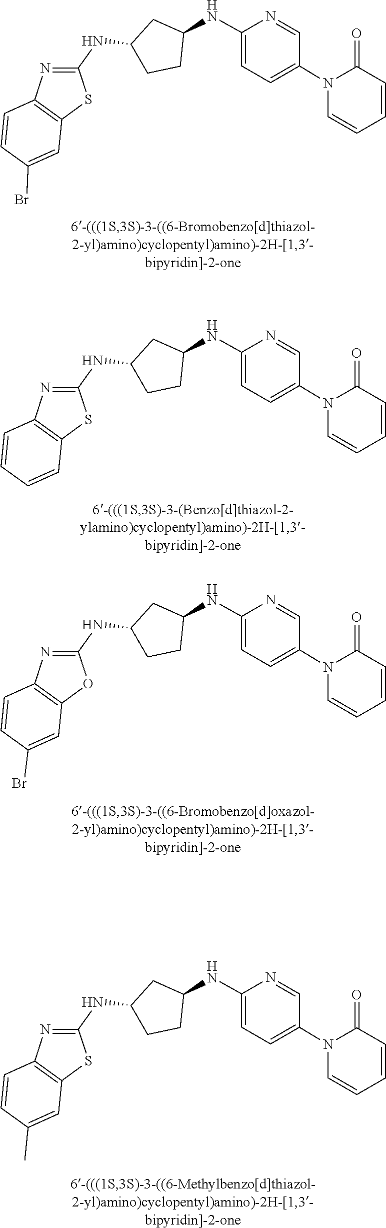 [SMILES: O=c1ccccn1-c1ccc(N[C@H]2CC[C@H](Nc3nc4ccc(Br)cc4s3)C2)nc1 ; O=c1ccccn1-c1ccc(N[C@H]2CC[C@H](Nc3nc4ccccc4s3)C2)nc1 ; O=c1ccccn1-c1ccc(N[C@H]2CC[C@H](Nc3nc4ccc(Br)cc4o3)C2)nc1 ; Cc1ccc2nc(N[C@H]3CC[C@H](Nc4ccc(-n5ccccc5=O)cn4)C3)sc2c1] 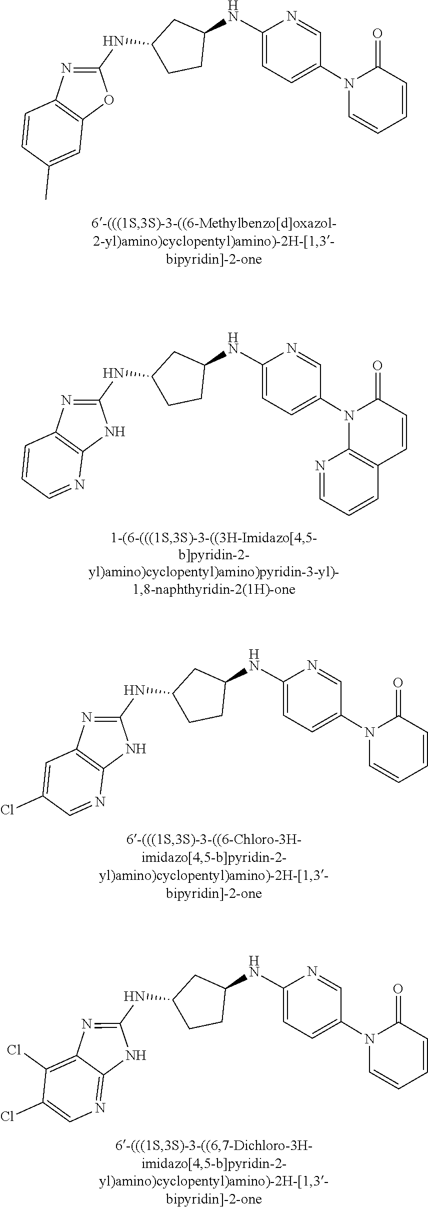 [SMILES: Cc1ccc2nc(N[C@H]3CC[C@H](Nc4ccc(-n5ccccc5=O)cn4)C3)oc2c1 ; O=c1ccc2cccnc2n1-c1ccc(N[C@H]2CC[C@H](Nc3nc4cccnc4[nH]3)C2)nc1 ; O=c1ccccn1-c1ccc(N[C@H]2CC[C@H](Nc3nc4cc(Cl)cnc4[nH]3)C2)nc1 ; O=c1ccccn1-c1ccc(N[C@H]2CC[C@H](Nc3nc4c(Cl)c(Cl)cnc4[nH]3)C2)nc1] 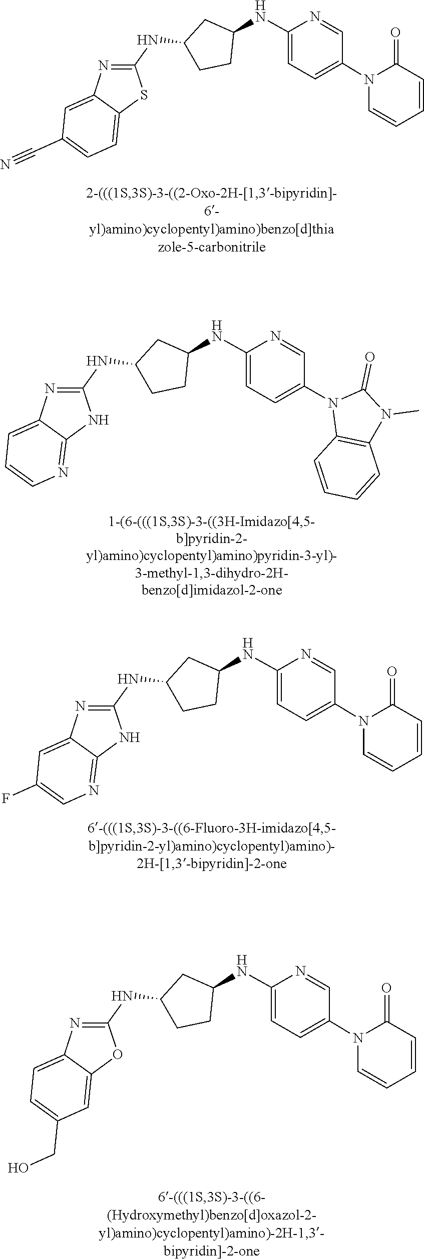 [SMILES: N#Cc1ccc2sc(N[C@H]3CC[C@H](Nc4ccc(-n5ccccc5=O)cn4)C3)nc2c1 ; Cn1c(=O)n(-c2ccc(N[C@H]3CC[C@H](Nc4nc5cccnc5[nH]4)C3)nc2)c2ccccc21 ; O=c1ccccn1-c1ccc(N[C@H]2CC[C@H](Nc3nc4cc(F)cnc4[nH]3)C2)nc1 ; O=c1ccccn1-c1ccc(N[C@H]2CC[C@H](Nc3nc4ccc(CO)cc4o3)C2)nc1] 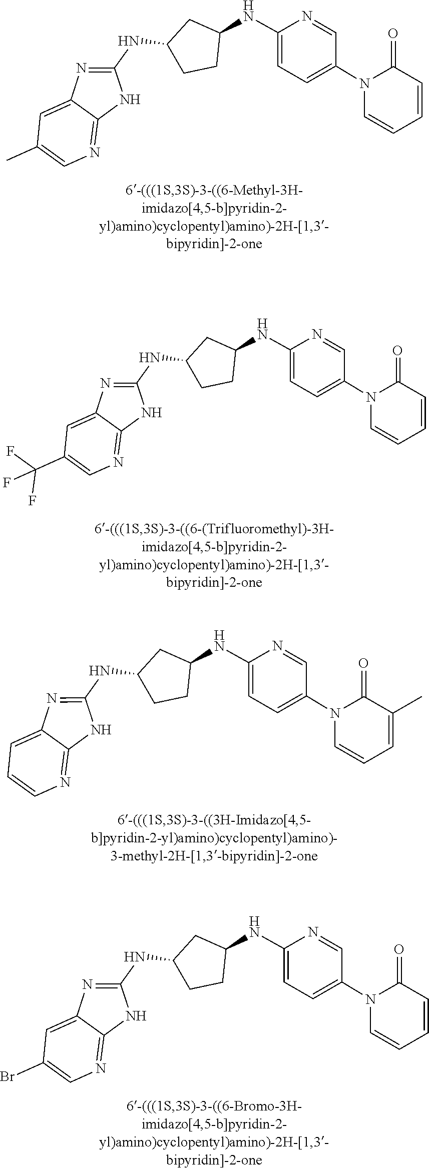 [SMILES: Cc1cnc2[nH]c(N[C@H]3CC[C@H](Nc4ccc(-n5ccccc5=O)cn4)C3)nc2c1 ; O=c1ccccn1-c1ccc(N[C@H]2CC[C@H](Nc3nc4cc(C(F)(F)F)cnc4[nH]3)C2)nc1 ; Cc1cccn(-c2ccc(N[C@H]3CC[C@H](Nc4nc5cccnc5[nH]4)C3)nc2)c1=O ; O=c1ccccn1-c1ccc(N[C@H]2CC[C@H](Nc3nc4cc(Br)cnc4[nH]3)C2)nc1] 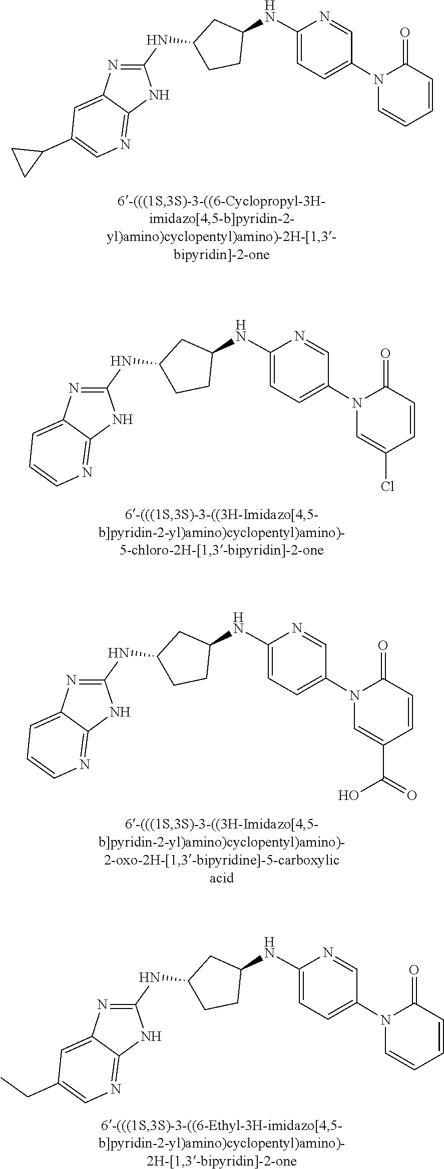 [SMILES: O=c1ccccn1-c1ccc(N[C@H]2CC[C@H](Nc3nc4cc(C5CC5)cnc4[nH]3)C2)nc1 ; O=c1ccc(Cl)cn1-c1ccc(N[C@H]2CC[C@H](Nc3nc4cccnc4[nH]3)C2)nc1 ; O=C(O)c1ccc(=O)n(-c2ccc(N[C@H]3CC[C@H](Nc4nc5cccnc5[nH]4)C3)nc2)c1 ; CCc1cnc2[nH]c(N[C@H]3CC[C@H](Nc4ccc(-n5ccccc5=O)cn4)C3)nc2c1] 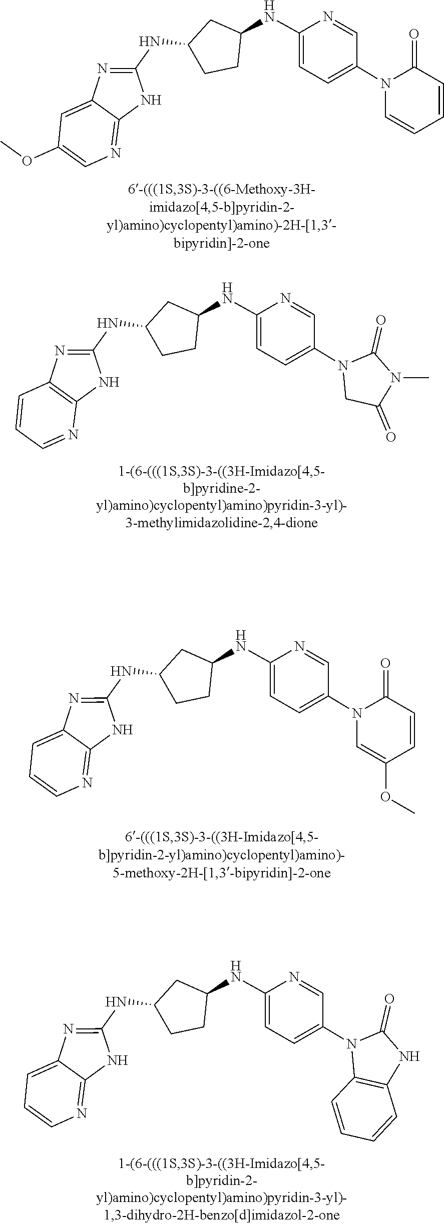 [SMILES: COc1cnc2[nH]c(N[C@H]3CC[C@H](Nc4ccc(-n5ccccc5=O)cn4)C3)nc2c1 ; CN1C(=O)CN(c2ccc(N[C@H]3CC[C@H](Nc4nc5cccnc5[nH]4)C3)nc2)C1=O ; COc1ccc(=O)n(-c2ccc(N[C@H]3CC[C@H](Nc4nc5cccnc5[nH]4)C3)nc2)c1 ; O=c1[nH]c2ccccc2n1-c1ccc(N[C@H]2CC[C@H](Nc3nc4cccnc4[nH]3)C2)nc1] 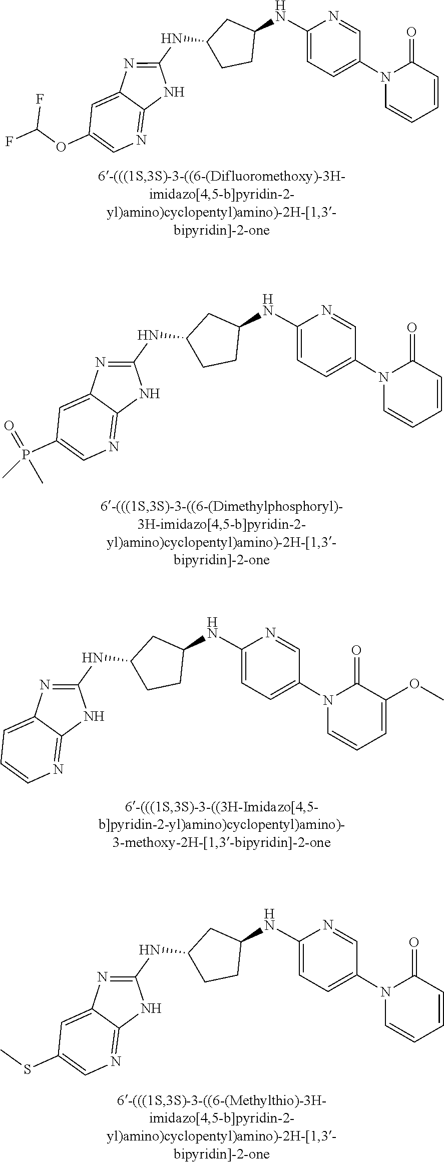 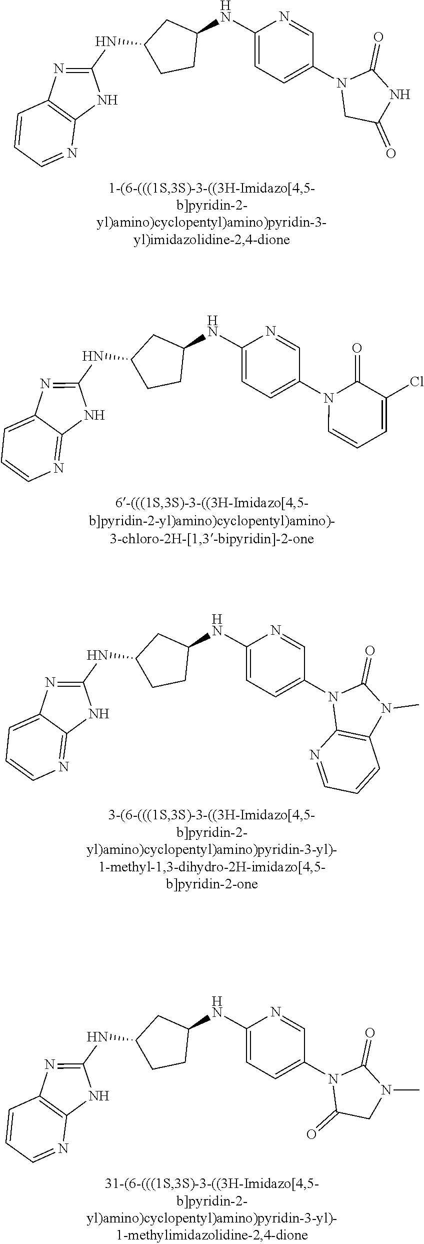 [SMILES: O=C1CN(c2ccc(N[C@H]3CC[C@H](Nc4nc5cccnc5[nH]4)C3)nc2)C(=O)N1 ; O=c1c(Cl)cccn1-c1ccc(N[C@H]2CC[C@H](Nc3nc4cccnc4[nH]3)C2)nc1 ; Cn1c(=O)n(-c2ccc(N[C@H]3CC[C@H](Nc4nc5cccnc5[nH]4)C3)nc2)c2ncccc21 ; CN1CC(=O)N(c2ccc(N[C@H]3CC[C@H](Nc4nc5cccnc5[nH]4)C3)nc2)C1=O] 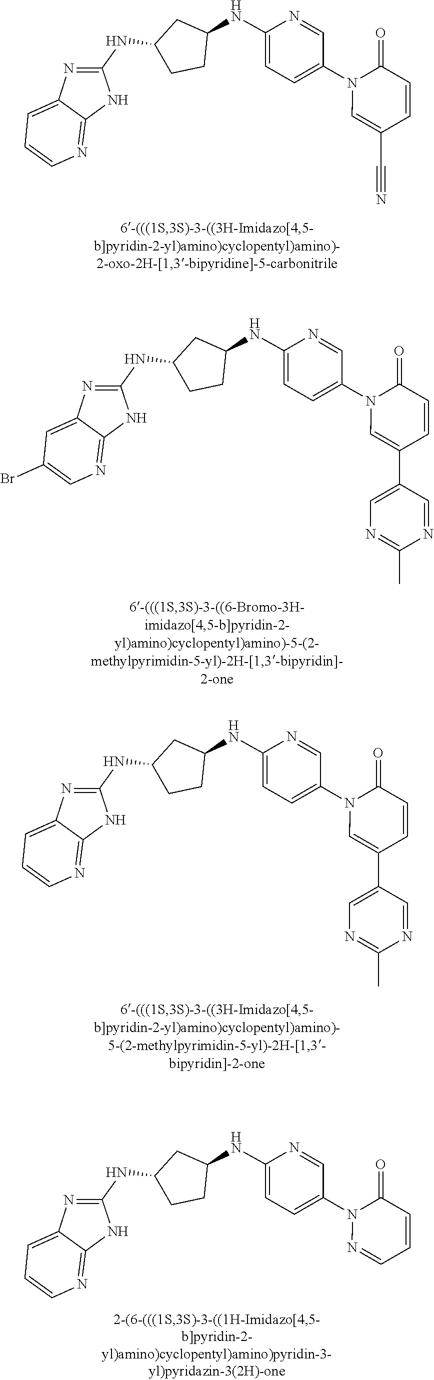 [SMILES: N#Cc1ccc(=O)n(-c2ccc(N[C@H]3CC[C@H](Nc4nc5cccnc5[nH]4)C3)nc2)c1 ; Cc1ncc(-c2ccc(=O)n(-c3ccc(N[C@H]4CC[C@H](Nc5nc6cc(Br)cnc6[nH]5)C4)nc3)c2)cn1 ; Cc1ncc(-c2ccc(=O)n(-c3ccc(N[C@H]4CC[C@H](Nc5nc6cccnc6[nH]5)C4)nc3)c2)cn1 ; O=c1cccnn1-c1ccc(N[C@H]2CC[C@H](Nc3nc4cccnc4[nH]3)C2)nc1] 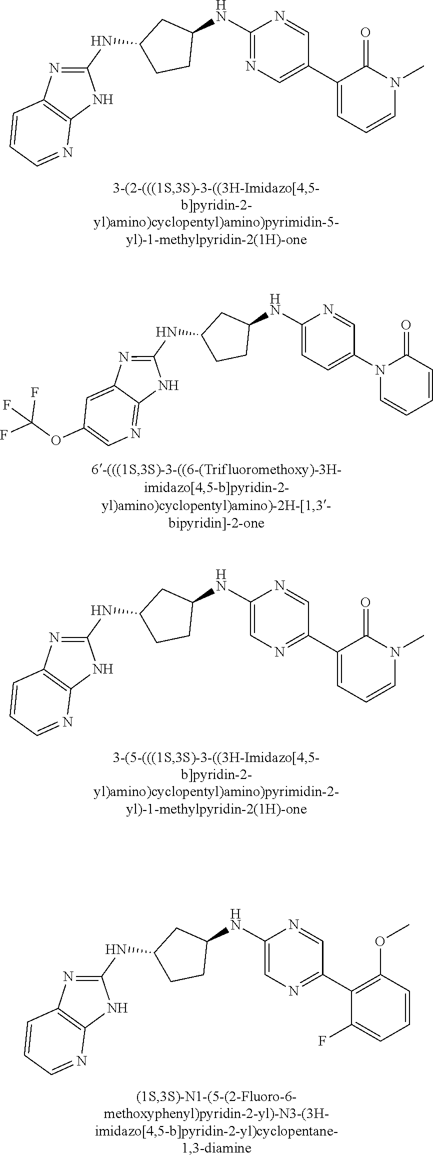 [SMILES: Cn1cccc(-c2cnc(N[C@H]3CC[C@H](Nc4nc5cccnc5[nH]4)C3)nc2)c1=O ; O=c1ccccn1-c1ccc(N[C@H]2CC[C@H](Nc3nc4cc(OC(F)(F)F)cnc4[nH]3)C2)nc1 ; Cn1cccc(-c2cnc(N[C@H]3CC[C@H](Nc4nc5cccnc5[nH]4)C3)cn2)c1=O ; COc1cccc(F)c1-c1cnc(N[C@H]2CC[C@H](Nc3nc4cccnc4[nH]3)C2)cn1] 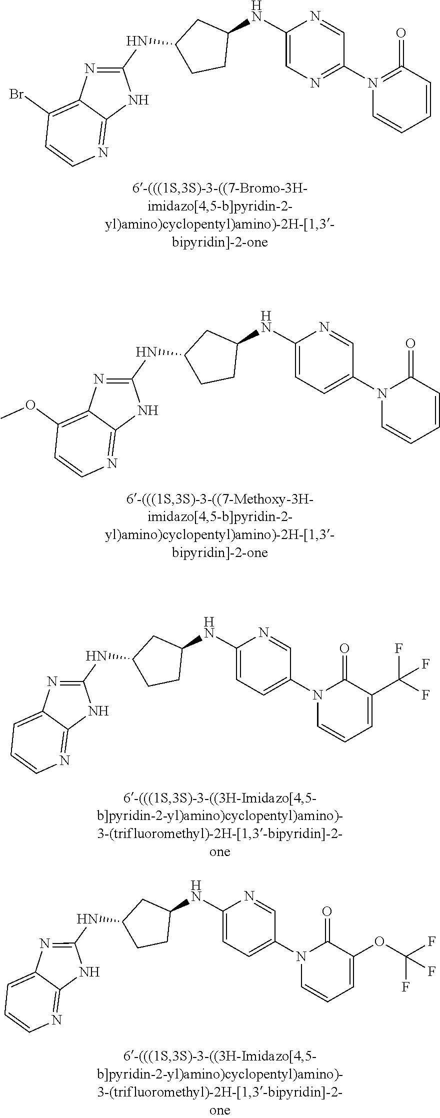 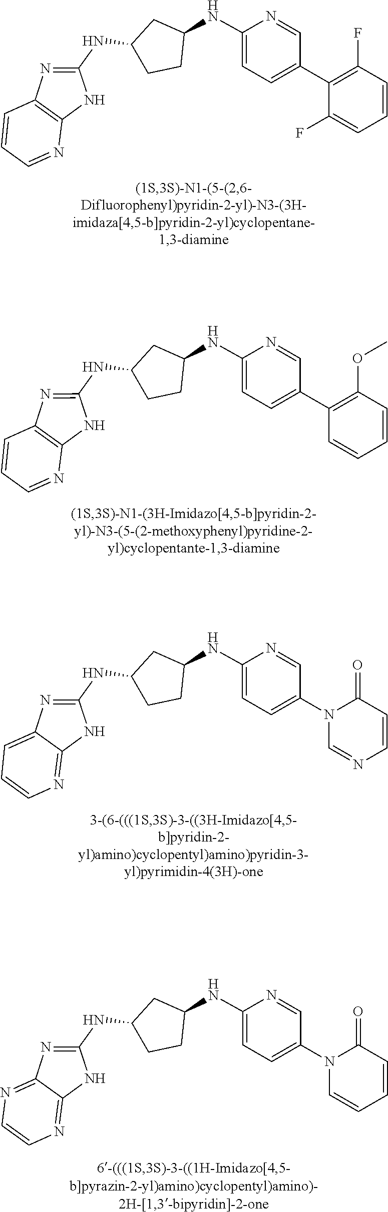 [SMILES: Fc1cccc(F)c1-c1ccc(N[C@H]2CC[C@H](Nc3nc4cccnc4[nH]3)C2)nc1 ; COc1ccccc1-c1ccc(N[C@H]2CC[C@H](Nc3nc4cccnc4[nH]3)C2)nc1 ; O=c1ccncn1-c1ccc(N[C@H]2CC[C@H](Nc3nc4cccnc4[nH]3)C2)nc1 ; O=c1ccccn1-c1ccc(N[C@H]2CC[C@H](Nc3nc4nccnc4[nH]3)C2)nc1] 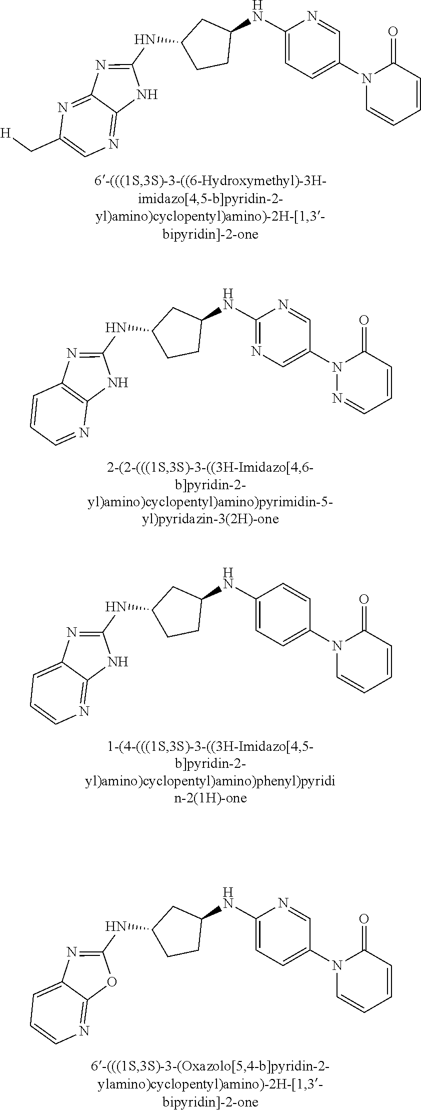 [SMILES: Cc1cnc2[nH]c(N[C@H]3CC[C@H](Nc4ccc(-n5ccccc5=O)cn4)C3)nc2n1 ; O=c1cccnn1-c1cnc(N[C@H]2CC[C@H](Nc3nc4cccnc4[nH]3)C2)nc1 ; O=c1ccccn1-c1ccc(N[C@H]2CC[C@H](Nc3nc4cccnc4[nH]3)C2)cc1 ; O=c1ccccn1-c1ccc(N[C@H]2CC[C@H](Nc3nc4cccnc4o3)C2)nc1] 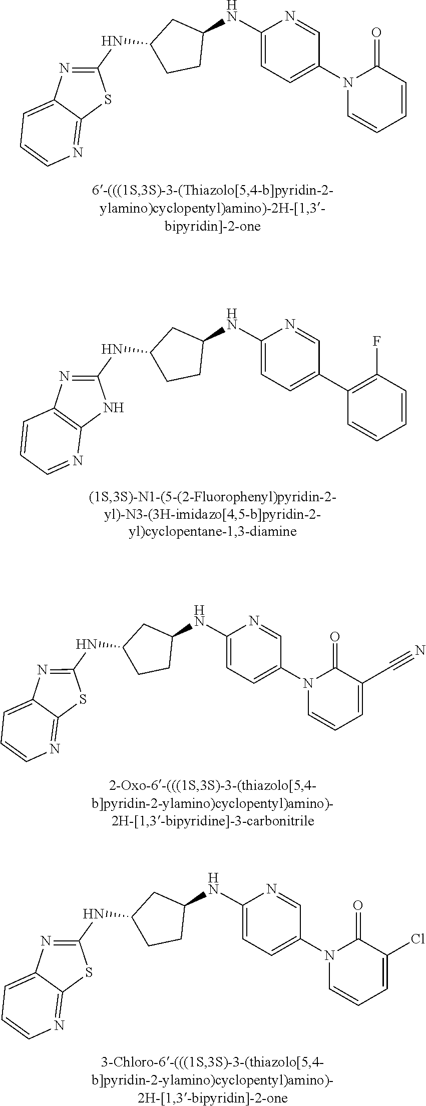 [SMILES: O=c1ccccn1-c1ccc(N[C@H]2CC[C@H](Nc3nc4cccnc4s3)C2)nc1 ; Fc1ccccc1-c1ccc(N[C@H]2CC[C@H](Nc3nc4cccnc4[nH]3)C2)nc1 ; N#Cc1cccn(-c2ccc(N[C@H]3CC[C@H](Nc4nc5cccnc5s4)C3)nc2)c1=O ; O=c1c(Cl)cccn1-c1ccc(N[C@H]2CC[C@H](Nc3nc4cccnc4s3)C2)nc1] 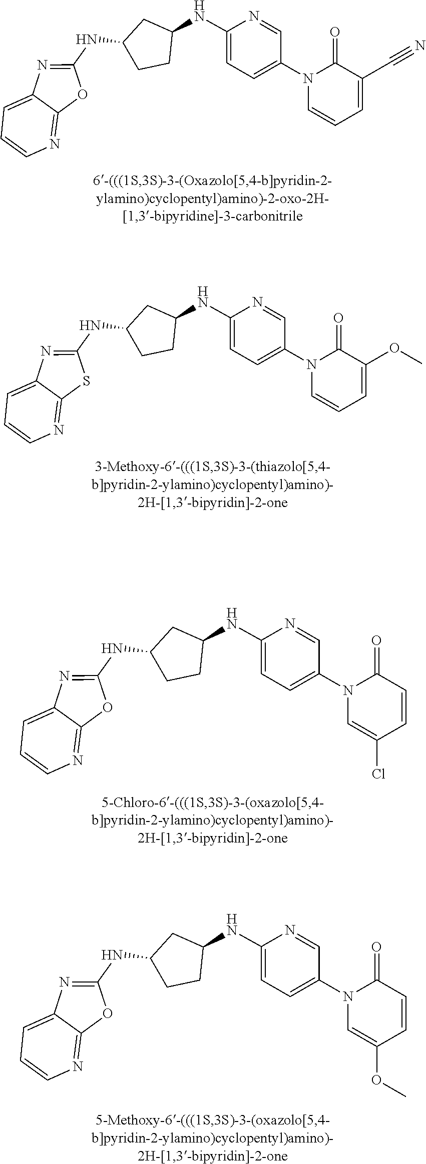 [SMILES: N#Cc1cccn(-c2ccc(N[C@H]3CC[C@H](Nc4nc5cccnc5o4)C3)nc2)c1=O ; COc1cccn(-c2ccc(N[C@H]3CC[C@H](Nc4nc5cccnc5s4)C3)nc2)c1=O ; O=c1ccc(Cl)cn1-c1ccc(N[C@H]2CC[C@H](Nc3nc4cccnc4o3)C2)nc1 ; COc1ccc(=O)n(-c2ccc(N[C@H]3CC[C@H](Nc4nc5cccnc5o4)C3)nc2)c1] 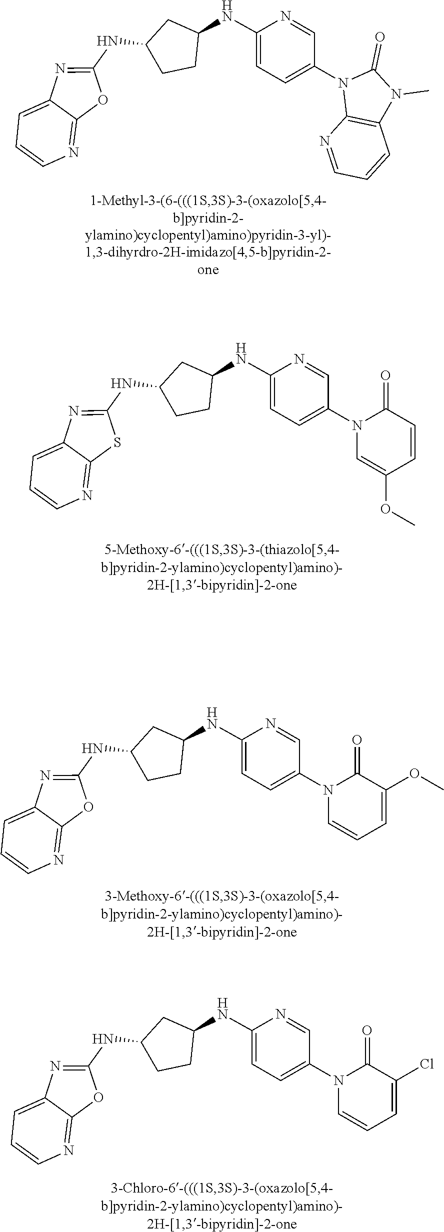 [SMILES: Cn1c(=O)n(-c2ccc(N[C@H]3CC[C@H](Nc4nc5cccnc5o4)C3)nc2)c2ncccc21 ; COc1ccc(=O)n(-c2ccc(N[C@H]3CC[C@H](Nc4nc5cccnc5s4)C3)nc2)c1 ; COc1cccn(-c2ccc(N[C@H]3CC[C@H](Nc4nc5cccnc5o4)C3)nc2)c1=O ; O=c1c(Cl)cccn1-c1ccc(N[C@H]2CC[C@H](Nc3nc4cccnc4o3)C2)nc1] 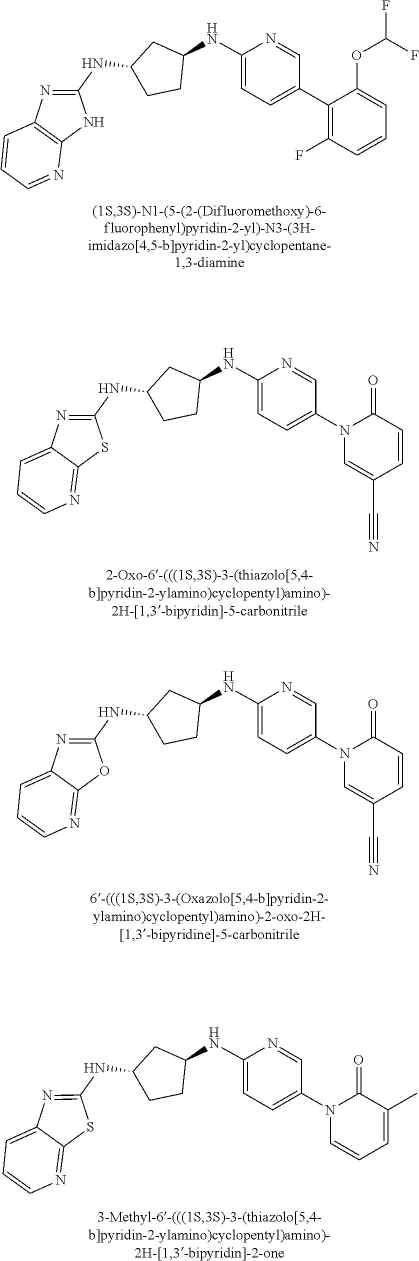 [SMILES: Fc1cccc(OC(F)F)c1-c1ccc(N[C@H]2CC[C@H](Nc3nc4cccnc4[nH]3)C2)nc1 ; N#Cc1ccc(=O)n(-c2ccc(N[C@H]3CC[C@H](Nc4nc5cccnc5s4)C3)nc2)c1 ; N#Cc1ccc(=O)n(-c2ccc(N[C@H]3CC[C@H](Nc4nc5cccnc5o4)C3)nc2)c1 ; Cc1cccn(-c2ccc(N[C@H]3CC[C@H](Nc4nc5cccnc5s4)C3)nc2)c1=O] 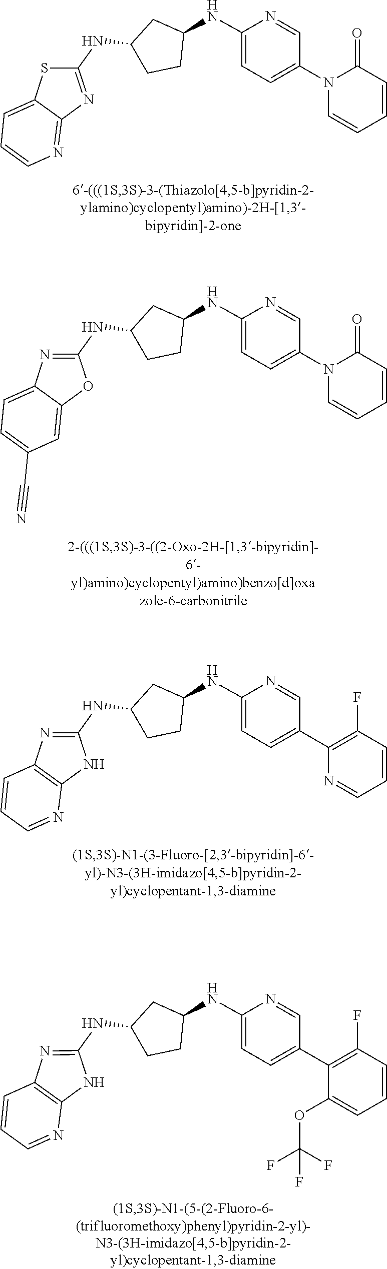 [SMILES: O=c1ccccn1-c1ccc(N[C@H]2CC[C@H](Nc3nc4ncccc4s3)C2)nc1 ; N#Cc1ccc2nc(N[C@H]3CC[C@H](Nc4ccc(-n5ccccc5=O)cn4)C3)oc2c1 ; Fc1cccnc1-c1ccc(N[C@H]2CC[C@H](Nc3nc4cccnc4[nH]3)C2)nc1 ; Fc1cccc(OC(F)(F)F)c1-c1ccc(N[C@H]2CC[C@H](Nc3nc4cccnc4[nH]3)C2)nc1] 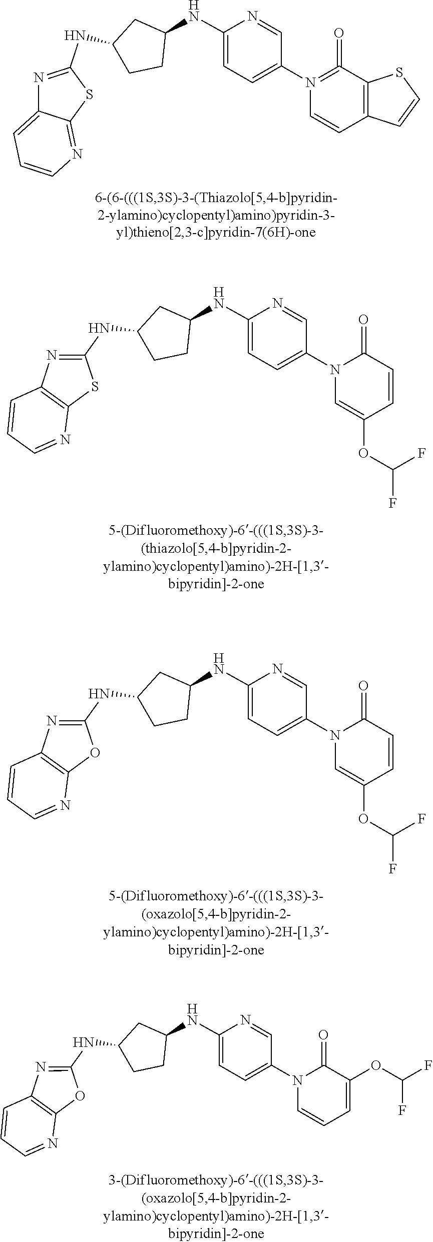 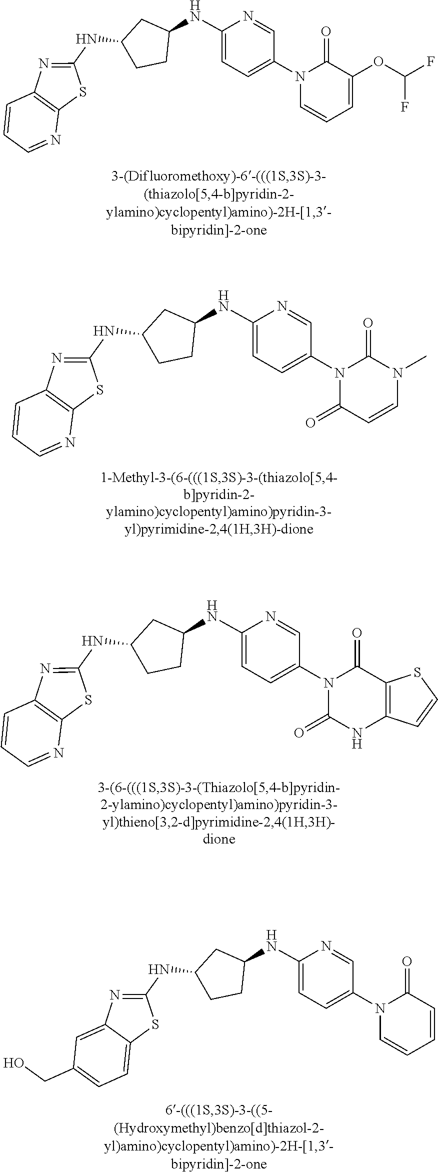 [SMILES: O=c1c(OC(F)F)cccn1-c1ccc(N[C@H]2CC[C@H](Nc3nc4cccnc4s3)C2)nc1 ; Cn1ccc(=O)n(-c2ccc(N[C@H]3CC[C@H](Nc4nc5cccnc5s4)C3)nc2)c1=O ; O=c1[nH]c2ccsc2c(=O)n1-c1ccc(N[C@H]2CC[C@H](Nc3nc4cccnc4s3)C2)nc1 ; O=c1ccccn1-c1ccc(N[C@H]2CC[C@H](Nc3nc4cc(CO)ccc4s3)C2)nc1] 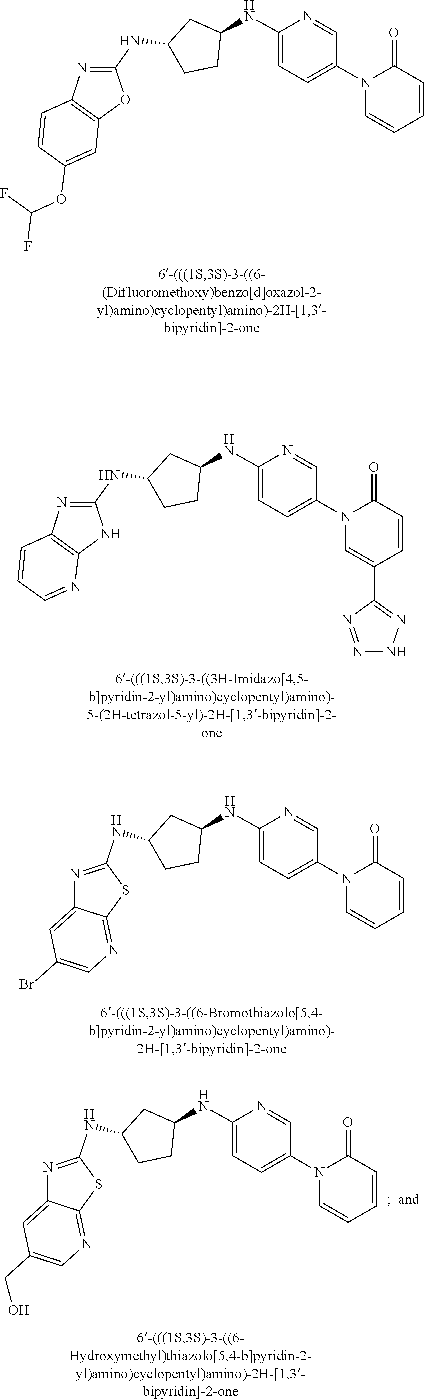 [SMILES: O=c1ccccn1-c1ccc(N[C@H]2CC[C@H](Nc3nc4ccc(OC(F)F)cc4o3)C2)nc1 ; O=c1ccc(-c2nn[nH]n2)cn1-c1ccc(N[C@H]2CC[C@H](Nc3nc4cccnc4[nH]3)C2)nc1 ; O=c1ccccn1-c1ccc(N[C@H]2CC[C@H](Nc3nc4cc(Br)cnc4s3)C2)nc1 ; O=c1ccccn1-c1ccc(N[C@H]2CC[C@H](Nc3nc4cc(CO)cnc4s3)C2)nc1] 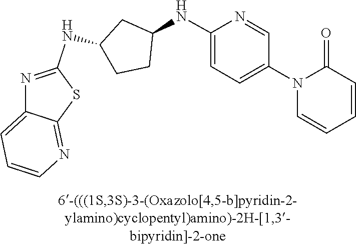 [SMILES: O=c1ccccn1-c1ccc(N[C@H]2CC[C@H](Nc3nc4cccnc4s3)C2)nc1]
or pharmaceutically acceptable salts thereof.

21. (canceled)

22. A pharmaceutical composition comprising the compound of claim 1, or a pharmaceutically acceptable salt thereof, and a pharmaceutically acceptable diluent, carrier, or excipient.

23. (canceled)

24. (canceled)

25. A method of treating PCSK9-mediated disease or disorder in a patient in need thereof, comprising administering to the patient a therapeutically effective amount of the compound or pharmaceutically acceptable salt thereof according to claim 1.

26. The method according to claim 25, wherein the disease or disorder is a cardiovascular disease or disorder.

27. The method according to claim 26, wherein the cardiovascular disease is selected from dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease heart failure, and congestive heart failure.

28. The method according to claim 27, wherein the compound is administered simultaneously, separately, or sequentially, in combination with an additional active ingredient selected from the group consisting of:
i) a statin;
ii) a cholesterol absorption inhibitor;
iii) a SGLT2 inhibitor;
iv) a P2Y12 inhibitor;
v) a citrate lyase inhibitor; and
anti-hypertensive drugs.


SEQUENCE LISTING (1 sequence)

SEQ ID NO: 1 - protein, 705 aa, synthetic construct
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQENLYFQGHHHHHH